(19) United States
(12) Patent Application Publication
(10) Pub. No.: US 2026/0242373 A1
(43) Pub. Date: Aug. 20, 2026

(54) NBD1 MODULATORS AND METHODS OF USING THE SAME

(71) Applicants: Sionna Therapeutics Inc., Waltham, MA (US); Genzyme Corporation, Cambridge, MA (US)

(72) Inventors: Junkai Liao, Andover, MA (US); Mark Munson, Acton, MA (US); Sukanthini Thurairatnam, Dover, MA (US); Bradford Hirth, Littleton, MA (US); Zhongli Gao, Waltham, MA (US); Gregory Donald Hurlbut, Boxborough, MA (US); Jinyu Liu, Union City, CA (US); Michael Kothe, Medway, MA (US); George Topalov, Pittsburgh, PA (US); John E. Macor, Washington Crossing, PA (US); Yi Li, Weston, MA (US); Andrew Good, Bideford (GB); David Borcherding, Bangor, PA (US)

(21) Appl. No.: 19/160,760

(22) PCT Filed: Mar. 1, 2024
(86) PCT No.: PCT/US2024/018145

§ 371 Date: Aug. 28, 2025

Related U.S. Application Data
(60) Provisional application No. 63/449,470, filed on Mar. 2, 2023.

(51) Int. Cl. C07D 471/04; A61K 31/4155; A61K 31/416; A61K 31/4178; A61K 31/4184; A61K 31/4196; A61K 31/4245; A61K 31/427; A61K 31/437; A61K 31/454; C07D 401/14; C07D 403/12; C07D 403/14; C07D 409/14; C07D 413/12; C07D 417/14

(52) U.S. Cl. CPC C07D 471/04; A61K 31/4155; A61K 31/416; A61K 31/4178; A61K 31/4184; A61K 31/4196; A61K 31/4245; A61K 31/427; A61K 31/437; A61K 31/454; C07D 401/14; C07D 403/12; C07D 403/14; C07D 409/14; C07D 413/12; C07D 417/14

(57) ABSTRACT

The present disclosure includes, among other things, CFTR modulators, pharmaceutical compositions, and methods of making and using the same.

### CROSS REFERENCE TO RELATED APPLICATIONS

This application claims the benefit of and priority to U.S. Provisional Patent Application No. 63/449,470, filed on Mar. 2, 2023, the disclosure of each of which is hereby incorporated by reference in its entirety for all purposes.

### BACKGROUND

Cystic fibrosis (CF), an autosomal recessive disorder, is caused by functional deficiency of the cAMP-activated plasma membrane chloride channel, cystic fibrosis transmembrane conductance regulator (CFTR), which results in pulmonary and other complications. The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). CFTR, a member of the ATP binding cassette (ABC) superfamily is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate, and thiocyanate into and out of the cell. CFTR may have a regulatory role over other electrolyte channels, including the epithelial sodium channel ENaC.

In cystic fibrosis patients, the absence or dysfunction of CFTR leads to exocrine gland dysfunction and a multisystem disease, characterized by pancreatic insufficiency and malabsorption, as well as abnormal mucociliary clearance in the lung, mucostasis, chronic lung infection and inflammation, decreased lung function and ultimately respiratory failure.

While more than 1,900 mutations have been identified in the CFTR gene, a detailed understanding of how each CFTR mutation may impact channel function is known for only a few. (Derichs, European Respiratory Review, 22:127, 58-65 (2013)). The most frequent CFTR mutation is the in-frame deletion of phenylalanine at residue 508 (ΔF508) in the first nucleotide binding domain (NBD1). Over 70% of cystic fibrosis patients have a deletion at residue 508 in at least one CFTR allele. The loss of this key phenylalanine renders NBD1 conformationally unstable at physiological temperature and compromises the integrity of the interdomain interface between NDB1 and CFTR's second transmembrane domain (ICL4). The ΔF508 mutation causes production of misfolded CFTR protein which, rather than traffic to the plasma membrane, is instead retained in the endoplasmic reticulum and targeted for degradation by the ubiquitin-proteasome system.

The loss of a functional CFTR channel at the plasma membrane disrupts ionic homeostasis and airway surface hydration leading to reduced lung function. Reduced periciliary liquid volume and increased mucus viscosity impede mucociliary clearance resulting in chronic infection and inflammation. In the lung, the loss of CFTR-function leads to numerous physiological effects downstream of altered anion conductance that result in the dysfunction of additional organs such as the pancreas, intestine and gall bladder.

By studying the mechanistic aspects of CFTR misfolding and corrections, small molecules have been identified as CF modulators, that can act as stabilizers.

Despite the identification of compounds that modulate CFTR, there is no cure for this fatal disease and identification of new compounds and new methods of therapy are needed as well as new methods for treating or lessening the severity of cystic fibrosis and other CFTR mediated conditions and diseases in a patient.

### SUMMARY

The present disclosure includes a compound of formula I:

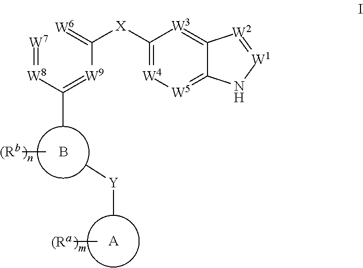

or a pharmaceutically acceptable salt thereof. Additionally, the present disclosure includes, among other things, pharmaceutical compositions, methods of using and methods of making a compound of formula I.

### DETAILED DESCRIPTION

In some embodiments, the present disclosure includes a compound of Formula T:

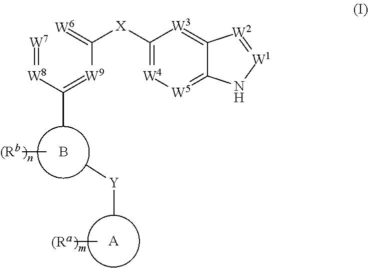

or a pharmaceutically acceptable salt thereof
wherein
W¹ is selected from the group consisting of —C(H)═, and —N═;
W² is selected from the group consisting of —C(H)═, —C(R^{d})═, and —N═;
W³ is selected from the group consisting of —C(H)═, —C(R^{d})═, and —N═;
W⁴ is selected from the group consisting of —C(H)═, —C(R^{d4})═, and —N═;
W⁵ is selected from the group consisting of —C(H)═, —C(R^{d5})═, and —N═;
W⁶ is selected from the group consisting of —C(H)═, —C(R^{c})═, and —N═;
W⁷ is selected from the group consisting of —C(H)═, —C(R^{c})═, and —N═;
W⁸ is selected from the group consisting of —C(H)═, —C(R^{c})═, and —N═;
W⁹ is selected from the group consisting of —C(H)═, —C(R^{c9})═, and —N═;
Ring A is optionally substituted 5-membered heteroaryl, containing 1-2 heteroatoms selected from N, S, and O,
Ring B is optionally substituted 5-membered heteroaryl containing up to three heteroatoms selected from N, S, and O;
each R^{a} is independently selected from the group consisting of halogen, —CN, —NO₂—OR¹, —SR¹, —N(R′)₂, —C(O)OR¹, C(O)N(R′)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa},
each R^{aa} is independently selected from the group consisting of halogen, —COOH, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R′)₂, —C(O)OR¹, C(O)N(R′)₂, —N(H)C(O)R¹, and —N(H)C(O)N(R¹)₂,
wherein two instances of R^{aa} are optionally taken together with any intervening atoms to form an optionally substituted 4-6 membered carbocyclyl or optionally substituted 4-6 membered heterocyclyl ring;
each R^{b} is independently selected from the group consisting of halogen, oxo, —CN, —NO₂—OR¹, —SR¹, —N(R′)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl,
wherein two instances of R^{b} are optionally taken together with any intervening atoms to form an optionally substituted 5-6 membered carbocyclyl or optionally substituted 5-6 membered heterocyclyl ring;
each R^{c} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{d} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl; R^{c9} is halogen;
R^{d4} is halogen;
R^{d5} is halogen;
each R¹ is independently selected from the group consisting of hydrogen, —(CH₂)₁₋₃R², —C(O)R², —(CH₂)₁₋₃OR², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
wherein two instances of R¹ are optionally taken together with any intervening atoms to form an optionally substituted 3-7 membered heterocyclyl ring;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R³ is independently selected from the group consisting of hydrogen, unsubstituted alkyl or haloalkyl
X is selected from the group consisting of —O—, —S—, —S(O)—, —S(O)₂—, —C(R′)(R²)—, —C(O)—, and —CH(OH)—;
Y is selected from the group consisting of optionally substituted C₁-C₃ alkylene, —O—, —S—, —S(O)—, —SO₂N(R²)—, and —S(O)₂—;
n is 0, 1, 2, or 3;
m is 1, 2, or 3,
wherein the compound is not

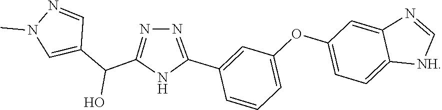 [SMILES: Cn1cc(C(O)c2nnc(-c3cccc(Oc4ccc5[nH]cnc5c4)c3)[nH]2)cn1]

In some embodiments, the present disclosure includes a compound Formula (I-a), (I-b), (I-c), (I-d), (I-e), or (I-f):

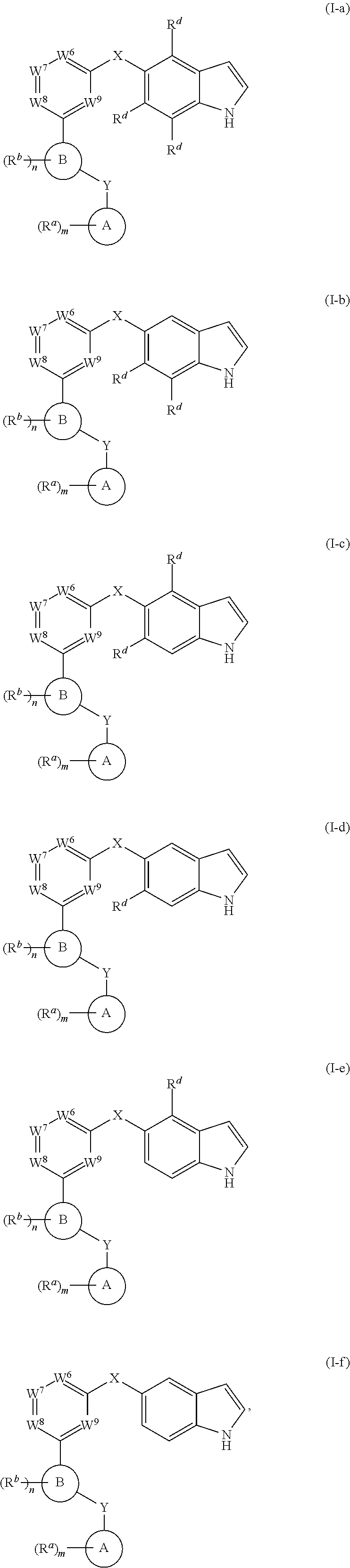

or a pharmaceutically acceptable salt thereof.

The present disclosure includes a compound of formula II:

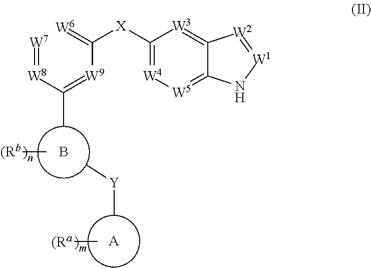

or a pharmaceutically acceptable salt thereof
wherein
W¹ is selected from the group consisting of —C(R^{d})═, and —N═;
W² is selected from the group consisting of —C(H)═, —C(R^{d})═, and —N═;
W³ is selected from the group consisting of —C(R^{d3})═, and —N═;
W⁴ is selected from the group consisting of —C(R^{d4})═, and —N═;
W⁵ is selected from the group consisting of —C(H)═, —C(R^{d5})═, and —N═;
W⁶ is selected from the group consisting of —C(H)═, —C(R^{c})═, and —N═;
W⁷ is selected from the group consisting of —C(H)═, —C(R^{c})═, and —N═;
W⁸ is selected from the group consisting of —C(H)═, —C(R^{c})═, and —N═;
W⁹ is selected from the group consisting of —C(H)═, —C(R^{c9})═, and —N═;
Ring A is optionally substituted phenyl or optionally substituted 6-membered heteroaryl, containing 1-2 nitrogen atoms;
Ring B is optionally substituted 5-membered heteroaryl containing up to three heteroatoms selected from N, S, and O;
each R^{a} is selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R³)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa}
each R^{aa} is independently selected from the group consisting of halogen, —COOH, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂,
wherein two instances of R^{aa} are optionally taken together with any intervening atoms to form an optionally substituted 4-6 membered carbocyclyl or optionally substituted 5-6 membered heterocyclyl ring;
each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{c} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{d} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —SOR¹, —SO(NR²)R¹, —SO₂N(R²)R¹, —N(H)C(O)N(R′)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
R^{c9} is halogen;
R^{d3} is hydrogen or R^{d};
R^{d4} is hydrogen or halogen;
wherein when R^{d4is} fluoro, R^{d3} is not hydrogen, fluoro, chloro, methyl, cyclopropyl, —CH₂CF₃, —CH₂CHF₂, —(CH₂)₀₋₃SO₂R¹, —(CH₂)₀₋₃SOR¹, —(CH₂)₀₋₃P(O)Me₂, —SR¹, thiazolyl, pyrazolyl, or (CH₂)₀₋₃ (3-7 membered heterocyclyl);
wherein when R^{d4} is hydrogen, R^{d3} is not hydrogen, methyl, —CH₂OH, —CH₂NH(tBu), or —C(O)NHMe;
R^{d5} is halogen;
each R¹ is independently selected from the group consisting of hydrogen, —(CH₂)₁₋₃R², —C(O)R², —(CH₂)₁₋₃OR², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
wherein two instances of R¹ are optionally taken together with any intervening atoms to form an optionally substituted 3-7 membered heterocyclyl ring;
each R² is independently selected from the group consisting of hydrogen, optionally substituted C₁-C₆ aliphatic, optionally substituted phenyl, optionally substituted 5-6-membered heteroaryl,
optionally substituted 3-7 membered carbocyclyl, and optionally substituted 3-7 membered heterocyclyl;
each R⁴ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa}
Y is selected from the group consisting of optionally substituted C₁-C₃ alkylene, optionally substituted C₃-C₇ cycloalkylene, optionally substituted 3-7 membered heterocyclylene, —O—, —S—, —S(O)—, —S(O)(NR²)—, and —S(O)₂—;
n is 0 or 1.
m is 1, 2, or 3.

The present disclosure includes a compound of Formula (II-a), (II-b), (II-c), (II-d), (II-e), or (II-f):

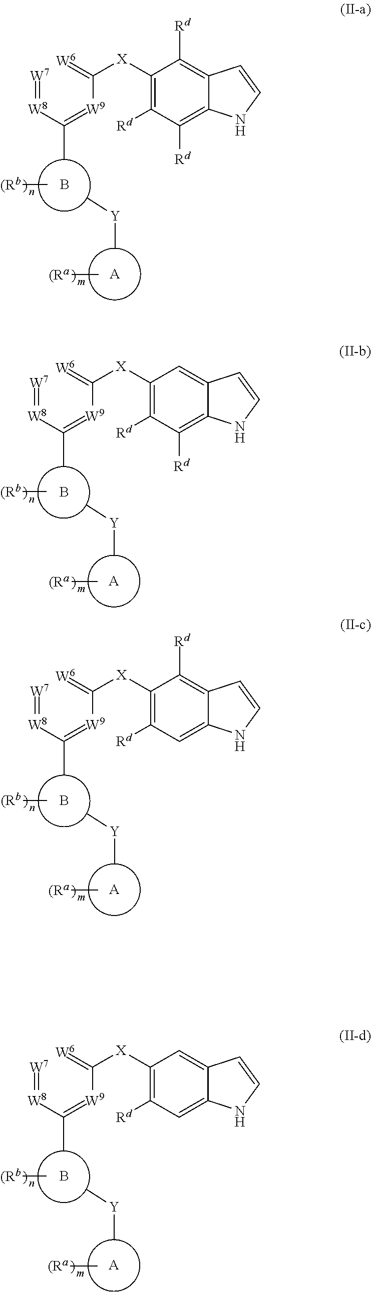

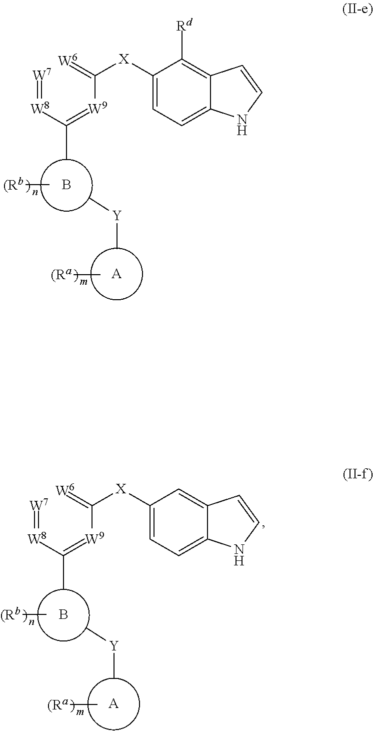

or a pharmaceutically acceptable salt thereof.

The present disclosure includes a compound of formula III:

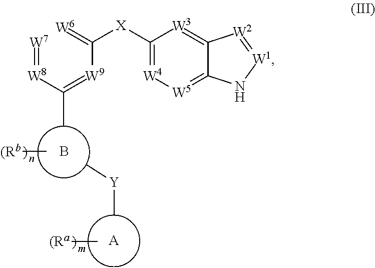

or a pharmaceutically acceptable salt thereof
wherein
W¹ is selected from the group consisting of —C(H)═, and —N═;
W² is selected from the group consisting of —C(H)═, —C(R^{d})═, and —N═;
W³ is selected from the group consisting of —C(H)═, —C(R^{d})═, and —N═;
W⁴ is selected from the group consisting of —C(H)═, —C(R^{d4})═, and —N═;
W⁵ is selected from the group consisting of —C(H)═, —C(R^{d5})═, and —N═;
W⁶ is selected from the group consisting of —C(H)═, —C(R^{c})═, and —N═;
W⁷ is selected from the group consisting of —C(H)═, —C(R^{c})═, and —N═;
W⁸ is selected from the group consisting of —C(H)═, —C(R^{c})═, and —N═;
W⁹ is selected from the group consisting of —C(H)═, —C(R^{c9})═, and —N═;
Ring A is optionally substituted phenyl or optionally substituted 6-membered heteroaryl, containing 1-2 nitrogen atoms;
each R^{a} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R′)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa},
each R^{aa} is independently selected from the group consisting of halogen, —COOH, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂,
wherein two instances of R^{aa} are optionally taken together with any intervening atoms to form an optionally substituted 4-6 membered carbocyclyl or heterocyclyl ring;
each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —N(H)C(O)N(R′)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{c} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{d} is independently selected from the group consisting of halogen, —CN, —NO₂—OR¹, —SR¹, —N(R′)₂, —C(O)OR¹, C(O)N(R¹)₂, —N(H)C(O)R¹, —SO₂R¹, —SO(NR²)R¹, —SO₂N(R²)R¹, —N(H)C(O)N(R′)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
R^{c9} is halogen;
R^{d4} is halogen;
R^{d5} is halogen;
each R¹ is independently selected from the group consisting of hydrogen, —(CH₂)₁₋₃R², —C(O)R², —(CH₂)₁₋₃OR², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
wherein two instances of R¹ are optionally taken together with any intervening atoms to form an optionally substituted 3-7 membered heterocyclyl ring;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
X is selected from the group consisting of optionally substituted C₁-C₃ alkylene, C═O, —S—, —S(O)—, —SO(NR²)—, and —S(O)₂—;
Y is optionally substituted C₁-C₃ alkylene optionally substituted C₃-C₇ cycloalkylene, or optionally substituted 3-7 membered heterocyclylene;
n is 0, or 1; and
mis 1, 2, or 3.

The present disclosure includes a compound of Formula (III-a), (III-b), (III-c), (III-d), (III-e), or (III-f):

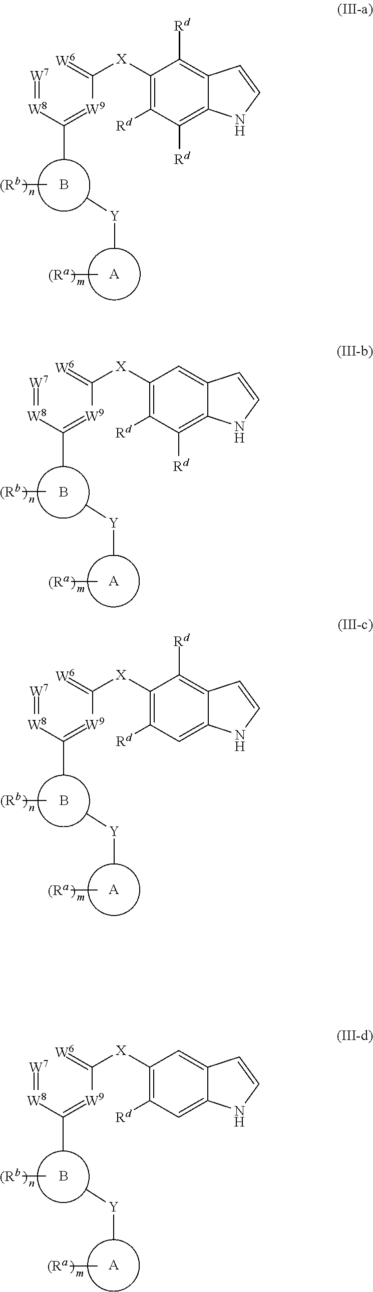

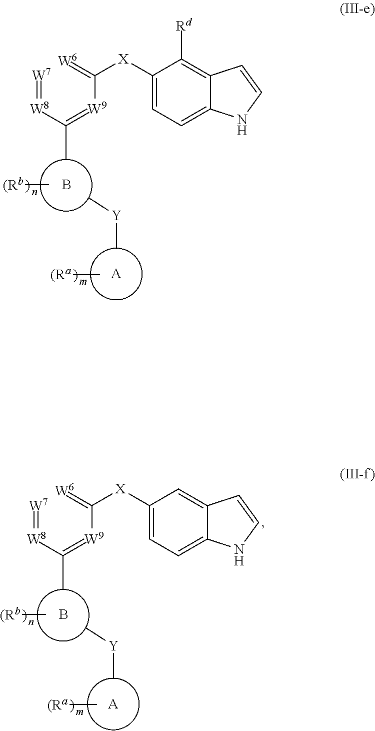

or a pharmaceutically acceptable salt thereof.

### X

In some embodiments, X is selected from the group consisting of —O—, —S—, —S(O)—, —S(O)₂—, —C(R¹)(R²)—, —C(O)—, and —CH(OH)—. In some embodiments, X is —O—. In some embodiments, X is —S—. In some embodiments, X is —S(O)—. In some embodiments, X is —S(O)₂—. In some embodiments, X is —CH₂—. In some embodiments, X is —C(H)(CH₃)—. In some embodiments, X is —C(O)—. In some embodiments, X is —CH(OH)—.

### Y

In some embodiments, Y is selected from the group consisting of optionally substituted C₁-C₃ alkylene, —O—, —S—, —S(O)—, —SO₂N(R²)—, and —S(O)₂—. In some embodiments, Y is —CH₂—. In some embodiments —O—. In some embodiments, Y is —S—. In some embodiments, Y is —S(O)—. In some embodiments, Y is —S(O)₂—. In some embodiments, Y is —SO₂N(R²)—. In some embodiments, Y is —S(O)₂—.

### Ring A

In some embodiments, Ring A is optionally substituted 5-membered heteroaryl, containing 1-2 heteroatoms selected from N, S, and O. In some embodiments, Ring A is an optionally substituted 5-membered heteroaryl selected from the group consisting of thiophenyl, pyrazolyl, pyrrolyl, and thiazolyl. In some embodiments, Ring A is optionally substituted thiophenyl. In some embodiments, Ring A is pyrazolyl. In some embodiments, Ring A is optionally substituted thiazolyl.

In some embodiments, Ring A is selected from the group consisting of

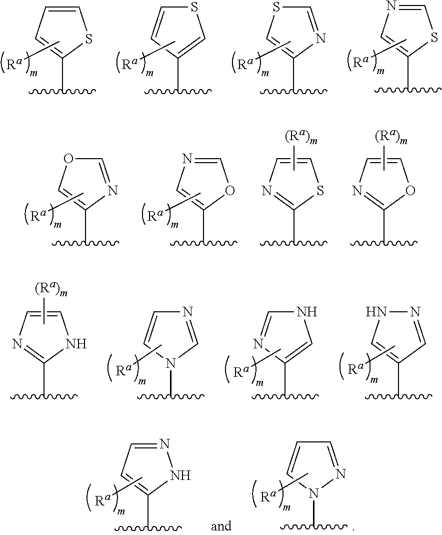

In some embodiments, Ring A is selected from the group consisting of

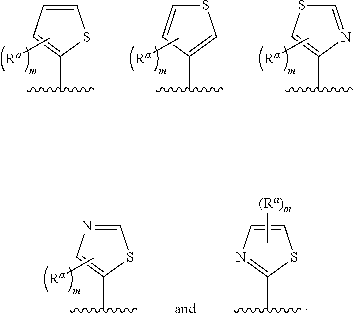

### Ring B

In some embodiments, Ring B is optionally substituted 5-membered heteroaryl. In some embodiments, Ring B is optionally substituted 5-membered heteroaryl comprising 1-3 nitrogen atoms. In some embodiments, Ring B is a optionally substituted 5-membered heteroaryl selected from the group consisting of pyrazolyl, triazolyl, imidazolyl, oxazolyl, thiazolyl, oxadiazolyl, thiadiazolyl, isooxadiazolyl and isothiadiazolyl. In some embodiments, Ring B is a optionally substituted pyrazolyl. In some embodiments, Ring B is a optionally substituted triazolyl. In some embodiments, Ring B is a optionally substituted imidazolyl. In some embodiments, Ring B is a optionally substituted oxazolyl. In some embodiments, Ring B is a optionally substituted thiazolyl. In some embodiments, Ring B is a optionally substituted oxadiazolyl. In some embodiments, Ring B is a optionally substituted thiadiazolyl. In some embodiments, Ring B is a optionally substituted isooxadiazolyl. In some embodiments, Ring B is a optionally substituted isothiadiazolyl. In some embodiments, Ring B is

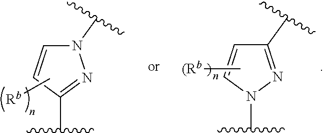

In some embodiments, Ring B is

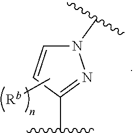

In some embodiments, Ring B is selected from the group consisting of

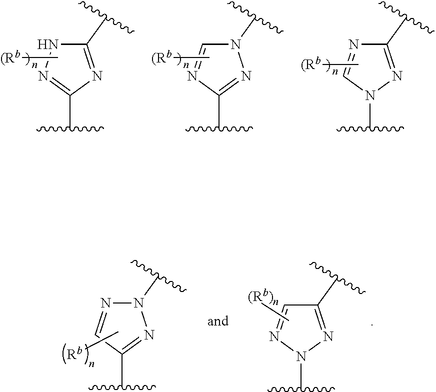

In some embodiments. Ring B is

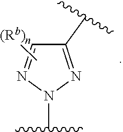

In some embodiments, Ring B is selected from the group consisting of

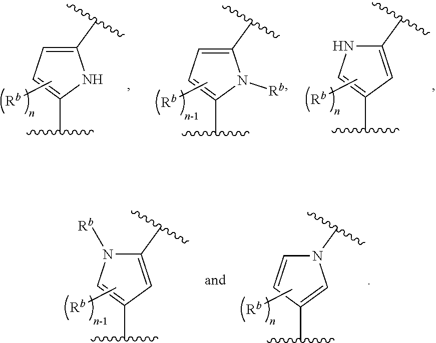

In some embodiments, Ring B is selected from the group consisting of

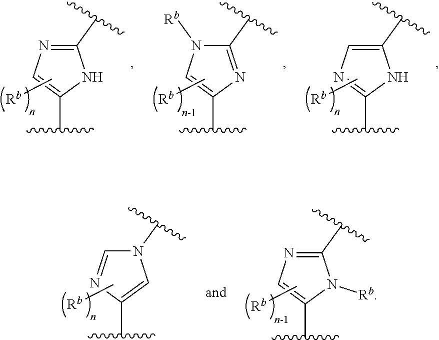

In some embodiments, Ring B is selected from the group consisting of

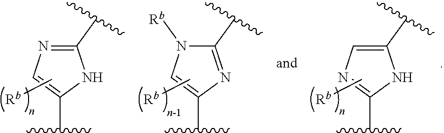

In some embodiments, Ring B is selected from the group consisting of

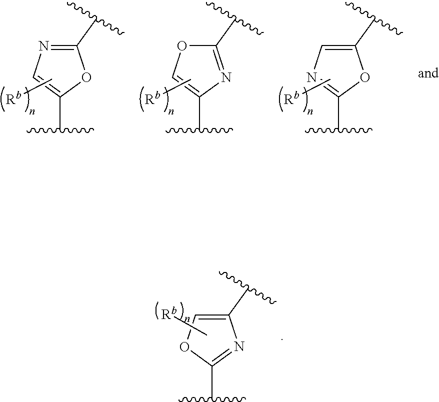

Ring B is selected from the group consisting of

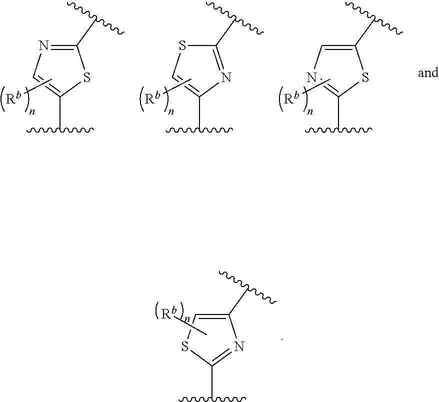

In some embodiments, Ring B is selected from the group consisting of

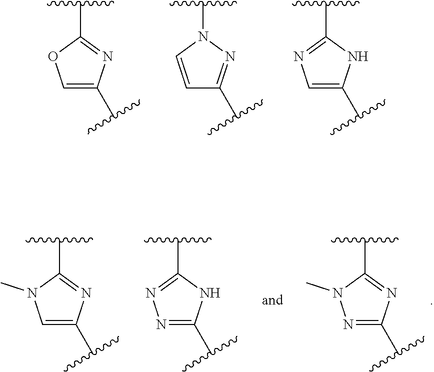

### R^{a}

In some embodiments, each R^{a} is selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa}. In some embodiments, each R^{a} is independently selected from halogen, optionally substituted C₁-C₆ alkyl, and optionally substituted C₁-C₆ alkenyl, wherein each R^{a} is independently substituted with 0-4 instances of R^{aa}. In some embodiments, wherein each R^{a} is independently —CH₂COOH, —CH₂CH₂COOH, and —C(H)═C(H)—COOH. In some embodiments, wherein R^{a} is —CH₂COOH. In some embodiments, wherein R^{a} is —CH₂CH₂COOH. In some embodiments, wherein R^{a} is —C(H)═C(H)—COOH. In some embodiments R^{a} is optionally substituted C₁-C₆ aliphatic. In some embodiments, R^{a} is optionally substituted C₁-C₃ aliphatic. In some embodiments, R^{a} is optionally substituted C₁-C₃ alkyl. In some embodiments, R^{a} is optionally substituted methyl. In some embodiments, R^{a} is halogen. In some embodiments, R^{a} is fluoro. In some embodiments, R^{a} is chloro.

### R^{b}

In some embodiments, each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO₂N(R²), —SO(NR²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, R^{b} is optionally substituted C₁-C₆ aliphatic. In some embodiments, R^{b} is optionally substituted C₁-C₆ alkyl. In some embodiments, R^{b} is optionally substituted C₁-C₃ alkyl. In some embodiments, R^{b} is optionally substituted methyl. In some embodiments, two instances of R^{b} are taken together, with any intervening atoms to form a 5-7 members optionally substituted carboxylic or heteroaryl ring. In some embodiments, R^{b} is —N(R¹)₂. In some embodiments, R^{b} is —N(H)CH₂CH₂OH. In some embodiments, R^{b} is piperidonyl. In some embodiments, R^{b} is pyrrolidinonyl. In some embodiments R^{b} is —OR¹. In some embodiments, R^{b} is —OMe or OEt. In some embodiments, R^{b} is —SR¹. In some embodiments, R^{b} is —SMe.

### R^{c}

In some embodiments, each R^{c} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO₂N(R²), —SO(NR²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{c} is independently substituted with 0-4 instances of R^{aa}.

In some embodiments, R^{c} is halogen. In some embodiments, R^{c} is fluoro.

### R^{d}

In some embodiments, each R^{d} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO₂N(R²), —SO(NR²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{d} is independently substituted with 0-4 instances of R^{aa}. In some embodiments, R^{d} is independently selected from the group consisting of halogen, —OR¹, —SR¹, —C(O)N(R¹)₂, —N(H)C(O)R¹, —SO₂R¹, —SO₂N(R²), —SO(NR²)R¹, and optionally substituted C₁-C₆ aliphatic, wherein each R^{d} is independently substituted with 0-4 instances of R^{aa}. In some embodiments, each R^{d} is halogen. In some embodiments, R^{d} is —N(H)C(O)R¹. In some embodiments, R^{d} is —CH₂OH. In some embodiments, R^{d} is —COOH. In some embodiments, R^{d} is

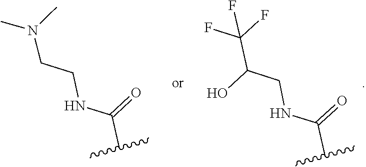

In some embodiment R^{d}—S(O)²CH³.

In some embodiments, R^{d4} is halogen. In some embodiments, R^{d4} is fluoro.

In some embodiments, R^{d5} is halogen. In some embodiments, R^{d}s is fluoro.

In some embodiments, the present disclosure includes compounds listed in Table 1.

TABLE 1

| Com- | |
|---|---|
| pound | |
| No. | Structure |
| 1 | 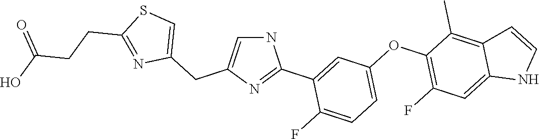 [SMILES: Cc1c(Oc2ccc(F)c(-c3nc(Cc4csc(CCC(=O)O)n4)c[nH]3)c2)c(F)cc2[nH]ccc12] |
| 2 | 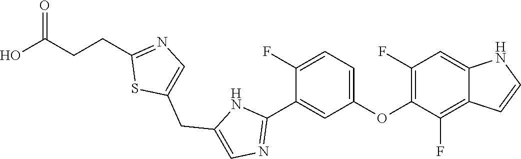 [SMILES: O=C(O)CCc1ncc(Cc2cnc(-c3cc(Oc4c(F)cc5[nH]ccc5c4F)ccc3F)[nH]2)s1] |
| 3 | 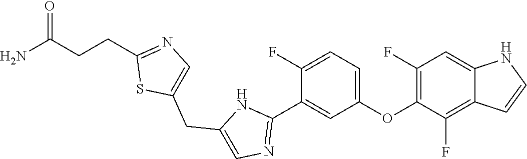 [SMILES: NC(=O)CCc1ncc(Cc2cnc(-c3cc(Oc4c(F)cc5[nH]ccc5c4F)ccc3F)[nH]2)s1] |
| 4 | 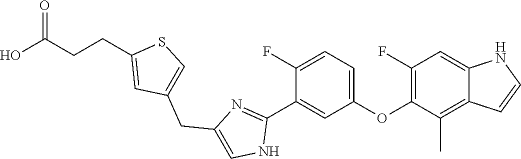 [SMILES: Cc1c(Oc2ccc(F)c(-c3nc(Cc4csc(CCC(=O)O)c4)c[nH]3)c2)c(F)cc2[nH]ccc12] |
| 5 | 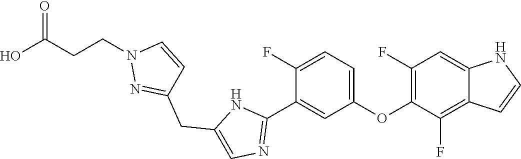 [SMILES: O=C(O)CCn1ccc(Cc2cnc(-c3cc(Oc4c(F)cc5[nH]ccc5c4F)ccc3F)[nH]2)n1] |
| 6 | 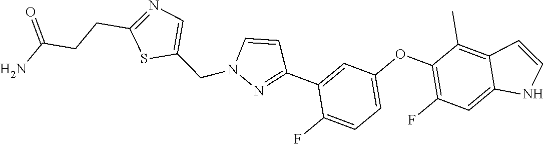 [SMILES: Cc1c(Oc2ccc(F)c(-c3ccn(Cc4cnc(CCC(N)=O)s4)n3)c2)c(F)cc2[nH]ccc12] |
| 7 | 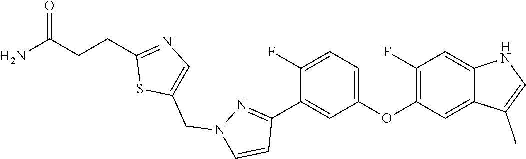 [SMILES: Cc1c[nH]c2cc(F)c(Oc3ccc(F)c(-c4ccn(Cc5cnc(CCC(N)=O)s5)n4)c3)cc12] |
| 8 | 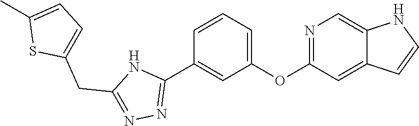 [SMILES: Cc1ccc(Cc2nnc(-c3cccc(Oc4cc5cc[nH]c5cn4)c3)[nH]2)s1] |
| 9 | 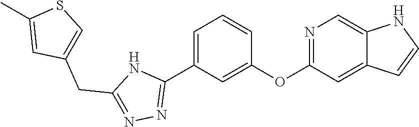 [SMILES: Cc1cc(Cc2nnc(-c3cccc(Oc4cc5cc[nH]c5cn4)c3)[nH]2)cs1] |
| 10 | 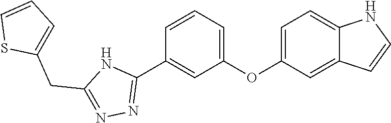 [SMILES: c1cc(Oc2ccc3[nH]ccc3c2)cc(-c2nnc(Cc3cccs3)[nH]2)c1] |
| 11 | 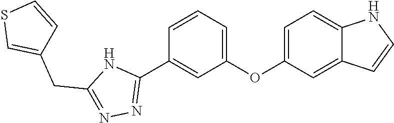 [SMILES: c1cc(Oc2ccc3[nH]ccc3c2)cc(-c2nnc(Cc3ccsc3)[nH]2)c1] |
| 12 | 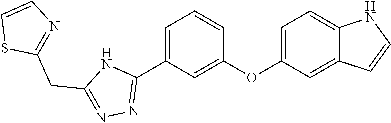 [SMILES: c1cc(Oc2ccc3[nH]ccc3c2)cc(-c2nnc(Cc3nccs3)[nH]2)c1] |
| 13 | 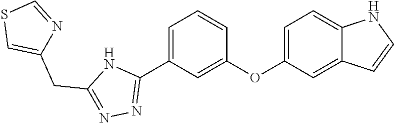 [SMILES: c1cc(Oc2ccc3[nH]ccc3c2)cc(-c2nnc(Cc3cscn3)[nH]2)c1] |
| 14 | 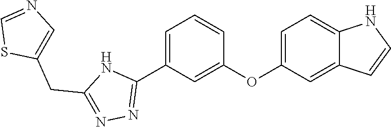 [SMILES: c1cc(Oc2ccc3[nH]ccc3c2)cc(-c2nnc(Cc3cncs3)[nH]2)c1] |
| 15 | 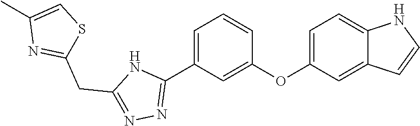 [SMILES: Cc1csc(Cc2nnc(-c3cccc(Oc4ccc5[nH]ccc5c4)c3)[nH]2)n1] |
| 16 | 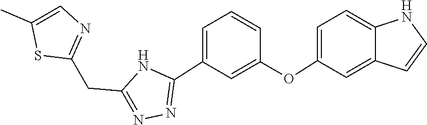 [SMILES: Cc1cnc(Cc2nnc(-c3cccc(Oc4ccc5[nH]ccc5c4)c3)[nH]2)s1] |
| 17 | 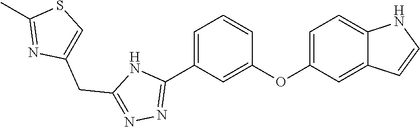 [SMILES: Cc1nc(Cc2nnc(-c3cccc(Oc4ccc5[nH]ccc5c4)c3)[nH]2)cs1] |
| 18 | 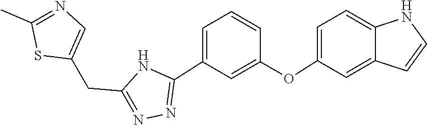 [SMILES: Cc1ncc(Cc2nnc(-c3cccc(Oc4ccc5[nH]ccc5c4)c3)[nH]2)s1] |
| 19 | 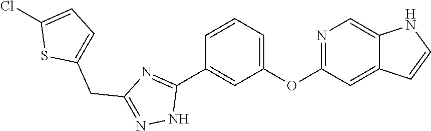 [SMILES: Clc1ccc(Cc2n[nH]c(-c3cccc(Oc4cc5cc[nH]c5cn4)c3)n2)s1] |
| 20 | 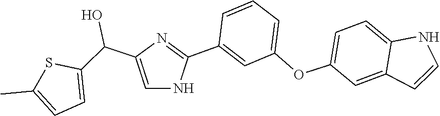 [SMILES: Cc1ccc(C(O)c2c[nH]c(-c3cccc(Oc4ccc5[nH]ccc5c4)c3)n2)s1] |
| 21 | 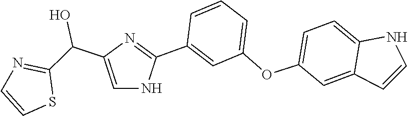 [SMILES: OC(c1c[nH]c(-c2cccc(Oc3ccc4[nH]ccc4c3)c2)n1)c1nccs1] |
| 22 | 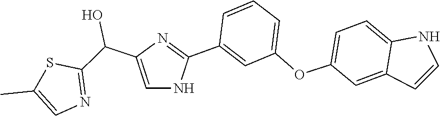 [SMILES: Cc1cnc(C(O)c2c[nH]c(-c3cccc(Oc4ccc5[nH]ccc5c4)c3)n2)s1] |
| 23 | 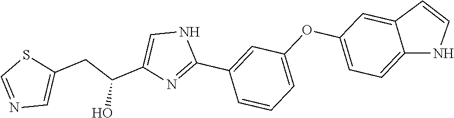 [SMILES: O[C@H](Cc1cncs1)c1c[nH]c(-c2cccc(Oc3ccc4[nH]ccc4c3)c2)n1] |
| 24 | 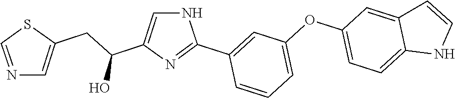 [SMILES: O[C@@H](Cc1cncs1)c1c[nH]c(-c2cccc(Oc3ccc4[nH]ccc4c3)c2)n1] |
| 25 | 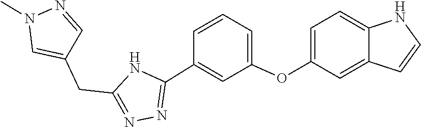 [SMILES: Cn1cc(Cc2nnc(-c3cccc(Oc4ccc5[nH]ccc5c4)c3)[nH]2)cn1] |
| 26 | 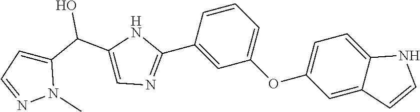 [SMILES: Cn1nccc1C(O)c1cnc(-c2cccc(Oc3ccc4[nH]ccc4c3)c2)[nH]1] |
| 27 | 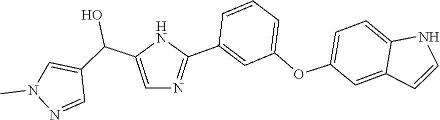 [SMILES: Cn1cc(C(O)c2cnc(-c3cccc(Oc4ccc5[nH]ccc5c4)c3)[nH]2)cn1] |
| 28 | 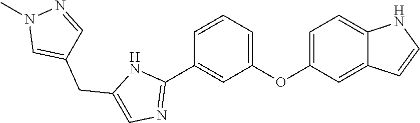 [SMILES: Cn1cc(Cc2cnc(-c3cccc(Oc4ccc5[nH]ccc5c4)c3)[nH]2)cn1] |
| 29 | 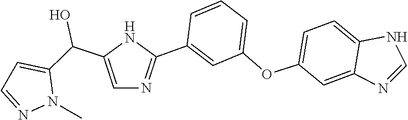 [SMILES: Cn1nccc1C(O)c1cnc(-c2cccc(Oc3ccc4[nH]cnc4c3)c2)[nH]1] |
| 30 | 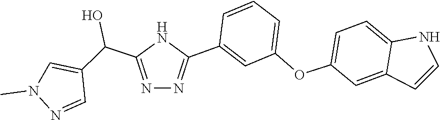 [SMILES: Cn1cc(C(O)c2nnc(-c3cccc(Oc4ccc5[nH]ccc5c4)c3)[nH]2)cn1] |
| 31 | 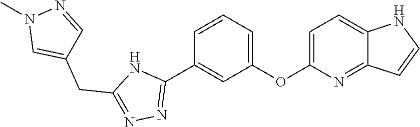 [SMILES: Cn1cc(Cc2nnc(-c3cccc(Oc4ccc5[nH]ccc5n4)c3)[nH]2)cn1] |
| 32 | 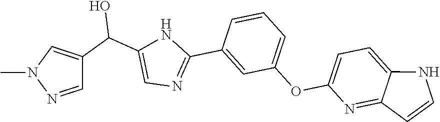 [SMILES: Cn1cc(C(O)c2cnc(-c3cccc(Oc4ccc5[nH]ccc5n4)c3)[nH]2)cn1] |
| 33 | 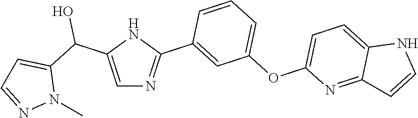 [SMILES: Cn1nccc1C(O)c1cnc(-c2cccc(Oc3ccc4[nH]ccc4n3)c2)[nH]1] |
| 34 | 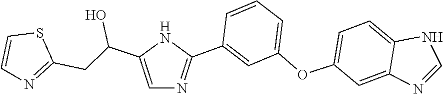 [SMILES: OC(Cc1nccs1)c1cnc(-c2cccc(Oc3ccc4[nH]cnc4c3)c2)[nH]1] |
| 35 | 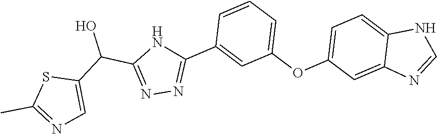 [SMILES: Cc1ncc(C(O)c2nnc(-c3cccc(Oc4ccc5[nH]cnc5c4)c3)[nH]2)s1] |
| 36 | 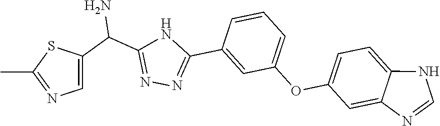 [SMILES: Cc1ncc(C(N)c2nnc(-c3cccc(Oc4ccc5[nH]cnc5c4)c3)[nH]2)s1] |
| 37 | 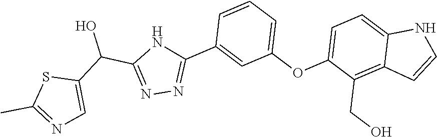 [SMILES: Cc1ncc(C(O)c2nnc(-c3cccc(Oc4ccc5[nH]ccc5c4CO)c3)[nH]2)s1] |
| 38 | 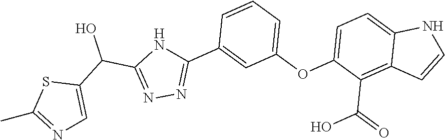 [SMILES: Cc1ncc(C(O)c2nnc(-c3cccc(Oc4ccc5[nH]ccc5c4C(=O)O)c3)[nH]2)s1] |
| 39 | 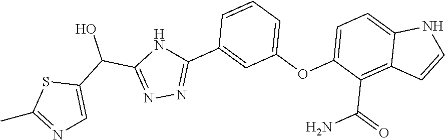 [SMILES: Cc1ncc(C(O)c2nnc(-c3cccc(Oc4ccc5[nH]ccc5c4C(N)=O)c3)[nH]2)s1] |
| 40 | 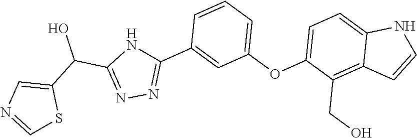 [SMILES: OCc1c(Oc2cccc(-c3nnc(C(O)c4cncs4)[nH]3)c2)ccc2[nH]ccc12] |
| 41 | 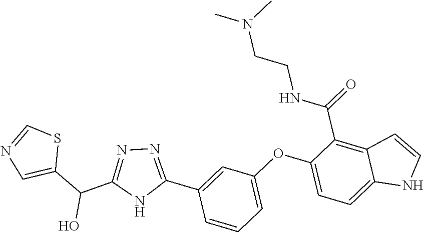 [SMILES: CN(C)CCNC(=O)c1c(Oc2cccc(-c3nnc(C(O)c4cncs4)[nH]3)c2)ccc2[nH]ccc12] |
| 42 | 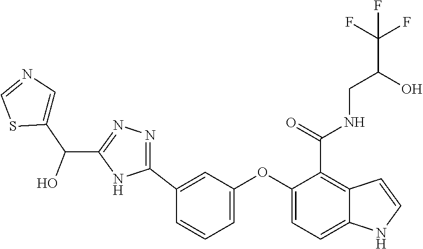 [SMILES: O=C(NCC(O)C(F)(F)F)c1c(Oc2cccc(-c3nnc(C(O)c4cncs4)[nH]3)c2)ccc2[nH]ccc12] |
| 43 | 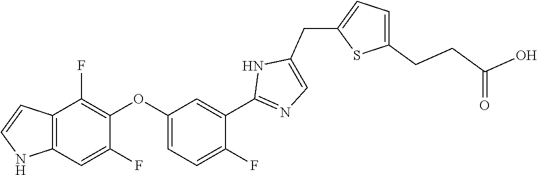 [SMILES: O=C(O)CCc1ccc(Cc2cnc(-c3cc(Oc4c(F)cc5[nH]ccc5c4F)ccc3F)[nH]2)s1] |
| 44 | 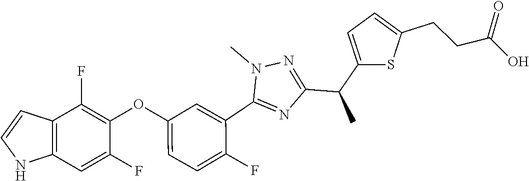 [SMILES: C[C@@H](c1nc(-c2cc(Oc3c(F)cc4[nH]ccc4c3F)ccc2F)n(C)n1)c1ccc(CCC(=O)O)s1] |
| 45 | 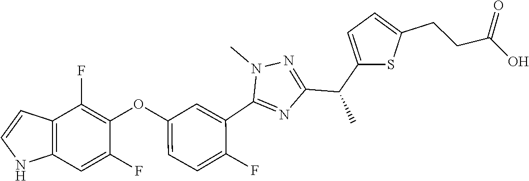 [SMILES: C[C@H](c1nc(-c2cc(Oc3c(F)cc4[nH]ccc4c3F)ccc2F)n(C)n1)c1ccc(CCC(=O)O)s1] |
| 46 | 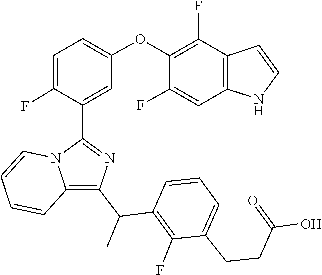 [SMILES: CC(c1cccc(CCC(=O)O)c1F)c1nc(-c2cc(Oc3c(F)cc4[nH]ccc4c3F)ccc2F)n2ccccc12] |
| 47 | 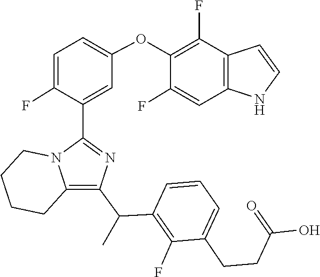 [SMILES: CC(c1cccc(CCC(=O)O)c1F)c1nc(-c2cc(Oc3c(F)cc4[nH]ccc4c3F)ccc2F)n2c1CCCC2] |
| 48 | 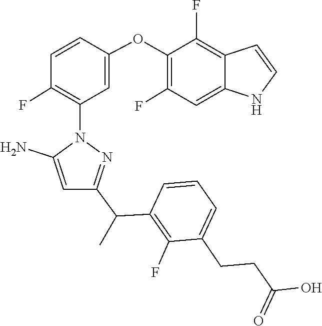 [SMILES: CC(c1cc(N)n(-c2cc(Oc3c(F)cc4[nH]ccc4c3F)ccc2F)n1)c1cccc(CCC(=O)O)c1F] |
| 49 | 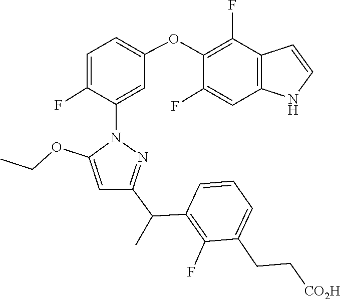 [SMILES: CCOc1cc(C(C)c2cccc(CCC(=O)O)c2F)nn1-c1cc(Oc2c(F)cc3[nH]ccc3c2F)ccc1F] |
| 50 | 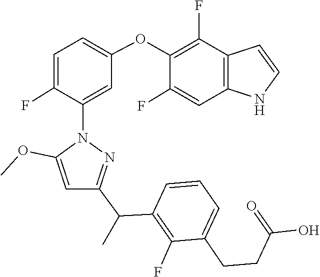 [SMILES: COc1cc(C(C)c2cccc(CCC(=O)O)c2F)nn1-c1cc(Oc2c(F)cc3[nH]ccc3c2F)ccc1F] |
| 51 | 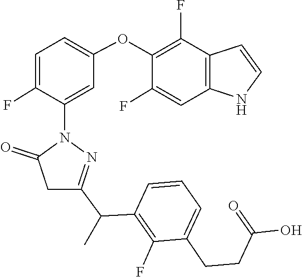 [SMILES: CC(C1=NN(c2cc(Oc3c(F)cc4[nH]ccc4c3F)ccc2F)C(=O)C1)c1cccc(CCC(=O)O)c1F] |
| 52 | 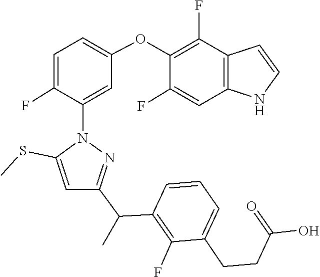 [SMILES: CSc1cc(C(C)c2cccc(CCC(=O)O)c2F)nn1-c1cc(Oc2c(F)cc3[nH]ccc3c2F)ccc1F] |
| 53 | 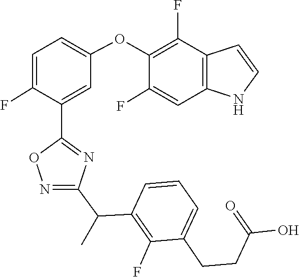 [SMILES: CC(c1noc(-c2cc(Oc3c(F)cc4[nH]ccc4c3F)ccc2F)n1)c1cccc(CCC(=O)O)c1F] |
| 54 | 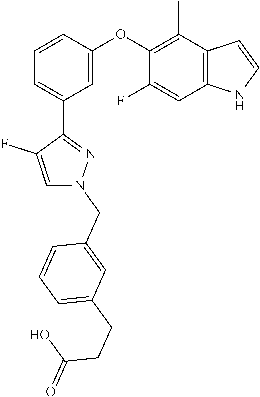 [SMILES: Cc1c(Oc2cccc(-c3nn(Cc4cccc(CCC(=O)O)c4)cc3F)c2)c(F)cc2[nH]ccc12] |
| 55 | 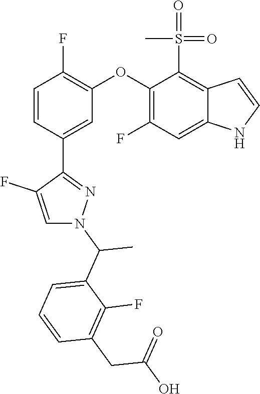 [SMILES: CC(c1cccc(CC(=O)O)c1F)n1cc(F)c(-c2ccc(F)c(Oc3c(F)cc4[nH]ccc4c3S(C)(=O)=O)c2)n1] |
| 56 | 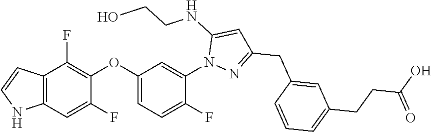 [SMILES: O=C(O)CCc1cccc(Cc2cc(NCCO)n(-c3cc(Oc4c(F)cc5[nH]ccc5c4F)ccc3F)n2)c1] |
| 57 | 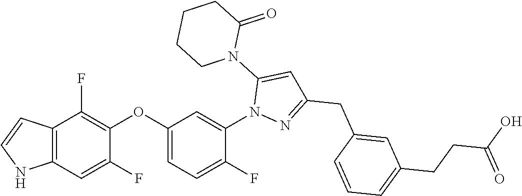 [SMILES: O=C(O)CCc1cccc(Cc2cc(N3CCCCC3=O)n(-c3cc(Oc4c(F)cc5[nH]ccc5c4F)ccc3F)n2)c1] |
| 58 | 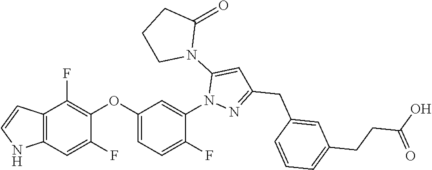 [SMILES: O=C(O)CCc1cccc(Cc2cc(N3CCCC3=O)n(-c3cc(Oc4c(F)cc5[nH]ccc5c4F)ccc3F)n2)c1] |
| 59 | 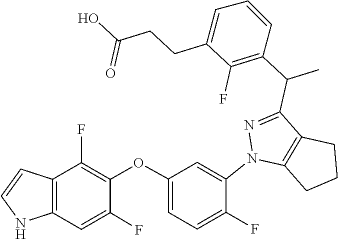 [SMILES: CC(c1cccc(CCC(=O)O)c1F)c1nn(-c2cc(Oc3c(F)cc4[nH]ccc4c3F)ccc2F)c2c1CCC2] |

or a pharmaceutically acceptable salt thereof.

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “haloalkyl” refers to a straight or branched alkyl group that is substituted with one or more halogen atoms.

The term “alkyl” as used herein is a branched or unbranched saturated hydrocarbon group having a specified number of carbon atoms. In some embodiments, alkyl refers to a branched or unbranched saturated hydrocarbon group having three carbon atoms (C₃). In some embodiments, alkyl refers to a branched or unbranched saturated hydrocarbon group having six carbon atoms (C₆). In some embodiments, the term “alkyl” includes, but is not limited to, methyl, ethyl, n-propyl, isopropyl, n-butyl, isobutyl, s-butyl, t-butyl, n-pentyl, isopentyl, s-pentyl, neopentyl, and hexyl.

As used herein, the term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group. The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 T electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —S(O)(NR^{∘})R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat and/or diagnose the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an CFTR-associated disease or disorder.

The terms “treat”, “treatment” or “treating” mean to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. Without being bound by any theory, in some embodiments, treating includes augmenting deficient CFTR activity. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein, “CFTR” means cystic fibrosis transmembrane conductance regulator. Defects in the function of the CFTR ion channel result from loss of function mutations of CFTR. Such mutations lead to exocrine gland dysfunction, abnormal mucociliary clearance, and cause cystic fibrosis. The most common CFTR mutation in Cystic Fibrosis (CF) patients leads to the specific deletion of three nucleotides of the codon for phenylalanine at position 508. This mutation, which is found in ~70% of CF patients worldwide, is referred to as “ΔF508”. The ΔF508 mutation decreases the stability of the CFTR NBD1 domain and limits CFTR interdomain assembly. Since CF is an autosomal recessive disease, a CF patient harboring the ΔF508 CFTR mutation must also carry a second defective copy of CFTR. Approximately 2000 different CF-causing CFTR mutations have been identified in CF patients. CF patients harboring the ΔF508 CFTR mutation can be homozygous for that mutation (ΔF508/ΔF508). CF patients can also be ΔF508 heterozygous, if the second CFTR allele such patients carry instead contains a different CFTR loss of function mutation. Such CFTR mutations include, but are not limited to, G542X, G551D, N1303K, W1282X, R553X, R1 17H, R1162X, R347P, G85E, R560T, A455E, ΔI507, G178R, S549N, S549R, G551S, G970R, G1244E, S1251N, 51255P, and G1349D.

As used herein, the term “CFTR modulator” refers to a compound that increases the activity of CFTR. In certain aspects, a CFTR modulator is a CFTR corrector or a CFTR potentiator or a dual-acting compound having activities of a corrector and a potentiator.

As used herein, the term “CFTR corrector” refers to a compound that increases the amount of functional CFTR protein to the cell surface and thus enhances CFTR channel function. The CFTR correctors partially “rescue” misfolding of CFTR, thereby enabling the maturation and functional expression of CFTR protein harboring a CF causing mutation on the cell surface. Examples of correctors include, but are not limited to, VX-809, VX-661, VX-152, VX-440, VX-983, and GLPG2222. Such compounds may interact directly with CFTR protein, modifying its folding and conformational maturation during synthesis.

As used herein, the term “CFTR potentiator” refers to a compound that increases the ion channel activity of CFTR protein located at the cell surface, resulting in enhanced ion transport. CFTR potentiators repair the defective channel functions caused by mutations.

Examples of potentiators include, but are not limited to, ivacaftor (VX770), deuterated ivacaftor (CPT 656), genistein and GLPG1837.

As used herein, the term “CFTR pharmacological chaperone” (PC) refers to compounds that stabilize the CFTR protein in its native state by binding directly to the protein.

As used herein, the term “CFTR proteostasis regulator” (PR) refers to compounds that enhance the protein folding efficiency within the cell. PRs can alter the activity of transcriptional, folding and/or membrane trafficking machinery, as well as impeding the degradation of partially folded, but functional, conformers at the endoplasmic reticulum (ER) or plasma membrane.

As used herein, “CFTR disease or condition” refers to a disease or condition associated with deficient CFTR activity, for example, cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, dry eye disease, protein C deficiency, A-beta.-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

### ALTERNATIVE EMBODIMENTS

In an alternative embodiment, compounds described herein may also comprise one or more isotopic substitutions. For example, hydrogen may be ²H (D or deuterium) or ³H (T or tritium); carbon may be, for example, ¹³C or ¹C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of the present disclosure and a pharmaceutically acceptable carrier, adjuvant, or vehicle. In some embodiments, the amount of compound in compositions contemplated herein is such that is effective to measurably modulate CFTR, or a mutant thereof, in a biological sample or in a patient. In certain embodiments, the amount of compound in compositions of this disclosure is such that is effective to measurably modulate CFTR, or a mutant thereof, in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, the amount of compound in compositions contemplated herein is such that is effective to measurably modulate a protein, particularly at CFTR, or a mutant thereof, in a biological sample or in a patient. In certain embodiments, the amount of compound in compositions of this disclosure is such that is effective to measurably modulate CFTR, or a mutant thereof, in a biological sample or in a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of the present disclosure may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of the present disclosure may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of the present disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of the present disclosure may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of the present disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

As discussed above, CFTR is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate and thiocyanate into and out of the cell. The most frequent CFTR mutation is the in-frame deletion of phenylalanine at residue 508 (ΔF508) in the first nucleotide binding domain (NBD1). The mutation has several deleterious effects on the production of CFTR in the ER, its correct folding, its movement to the plasma membrane and its normal function as an ion channel for the cell.

One such negative effect is that the NBD1 domain is partially or mis-folded which is recognized within the cell as an aberrant protein and tagged for disposal by ER-associated degradation (ERAD) via the ubiquitin-proteasome system (UPS). Should a partially or mis-folded CFTR protein emerge from the ER, the protein must travel to the plasma membrane through complex glycosylation in the Golgi compartment and be functionally inserted. In wild-type CFTR, only 20-40% of CFTR reaches the plasma membrane, indicating that CFTR has energetic instability of individual NBDs, a slow domain assembly, and relatively fast ERAD kinetics which all contribute to inefficient folding and sensitize CFTR to structural perturbations by mutations.

In wild-type CFTR, the NBD1 domain folds co-translationally while other domains fold post-translationally. Mutated ΔF508 CFTR has impaired NBD1 folding but its backbone structure and thermodynamic stability are similar to wild-type CFTR. With delayed folding kinetics, mutated ΔF508 CFTR NBD1 has an increased folding activation energy. Lack of proper folding results in hydrophobic residues being exposed to the surface of NBD1 which causes aggregation with other CFTR proteins. Thus, the aggregation temperature of mutated CFTR drops from 41° C. to 33° C. This level of instability creates a greater percentage of mis-folded mutant CFTR at physiological temperature (37° C. in humans). Mutant CFTR suffers from both kinetic and thermodynamic folding defects. CFTR stabilizers can address these folding defects, but complete energetic correction of mutant NBD1 folding has been shown to not result in the CFTR biosynthetic processing, underscoring the need for interface stability as well.

The disclosed CFTR correctors can interact with the NBD domain to stabilize the correct folded position R, such that CFTR is not labeled for elimination from the cell. The preservation of correct folding enables CFTR to function as a chloride ion channel at wild-type levels. In some embodiments, disclosed CFTR correctors can enhance the performance of wild-type CFTR.

CFTR stabilizers can function in combination with other therapeutic agents such as CFTR correctors that promote Δ508 CFTR exit from the ER and accumulation in the plasma membrane. Increasing the amount of CFTR cell surface expression can result in improved chloride conductance following channel activation by both potentiators and a cAMP agonist. Thus, disclosed herein are combinations of CFTR stabilizers with CFTR correctors and potentiators, optionally with cAMP agonists or another therapeutic agent as described below.

Disclosed herein are methods of treating deficient CFTR activity in a cell, comprising contacting the cell with a compound of the present disclosure, or a pharmaceutically acceptable salt thereof. In certain embodiments, contacting the cell occurs in a subject in need thereof, thereby treating a disease or disorder mediated by deficient CFTR activity.

Also, disclosed herein are methods of treating a disease or a disorder mediated by deficient CFTR activity comprising administering a compound of the present disclosure or a pharmaceutically acceptable salt thereof. In some embodiments, the subject is a mammal, preferably a human. In some embodiments, the disease is associated with the regulation of fluid volumes across epithelial membranes, particularly an obstructive airway disease such as CF or COPD.

Such diseases and conditions include, but are not limited to, cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear palsy, Pick's disease, several polyglutamine neurological disorders, Huntington's, spinocerebellar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

Such diseases and conditions include, but are not limited to, cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome. In some embodiments, a disease is cystic fibrosis.

Provided herein are methods of treating cystic fibrosis, comprising administering to a subject in need thereof, a compound as disclosed herein or a pharmaceutically acceptable salt thereof. Also provided herein are methods of lessening the severity of cystic fibrosis, comprising administering to a subject in need thereof, a compound as disclosed herein or a pharmaceutically acceptable salt thereof. In some embodiments, the subject is a human. In some embodiments, the subject is at risk of developing cystic fibrosis, and administration is carried out prior to the onset of symptoms of cystic fibrosis in the subject.

Provided herein are compounds as disclosed herein for use in treating a disease or condition mediated by deficient CFTR activity. Also provided herein are uses of a compound as disclosed herein for the manufacture of a medicament for treating a disease or condition mediated by deficient CFTR activity.

Provided herein are kits for use in measuring the activity of CFTR or a fragment thereof in a biological sample in vitro or in vivo. The kit can contain: (i) a compound as disclosed herein, or a pharmaceutical composition comprising the disclosed compound, and (ii) instructions for: a) contacting the compound or composition with the biological sample; and b) measuring activity of said CFTR or a fragment thereof. In some embodiments, the biological sample is biopsied material obtained from a mammal or extracts thereof; blood, saliva, urine, feces, semen, tears, other body fluids, or extracts thereof. In some embodiments, the mammal is a human.

Provided herein are compounds as disclosed herein for use in treating kidney disease. In some embodiments, a kidney disease is autosomal dominant polycystic kidney disease (ADPKD) or autosomal recessive polycystic kidney disease (ARPKD). In some embodiments, a kidney disease is autosomal dominant polycystic kidney disease (ADPKD). In some embodiments, a kidney disease is autosomal recessive polycystic kidney disease (ARPKD).

### Combination Treatments

As used herein, the term “combination therapy” means administering to a subject (e.g., human) two or more CFTR modulators, or a CFTR modulator and an agent such as antibiotics, ENaC inhibitors, GSNO (S-nitrosothiol, s-nitroglutathione) reductase inhibitors, and a CRISPR Cas correction therapy or system (as described in US 2007/0022507 and the like). In some embodiments, combination therapy includes administration of a compound described herein with a compound that modulates CFTR protein or ABC protein activities (e.g., as described in WO2018167690A1 and the like)

In certain embodiments, the method of treating a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

In certain embodiments, the method of preventing a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

Additional therapeutic agents include, for example, ENaC inhibitors, mucolytic agents, modulators of mucus rheology, bronchodilators, antibiotics, anti-infective agents, anti-inflammatory agents, ion channel modulating agents, therapeutic agents used in gene or mRNA therapy, agents that reduce airway surface liquid and/or reduce airway surface PH, CFTR correctors, and CFTR potentiators, or other agents that modulate CFTR activity. Other therapeutics include liposomal composition components such as those described in WO2012/170889, hybrid oligonucleotides that facilitate RNA cleavage such as those described in WO2016/130943, and single stranded oligonucleotides that modulate gene expression as described in WO2016/130929.

In some embodiments, at least one additional therapeutic agent is selected from one or more CFTR modulators, one or more CFTR correctors and one or more CFTR potentiators.

Non-limiting examples of additional therapeutics include VX-770 (Ivacaftor), VX-809 (Lumacaftor, 3-(6-(I-(2,2-5 difluorobenzo[d][1, 3]dioxol-5-yl)cyclopropanecarboxamido)-3-methylpyridin-2-yl) benzoic acid, VX-661 (Tezacaftor, I-(2,2-difluoro-1, 3-benzodioxol-5-yl)-N—[I-[(2R)-2,3-dihydroxypropyl]-6-fluoro-2-(2-hydroxy-1, I-dimethylethyl)-1H-indol-5-yl]-cyclopropanecarboxamide), VX-983, VX-152, VX-440, VX-445, VX-659, VX-371, Orkambi, Ataluren (PTC 124) (3-[5-(2-fluorophenyl)-1, 2,4-oxadiazol-3-yl]benzoic acid), PTI-130 (Proteostasis), PTI-801, PTI-808, PTI-428, N91115.74 (cavosonstat), QBW251 (Novartis) compounds described in WO2011113894, compounds N30 Pharmaceuticals (e.g., WO 2014/186704), deuterated ivacaftor (e.g., CTP-656 or VX-561), GLPG 2222, GLPG2451, GLPG3067, GLPG2851, GLPG2737, GLPG 1837 (N-(3-carbamoyl-5,5,7,7-tetramethyl-5,7-dihydro-4H-thieno[2,3-c]pyran-2-yl)-1H-pyrazole-5-carboxamide), GLPG 2665 (Galapagos), FDL 169 (Flatley Discovery lab), FDL 176, FDL438, FDL304, FD2052160, FD1881042, FD2027304, FD2035659, FD2033129, FD1860293, CFFT-Pot01, CFFT-Pot-02, P-1037, glycerol, phenylbutyrate, and the like.

Non-limiting examples of additional therapeutics include compounds disclosed in US Patent Application Nos. PCT/US20/63586, PCT/US20/63589, and PCT/US20/63590, each of which is incorporated by reference in its entirety.

Non-limiting examples of anti-inflammatory agents are N6022 (3-(5-(4-(1H-imidazol-I-yl)10 phenyl)-I-(4-carbamoyl-2-methylphenyl)-¹H-pyrrol-2-yl) propanoic acid), Ibuprofen, Lenabasum (anabasum), Acebilustat (CTX-4430), LAU-7b, POL6014, docosahexaenoic acid, alpha-1 anti-trypsin, sildenafil. Additional therapeutic agents also include, but are not limited to a mucolytic agent, a modifier of mucus rheology (such as hypertonic saline, mannitol, and oligosaccharide based therapy), a bronchodilator, an anti-infective (such as tazobactam, piperacillin, rifampin, meropenem, ceftazidime, aztreonam, tobramycin, fosfomycin, azithromycin, amitriptyline, vancomycin, gallium and colistin), an anti-infective agent, an anti-inflammatory agent, a CFTR modulator other than a compound of the present disclosure, and a nutritional agent. Additional therapeutic agents can include treatments for comorbid conditions of cystic fibrosis, such as exocrine pancreatic insufficiency which can be treated with Pancrelipase or Liprotamase.

Examples of CFTR potentiators include, but are not limited to, Ivacaftor (VX-770), CTP-656, NVS-QBW251, FD1860293, GLPG2451, GLPG1837, and N-(3-carbamoyl-5,5,7,7-tetramethyl-5,7-dihydro-4H-thieno[2,3-c]pyran-2-yl)-1H-pyrazole-5-carboxamide. Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, WO2013038390, WO2014180562, WO2015018823, and U.S. patent application Ser. Nos. 14/271,080, 14/451,619 and 15/164,317.

Non-limiting examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661), VX-983, GLPG2222, GLPG2665, GLPG2737, VX-152, VX-440, FDL169, FDL304, FD2052160, and FD2035659. Examples of correctors are also disclosed in US20160095858A1, and U.S. application Ser. Nos. 14/925,649 and 14/926,727.

In certain embodiments, the additional therapeutic agent is a CFTR amplifier. CFTR amplifiers enhance the effect of known CFTR modulators, such as potentiators and correctors. Examples of CFTR amplifier include PTI130 and PTI-428. Examples of amplifiers are also disclosed in publications: WO2015138909 and WO2015138934.

In certain embodiments, the additional therapeutic agent is an agent that reduces the activity of the epithelial sodium channel blocker (ENaC) either directly by blocking the channel or indirectly by modulation of proteases that lead to an increase in ENaC activity (e.g., serine proteases, channel-activating proteases). Exemplary of such agents include camostat (a trypsin-like protease inhibitor), QAU145, 552-02, GS-9411, INO-4995, Aerolytic, amiloride, AZD5634, and VX-371. Additional agents that reduce the activity of the epithelial sodium channel blocker (ENaC) can be found, for example, in PCT Publication No. WO2009074575 and WO2013043720; and U.S. Pat. No. 8,999,976.

In one embodiment, the ENaC inhibitor is VX-371.

In one embodiment, the ENaC inhibitor is SPX-101 (S18).

In certain embodiments, the combination of a compound of the present disclosure, with a second therapeutic agent may have a synergistic effect in the treatment of cancer and other diseases or disorders mediated by adenosine. In other embodiments, the combination may have an additive effect.

### Exemplification

### Analytical Procedures

¹H NMR spectra were recorded with a Bruker AC 400 MHz apparatus. Chemical shifts (6) are quoted in parts per million (ppm) and coupling constants (J) in hertz (Hz).

LC-MS spectra were obtained, unless noted otherwise, with a UPLC Acquity device of Waters for the liquid chromatography analysis, coupling with a ZMD (Waters) mass spectrometer. This system was piloted by MassLynx v4.1 software. Detection was made in UV at 220 nm.

Operational conditions for liquid chromatography analysis are as follows:
Column: Assentis Express Cis 50×2.1 mm, 2.7 μm Supelco
Eluent: Way A: H₂O+0.02% TFA;
   Way B: CH₃CN+0.014% TFA;
Gradient: T₀ min: 2% B, T₁ min: 98% B, T_{1.3} min: 98% B, T_{1.33} min: 2% B, T_{1.5} min: following injection;
Flow: 1 mL/min;
Temperature: 55° C.
SQD: ESI+30V
UV detection wavelength: 220 nm
Injection volume: 0.2 μl.

Preparatory HPLC purification was carried out under the following conditions:
Instrument: Gilson 281 (PHGO11)
Column Xtimate C18 21.2*250 mm, 10 μm
Mobile Phase: A: water (10 mM NH₄HCO3 spiked with 0.025% NH₃·H₂O); B: acetonitrile
Gradient: 5% B for 3 min, then 5-37% B in 10 min, stop at 18 min
Flow Rate: 30.00 ml/min
Detection Wavelength (nm): 214/254
Retention Time (min): 8

### Abbreviations

AcOH: acetic acid
AIBN: azobisisobutyronitrile
BINAP: 2,2′-bis(diphenylphosphino)-1,1′-binaphthyl
Boc: tert-butyloxycarbonyl
n-BuOH: n-butanol
DABAL-Me3: bis(trimethylaluminum)-1,4-diazabicyclo[2.2.2]octane adduct
DBU: 1,8-diazabicyclo[5.4.0]undec-7-ene
DCM: dichloromethane
DCE: 1,2-dichloroethane
DEA: diethyl amine
DIPEA: N,N-diisopropylethylamine
DMAP: 4-dimethylaminopyridine
DMP: N,N-dimethylformamide
DMF-DMA: N,N-dimethylformamide dimethyl acetal
DMSO: dimethyl sulfoxide
dppf: 1,1′-bis(diphenylphosphino)ferrocene
DTT: dithiothreitol
Ee: enantiomeric excess
Eq: equivalents
ESI: electron spray ionization
EtOAc: ethyl acetate
EtOH: ethanol
FA: formic acid
HATU: 1-[bis(dimethylamino)methylene]-1H-1,2,3-triazolo[4,5-b]pyridinium 3-oxide hexafluorophosphate
HPLC: high performance liquid chromatography
In vacuo: under vacuum; under reduced pressure
LAH: lithium aluminum hydride
LC-MS: liquid chromatography-mass spectrometry
LDA: lithium diisopropylamide
LHMDS (LiHMDS): lithium bis(trimethylsilyl)amide
MeOH: methanol
NBS: N-bromosuccinimide
NIS: N-iodosuccinimide
NMP: N-methyl-2-pyrrolidone
Pd/C: palladium on carbon
Prep-HPLC: preparative HPLC
SFC: supercritical fluid chromatography
TBAF: tetra-n-butylammonium fluoride
TBS: tert-butyldimethylsilyl
TFA: trifluoroacetic acid
TIPS: triisopropylsilyl
THE: tetrahydrofuran
THP: tetrahydropyran
TLC: thin layer chromatography
Ts: tosyl

### Preparation of Intermediates

### Intermediate 1

### 2-Fluoro-5-((6-fluoro-4-methyl-1H-indol-5-yl)oxy)benzimidamide

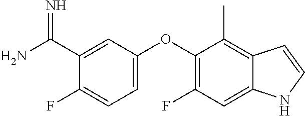 [SMILES: Cc1c(Oc2ccc(F)c(C(=N)N)c2)c(F)cc2[nH]ccc12]

A solution of 2-fluoro-5-((6-fluoro-4-methyl-1H-indol-5-yl)oxy)benzonitrile (Intermediate 1D) (600 mg, 2.1 mmol) and LHMDS (1M in THF) (8.4 mL, 8.4 mmol) in THE (12 mL) was stirred at room temperature overnight. The reaction was quenched with water (15 mL), and extracted with a mixture of EtOAc and THE (1:1). The organic phase was dried over anhydrous Na₂SO₄ and concentrated in vacuo to give the title compound (635 mg, crude) as brown solid. This crude product was used directly in the next reaction step without further purification. MS (ESI): 302.1 m/z (M+H)⁺.

### Intermediate 1A

### 1,2-Difluoro-3,4-dimethyl-5-nitrobenzene

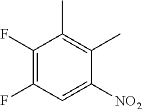

A mixture of 3-bromo-1,2-difluoro-4-methyl-5-nitrobenzene (10 g, 40 mmol), MeB(OH)₂ (12 g, 200 mmol), Pd(dppt)Cl₂ (2.9 mg, 4.0 mmol) and NaHCO₃ (10 g, 120 mmol) in 1,4-dioxane (160 mL) and H₂O (40 mL) was stirred under an N₂ atmosphere at 80° C. for 3 days. The mixture was filtered, and ethyl acetate (300 mL) was added to the filtrate. The organic phase was washed with H₂O (100 mL×2) and brine (50 mL×1), dried over anhydrous sodium sulfate, filtered and concentrated in vacuo. The residue was purified by silica gel column chromatography (eluting with 12:1 petroleum ether:ethyl acetate) to afford 1,2-difluoro-3,4-dimethyl-5-nitrobenzene (6.4 g, 85%) as a light-yellow oil. ¹H NMR (400 MHz, CDCl₃-d): d 7.59 (t, J=8.4 Hz, 1H), 2.42 (s, 3H), 2.32 (d, J=2.4 Hz, 3H) ppm.

### Intermediate 1B

### 2-(3-Bromo-4-fluorophenoxy)-1-fluoro-3,4-dimethyl-5-nitrobenzene

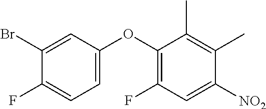 [SMILES: Cc1c([N+](=O)[O-])cc(F)c(Oc2ccc(F)c(Br)c2)c1C]

To a solution of 1,2-difluoro-3,4-dimethyl-5-nitrobenzene (14 g, 74.9 mmol) in DMF (100 mL) was added 3-bromo-4-fluorophenol (14.9 g, 32.5 mmol) and K₂CO₃ (20.7 g, 149.7 mmol). The resulting mixture was stirred at 100° C. for 2 hours, cooled to room temperature and diluted with ethyl acetate (250 mL). The organic layer was washed with water (100 mL×2), brine (100 mL×2), dried over anhydrous sodium sulfate, filtered, and concentrated in vacuo. The residue was purified by silica gel column chromatography (eluting with 100/1 petroleum ether/ethyl acetate) to afford 2-(3-bromo-4-fluorophenoxy)-1-fluoro-3,4-dimethyl-5-nitrobenzene (22 g, 83%) as a pale solid. ¹H NMR (400 MHz, CDCl₃): δ 7.57 (d, J=9.2 Hz, 1H), 7.10-7.00 (m, 2H), 6.82-6.75 (m, 1H), 2.44 (s, 3H), 2.26 (s, 3H) ppm.

### Intermediate 1C

### 5-(3-Bromo-4-fluorophenoxy)-6-fluoro-4-methyl-1H-indole

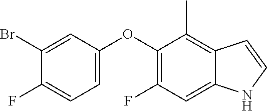 [SMILES: Cc1c(Oc2ccc(F)c(Br)c2)c(F)cc2[nH]ccc12]

A mixture of 2-(3-bromo-4-fluorophenoxy)-1-fluoro-3,4-dimethyl-5-nitrobenzene (10.0 g, 28.0 mmol), DMF-DMA (16.7 g, 140.1 mmol) and pyrrolidine (4.00 g, 56.0 mmol) was stirred at 100° C. for 2 hours and concentrated in vacuo. tThe residue was dissovled in a mixture of acetic acid (100 mL) and toluene (60 mL), and to the solution was added Fe powder (7.84 g, 140.1 mmol). The reaction mixture was stirred at 100° C. overnight, and then cooled to room temperature, filtered through a pad of Celite and the filter cake washed with ethyl acetate (100 mL×3). The combined organic phase was washed with brine (200 mL), dried over anhydrous sodium sulfate, filtered and concentrated in vacuo. The residue was purified by silica gel column chromatography (eluting with 15/1 petroleum ether/ethyl acetate) to afford 5-(3-bromo-4-fluorophenoxy)-6-fluoro-4-methyl-1H-indole (6.0 g, 64%) as a green solid. ¹H NMR (400 MHz, CDCl₃): δ 8.29 (s, 1H), 7.30-7.23 (m, 1H), 7.08 (d, J=10.0 Hz, 1H), 7.04-6.99 (m, 2H), 6.84-6.77 (m, 1H), 6.57 (s, 1H), 2.39 (s, 3H) ppm.

### Intermediate 1D

### 2-Fluoro-5-((6-fluoro-4-methyl-1H-indol-5-yl)oxy)benzonitrile

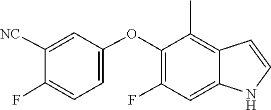 [SMILES: Cc1c(Oc2ccc(F)c(C#N)c2)c(F)cc2[nH]ccc12]

A mixture of 5-(3-bromo-4-fluorophenoxy)-6-fluoro-4-methyl-1H-indole (6.0 g, 17.8 mmol), Zn(CN)₂ (3.10 mg, 26.7 mmol), Pd(dppt)Cl₂ (1.45 g, 1.78 mmol) and Zn (114 mg, 1.78 mmol) in DMF (25 mL) was heated at 150° C. for 45 minutes in a microwave reactor. The reaction mixture was cooled to room temperature, filtered through a pad of Celite and the filter cake was washed with ethyl acetate (80 mL×3). The combined organic phase was washed with water (80 mL×2), brine (150 mL), dried over anhydrous sodium sulfate, filtered, and concentrated in vacuo. The residue was purified by silica gel column chromatography (eluting with petroleum 6/1 ether/ethyl acetate) to afford 2-fluoro-5-((6-fluoro-4-methyl-1H-indol-5-yl)oxy)benzonitrile (4.2 g, 83%) as a yellow solid. MS (ESI): 285.1 m/z (M+H)⁺. ¹H NMR (400 MHz, CDCl₃): δ 8.28 (s, 1H), 7.27 (s, 1H), 7.20-7.07 (m, 3H), 6.98 (dd, J=4.8 Hz & 2.8 Hz, 1H), 6.58 (s, 1H), 2.39 (s, 3H) ppm.

### Intermediate 2

### 5-((4,6-Difluoro-1H-indol-5-yl)oxy)-2-fluorobenzonitrile

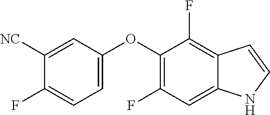 [SMILES: N#Cc1cc(Oc2c(F)cc3[nH]ccc3c2F)ccc1F]

A mixture of 5-[4-amino-2,6-difluoro-3-(2-trimethylsilylethynyl)phenoxy]-2-fluoro-benzonitrile (Intermediate 2D) (28.0 g, 77.7 mmol) and CuI (29.6 g, 155 mmol) in DMF (250 mL) was flushed with argon for 2 min and heated for 5 hours at 100° C. under argon. The insoluble materials were removed by suction filtration and the filtrate was diluted with ethyl acetate (1500 mL). The organic phase was washed with brine (300 mL×5), dried over anhydrous Na₂SO₄, filtered, and concentrated. The residue was purified by silica gel column chromatography (eluting with 4/1 petroleum ether/ethyl acetate) to afford the desired product, 5-[(4,6-difluoro-1H-indol-5-yl)oxy]-2-fluorobenzonitrile (18 g, yield 80%) as a yellow solid. ¹H NMR (500 MHz, DMSO-d6): δ 11.64 (br, 1H), 7.62 (dd, J=5.0, 3.5 Hz, 1H), 7.51-7.47 (m, 2H), 7.39-7.36 (m, 1H), 7.32 (d, J=10.0 Hz, 1H), 6.58 (t, J=2.0 Hz, 1H) ppm.

### Intermediate 2A

### 5-(2,6-Difluoro-4-nitrophenoxy)-2-fluorobenzonitrile

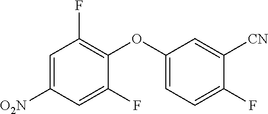 [SMILES: N#Cc1cc(Oc2c(F)cc([N+](=O)[O-])cc2F)ccc1F]

To a solution of 1,2,3-trifluoro-5-nitrobenzene (45 g, 0.254 mol) and 2-fluoro-5-hydroxybenzonitrile (38.3 g, 0.28 mol) in DMF (200 mL) was added K₂CO₃ (70 g, 0.5 mol). The resulting mixture was heated for 2 hours at 100° C. To the cooled reaction was added water (1500 mL), and the mixture was extracted with EtOAc (1000 mL×2). The combined organic phase was washed with brine (500 mL×3), dried over anhydrous Na₂SO₄, filtered and concentrated under reduced pressure to afford the desired product, 5-(2,6-difluoro-4-nitrophenoxy)-2-fluorobenzonitrile (74 g, 99%) as a yellow solid.

¹H NMR (500 MHz, DMSO-d6): δ 8.03-7.98 (m, 2H), 7.28-7.23 (m, 2H), 7.21-7.19 (m, 1H) ppm.

### Intermediate 2B

### 5-(4-Amino-2,6-difluorophenoxy)-2-fluorobenzonitrile

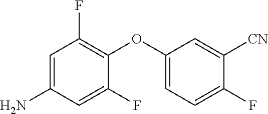 [SMILES: N#Cc1cc(Oc2c(F)cc(N)cc2F)ccc1F]

To a suspension of 5-(2,6-difluoro-4-nitrophenoxy)-2-fluorobenzonitrile (37 g, 126 mmol) in EtOH (450 mL) was added iron power (28.1 g, 543 mmol) and a solution of NH₄Cl (53.8 g, 1.01 mol) in water (150 mL). The resulting mixture was heated to reflux for 4 hours. The reaction mixture was filtered through a pad of Celite, and the filtrate was concentrated under reduced pressure. The residue was dissolved in ethyl acetate (1 L). The organic phase was washed with water (200 mL×3), brine (200 mL), dried over anhydrous Na₂SO₄, filtered and concentrated in vacuo to afford the desired product, 5-(4-amino-2,6-difluorophenoxy)-2-fluorobenzonitrile (33.2 g, quantitative yield) as a yellow solid.

¹H NMR (500 MHz, CDCl₃): δ 7.23-7.20 (m, 1H), 7.14 (t, J=8.5 Hz, 1H), 7.08 (dd, J=5.0, 3.5 Hz, 1H), 6.33-6.28 (m, 2H), 3.90 (br, 2H) ppm.

### Intermediate 2C

### 5-(4-Amino-2,6-difluoro-3-iodophenoxy)-2-fluorobenzonitrile

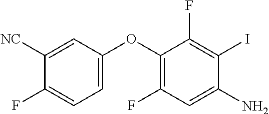 [SMILES: N#Cc1cc(Oc2c(F)cc(N)c(I)c2F)ccc1F]

A solution of 5-(4-amino-2,6-difluorophenoxy)-2-fluorobenzonitrile (33.2 g, 126 mmol) and N-iodosuccinimide (31.1 g, 126 mmol) in AcOH (250 mL) was stirred at room temperature for 1 hour. The solution was concentrated under reduced pressure. The residue was suspended in saturated aqueous NaHCO₃ (500 mL), and the resulting mixture was extracted with ethyl acetate (500 mL×3). The organic phase was washed with brine (200 mL×3), dried over anhydrous Na₂SO₄, filtered and concentrated. The residue was purified by silica gel column chromatography (eluting with 4/1 petroleum ether/ethyl acetate) to afford the desired product, 5-(4-amino-2,6-difluoro-3-iodophenoxy)-2-fluorobenzonitrile (45.5 g, 92%) as a yellow solid.

¹H NMR (500 MHz, DMSO-d₆): δ 7.63 (dd, J=5.0, 3.5 Hz, 1H), 7.49 (t, J=9.0 Hz, 1H), 7.39-7.36 (m, 1H), 6.63 (dd, J=13, 2.0 Hz, 1H), 5.88 (br s, 2H) ppm.

### Intermediate 2D

### 5-[4-Amino-2,6-difluoro-3-(2-trimethylsilylethynyl)phenoxy]-2-fluorobenzonitrile

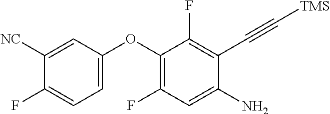 [SMILES: C[Si](C)(C)C#Cc1c(N)cc(F)c(Oc2ccc(F)c(C#N)c2)c1F]

To a stirred solution of 5-(4-amino-2,6-difluoro-3-iodophenoxy)-2-fluorobenzonitrile (61 g, 156 mmol) in DMF (300 mL) was added Pd(dppf)Cl₂ (3.43 g, 4.7 mmol), CuI (2.98 g, 15.6 mmol) and Et₃N (23.7 g, 235 mmol), followed by addition of ethynyl(trimethyl)silane (28 mL, 187 mmol). The resulting mixture was stirred at ambient temperature overnight under a nitrogen atmosphere. The reaction was quenched with water (500 mL) and extracted with ethyl acetate (500 mL×3). The combined organic phase was washed with brine (150 mL×3), dried over anhydrous Na₂SO₄, filtered, and concentrated in vacuo. The residue was purified by silica gel column chromatography (eluting with 1/3/3 petroleum ether/EtOAc/DCM) to afford the desired product, 5-[4-amino-2,6-difluoro-3-(2-trimethylsilylethynyl)phenoxy]-2-fluorobenzonitrile (42 g, 74.5%) as a yellow solid. MS m/z: 361 [M+H]⁺.

### Intermediate 3

### 2-Fluoro-5-((6-fluoro-4-methyl-1-tosyl-1H-indol-5-yl)oxy)-N-(prop-2-yn-1-yl)benzimidamide

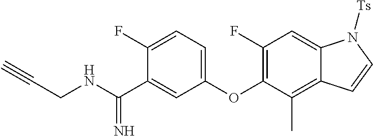 [SMILES: C#CCNC(=N)c1cc(Oc2c(F)cc3c(ccn3S(=O)(=O)c3ccc(C)cc3)c2C)ccc1F]

In glove box, to a microwave reaction tube were added 2-fluoro-5-((6-fluoro-4-methyl-1-tosyl-1H-indol-5-yl)oxy)benzonitrile (Intermediate 3A) (345 mg, 0.79 mmol), prop-2-yn-1-amine (348 mg, 6.3 mmol), and dry THF (6 mL). DABAL-Me3 (350 mg, 1.38 mmol) was then added carefully. The tube was sealed. The reaction was stirred at 130° C. for 1.5 hours in a microwave reactor. Reaction progress was monitored by LC-MS until reaction completion. The cooled reaction mixture was poured into ice-water (30 mL) and extracted with ethyl acetate (20 mL×3). The combined organic extracts were washed with brine (20 mL×3), dried over anhydrous sodium sulfate, filtered, and concentrated. The residue was purified by flash column chromatography on silica gel, eluting with 0-10% MeOH in DCM containing 2% 7N NH₃ in MeOH) to afford the pure product (298 mg, 77%) as a yellow solid. MS m/z: 494 [M+H]⁺. ¹H NMR (400 MHz, DMSO-d6): δ 7.96 (d, J=8.4 Hz, 2H), 7.88 (d, J=3.8 Hz, 1H), 7.77 (d, J=10.7 Hz, 1H), 7.43 (d, J=8.1 Hz, 2H), 7.17 (t, J=9.6 Hz, 1H), 6.97 (d, J=3.7 Hz, 1H), 6.88-6.86 (m, 2H), 6.84 (brs, 2H) 3.82 (s, 2H), 2.99 (s, 1H), 2.35 (s, 3H), 2.29 (s, 3H)

### Intermediate 3A

### 2-Fluoro-5-((6-fluoro-4-methyl-1-tosyl-1H-indol-5-yl)oxy)benzonitrile

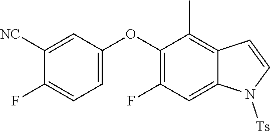 [SMILES: Cc1ccc(S(=O)(=O)n2ccc3c(C)c(Oc4ccc(F)c(C#N)c4)c(F)cc32)cc1]

To a stirred and chilled (0° C.) solution of 2-fluoro-5-((6-fluoro-4-methyl-1H-indol-5-yl)oxy)benzonitrile (Intermediate 1D) (527 mg, 1.86 mmol, 1 eq) in THE (5 mL) was added under a nitrogen atmosphere NaH (60% dispersion in mineral oil, 111 mg, 2.79 mmol, 1.5 eq.). The mixture was stirred for 30 min and TsCl (423.7 g, 2.23 mmol, 1.2 eq) was added. The mixture was stirred at room temperature overnight. The mixture was quenched with water (50 mL), and extracted with DCM (15 mL×3). The combined organic layers were dried over anhydrous sodium sulfate, filtered, and concentrated. The crude was purified by flash column chromatography on silica (eluting with 0-80% ethyl acetate in petroleum ether) to afford the product, 2-fluoro-5-((6-fluoro-4-methyl-1-tosyl-1H-indol-5-yl)oxy)benzonitrile (685 mg, 84%) as a white solid. MS: m/z=439 [M+H]⁺; 461 [M+Na]⁺.

¹H NMR (400 MHz, CDCl₃): δ 7.80 (d, J=8.3 Hz, 2H), 7.72 (d, J=10.4 Hz, 1H), 7.61 (d, J=3.7 Hz, 1H), 7.29 (d, J=8.1 Hz, 2H), 7.14-7.10 (m, 2H), 6.93 (d, J=4.1 Hz, 1H), 6.67 (d, J=3.7 Hz, 1H), 2.39 (s, 3H), 2.30 (s, 3H).

### Intermediate 4

### 5-((4,6-Difluoro-1H-indol-5-yl)oxy)-2-fluorobenzimidamide

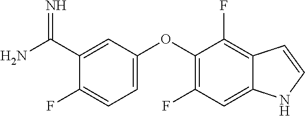 [SMILES: N=C(N)c1cc(Oc2c(F)cc3[nH]ccc3c2F)ccc1F]

To a stirred solution of 5-[(4,6-difluoro-1H-indol-5-yl)oxy]-2-fluoro-benzonitrile (Intermediate 2) (440 mg, 1.53 mmol) in THF (10 mL) was added the solution of LiHMDS (6.12 mL, 1 M in THF) at 0° C. The resulting mixture was stirred at 25° C. overnight. The reaction was quenched with water (0.2 mL) and diluted with ethyl acetate (30 mL). The mixture was washed with brine (10 mL×2), dried over anhydrous Na₂SO₄, filtered, and concentrated in vacuo to give the crude title compound (500 mg), which was used directly in the next step. MS m/z: 306.1 [M+H]⁺.

### Intermediate 5

### 3-((1H-indol-5-yl)oxy)benzohydrazide

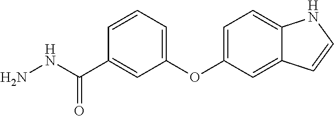 [SMILES: NNC(=O)c1cccc(Oc2ccc3[nH]ccc3c2)c1]

To a solution of ethyl 3-((1-(tert-butyldimethylsilyl)-1H-indol-5-yl)oxy)benzoate (Intermediate 5C) (1.0 g, 2.53 mmol) in MeOH (10 mL) was added hydrazine hydrate (4 mL). The mixture was stirred at 70° C. for 2 h. The mixture was concentrated in vacuo, and water (20 mL) was added. The aqueous layer was extracted with ethyl acetate (30 mL×3), and the combined organic phase was washed with brine (30 mL), dried with anhydrous sodium sulfate, filtered, and concentrated in vacuo to give 3-((1H-indol-5-yl)oxy)benzohydrazide (642 mg, 95%) as a yellow solid. MS m/z: 268.0 (M+H)⁺.

### Intermediate 5A

### 5-Bromo-1-(tert-butyldimethylsilyl)-1H-indole

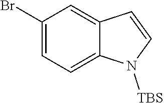

To a stirred solution of 5-bromoindole (10.0 g, 51.5 mmol) in THF (100 mL) was added LiHMDS (62 mL, 1N in THF, 62.0 mmol) at −78° C. The resulting mixture was stirred at ~78° C. for 0.5 h, and then added tert-butyldimethylsilyl chloride (13.1 g, 87.6 mmol) at −78° C. The resulting reaction mixture was stirred at −78° C. for 3 h, quenched with aq. sat. NH₄Cl (200 mL). The aqueous layer was extracted with ethyl acetate (100 mL×3). The combined organic layer was washed with brine, dried over anhydrous sodium sulfate, filtered and concentrated in vacuo. The crude product was purified by silica gel column chromatography (eluting with 10/1-8/1 petroleum ether/ethyl acetate) to afford 5-bromo-1-(tert-butyldimethylsilyl)-1H-indole (14.0 g, 89%) as yellow oil. MS m/z: 309.8 (M+H⁺).

### Intermediate 5B

### (1-(Tert-Butyldimethylsilyl)-1H-indol-5-yl)boronic acid

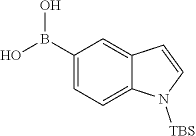

To a stirred solution of 5-bromo-1-(tert-butyldimethylsilyl)-1H-indole (14.0 g, 1.26 mmol) in anhydrous THE (100 ml) was added n-BuLi (0.8 ml, 2.4 N in hexane, 1.89 mmol) over 30 min at −78° C. The resulting mixture was stirred at −78° C. for 1 h, and then (i-PrO)₃B (412 mg, 1.89 mmol) was added dropwise at −78° C. The reaction mixture was stirred at −78° C. for 3 h, and then quenched with aq. Sat. NH₄Cl (200 mL). The aqueous phase was extracted with ethyl acetate (30 mL×3). The combined organic phase was washed with brine, dried over anhydrous sodium sulfate, filtered and concentrated in vacuo. The crude product was purified by silica gel column chromatography (eluting with petroleum ether/ethyl acetate=10/1) to give (1-(tert-butyldimethylsilyl)-1H-indol-5-yl)boronic acid (350 mg, 70%) as a light-yellow solid.

### Intermediate 5C

### Ethyl 3-((1-(tert-butyldimethylsilyl)-1H-indol-5-yl)oxy)benzoate

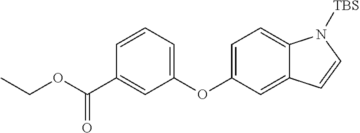

A mixture of (1-(tert-butyldimethylsilyl)-1H-indol-5-yl)boronic acid (8.6 g, 31.3 mmol), (6.6 g, 62.5 mmol), Et₃N (18.9 g, 187.6 mmol), and copper acetate (7.4 g, 40.6 mmol) in dichloromethane (340 mL) was stirred at room temperature for 12 h. The resulting mixture was filtered through celite and concentrated in vacuo, and the residue was dissolved in ethyl acetate (500 mL). The organic phase was washed with brine (200 mL), dried over anhydrous sodium sulfate, filtered and concentrated in vacuo. The residue was purified by silica gel column chromatography (eluting with petroleum ether/ethyl acetate=10/1) to give ethyl 3-((1-(tert-butyldimethylsilyl)-1H-indol-5-yl)oxy)benzoate (1.2 g, 10%, LCMS Purity: 84%) as a yellow solid. MS m/z: 396.2 (M+H⁺).

### Intermediate 6

### 2-(3-((1H-Indol-5-yl)oxy)phenyl)-1H-imidazole-5-carbaldehyde

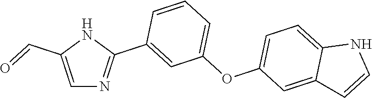 [SMILES: O=Cc1cnc(-c2cccc(Oc3ccc4[nH]ccc4c3)c2)[nH]1]

To a stirring solution of (2-(3-((1H-indol-5-yl)oxy)phenyl)-1H-imidazol-5-yl)methanol (Intermediate 6E) (300 mg, 0.98 mmol) in DCM (10 mL) was added MnO₂ (856 mg, 9.8 mmol). The resulting mixture was stirred at room temperature for 4 h, filtered through a pad of celite. The filtrate was concentrated in vacuo. The residue was purified by silica gel column chromatography (eluting with dichloromethane/methanol=50/1) to afford 2-(3-((1H-indol-5-yl)oxy)phenyl)-1H-imidazole-5-carbaldehyde (230 mg, 77%) as a yellow solid. MS m/z: 304.3 (M+H⁺).

### Intermediate 6A

### 3-(3-Methyl-4-nitrophenoxy)benzonitrile

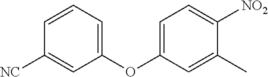 [SMILES: Cc1cc(Oc2cccc(C#N)c2)ccc1[N+](=O)[O-]]

To a stirred solution of 3-hydroxybenzonitrile (1.0 g, 8.4 mmol) and K₂CO₃ (3.5 g, 25.2 mmol) in DMF (20 mL) was added 4-fluoro-2-methyl-1-nitrobenzene (1.3 g, 8.4 mmol). The resulting mixture was stirred at 100° C. for 6 h, cooled to room temperature, quenched with water (60 mL), and extracted with ethyl acetate (60 mL×3). The combined organic phase was washed with brine (60 mL), dried over anhydrous sodium sulfate, filtered, and concentrated in vacuo. The residue was purified by silica gel column chromatography (eluting with petroleum ether/ethyl acetate=10/1) to give 3-(3-methyl-4-nitrophenoxy)benzonitrile (1.6 g, 76%) as a white solid. MS m/z: 255.2 (M+H⁺).

### Intermediate 6B

### E-3-(3-(2-(Dimethylamino)vinyl)-4-nitrophenoxy)benzonitrile

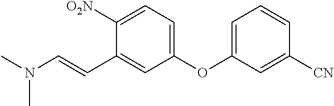 [SMILES: CN(C)/C=C/c1cc(Oc2cccc(C#N)c2)ccc1[N+](=O)[O-]]

To a stirred solution of 3-(3-methyl-4-nitrophenoxy)benzonitrile (1.6 g, 6.3 mmol) in DMF (20 mL) was added DMF-DMA (3.0 g, 25.2 mmol). The resulting mixture was stirred at 130° C. overnight, and then concentrated in vacuo to afford E-3-(3-(2-(dimethylamino)vinyl)-4-nitrophenoxy)benzonitrile (1.95 g, crude) as yellow oil, which was used directly for the next step. MS m/z: 310.2 (M+H⁺).

### Intermediate 6C

### 3-((1H-Indol-5-yl)oxy)benzonitrile

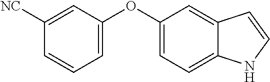 [SMILES: N#Cc1cccc(Oc2ccc3[nH]ccc3c2)c1]

To a stirred solution of E-3-(3-(2-(dimethylamino)vinyl)-4-nitrophenoxy)benzonitrile (1.95 g, 6.3 mmol) in acetic acid (20 mL, 80%) was added Zn (4.1 g, 63.0 mmol). The resulting mixture was stirred at 80° C. overnight, cooled to room temperature, filtered through a pad of celite and concentrated in vacuo. The residue was dissolved in ethyl acetate (100 ml), the organic phase was washed with water (50 mL×3), brine (50 mL), dried over anhydrous sodium sulfate, filtered and concentrated in vacuo. The residue was purified by silica gel column chromatography (eluting with petroleum ether/ethyl acetate=5/1) to give 3-((1H-indol-5-yl)oxy)benzonitrile (480 mg, 33% of 2 steps) as a yellow solid. MS m/z: 235.2 (M+H⁺).

### Intermediate 6D

### 3-((1H-Indol-5-yl)oxy)benzimidamide

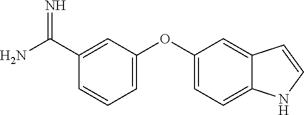 [SMILES: N=C(N)c1cccc(Oc2ccc3[nH]ccc3c2)c1]

To a stirring solution of 3-((1H-indol-5-yl)oxy)benzonitrile (480 mg, 2.05 mmol) in THE (10 mL) was added LiHMDS (8.2 mL, 1.0 N in THF, 8.2 mmol) at room temperature. The resulting mixture was stirred at room temperature for 6 h and quenched with aq. Sat. NH₄Cl (10 mL). The organic layer was separated, and then the aqueous phase was extracted by DCM (15 mL×6). The combined organic layers were washed with brine (30 mL), dried over anhydrous sodium sulfate, filtered, and concentrated in vacuo to give 3-((1H-indol-5-yl)oxy)benzimidamide (400 mg, 78%) as a yellow solid, which was used directly for the next step. MS m/z: 252.3 (M+H⁺).

### Intermediate 6E

### (2-(3-((1H-Indol-5-yl)oxy)phenyl)-1H-imidazol-5-yl)methanol

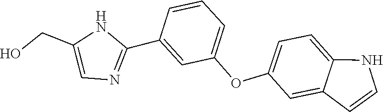 [SMILES: OCc1cnc(-c2cccc(Oc3ccc4[nH]ccc4c3)c2)[nH]1]

To a stirring solution of 3-((1H-indol-5-yl)oxy)benzimidamide (400 mg, 1.59 mmol) in concentrated aqueous ammonia (10 mL) was added 1,3-dihydroxypropan-2-one (143 mg, 1.59 mmol) and NH₄Cl (341 mg, 6.37 mmol) at room temperature. The resulting mixture was stirred at 80° C. for 4 h, cooled to room temperature, and extracted with DCM (15 mL×6). The combined organic layers were washed with brine (30 mL), dried over anhydrous sodium sulfate, filtered, and concentrated in vacuo to give (2-(3-((1H-indol-5-yl)oxy)phenyl)-1H-imidazol-5-yl)methanol (300 mg, 62%) as a yellow solid, which was used directly for the next step. MS m/z: 306.3 (M+H⁺).

### Intermediate 7

### 2-(3-((1H-Benzo[d]pyridine-5-yl)oxy)phenyl)-1H-imidazole-5-carbaldehyde

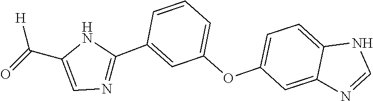 [SMILES: O=Cc1cnc(-c2cccc(Oc3ccc4[nH]cnc4c3)c2)[nH]1]

A mixture of (2-(3-((1H-benzo[d]pyridine-5-yl)oxy)phenyl)-1H-imidazol-4-yl)methanol (500 mg, 1.63 mmol) and 2-iodoxybenzoic acid (1.37 g, 4.89 mmol) in DMSO (5 mL) was stirred at room temperature for 5 h. The reaction mixture was quenched with water (10 mL), and the aqueous phase was extracted with ethyl acetate (20 mL×3). The combined organic phase was washed with brine (30 mL), dried over anhydrous sodium sulfate, filtered, and concentrated in vacuo to give 2-(3-((1H-benzo[d]pyridine-5-yl)oxy)phenyl)-1H-imidazole-5-carbaldehyde (320 mg, 65%) as a yellow solid, which was used directly for the next step. MS: m/z: 305.2 (M+H)⁺.

### Intermediate 7A

### 3-(3-Amino-4-nitrophenoxy)benzonitrile

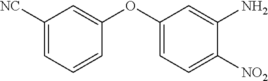 [SMILES: N#Cc1cccc(Oc2ccc([N+](=O)[O-])c(N)c2)c1]

To a suspension of 5-fluoro-2-nitrobenzenamine (10.0 g, 64.1 mmol) and K₂CO₃ (17.7 g 128.2 mmol) in DMF (100 mL) was added 3-hydroxybenzonitrile (8.40 g 70.5 mmol). The resulting mixture was stirred at 100° C. for 3 h, and then quenched with water (200 mL). The formed precipitation was filtered, and washed with water (100 ml×2) to give 3-(3-amino-4-nitrophenoxy)benzonitrile (15.0 g, 94%) as a yellow solid, which was used directly for the next step. MS m/z: 256.0 (M+H⁺).

### Intermediate 7B

### 3-(3,4-Diaminophenoxy)benzonitrile

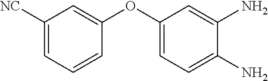 [SMILES: N#Cc1cccc(Oc2ccc(N)c(N)c2)c1]

To a suspension of 3-(3-amino-4-nitrophenoxy)benzonitrile (10.0 g, 39.2 mmol) in ethyl acetate (200 mL) was added the Pd/C (1.00 g, 10% Pd in carbon). The mixture was stirred at room temperature overnight under H₂ atmosphere, filtered through a pad of celite, and washed with ethyl acetate (20 mL×3). The filtration was concentrated in vacuo to give 3-(3,4-diaminophenoxy)benzonitrile (9.05 g, crude) as yellow oil, which was used directly for the next step. MS m/z: 226.2 (M+H⁺).

### Intermediate 7C

### 3-((1H-Benzo[d]pyridine-5-yl)oxy)benzonitrile

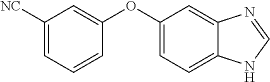 [SMILES: N#Cc1cccc(Oc2ccc3[nH]cnc3c2)c1]

To a solution of 3-(3,4-diaminophenoxy)benzonitrile (9.05 g, 40.2 mmol) in EtOH (100 mL) was added TsOH (1.38 g, 8.04 mmol) and CH(OEt)₃ (20.1 mL, 120.6 mmol). The resulting mixture was refluxed for 3 h, and then concentrated in vacuo. The residue was purified by silica gel column chromatography (eluting with dichloromethane/methanol=40/1-20/1) to give 3-((1H-benzo[d]pyridine-5-yl)oxy)benzonitrile (8.36 g, 88% of 2 steps) as yellow solid. MS: m/z: 236.1 (M+H)⁺.

### Intermediate 7D

### 3-((1H-benzo[d]imidazol-5-yl)oxy)benzimidamide

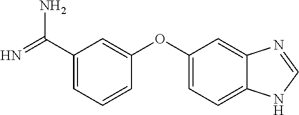 [SMILES: N=C(N)c1cccc(Oc2ccc3[nH]cnc3c2)c1]

To a solution of 3-((1H-benzo[d]pyridine-5-yl)oxy)benzonitrile (4.01 g, 17.0 mmol) in anhydrous tetrahydrofuran (40 mL) was added a solution of LiHMDS (68.0 mL, 1.0 M in THF) at 0° cover 30 minutes under N₂ atmosphere. The reaction mixture was stirred at room temperature overnight. The reaction mixture was quenched with sat. aq. NH₄Cl (100 mL), the organic phase was concentrated in vacuo. The formed precipitation was filtered, and washed with water (30 mL×2) to give 3-((1H-benzo[d]pyridine-5-yl)oxy)benzimidamide (3.62 g, 84%) as yellow solid, which was used directly for the next step. MS: m/z: 253.2 (M+H⁺).

### Intermediate 7E

### (2-(3-((1H-benzo[d]pyridine-5-yl)oxy)phenyl)-1H-imidazol-4-yl)methanol

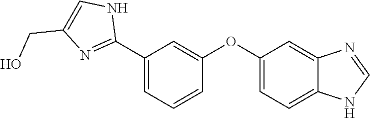 [SMILES: OCc1c[nH]c(-c2cccc(Oc3ccc4[nH]cnc4c3)c2)n1]

To a mixture of 3-((1H-benzo[d]pyridine-5-yl)oxy)benzimidamide (5.00 g, 20.0 mmol), 1,3-dihydroxypropan-2-one (5.00 g, 55.6 mmol) in NH₄OH (50 mL) was added NH₄Cl (5.00 g, 100 mmol). The resulting mixture was heated to 80° C. for 3 h, and then diluted with water (100 mL) and extracted with ethyl acetate (100 mL×3). The combined organic phase was washed with brine (100 mL), dried over anhydrous sodium sulfate, filtered, and concentrated in vacuo. The residue was purified by silica gel column chromatography (eluting with dichloromethane/methanol=30/1-10/1) to give (2-(3-((1H-benzo[d]pyridine-5-yl)oxy)phenyl)-1H-imidazol-4-yl)methanol (2.40 g, 40%) as an orange solid. MS m/z: 307.1 (M+H)⁺. ¹H NMR (500 MHz, MeOD-d₄): d 8.13 (s, 1H), 7.58 (d, J=9.0 Hz, 1H), 7.55-7.52 (m, 1H), 7.41 (dd, J=2.5 Hz & 2.5 Hz, 1H), 7.37 (dd, J=7.5 Hz & 7.5 Hz, 1H), 7.22 (d, J=2.0 Hz, 1H), 7.01 (dd, J=9.0 Hz & 2.5 Hz, 1H), 6.99 (s, 1H), 6.97-6.63 (m, 1H), 4.52 (s, 2H) ppm.

### Intermediate 8

### Ethyl 3-((1-tosyl-1H-indol-5-yl)oxy)benzimidate

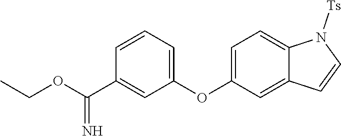 [SMILES: CCOC(=N)c1cccc(Oc2ccc3c(ccn3S(=O)(=O)c3ccc(C)cc3)c2)c1]

To a stirred solution of 3-((1-tosyl-1H-indol-5-yl)oxy)benzonitrile (240 mg, 0.61 mmol) in ethanol (4.0 mL) was added sulfurous dichloride (3.0 mL) dropwise at 0° C. under nitrogen atmosphere. The resulting solution was stirred at 0° C. for 10 minutes. Then the reaction mixture was stirred at room temperature overnight. After reaction, the solution was evaporated to give the crude product ethyl 3-((1-tosyl-1H-indol-5-yl)oxy)benzimidate (250 mg, purity: 76.8%) as a yellow solid. MS m/z: 435.1 (M+H⁺)

### Intermediate 8A

### 3-((1-Tosyl-1H-indol-5-yl)oxy)benzonitrile

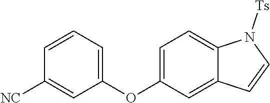 [SMILES: Cc1ccc(S(=O)(=O)n2ccc3cc(Oc4cccc(C#N)c4)ccc32)cc1]

To a stirred solution of 3-((1H-indol-5-yl)oxy)benzonitrile (Intermediate 6C) (234 mg, 1.0 mmol) in anhydrous acetone (10 mL) was added potassium hydroxide (112 mg, 2.0 mmol). After ten minutes, 4-methylbenzene-1-sulfonyl chloride (380 mg, 2.0 mmol) was added into the mixture and the resulting mixture was stirred at room temperature for 3.0 hours. The reaction was monitored by TLC. All of volatiles were removed under reduced pressure and the residue obtained was purified by silica gel column chromatography (PE:EA=10:1 to 5:1) to give 3-((1-tosyl-1H-indol-5-yl)oxy)benzonitrile (300 mg, 77%) as yellow solid. MS m/z: 389.0 (M+H⁺)

### Intermediate 9

### Ethyl 3-((1H-benzo[d]imidazol-5-yl)oxy)benzimidate

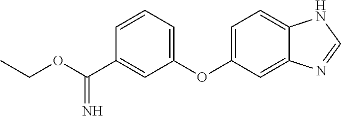 [SMILES: CCOC(=N)c1cccc(Oc2ccc3[nH]cnc3c2)c1]

Acetyl chloride (3.0 mL) was added dropwise into a stirred solution of compound 3-((1H-benzo[d]pyridine-5-yl)oxy)benzonitrile (Intermediate 7C) (235 mg, 1.0 mmol) in ethanol (5.0 mL) at 0° C. under nitrogen atmosphere. The solution was stirred at 0° C. for 10 mins. Then the reaction mixture was stirred at room temperature overnight. After completion of reaction, the solution was evaporated to give the crude product ethyl 3-((1H-benzo[d]pyridine-5-yl)oxy)benzimidate (350 mg, purity: 45.0%) as a yellow solid which was used directly for the next step directly. MS-ESI: [M+H]⁺ 282.3

### Intermediate 10

### 3-((1H-Pyrrolo[3,2-b]pyridine-5-yl)oxy)benzonitrile

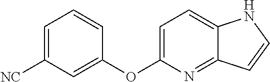 [SMILES: N#Cc1cccc(Oc2ccc3[nH]ccc3n2)c1]

To a stirred solution of 3-((6-methyl-5-nitropyridin-2-yl)oxy)benzonitrile (5.1 g, 20.0 mmol) in DMF (25 mL) was added DMF-DMA (19.0 g, 160.0 mmol), the resulting mixture was stirred at 130° C. for 1 hour under N₂ atmosphere. The mixture was concentrated in vacuo. The residue was dissolved in ethyl acetate (25 mL), and Pd/C (700 mg) was added. The resulting mixture was stirred at 50° C. overnight under hydrogen balloon, and then filtered through a pad of celite. The filtrate was concentrated in vacuo. The residue was purified by silica gel column chromatography (eluting with 3/2/1 petroleum ether/ethyl acetate/dichloromethane) to afford 3-((1H-pyrrolo[3,2-b]pyridine-5-yl)oxy)benzonitrile (2.6 g, 55%) as a yellow solid. MS m/z: 236.2 (M+H⁺).

### Intermediate 10A

### 3-((6-Methyl-5-nitropyridin-2-yl)oxy)benzonitrile

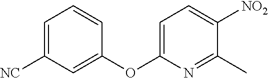 [SMILES: Cc1nc(Oc2cccc(C#N)c2)ccc1[N+](=O)[O-]]

A mixture of 6-chloro-2-methyl-3-nitropyridine (5.0 g, 29.1 mmol), 3-hydroxybenzonitrile (3.8 g, 31.9 mmol) and K₂CO₃ (8.0 g, 58.1 mmol) in DMF (25 mL) was stirred at 100° C. for 1 hour under N₂ atmosphere. The mixture was concentrated in vacuo, and ethyl acetate (100 mL) was added. The organic layer was washed with water (50 mL) and brine (50 mL), dried over anhydrous sodium sulfate, filtered, and concentrated in vacuo. The residue was purified by silica gel column chromatography (eluting with dichloromethane/methanol=50/1) to get 3-((6-methyl-5-nitropyridin-2-yl)oxy)benzonitrile (7.1 g, 96%) as a red solid. MS m/z: 256.2 (M+H⁺).

### Intermediate 11

### Methyl 5-(3-(ethoxy(imino)methyl)phenoxy)-1H-indole-4-carboxylate

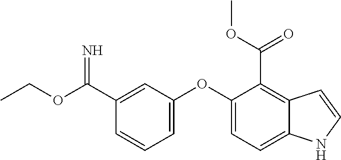 [SMILES: CCOC(=N)c1cccc(Oc2ccc3[nH]ccc3c2C(=O)OC)c1]

To a stirred solution of methyl 5-(3-cyanophenoxy)-1H-indole-4-carboxylate (1.2 g, 4.0 mmol) in ethanol (10 mL) was added AcCl (20.0 ml) at 0° C. The resulting mixture was allowed to stir at 25° C. overnight. The mixture was concentrated in vacuo to give crude methyl 5-(3-(ethoxy(imino)methyl)phenoxy)-1H-indole-4-carboxylate (1.3 g) as a yellow solid, which was used for next step directly. MS m/z: 339.1 (M+H⁺).

### Intermediate 11A

### 6-fluoro-2-methyl-3-nitrobenzoic acid

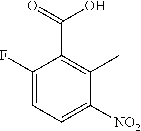

To a mixture of 2-fluoro-6-methylbenzoic acid (1.0 eq, 6.5 mmol) in concentrated H₂SO₄ (10 mL) was added KNO₃ (6.6 g, 6.5 mmol) at 0° C. The resulting mixture was stirred at 0° C. for two hours, then poured into ice-water and the formed precipitated was collected by suction and dried under reduced pressure to give 6-fluoro-2-methyl-3-nitrobenzoic acid (1.1 g, 84%) as a yellow solid, which was used for the next step directly.

### Intermediate 11B

### Methyl 6-fluoro-2-methyl-3-nitrobenzoate

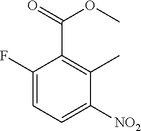

To a stirred solution of 6-fluoro-2-methyl-3-nitrobenzoic acid (1.0 g, 5.0 mmol) in DMF (10 mL) was added triethylamine (0.8 mL, 20.0 mmol), HATU (2.1 g, 5.5 mmol) and MeOH (0.4 mL, 10.0 mmol) at room temperature. The resulting mixture was stirred at room temperature overnight, then quenched with water (20 mL), and extracted with ethyl acetate (10 mL×3), the combined organic phase was washed with water (10 mL×3), brine (10 mL×2), dried over anhydrous Na₂SO₄, filtered, and concentrated in vacuo. The residue was purified by silica gel column chromatography (eluting with petroleum ether/ethyl acetate=10/1) to give methyl 6-fluoro-2-methyl-3-nitrobenzoate (890 mg, 83%) as a yellow solid. ¹H NMR (400 MHz, CDCl₃) δ 8.03-8.0 (m, 1H), 7.15-7.11 (m, 1H), 4.00 (s, 3H), 2.55 (s, 3H) ppm.

### Intermediate 11C

### Methyl 6-(3-cyanophenoxy)-2-methyl-3-nitrobenzoate

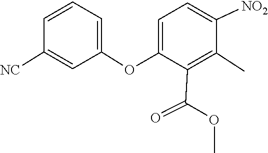 [SMILES: COC(=O)c1c(Oc2cccc(C#N)c2)ccc([N+](=O)[O-])c1C]

To a stirred solution of methyl 6-fluoro-2-methyl-3-nitrobenzoate (5.3 g, 24.9 mmol) in DMF (50 mL) was added 3-hydroxybenzonitrile (3.3 g, 27.4 mmol) and K₂CO₃ (6.9 g, 49.8 mmol). The resulting mixture was stirred at 80° C. overnight, then quenched with water (100 mL), and extracted with ethyl acetate (50 mL×3). The combined organic phase was washed with brine (50 mL×3), dried over anhydrous Na₂SO₄, filtered and concentrated in vacuo. The residue was purified by silica gel column chromatography (eluting with petroleum ether/ethyl acetate=10/1) to give methyl 6-(3-cyanophenoxy)-2-methyl-3-nitrobenzoate (7.3 g, 94%) as a yellow solid.

### Intermediate 11D

### Methyl 5-(3-cyanophenoxy)-1H-indole-4-carboxylate

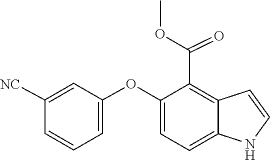 [SMILES: COC(=O)c1c(Oc2cccc(C#N)c2)ccc2[nH]ccc12]

A mixture of methyl 6-(3-cyanophenoxy)-2-methyl-3-nitrobenzoate (3.0 g, 9.6 mmol) and DMF-DMA (10.2 mL, 76.8 mmol) in DMF (20 mL) was heated to 120° C. for 1 h, then concentrated in vacuo. The residue was dissolved in AcOH (20 mL), and Zn powder (6.2 g, 96.0 mmol) was added. The reaction mixture was stirred at 80° C. overnight, then filtered through a pad of Celite. The filtrate was concentrated in vacuo. The residue was dissolved in ethyl acetate (50 mL), washed with brine (20 mL×3), dried over sodium sulfate, and concentrated in vacuo. The residue was purified by silica gel column chromatography (eluting with 1/1 petroleum ether/ethyl acetate) to give methyl 5-(3-cyanophenoxy)-1H-indole-4-carboxylate (2.4 g, 86%) as a yellow solid. MS m/z: 293.1 (M+H⁺).

### Intermediate 12

### 5-((4,6-Difluoro-1H-indol-5-yl)oxy)-2-fluorobenzoic acid

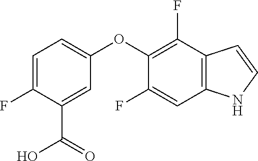 [SMILES: O=C(O)c1cc(Oc2c(F)cc3[nH]ccc3c2F)ccc1F]

To a solution of 5-[(4,6-difluoro-1H-indol-5-yl)oxy]-2-fluoro-benzonitrile (Intermediate 2, 1.00 eq, 10.00 g, 34.7 mmol) in ethanol (80 mL) and water (80 mL) was added potassium hydroxide (5.00 eq, 0.97 g, 173 mmol). The reaction mixture was stirred overnight at 90° C., cooled to room temperature, diluted with 200 mL of water, acidified with hydrochloric acid to pH=5, and extracted with ethyl acetate (300 mL×3). The combined organic extracts were washed with brine, dried over Na₂SO₄ and concentrated to give 5-[(4,6-difluoro-1H-indol-5-yl)oxy]-2-fluoro-benzoic acid (9.60 g, 31.2 mmol, 90.1%) as a yellow solid. MS (ESI) m/z: 308.0 (M+H⁺).

### Intermediate 13

### 4,6-difluoro-5-(4-fluoro-3-hydrazineylphenoxy)-1-tosyl-1H-indole

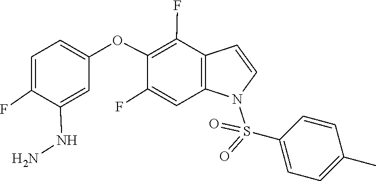 [SMILES: Cc1ccc(S(=O)(=O)n2ccc3c(F)c(Oc4ccc(F)c(NN)c4)c(F)cc32)cc1]

To a solution of 5-[4,6-difluoro-1-(p-tolylsulfonyl)indol-5-yl]oxy-2-fluoro-aniline (Intermediate 13D, 3.00 g, 6.94 mmol) in acetic acid (30 mL) and 12N aqueous HCl (60 mL) was added dropwise at 0° C. a solution of sodium nitrite (1.00 eq, 479 mg, 6.94 mmol) in water (5 mL). The mixture was stirred for 1 hour at 0° C. and stannous chloride dihydrate (3.92 g) in 12N aqueous HCl (30 mL) was added dropwise. The reaction mixture was stirred at room temperature for 1 hour. A large amount of precipitate formed. The precipitate was isolated by filtration, and the solid was washed twice with water, and dried at 30° C. overnight to give the product, [5-[4,6-difluoro-1-(p-tolylsulfonyl)indol-5-yl]oxy-2-fluoro-phenyl]hydrazine hydrochloride salt (2.80 g, 5.95 mmol, 88%) as a yellow solid. MS (ESI) m/z: 448 (M+H⁺).

### Intermediate 13A

### 5-(2,6-difluoro-3-methyl-4-nitrophenoxy)-2-fluoroaniline

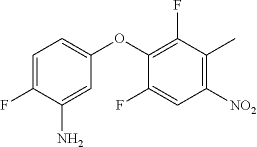 [SMILES: Cc1c([N+](=O)[O-])cc(F)c(Oc2ccc(F)c(N)c2)c1F]

To a stirred solution of 1,2,3-trifluoro-4-methyl-5-nitro-benzene (1.00 eq, 13.23 g, 69.2 mmol) and 3-amino-4-fluoro-phenol (1.00 eq, 8.80 g, 69.2 mmol) in DMF (150 mL) was added potassium carbonate (2.00 eq, 19.14 g, 138 mmol). The resulting mixture was stirred for 16 hours at room temperature and then diluted with water (1000 mL). The aqueous layer was extracted with ethyl acetate (500 mL×2). The organic extracts were combined, washed successively with water, aqueous sodium sulfite and brine, dried over anhydrous Na₂SO₄, filtered, and concentrated. The residue was purified by flash column chromatography, eluting with 0-15% ethyl acetate in petroleum ether, to give the product, 5-(2,6-difluoro-3-methyl-4-nitro-phenoxy)-2-fluoro-aniline (15.50 g, 48.9 mmol, 70.6%) as a colorless oil. ¹H NMR (400 MHz, DMSO-d₆) δ 8.19 (d, 1H), 6.93 (q, 1H), 6.25 (q, 1H), 6.05 (m, 1H), 5.32 (s, 2H), 2.56 (s, 3H) ppm.

### Intermediate 13B

### (E)-5-(3-(2-(dimethylamino)vinyl)-2,6-difluoro-4-nitrophenoxy)-2-fluoroaniline

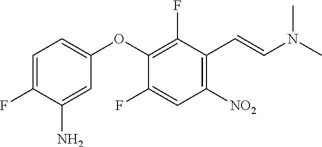 [SMILES: CN(C)/C=C/c1c([N+](=O)[O-])cc(F)c(Oc2ccc(F)c(N)c2)c1F]

A solution of DMF-DMA (5.00 eq, 19.14 g, 138 mmol) and 5-(2,6-difluoro-3-methyl-4-nitro-phenoxy)-2-fluoro-aniline (1.00 eq, 15.50 g, 52.0 mmol) in DMF (150 mL) was stirred for 16 hours at 100° C. The reaction mixture was cooled and diluted with water (200 mL). The aqueous layer was extracted with ethyl acetate (200 mL×2). The organic extracts were combined, washed successively with water, aqueous sodium sulfite and brine, dried over anhydrous Na₂SO₄, filtered, and concentrated. The residue was purified by flash column chromatography, eluting with 0-15% ethyl acetate in petroleum ether, to give the product, 5-[3-[(E)-2-(dimethylamino)vinyl]-2,6-difluoro-4-nitro-phenoxy]-2-fluoro-aniline (17.00 g, 43.3 mmol, 92.8%) as a red oil. The crude product was used directly in the next step without further characterization.

### Intermediate 13C

### 5-((4,6-difluoro-1H-indol-5-yl)oxy)-2-fluoroaniline

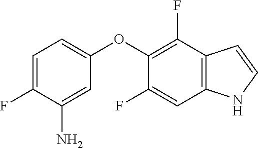 [SMILES: Nc1cc(Oc2c(F)cc3[nH]ccc3c2F)ccc1F]

A suspension of iron powder (20.0 eq, 53.80 g, 963 mmol) and 5-[3-[(E)-2-(dimethylamino)vinyl]-2,6-difluoro-4-nitro-phenoxy]-2-fluoro-aniline (1.00 eq, 17.00 g, 48.1 mmol) in acetic acid (100 ml) and toluene (50 mL) was stirred at 80° C. for 16 hours, then cooled to room temperature. Water (200 mL) was added to the reaction mixture. The mixture was extracted with ethyl acetate (200 mL×2). The organic extracts were combined, washed successively with water, aqueous sodium sulfite and brine, dried over anhydrous Na₂SO₄, filtered, and concentrated. The residue was purified by flash column chromatography on silica gel, eluting with 0-15% EtOAc in petroleum ether, to give the product, 5-[(4,6-difluoro-1H-indol-5-yl)oxy]-2-fluoro-aniline (4.00 g, 10.8 mmol, 23%) as a red oil. MS (ESI) m/z: 448 (M-F)+.

### Intermediate 13D

### 5-((4,6-difluoro-1-tosyl-1H-indol-5-yl)oxy)-2-fluoroaniline

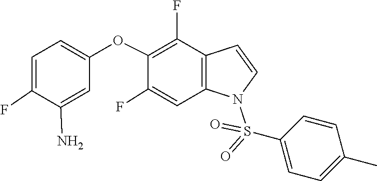 [SMILES: Cc1ccc(S(=O)(=O)n2ccc3c(F)c(Oc4ccc(F)c(N)c4)c(F)cc32)cc1]

To a solution of 5-[(4,6-difluoro-1H-indol-5-yl)oxy]-2-fluoro-aniline (1.00 eq, 1.00 g, 3.59 mmol) in anhydrous THE (10 mL) was added dropwise at −78° C. a solution of sodium bis(trimethylsilylamide) in THE (1M, 3.6 mL). The mixture was stirred at −78° C. for 1 hour and p-toluenesulfonyl chloride (1.00 eq, 0.69 g, 3.59 mmol) was added. The reaction mixture was allowed to warm to room temperature and stirred for 1 hour, then quenched through the addition of water. The aqueous mixture was extracted with ethyl acetate (20 mL×2). The organic extracts were combined, washed successively with water, aqueous sodium sulfite and brine, dried over anhydrous Na₂SO₄, filtered, and concentrated. The residue was purified by flash column chromatography on silica gel, eluting with 0-15% ethyl acetate in petroleum ether, to give the product, 5-[4,6-difluoro-1-(p-tolylsulfonyl)indol-5-yl]oxy-2-fluoro-aniline (1.10 g, 2.42 mmol, 57%) as a yellow solid. MS (ESI) m/z: 433 (M+H⁺).

### Intermediate 14

### 6-fluoro-4-methyl-5-(3-(4,4,5,5-tetramethyl-1,3,2-dioxaborolan-2-yl)phenoxy)-1H-indole

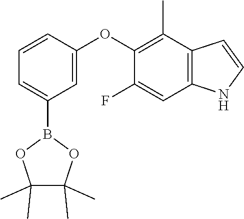 [SMILES: Cc1c(Oc2cccc(B3OC(C)(C)C(C)(C)O3)c2)c(F)cc2[nH]ccc12]

A mixture of 5-(3-bromophenoxy)-6-fluoro-4-methyl-1H-indole (Intermediate 14B, 2.50 g, 7.84 mmol), 4,4,4′,4′,5,5,5′,5′-octamethyl-2,2′-bi(1,3,2-dioxaborolane) (2.99 g, 11.8 mmol), potassium acetate (1.54 g, 15.7 mmol) and Pd(dppf)Cl₂ (639 mg, 0.78 mmol) in 1,4-dioxane (30 mL) was stirred at 90° C. overnight under a N₂ atmosphere. The reaction was cooled and quenched with water (50 mL), filtered through a pad of Celite, and the aqueous phase was extracted with EtOAc (50 mL×3). The combined organic extracts were washed with brine, dried over anhydrous Na₂SO₄, filtered and concentrated. The residue was purified by silica gel column chromatography (eluting with 3/1 petroleum ether/ethyl acetate) to afford 6-fluoro-4-methyl-5-(3-(4,4,5,5-tetramethyl-1,3,2-dioxaborolan-2-yl)phenoxy)-1H-indole (1.78 g, 62%) as a pale solid. MS(ESI): m/z 368.2 (M+H⁺).

### Intermediate 14A

### 2-(3-bromophenoxy)-1-fluoro-3,4-dimethyl-5-nitrobenzene

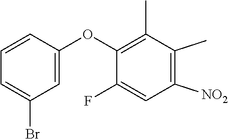 [SMILES: Cc1c([N+](=O)[O-])cc(F)c(Oc2cccc(Br)c2)c1C]

A mixture of 1,2-difluoro-3,4-dimethyl-5-nitrobenzene (6.0 g, 32.1 mmol), 3-bromophenol (5.79 g, 33.7 mmol) and K₂CO₃ (8.86 g, 64.2 mmol) in DMF (200 mL) was stirred at 100° C. for 1 hour. The resulting mixture was filtered through Celite, and the filtrate was concentrated. The residue was dissolved in ethyl acetate (200 mL), the organic phase was washed with water (100 mL×2), brine (50 mL), dried with anhydrous sodium sulfate, filtered and concentrated in vacuo to give a residue. The residue was purified by silica gel column chromatography (eluting with 3/1 petroleum ether/ethyl acetate) to afford 2-(3-bromophenoxy)-1-fluoro-3,4-dimethyl-5-nitrobenzene (10 g, 91%) as a yellow solid. MS(ESI): m/z 340.2, 342.2 (M+H⁺).

### Intermediate 14B

### 5-(3-bromophenoxy)-6-fluoro-4-methyl-1H-indole

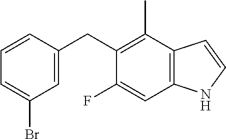 [SMILES: Cc1c(Cc2cccc(Br)c2)c(F)cc2[nH]ccc12]

A mixture of 2-(3-bromophenoxy)-1-fluoro-3,4-dimethyl-5-nitrobenzene (6.8 g, 20.1 mmol), N,N-dimethylformamide dimethyl acetal (11.9 g, 0.10 mol) and pyrrolidine (2.85 g, 40.1 mmol) was stirred for 4 hours at 100° C. The mixture was concentrated in vacuo and water (100 mL) was added. The resulting mixture was extracted with ethyl acetate (80 mL×3). The combined organic extracts were concentrated in vacuo, and the residue was dissolved in acetic acid/toluene (5/3, 100 mL). To the resulting solution was added iron powder (11.2 g, 0.20 mol), and the mixture was stirred overnight at 100° C. The mixture was filtered through Celite, and the filtrate was concentrated in vacuo to give a residue. The residue was purified by silica gel column chromatography (eluting with 3/1 petroleum ether/ethyl acetate) to afford 5-(3-bromophenoxy)-6-fluoro-4-methyl-1H-indole (4.19 g, 65%) as a yellow solid. MS (ESI): m/z 320.2, 322.2 (M+H+).

### Intermediate 15

### 5-(3-bromo-4-fluoro-phenoxy)-4,6-difluoro-1H-indole

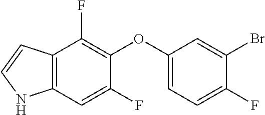 [SMILES: Fc1ccc(Oc2c(F)cc3[nH]ccc3c2F)cc1Br]

To a stirred solution of 4-(3-bromo-4-fluoro-phenoxy)-3,5-difluoro-2-(2-trimethylsilylethynyl) aniline (Intermediate 15D) (1.00 eq, 24.00 g, 57.9 mmol) in DMF (260 mL) was added copper (I) iodide (2.00 eq, 22.07 g, 116 mmol) at room temperature. The mixture was heated at 120° C. for 16 h, cooled to room temperature, filtered through a Celite pad, and the pad washed with EtOAc (200 mL). The organic filtrate was washed with water (300 mL), brine (100 mL), dried over anhydrous Na₂SO₄, filtered and concentrated. The crude was purified by flash column chromatography, eluting with 10% EtOAc in hexanes, to afford 5-(3-bromo-4-fluoro-phenoxy)-4,6-difluoro-1H-indole (12.00 g, 35.1 mmol, 61%). ¹H NMR (300 MHz, DMSO-d6) δ 7.48 (dd, 1H), 7.38-7.28 (m, 3H), 6.98 (dt, 1H), 6.59-6.57 (m, 1H) ppm.

### Intermediate 15A

### 2-(3-bromo-4-fluoro-phenoxy)-1,3-difluoro-5-nitro-benzene

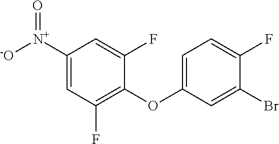

To a solution of 1,2,3-trifluoro-5-nitro-benzene (1.00 eq, 10.00 g, 56.5 mmol) in DMF (80 mL) was added 3-bromo-4-fluoro-phenol (1.10 eq, 11.86 g, 62.1 mmol) and K₂CO₃ (2.00 eq, 15.59 g, 113 mmol) at rt. The reaction was stirred at rt for 2 h, quenched with ice-cold water (500 mL), and extracted with EtOAc (2×300 mL). The combined organic extracts were washed with brine (100 mL), dried over anhydrous Na₂SO₄, filtered, and concentrated under reduced pressure to give 2-(3-bromo-4-fluoro-phenoxy)-1,3-difluoro-5-nitro-benzene (19.50 g, 56.0 mmol, 99% yield) as an off-white solid. ¹H NMR (300 MHz, CDCl₃) δ 8.01-7.95 (m, 2H), 7.18 (dd, 1H), 7.11 (dd, 1H), 6.93 (dt, 1H) Intermediate 15B

### 4-(3-bromo-4-fluoro-phenoxy)-3,5-difluoro-aniline

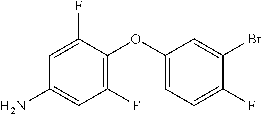 [SMILES: Nc1cc(F)c(Oc2ccc(F)c(Br)c2)c(F)c1]

To a stirred solution of 2-(3-bromo-4-fluoro-phenoxy)-1,3-difluoro-5-nitro-benzene (1.00 eq, 19.50 g, 56.0 mmol) in ethanol (195 mL) was added ammonium chloride (8.00 eq, 23.90 g, 447 mmol) in water (80 mL) at rt, followed by portionwise addition of iron powder (4.00 eq, 12.50 g, 224 mmol). The reaction mixture was heated at 80° C. for 2 h, cooled to rt, filtered through a Celite bed, and washed with EtOAc (100 mL). The filtrate was washed with water (500 mL), brine (200 mL), dried over anhydrous Na₂SO₄, filtered, and concentrated under reduced pressure to give 4-(3-bromo-4-fluoro-phenoxy)-3,5-difluoro-aniline (18.00 g, 56.6 mmol, quantitive yield) as a thick syrup. MS (ESI): m/z 318, 320 (M+H⁺).

### Intermediate 15C

### 4-(3-bromo-4-fluoro-phenoxy)-3,5-difluoro-2-iodo-aniline

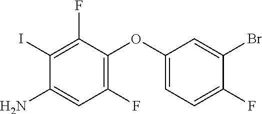 [SMILES: Nc1cc(F)c(Oc2ccc(F)c(Br)c2)c(F)c1I]

To a stirred solution of 4-(3-bromo-4-fluoro-phenoxy)-3,5-difluoro-aniline (1.00 eq, 49.00 g, 154 mmol) in acetic acid (490 mL) was added at 10-15° C. NIS (1.00 eq, 34.50 g, 154 mmol) portion wise. The reaction mass was allowed to warm up to room temperature and stirred for 1 h. Reaction progress was monitored by TLC and LC-MS. Upon reaction completion, the volatiles were removed under reduced pressure. The residue was suspended in saturated aqueous NaHCO₃ (500 mL) and extracted with ethyl acetate (500 mL×3). The organic phase was washed with brine (200 mL), dried over anhydrous Na₂SO₄, filtered and concentrated. The crude product was purified by flash column chromatography, eluting with 3% ethyl acetate in hexanes, to obtained 4-(3-bromo-4-fluoro-phenoxy)-3,5-difluoro-2-iodo-aniline (48.00 g, 108 mmol, 53% yield) as an off white solid. ¹H NMR (300 MHz, CDCl₃) δ 7.11-7.07 (m, 1H), 7.03 (d, 1H), 6.89-6.84 (m, 1H), 6.47 (dd, 1H), 4.36 (bs, 2H)

### Intermediate 15D

### 4-(3-bromo-4-fluoro-phenoxy)-3,5-difluoro-2-(2-trimethylsilylethynyl)aniline

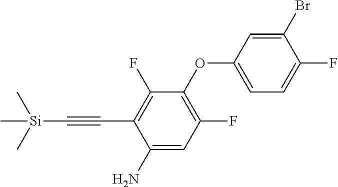 [SMILES: C[Si](C)(C)C#Cc1c(N)cc(F)c(Oc2ccc(F)c(Br)c2)c1F]

To a stirred and argon degassed solution of 4-(3-bromo-4-fluoro-phenoxy)-3,5-difluoro-2-iodo-aniline (1.00 eq, 46.00 g, 104 mmol) in DMF (120 mL) was added trimethylsilylacetylene (1.50 eq, 15.26 g, 155 mmol) followed by CuI (0.200 eq, 3.95 g, 20.7 mmol) and TEA (5.00 eq, 52.42 g, 518 mmol). The mixture was sparged further with argon for 10 minutes, and Pd(dppf)Cl₂ (0.100 eq, 7.58 g, 10.4 mmol) was added. The reaction mass was stirred at rt for 40 h, then quenched with water (400 mL) and EtOAc (400 mL). The mixture was filtered through a pad of Celite. The pad was washed with EtOAc (100 mL). The separated organic portion of the filtrate was washed with brine (200 mL), dried over anhydrous Na₂SO₄, filtered, and concentrated. The crude was purified by flash column chromatography on silica gel, eluting with 5% EtOAc in hexanes, to give 4-(3-bromo-4-fluoro-phenoxy)-3,5-difluoro-2-(2-trimethylsilylethynyl)aniline (25.00 g, 60.3 mmol, 58.24% yield) as a thick syrup. ¹NMR (300 MHz, DMSO-d₆) δ 7.36-7.29 (m, 2H), 7.01-6.96 (m, 1H), 6.52 (dd, 1H), 5.97 (bs, 2H), 0.2 (s, 9H).

### Example 1. Synthesis of 3-(4-((2-(2-fluoro-5-((6-fluoro-4-methyl-1H-indol-5-yl)oxy)phenyl)-1H-imidazol-4-yl)methyl)thiazol-2-yl)propanoic acid

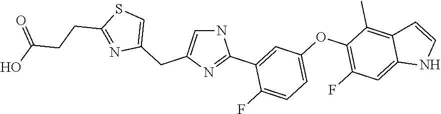 [SMILES: Cc1c(Oc2ccc(F)c(-c3nc(Cc4csc(CCC(=O)O)n4)c[nH]3)c2)c(F)cc2[nH]ccc12]

### 3-(4-Bromothiazol-2-yl)propanoic acid

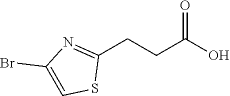 [SMILES: O=C(O)CCc1nc(Br)cs1]

Step A: To formic acid (719 mg, 15.6 mmol) was added Et₃N (0.86 mL, 6.2 mmol) at 0° C. The mixture was stirred at room temperature for 15 min, then diluted with DMF (10 mL). To this solution was added 4-bromothiazole-2-carbaldehyde (1.0 g, 5.2 mmol) and 2,2-dimethyl-1,3-dioxane-4,6-dione (718 mg, 5.2 mmol). The resulting mixture was heated at 100° C. overnight. The reaction mixture was quenched with water (20 mL), acidified to pH=2, and extracted with ethyl acetate (10 mL×3). The organic phase was washed with 1 N aqueous NaOH (10 mL×3). The aqueous phase was acidified with concentrated hydrochloric acid to pH=2 and extracted with ethyl acetate (10 mL×3). The combined organic phase was washed with brine (10 mL), dried over anhydrous Na₂SO₄, filtered, and concentrated in vacuo to afford 3-(4-bromothiazol-2-yl)propanoic acid (1.1 g, 92%) as a yellow oil, which was used directly in the next step. MS (ESI): 236.0 m/z (M−H)⁻.

### Methyl 3-(4-bromothiazol-2-yl)propanoate

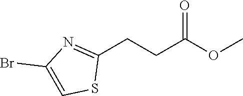

Step B: To a solution of 3-(4-bromothiazol-2-yl)propanoic acid (1.1 g, 4.7 mmol) in MeOH (15 mL) was added H₂SO₄ (1 mL) at room temperature. The resulting mixture was stirred at 80° C. for 4 h and The mixture was concentrated. The residue was diluted with EtOAc (15 mL), washed with water (5 mL×3), and brine (5 mL). The organic phase was dried over anhydrous Na₂SO₄, filtered and concentrated. The residue was purified by silica gel column chromatography (eluting with 10/1 petroleum ether/EtOAc) to afford methyl 3-(4-bromothiazol-2-yl)propanoate (780 mg, 60%) as a yellow oil. MS (ESI): 250.1 m/z (M+H)⁺.

### Methyl 3-(4-((2-(2-fluoro-5-((6-fluoro-4-methyl-1-tosyl-1H-indol-5-yl)oxy)phenyl)-1H-imidazol-4-yl)methyl)thiazol-2-yl)propanoate

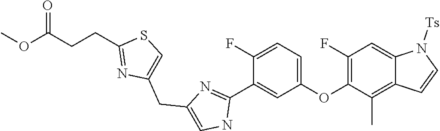 [SMILES: COC(=O)CCc1nc(Cc2c[nH]c(-c3cc(Oc4c(F)cc5c(ccn5S(=O)(=O)c5ccc(C)cc5)c4C)ccc3F)n2)cs1]

Step C: A mixture of methyl 3-(4-bromothiazol-2-yl)propanoate (850 mg, 3.64 mmol), 2-fluoro-5-((6-fluoro-4-methyl-1-tosyl-1H-indol-5-yl)oxy)-N-(prop-2-yn-1-yl)benzimidamide (500 mg, 1.01 mmol), Pd(PPh₃)₄ (116 mg, 0.10 mmol), CuI (38 mg, 0.20 mmol) and K₂CO₃ (697 mg, 5.05 mmol) in DMF (7 mL) was stirred at 80° C. in a glove box overnight. The insoluble materials were removed by suction filtration. The filtrate was diluted with EtOAc (30 mL), washed with water (10 mL×2), brine (10 mL), dried over anhydrous Na₂SO₄, filtered and concentrated. The residue was purified by silica gel column chromatography (eluting with 2/1 dichloromethane/EtOAc) to afford methyl 3-(4-((2-(2-fluoro-5-((6-fluoro-4-methyl-1-tosyl-1H-indol-5-yl)oxy)phenyl)-1H-imidazol-4-yl)methyl)thiazol-2-yl)propanoate (150 mg. 22%) as a yellow solid. MS (ESI): 662.7 m/z (M+H)⁺.

### 3-(4-((2-(2-Ffluoro-5-((6-fluoro-4-methyl-1H-indol-5-yl)oxy)phenyl)-1H-imidazol-4-yl)methyl)thiazol-2-yl)propanoic acid

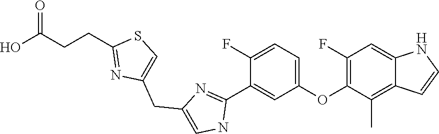 [SMILES: Cc1c(Oc2ccc(F)c(-c3nc(Cc4csc(CCC(=O)O)n4)c[nH]3)c2)c(F)cc2[nH]ccc12]

Step D: To a stirred solution of methyl 3-(4-((2-(2-fluoro-5-((6-fluoro-4-methyl-1-tosyl-1H-indol-5-yl)oxy)phenyl)-1H-imidazol-4-yl)methyl)thiazol-2-yl)propanoate (150 mg, 0.23 mmol) in THE (2 mL) was added a solution of LiOH (200 mg, 5.0 mmol) in water (2 mL). The resulting mixture was stirred in a sealed tube at 120° C. under microwave irradiation. The reaction was diluted with water (10 mL) and the THF was removed in vacuo. The aqueous phase was acidified to pH=4, and extracted with EtOAc (5 mL×3). The combined organic phase was washed with brine (5 mL), dried over Na₂SO₄, filtered, and concentrated. The residue was purified by prep-HPLC to afford the title compound (35.4 mg, 26%) as a white solid. MS (ESI): 495.1 m/z (M+H)⁺. ¹H-NMR (400 MHz, CD₃OD) d 7.42 (s, 1H), 7.28 (s, 1H), 7.21-7.10 (m, 2H), 7.02 (s, 1H), 6.91-6.87 (m, 2H), 6.53 (s, 1H), 4.07 (s, 2H), 3.28 (t, J=7.2 Hz, 2H), 2.79 (t, J=7.2 Hz, 2H), 2.40 (s, 3H) ppm.

### Example 2. Synthesis of 3-(5-((2-(5-((4,6-difluoro-1H-indol-5-yl)oxy)-2-fluorophenyl)-1H-imidazol-5-yl)methyl)thiazol-2-yl)propanoic acid

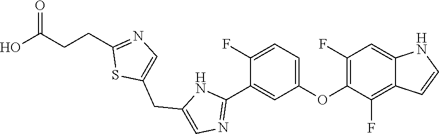 [SMILES: O=C(O)CCc1ncc(Cc2cnc(-c3cc(Oc4c(F)cc5[nH]ccc5c4F)ccc3F)[nH]2)s1]

### 5-((4,6-Difluoro-1-tosyl-1H-indol-5-yl)oxy)-2-fluorobenzonitrile

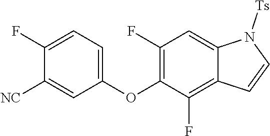 [SMILES: Cc1ccc(S(=O)(=O)n2ccc3c(F)c(Oc4ccc(F)c(C#N)c4)c(F)cc32)cc1]

Step A: To a stirred solution of 5-((4,6-difluoro-1-tosyl-1H-indol-5-yl)oxy)-2-fluorobenzonitrile (Intermediate 2) (1.0 g, 3.5 mmol) in DMF (30 mL) was slowly added NaH (0.21 g, 5.2 mmol) at 0° C. The mixture was stirred at 0° C. for 30 minutes and a solution of TsCl (0.8 g, 4.2 mmol) in DMF (10 mL) was added dropwise. The mixture was stirred at room temperature for 16 hours. The reaction mixture was diluted with EtOAc (150 mL), washed with water (90×2 mL) and brine, dried over anhydrous sodium sulfate, filtered and concentrated. The crude product was purified by silica gel chromatography eluting with 0-40% EtOAc in petroleum ether to afford the title compound (1.25 g; 80%) as a yellow solid. MS (ESI): 465.0 m/z (M+Na)⁺.

### 5-((4,6-Difluoro-1-tosyl-1H-indol-5-yl)oxy)-2-fluoro-N-(prop-2-yn-1-yl)benzimidamide

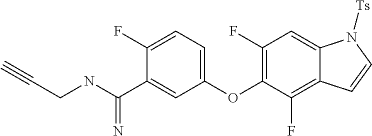 [SMILES: C#CCNC(=N)c1cc(Oc2c(F)cc3c(ccn3S(=O)(=O)c3ccc(C)cc3)c2F)ccc1F]

Step B: In a glove box, to a microwave reaction tube was added 5-((4,6-difluoro-1-tosyl-1H-indol-5-yl)oxy)-2-fluorobenzonitrile (0.88 g, 2.0 mmol), prop-2-yn-1-amine (0.88 g, 16 mmol), THE (7 mL) and DABAL-Me3 (0.88 g, 3.5 mmol). The tube was sealed. The reaction was stirred in microwave at 130° C. for 1.5 hours. Upon completion the mixture was diluted with ethyl acetate (120 mL), washed with brine (20 mL×3), dried over sodium sulfate, and concentrated. The residue was purified by flash column chromatography (40 g silica gel column, eluting with 0-6% MeOH in DCM containing 1% 7N ammonia in MeOH) to provide the pure product (0.77 g, 77%) as a yellow solid. MS (ESI): 498.1 m/z (M+H)⁺.

### Ethyl 3-(5-((2-(5-((4,6-difluoro-1-tosyl-1H-indol-5-yl)oxy)-2-fluorophenyl)-1H-imidazol-5-yl)methyl)thiazol-2-yl)propanoate

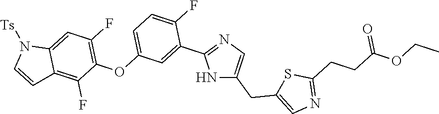 [SMILES: CCOC(=O)CCc1ncc(Cc2cnc(-c3cc(Oc4c(F)cc5c(ccn5S(=O)(=O)c5ccc(C)cc5)c4F)ccc3F)[nH]2)s1]

Step C: In a glove box, to a reaction tube was added 5-((4,6-difluoro-1-tosyl-1H-indol-5-yl)oxy)-2-fluoro-N-(prop-2-yn-1-yl)benzimidamide (0.7 g, 1.4 mmol), ethyl 3-(5-bromothiazol-2-yl)propanoate (0.44 g, 1.7 mmol), Pd(PPh₃)₄ (0.16 g, 0.14 mmol), Cut (53 mg, 0.28 mmol), K₂CO₃ (0.97 g, 0.5 mmol) and DMF (6 mL). The mixture was stirred at 80° C. overnight, and cooled to room temperature. The reaction mixture was diluted with ethyl acetate (100 mL), washed with H₂O (40 mL×2), brine (35 mL×2), dried over sodium sulfate, and concentrated. The residue was purified by flash column chromatography (silica gel column, eluting with 0-70% ethyl acetate in petroleum ether) to provide the pure product ethyl 3-(5-((2-(5-((4,6-difluoro-1-tosyl-1H-indol-5-yl)oxy)-2-fluorophenyl)-1H-imidazol-5-yl)methyl)thiazol-2-yl)propanoate (0.26 g, 27%) as a yellow solid. MS (ESI): 681.1 m/z (M+H)⁺.

### 3-(5-((2-(5-((4,6-Difluoro-1H-indol-5-yl)oxy)-2-fluorophenyl)-1H-imidazol-5-yl)methyl)thiazol-2-yl)propanoic acid

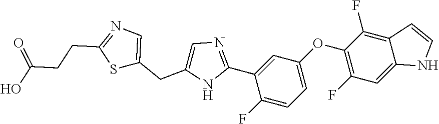 [SMILES: O=C(O)CCc1ncc(Cc2cnc(-c3cc(Oc4c(F)cc5[nH]ccc5c4F)ccc3F)[nH]2)s1]

Step D: To a stirred solution of ethyl 3-(5-((2-(5-((4,6-difluoro-1-tosyl-1H-indol-5-yl)oxy)-2-fluorophenyl)-1H-imidazol-5-yl)methyl)thiazol-2-yl)propanoate (0.245 g, 0.046 mmol) in H₂O (2.5 mL) and THE (2.5 mL) was added LiOH monohydrate (81.6 mg). The reaction was stirred in a microwave reactor at 120° C. for 30 minutes, and cooled to room temperature. The reaction mixture was acidified with 1 N aqueous HCl to pH=4, diluted with ethyl acetate (75 mL), washed with brine (10 mL×2), dried over anhydrous sodium sulfate, filtered, and concentrated. The residue was purified by flash column chromatography (silica gel, eluting with 0-10% MeOH in DCM) to provide the title compound (95 mg, 53%) as a white solid. MS (ESI): 499.1 m/z (M+H)⁺. ¹H NMR (400 MHz, CD₃OD): δ 7.49-7.52 (m, 1H), 7.42 (s, 1H), 7.31 (d, 1H), 7.14-7.24 (m, 2H), 6.98-7.02 (m, 2H), 6.57 (d, 1H), 4.13 (s, 2H), 3.24 (t, 2H), 2.73 (t, 2H) ppm.

### Example 3. Synthesis of 3-(5-((2-(5-((4,6-difluoro-1H-indol-5-yl)oxy)-2-fluorophenyl)-1H-imidazol-5-yl)methyl)thiazol-2-yl)propanamide

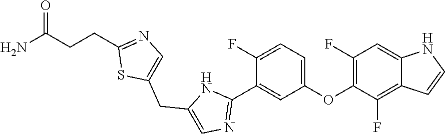 [SMILES: NC(=O)CCc1ncc(Cc2cnc(-c3cc(Oc4c(F)cc5[nH]ccc5c4F)ccc3F)[nH]2)s1]

To a stirred solution of 3-(5-((2-(5-((4,6-difluoro-1H-indol-5-yl)oxy)-2-fluorophenyl)-1H-imidazol-5-yl)methyl)thiazol-2-yl)propanoic acid (39.8 mg, 0.08 mmol), NH₄Cl (85.6 mg, 1.6 mmol), HATU (91.2 mg, 0.24 mmol) in DMF (4 mL) was added Et₃N (0.2 mL, 1.6 mmol). The mixture was stirred at room temperature for 2 hours. The solids were filtered off. The filtrate was concentrated. The residue was dissolved in ethyl acetate (60 mL), washed with brine (10 mL×3), dried over anhydrous sodium sulfate, filtered, and concentrated. The crude product was purified by flash column chromatography on silica, eluting with 0-10% MeOH in DCM, to provide the title compound (12.4 mg, 31%) as a white solid. ¹H NMR (400 MHz, CD₃OD): δ 7.49-7.52 (m, 1H), 7.42 (s, 1H), 7.31 (d, 1H), 7.14-7.24 (m, 2H), 6.98-7.02 (m, 2H), 6.57 (d, 1H), 4.13 (s, 2H), 3.24 (t, 2H), 2.73 (t, 2H) ppm.

### Example 4. Synthesis of 3-(4-((2-(2-fluoro-5-((6-fluoro-4-methyl-1H-indol-5-yl)oxy)phenyl)-1H-imidazol-4-yl)methyl)thiophen-2-yl)propanoic acid

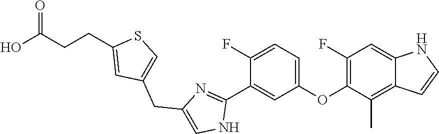 [SMILES: Cc1c(Oc2ccc(F)c(-c3nc(Cc4csc(CCC(=O)O)c4)c[nH]3)c2)c(F)cc2[nH]ccc12]

### 3-(4-Bromothiophen-2-yl)propanoic acid

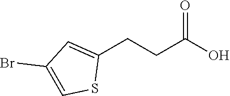 [SMILES: O=C(O)CCc1cc(Br)cs1]

Step A: To formic acid (3.61 g, 78.54 mmol) was added triethylamine (3.17 g, 31.42 mmol) at 0° C. and stirred for 15 min at room temperature. The mixture was then diluted with DMF (12 mL). To this solution were added 4-bromothiophene-2-carbaldehyde (5 g, 26.18 mmol), 2,2-dimethyl-1,3-dioxane-4,6-dione (3.77 g, 26.18 mmol). The mixture was then heated to 100° C. overnight and then poured into ice-water (50 ml). The mixture was acidified to pH=2 and extracted with ethyl acetate (30 mL×2). The organic layer was washed with 1 N aqueous sodium hydroxide (40 mL×2) and separated. The aqueous layer was acidified to pH=2 with concentrated hydrochloric acid and extracted with ethyl acetate (40 ml×2). The combined organic layer was dried over anhydrous sodium sulfate, filtered, and concentrated to give 3-(4-bromothiophen-2-yl)propanoic acid (6.1 g, crude) as a colorless oil, which was used directly in the next step. MS (ESI): 234.7 and 236.7 m/z (M+H)⁺.

### Methyl 3-(4-bromothiophen-2-yl)propanoate

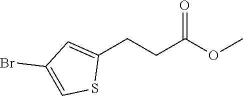

Step B: To a stirred solution of 3-(4-bromothiophen-2-yl)propanoic acid (6.1 g, 25.96 mmol) in MeOH (20 mL) was added concentrated sulfuric acid (2 mL). The mixture was stirred at 65° C. for 2 hours and concentrated. The residue, which was poured into ice-water (30 ml) and extracted with ethyl acetate (30 mL×2). The combined organic phase was washed with brine (15 mL×2), dried over anhydrous sodium sulfate, filtered and concentrated in vacuo. The residue was purified by flash column chromatography on silica, (eluting with 20/1 petroleum ether/ethyl acetate) to give methyl 3-(4-bromothiophen-2-yl)propanoate (5.3 g, 81% over two steps) as colorless oil. MS (ESI): 248.7, 250.7 m/z (M+H)⁺.

### Methyl 3-(4-allylthiophen-2-yl)propanoate

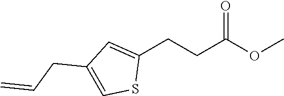

Step C: To a stirred solution of methyl 3-(4-bromothiophen-2-yl)propanoate (5.3 g, 21.28 mmol) and 2-allyl-4,4,5,5-tetramethyl-1,3,2-dioxaborolane (5.36 g, 31.93 mmol) in THE (50 mL) was added under a nitrogen atmosphere Pd(PPh₃)₄ (1.97 g. 1.7 mmol) and Cs₂CO₃ (85.12 g, 27.75 mmol) under a nitrogen atmosphere. The mixture was stirred and refluxed under a N₂ atmosphere overnight. After cooling, the reaction mixture was filtered and the solids washed with ethyl acetate (30 ml). The combined filtrate was washed with water (30 mL), and brine (25 ml), dried over anhydrous sodium sulfate, filtered, and concentrated in vacuo. The residue, which was purified by flash column chromatography on silica, eluting with 20/1 petroleum ether/ethyl acetate, to provide methyl 3-(4-allylthiophen-2-yl)propanoate (2.7 g, 60%) as colorless oil. MS (ESI): 210.9 m/z (M+H)⁺.

### Methyl 3-(5-bromo-4-(3-bromo-2-hydroxypropyl)thiophen-2-yl)propanoate

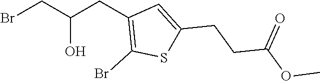 [SMILES: COC(=O)CCc1cc(CC(O)CBr)c(Br)s1]

Step D: To a stirred solution of methyl 3-(4-allylthiophen-2-yl)propanoate (480 mg, 2.28 mmol) in acetone (8 mL) was added H₂O (82 mg, 4.57 mmol) and N-bromosuccinimide (854 mg, 4.8 mmol). The mixture was stirred for 2 hours at room temperature then diluted with ethyl acetate (30 mL), washed with saturated aqueous NaHCO₃ (15 mL×1) and brine (10 mL×2), dried over anhydrous sodium sulfate, filtered, and concentrated. The residue was purified by flash column chromatography on silica, eluting with 0-30% ethyl acetate in petroleum ether, to provide methyl 3-(5-bromo-4-(3-bromo-2-hydroxypropyl)thiophen-2-yl)propanoate (400 mg, 47%) as a colorless oil. MS (ESI): 389, 387 m/z (M+H)⁺.

### Methyl 3-(5-bromo-4-(3-bromo-2-oxopropyl)thiophen-2-yl)propanoate

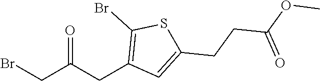 [SMILES: COC(=O)CCc1cc(CC(=O)CBr)c(Br)s1]

Step E: To a stirred solution of methyl 3-(5-bromo-4-(3-bromo-2-hydroxypropyl)thiophen-2-yl)propanoate (660 mg, 1.71 mmol) in DCM (10 mL) was added Dess-Martin periodinane (1.53 g, 3.59 mmol). The mixture was stirred at ambient temperature for 6 hours. The reaction mixture was filtered, and the solids was rinsed with DCM (50 mL). The combined filtrate was washed with saturated aqueous NaHCO₃ (15 mL×1), brine (15 mL×2), dried over Na₂SO₄, filtered, and concentrated. The residue was purified by flash column chromatography on silica, eluting with 0-40% ethyl acetate in petroleum ether, to provide methyl 3-(5-bromo-4-(3-bromo-2-oxopropyl)thiophen-2-yl)propanoate (420 mg, 63%) as an oil. ¹H NMR (400 MHz, MeOH-d₄): δ 6.3 (s, 1H), 3.96 (s, 2H), 3.88 (s, 2H), 3.72 (s, 3H), 3.1 (t, J=7.6 Hz, 2H), 2.68 (t, J=7.6 Hz, 2H) ppm.

### Methyl 3-(5-bromo-4-((2-(2-fluoro-5-((6-fluoro-4-methyl-1H-indol-5-yl)oxy)phenyl)-1H-imidazol-4-yl)methyl)thiophen-2-yl)propanoate

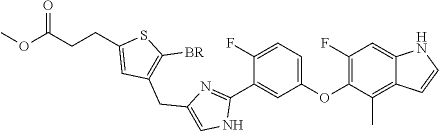

### Step F: A mixture of methyl 3-(5-bromo-4-(3-bromo-2-oxopropyl)thiophen-2-yl)propanoate

(420 mg, 1.1 mmol) and 2-fluoro-5-((6-fluoro-4-methyl-1H-indol-5-yl)oxy)benzimidamide (Intermediate 1) (329 mg, 1.1 mmol) in DMF (5 ml) was stirred at 75° C. overnight. The mixture was concentrated in vacuo. The residue was dissolved in ethyl acetate (70 mL). The organic phase was, washed with H₂O (15 mL×2) and brine (15 mL×2), dried over anhydrous Na₂SO₄, filtered, and concentrated in vacuo. The residue was purified by flash column chromatography on silica, eluting with 2/1/1 petroleum ether/ethyl acetate/dichloromethane), to provide methyl 3-(5-bromo-4-((2-(2-fluoro-5-((6-fluoro-4-methyl-1H-indol-5-yl)oxy)phenyl)-1H-imidazol-4-yl)methyl)thiophen-2-yl)propanoate (200 mg, 31%) as a yellow solid. ¹H NMR (400 MHz, MeOH-d₄): δ 7.43 (s, 1H), 7.28 (m, 1H), 7.13 (m, 2H), 6.89 (m, 1H), 6.75 (m, 1H), 6.61 (s, 1H), 6.53 (s, 1H), 3.82 (s, 1H), 3.75 (s, 2H), 3.68 (s, 3H), 3.02 (t, J=7 Hz, 2H), 2.50 (t, J=7 Hz, 1H), 2.41 (s, 3H) ppm.

### Methyl 3-(4-((2-(2-fluoro-5-((6-fluoro-4-methyl-1H-indol-5-yl)oxy)phenyl)-1H-imidazol-4-yl)methyl)thiophen-2-yl)propanoate

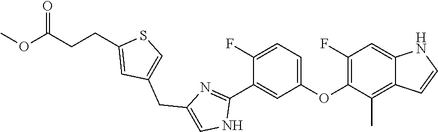 [SMILES: COC(=O)CCc1cc(Cc2c[nH]c(-c3cc(Oc4c(F)cc5[nH]ccc5c4C)ccc3F)n2)cs1]

Step G: To a stirred solution of methyl 3-(5-bromo-4-((2-(2-fluoro-5-((6-fluoro-4-methyl-1H-indol-5-yl)oxy)phenyl)-1H-imidazol-4-yl)methyl)thiophen-2-yl)propanoate (200 mg, 0.34 mmol) in MeOH (20 mL) was added Pd/C (100 mg, 10 wt. %) and solid NaHCO₃ (57 mg, 0.68 mmol). The mixture was purged with H₂ and stirred at room temperature under H₂ for 4 hours. The reaction was monitored by LC-MS until completion. The mixture was filtered through a pad of Celite and the pad was washed with THE (5 mL×3). The combined filtrates were concentrated to give methyl 3-(4-((2-(2-fluoro-5-((6-fluoro-4-methyl-1H-indol-5-yl)oxy)phenyl)-1H-imidazol-4-yl)methyl)thiophen-2-yl)propanoate (150 mg, 86%) as a yellow solid. MS (ESI): 507.7 m/z (M+H)⁺.

### 3-(4-((2-(2-Fluoro-5-((6-fluoro-4-methyl-1H-indol-5-yl)oxy)phenyl)-1H-imidazol-4-yl)methyl)thiophen-2-yl)propanoic acid

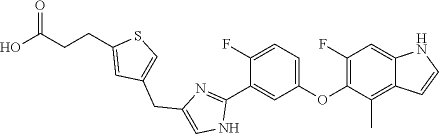 [SMILES: Cc1c(Oc2ccc(F)c(-c3nc(Cc4csc(CCC(=O)O)c4)c[nH]3)c2)c(F)cc2[nH]ccc12]

Step H: To a stirred solution of methyl 3-(4-((2-(2-fluoro-5-((6-fluoro-4-methyl-1H-indol-5-yl)oxy)phenyl)-1H-imidazol-4-yl)methyl)thiophen-2-yl)propanoate (150 mg, 0.3 mmol) in THE (5 mL) was added a solution of LiOH (50 mg, 2.1 mmol) in water (1 mL), and the resulting mixture was stirred overnight at room temperature. 1N hydrochloric acid was added to the above mixture to adjust the pH to 6. The suspension was filtered to give a crude product, which was purified by prep-HPLC, to afford 3-(4-((2-(2-fluoro-5-((6-fluoro-4-methyl-1H-indol-5-yl)oxy)phenyl)-1H-imidazol-4-yl)methyl)thiophen-2-yl)propanoic acid (23 mg, 21%) as a white solid. MS (ESI): 494.0 m/z (M+H)⁺. ¹H NMR (400 MHz, MeOH-d₄): δ 7.42 (s, 1H), 7.28 (m, 1H), 7.18-7.10 (m, 2H), 6.85-6.75 (m, 4H), 6.53 (s, 1H), 3.87 (m, 2H), 3.07 (m, 2H), 2.62 (m, 2H), 2.47 (s, 3H) ppm.

### Example 5. Synthesis of 3-(3-((2-(5-((4,6-difluoro-1H-indol-5-yl)oxy)-2-fluorophenyl)-1H-imidazol-5-yl)methyl)-1H-pyrazol-1-yl)propanoic acid

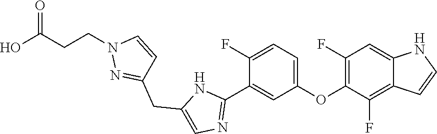 [SMILES: O=C(O)CCn1ccc(Cc2cnc(-c3cc(Oc4c(F)cc5[nH]ccc5c4F)ccc3F)[nH]2)n1]

### Methyl 3-(3-bromo-1H-pyrazol-1-yl)propanoate

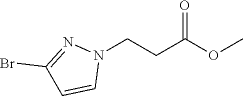

Step A: A mixture of 3-bromo-1H-pyrazole (5.15 g, 35 mmol), methyl 3-bromopropanoate (8.78 g, 52.6 mmol) and K₂CO₃ (9.69 g, 70.1 mmol) in DMF (75 mL) was stirred at room temperature overnight. The reaction was quenched with water (150 mL) and extracted with ethyl acetate (120 mL×3). The combined organic phase was washed with brine (30 mL), dried over anhydrous sodium sulfate, filtered, and concentrated in vacuo. The residue was purified by silica gel column chromatography eluting with 0-25% EtOAc in petroleum ether to give methyl 3-(3-bromo-1H-pyrazol-1-yl)propanoate (6.7 g, 77.9%) as an oil. MS (ESI): m/z 233.1, 235.1 [M+H]⁺.

### Methyl 3-(3-allyl-1H-pyrazol-1-yl)propanoate

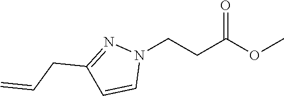

Step B: To a stirred solution of methyl 3-(3-bromopyrazol-1-yl)propanoate (4.2 g, 18 mmol), allyltributyltin (11.9 g, 36 mmol) and lithium chloride (2.29 g, 54 mmol) in DMF (60 mL) was added Pd(PPh₃)₂Cl₂ (1.32 g, 1.8 mmol), and the reaction mixture was heated at 100° C. for 16 hours. After the reaction mixture was cooled to room temperature, the mixture was diluted with EtOAc (400 mL). The organic layer was washed with water (150 mL×2), dried with Na₂SO₄, filtered, and concentrated. The residue was purified by flash column chromatography on silica, eluting with 0-65% EtOAc in petroleum ether to give the product, methyl 3-(3-allyl-1H-pyrazol-1-yl)propanoate (1.35 g, 25%) as a yellow oil. ¹H NMR (400 MHz, CDCl₃): δ 7.35 (d, 1H), 6.00 (dd, 1H), 5.94-5.98 (m, 1H), 5.04-5.14 (m, 2H), 4.35 (t, 2H), 3.67 (s, 3H), 3.38 (d, 2H), 2.87 (t, 2H) ppm.

### Methyl 3-(4-bromo-3-(3-bromo-2-hydroxypropyl)-1H-pyrazol-1-yl)propanoate

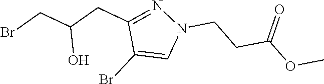 [SMILES: COC(=O)CCn1cc(Br)c(CC(O)CBr)n1]

Step C: To a stirred solution of methyl 3-(3-allylpyrazol-1-yl)propanoate (1.3 g, 6.7 mmol) in DMSO (30 mL) was added H₂O (121 mg) and N-bromosuccinimide (2.62 g, 14.7 mmol) at 0° C. The reaction mixture was stirred in an ice-water bath for 2 hours. H₂O (90 mL) was added, and the mixture was extracted with EtOAc (50 mL×3). The combined organic phase was washed with water (20 mL×2), brine, dried over Na₂SO₄, filtered, and concentrated. The residue was purified by flash column chromatography on silica, eluting with 0-40% ethyl acetate in petroleum ether, to give the product, methyl 3-(4-bromo-3-(3-bromo-2-hydroxypropyl)-1H-pyrazol-1-yl)propanoate (0.99 g, 40%), which was used in the next step without further characterization.

### Methyl 3-(4-bromo-3-(3-bromo-2-oxopropyl)-1H-pyrazol-1-yl)propanoate

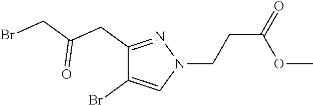 [SMILES: COC(=O)CCn1cc(Br)c(CC(=O)CBr)n1]

Step D: To a stirred solution of methyl 3-(4-bromo-3-(3-bromo-2-hydroxypropyl)-1H-pyrazol-1-yl)propanoate (0.99 g, 2.65 mmol) in DCM (20 mL) was added Dess-Martin periodinane (1.35 g, 3.1 mmol). The reaction mixture was stirred at room temperature for 4 hours and the solids were filtered off. The organic phase was washed with saturated NaHCO₃ solution, brine, dried over anhydrous Na₂SO₄, filtered, and concentrated. The residue was purified by flash column chromatography on silica, eluting with 0-30% ethyl acetate in petroleum ether, to provide the product, methyl 3-(4-bromo-3-(3-bromo-2-oxopropyl)-1H-pyrazol-1-yl)propanoate (0.51 g, 43%). MS (ESI): 368.5 m/z (M+H)⁺.

### Methyl 3-(4-bromo-3-((2-(5-((4,6-difluoro-1H-indol-5-yl)oxy)-2-fluorophenyl)-1H-imidazol-5-yl)methyl)-1H-pyrazol-1-yl)propanoate

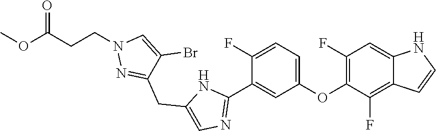 [SMILES: COC(=O)CCn1cc(Br)c(Cc2cnc(-c3cc(Oc4c(F)cc5[nH]ccc5c4F)ccc3F)[nH]2)n1]

Step E: To a stirred solution of 5-((4,6-difluoro-1H-indol-5-yl)oxy)-2-fluorobenzimidamide (Intermediate 4) (0.214 g, 0.7 mmol) and methyl 3-(4-bromo-3-(3-bromo-2-oxopropyl)-1H-pyrazol-1-yl)propanoate (0.258 g, 0.7 mmol) in DMF (12 mL) was added NaHCO₃ (0.118 g, 1.4 mmol). The mixture was heated at 80° C. and stirred for 16 hours under N₂. After cooling to room temperature, the mixture was diluted with EtOAc (120 mL), then washed with water (30 mL×2), and brine (30 mL), dried over anhydrous Na₂SO₄, filtered, and concentrated. The residue was purified by silica gel column chromatography, eluting with 0-90% ethyl acetate in petroleum ether to provide the product, methyl 3-(4-bromo-3-((2-(5-((4,6-difluoro-1H-indol-5-yl)oxy)-2-fluorophenyl)-1H-imidazol-5-yl)methyl)-1H-pyrazol-1-yl)propanoate (0.252 g, 59%) as a solid. MS (ESI): 574, 576 m/z (M+H)⁺.

### Methyl 3-(3-((2-(5-((4,6-difluoro-1H-indol-5-yl)oxy)-2-fluorophenyl)-1H-imidazol-5-yl)methyl)-1H-pyrazol-1-yl)propanoate

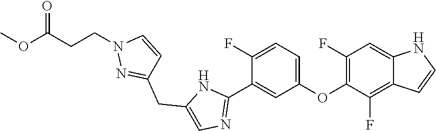 [SMILES: COC(=O)CCn1ccc(Cc2cnc(-c3cc(Oc4c(F)cc5[nH]ccc5c4F)ccc3F)[nH]2)n1]

Step F: To a stirred solution of methyl 3-(4-bromo-3-((2-(5-((4,6-difluoro-1H-indol-5-yl)oxy)-2-fluorophenyl)-1H-imidazol-5-yl)methyl)-1H-pyrazol-1-yl)propanoate (0.25 g; 0.435 mmol) in MeOH (10 mL) was added 10% Pd on carbon (50 mg). The mixture was purged with H₂ and stirred under an H₂ atmosphere pressure at ambient temperature for 6 hours. The mixture was filtered, and the solids rinsed with MeOH (20 mL). The combined filtrates were concentrated to give the product, methyl 3-(3-((2-(5-((4,6-difluoro-1H-indol-5-yl)oxy)-2-fluorophenyl)-1H-imidazol-5-yl)methyl)-1H-pyrazol-1-yl)propanoate (0.18 g, 78% yield) as a solid. MS (ESI): 495.8 m/z (M+H)⁺.

### 3-(3-((2-(5-((4,6-Difluoro-1H-indol-5-yl)oxy)-2-fluorophenyl)-1H-imidazol-5-yl)methyl)-1H-pyrazol-1-yl)propanoic acid

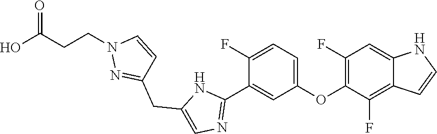 [SMILES: O=C(O)CCn1ccc(Cc2cnc(-c3cc(Oc4c(F)cc5[nH]ccc5c4F)ccc3F)[nH]2)n1]

Step G: To a stirred solution of methyl 3-(3-((2-(5-((4,6-difluoro-1H-indol-5-yl)oxy)-2-fluorophenyl)-1H-imidazol-5-yl)methyl)-1H-pyrazol-1-yl)propanoate (150 mg, 0.3 mmol) in THE (9 mL) and methanol (3 mL) was added a solution of LiOH monohydrate (58.6 mg) in water (3 mL), and the reaction was stirred at room temperature for 3 hours. The pH of the reaction was adjusted to 6 through the addition of 1M hydrochloric acid, the mixture was then diluted with EtOAc (100 mL), washed with water and brine, dried over Na₂SO₄, filtered and concentrated. The crude product was purified by prep-HPLC to afford 3-(3-((2-(5-((4,6-difluoro-1H-indol-5-yl)oxy)-2-fluorophenyl)-1H-imidazol-5-yl)methyl)-1H-pyrazol-1-yl)propanoic acid (102.6 mg, 60%) as a white solid. MS (ESI): 481.9 m/z (M+H)⁺. ¹H NMR (400 MHz, CD₃OD): d 7.48-7.62 (m, 2H), 7.28 (d, 1H), 7.11-7.21 (m, 2H), 6.95-6.98 (m, 1H), 6.82 (s, 1H), 6.54 (d, 1H), 6.07 (d, 1H), 4.34 (t, 2H), 3.92 (s, 2H), 2.80 (t, 2H) ppm.

### Example 6. Synthesis of 3-(5-((3-(2-fluoro-5-((6-fluoro-4-methyl-1H-indol-5-yl)oxy)phenyl)-1H-pyrazol-1-yl)methyl)thiazol-2-yl)propanamide

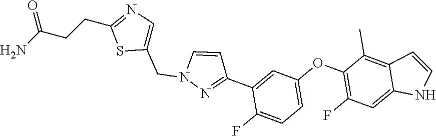 [SMILES: Cc1c(Oc2ccc(F)c(-c3ccn(Cc4cnc(CCC(N)=O)s4)n3)c2)c(F)cc2[nH]ccc12]

### 5-(3-Bromo-4-fluorophenoxy)-6-fluoro-4-methyl-1-tosyl-1H-indole

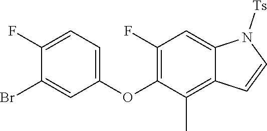 [SMILES: Cc1ccc(S(=O)(=O)n2ccc3c(C)c(Oc4ccc(F)c(Br)c4)c(F)cc32)cc1]

Step A: To a stirred solution of 5-(3-bromo-4-fluorophenoxy)-6-fluoro-4-methyl-1H-indole (Intermediate 1C) (2.2 g, 6.5 mmol) in THE (90 mL) was added slowly at 0° C. NaH (60% dispersion in mineral oil, 0.39 g, 9.8 mmol). The reaction was stirred at 0° C. for 30 minutes and a solution of TsCl (1.48 g, 7.8 mmol) in THE (10 mL) was added. The mixture was stirred at room temperature for 16 hours and. The reaction mixture was concentrated. The residue was dissolved with EtOAc (150 mL), washed with water and brine, dried over sodium sulfate, filtered, and concentrated. The crude product was purified by silica gel chromatography, eluting with 0-20% EtOAc in petroleum ether (30 min) to give 5-(3-bromo-4-fluorophenoxy)-6-fluoro-4-methyl-1-tosyl-1H-indole (1.9 g; 59%) as an oil. ¹HNMR (400 MHz, CDCl₃) 7.79 (d, 2H), 7.71 (d, 1H), 7.59 (d, 1H), 7.29 (m, 2H), 6.96-6.03 (m, 2H), 6.73-6.33 (m, 1H), 6.66 (d, 1H), 2.39 (s, 3H), 2.30 (s, 3H) ppm.

### 6-Fluoro-5-(4-fluoro-3-(1H-pyrazol-3-yl)phenoxy)-4-methyl-1-tosyl-1H-indole

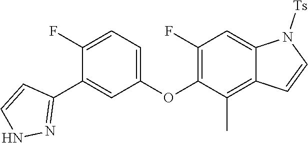 [SMILES: Cc1ccc(S(=O)(=O)n2ccc3c(C)c(Oc4ccc(F)c(-c5cc[nH]n5)c4)c(F)cc32)cc1]

Step B: 5-(3-bromo-4-fluorophenoxy)-6-fluoro-4-methyl-1-tosyl-1H-indole (1.18 g, 2.4 mmol), 3-(4,4,5,5-tetramethyl-1,3,2-dioxaborolan-2-yl)-1H-pyrazole (0.7 g; 3.6 mmol) and K₂CO₃ (0.82 g; 6 mmol) were dissolved in dioxane (60 mL) and H₂O (15 mL), then Pd(dtbpf)Cl₂ (0.12 g; 0.2 mmol) was added. The reaction mixture was stirred overnight at 100° C. under a N₂ atmosphere. Solvent was removed and then EtOAc (180 mL) was added. The mixture was washed with water (50 mL), brine (25 mL), dried over Na₂SO₄, filtered, and concentrated. The crude product was purified by silica gel column chromatography, eluting with 0-50% ethyl acetate in petroleum ether, to give the product, 6-fluoro-5-(4-fluoro-3-(1H-pyrazol-3-yl)phenoxy)-4-methyl-1-tosyl-1H-indole (0.6 g, 52%) as a solid. MS (ESI): 480.1 m/z (M+H)⁺. ¹HNMR (400 MHz, DMSO-d6): δ 13.03 (s, 1H), 7.98 (d, 2H), 7.90 (d, 1H), 7.78-7.84 (m, 2H), 7.44 (d, 2H), 7.20-7.33 (m, 2H), 6.99 (d, 1H), 6.83-6.85 (m, 1H), 6.62 (d, 1H), 2.36 (s, 3H), 2.31 (s, 3H) ppm.

### Ethyl (E)-3-(thiazol-2-yl)acrylate

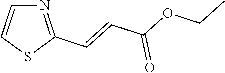

Step C: A solution of triethyl phosphonoacetate (26 g, 116 mmol) in THF (50 mL) was added dropwise to a suspension of NaH (60% dispersion in oil, 4.3 g, 106 mmol) in THE (80 mL) at 0° C. The reaction mixture was stirred at 0° C. for 30 minutes and a solution of 2-formylthiazole (10 g, 88.4 mmol) in THE (80 mL) was added. The mixture was stirred at room temperature for 16 hours and concentrated. The residue was diluted with EtOAc (1000 mL), washed with water and brine, dried over anhydrous sodium sulfate, filtered, and concentrated. The crude product was purified by silica gel column chromatography, eluting with 0-30% EtOAc in petroleum ether, to give the product, ethyl (E)-3-(thiazol-2-yl)acrylate (14.5 g; 90%) as an oil. MS (ESI): 184.1 m/z (M+H)⁺.

### Ethyl 3-(thiazol-2-yl)propanoate

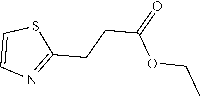

Step D: To a stirred solution of ethyl (E)-3-(thiazol-2-yl)acrylate (14.5 g, 79.1 mmol) in EtOH (250 mL) was added Pd/C (10%, 1.45 g). The mixture was degassed with H₂ and stirred overnight at 38° C. under an H₂ atmosphere. The reaction mixture was filtered through a Celite pad and rinsed with EtOH. The filtrate was concentrated to give the desired product, ethyl 3-(thiazol-2-yl)propanoate (14 g, 96%) as an oil. MS (ESI): 186.1 m/z (M+H)⁺.

### Ethyl 3-(5-bromothiazol-2-yl)propanoate

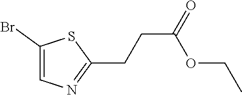

Step E: To a stirred solution of ethyl 3-(thiazol-2-yl)propanoate (14 g, 75.6 mmol) in DMF (200 mL) was added N-bromosuccinimide (16.1 g, 90.7 mmol). The mixture was stirred overnight at 60° C. The mixture was diluted with H₂O (600 mL), extracted with EtOAc (250 mL×3). The combined organic layers were washed with water (100 mL), brine (150 mL), dried over Na₂SO₄, filtered and concentrated. The residue was purified by column chromatography on silica gel, eluting with 0-30% EtOAc in petroleum ether to give the target compound, ethyl 3-(5-bromothiazol-2-yl)propanoate (14.9 g; 75%) as an oil. MS (ESI): 264.0, 266 m/z (M+H)⁺.

### Ethyl 3-(5-vinylthiazol-2-yl)propanoate

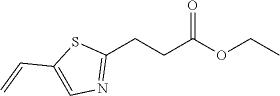

Step F: Ethyl 3-(5-bromothiazol-2-yl)propanoate (2.77 g; 10.5 mmol), 4,4,5,5-tetramethyl-2-vinyl-1,3,2-dioxaborolane (2.9 g; 18.9 mmol) and K₂CO₃ (2.9 g; 21 mmol) were dissolved in dioxane (60 mL) and H₂O (15 mL), then Pd(dppf)Cl₂ (0.41 g; 0.5 mmol) was added. The reaction mixture was stirred overnight at 100° C. under N₂ atmosphere and then concentrated. The residue was dissolved in EtOAc (65 mL), washed with water, brine, dried over Na₂SO₄, filtered, and concentrated. The crude product was purified by silica gel column chromatography, eluting with 0-30% ethyl acetate in petroleum ether, to give the desired product, ethyl 3-(5-vinylthiazol-2-yl)propanoate (1.7 g, 77%) as an oil. MS (ESI): 212.1 m/z (M+H)⁺.

### Ethyl 3-(5-formylthiazol-2-yl)propanoate

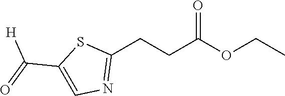

Step G: To a stirred solution of ethyl 3-(5-vinylthiazol-2-yl)propanoate (1.7 g, 8 mmol) in 2:1 THF:H₂O (75 mL) was added NaIO₄ (5.15 g, 24 mmol) and OsO₄ (3 mol-%). The mixture was stirred at room temperature overnight under a nitrogen atmosphere. Saturated aqueous Na₂S203 (80 mL) was added and the mixture was extracted with EtOAc (65 mL×3). The organic layer was washed with brine (20 mL), dried over Na₂SO₄, filtered, and concentrated. The residue was purified by silica gel column chromatography, eluting 0-40% ethyl acetate in petroleum ether to give the product, ethyl 3-(5-formylthiazol-2-yl)propanoate (1.29 g, 76%) as a colorless oil. MS (ESI): 214.1 m/z (M+H)⁺.

### Ethyl 3-(5-(hydroxymethyl)thiazol-2-yl)propanoate

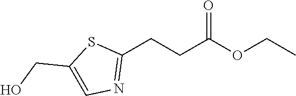

Step H: To a stirred solution of ethyl 3-(5-formylthiazol-2-yl)propanoate (1.28 g, 6 mmol) in THE/EtOH (45 mL/15 mL) was added NaBH₄ (0.34 g, 9 mmol) in portion wise at 0° C. The reaction mixture was then stirred at 0° C. for 2 hours. The mixture was quenched with water and extracted with DCM. The organic layer was washed with brine, dried over anhydrous Na₂SO₄, filtered, and concentrated. The residue was purified by silica gel column chromatography, eluting with 0-80% ethyl acetate in petroleum ether, to give the desired product, ethyl 3-(5-(hydroxymethyl)thiazol-2-yl)propanoate (0.88 g, 67%) as a colorless oil. MS (ESI): 216.1 m/z (M+H)⁺.

### Ethyl 3-(5-(chloromethyl)thiazol-2-yl)propanoate

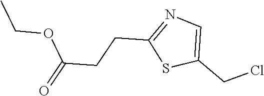

Step I: Methanesulfonyl chloride (60 μL, 0.70 mmol) was added to a solution of ethyl 3-(5-(hydroxymethyl)thiazol-2-yl)propanoate (106 mg, 0.5 mmol) and DIPEA (0.25 mL, 1.5 mmol) in DCM (10 mL). The mixture was stirred at room temperature for 2 hours, diluted with DCM (80 mL), washed with water (30 mL), dried over anhydrous sodium sulfate, filtered and concentrated to give the product, ethyl 3-(5-(chloromethyl)thiazol-2-yl)propanoate (0.11 g, 95%) as a colorless oil. MS (ESI): 234.11 m/z (M+H)⁺.

### Ethyl 3-(5-((3-(2-fluoro-5-((6-fluoro-4-methyl-1-tosyl-1H-indol-5-yl)oxy)phenyl)-1H-pyrazol-1-yl)methyl)thiazol-2-yl)propanoate

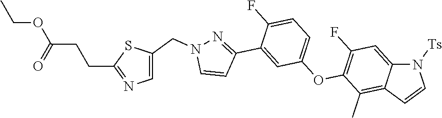 [SMILES: CCOC(=O)CCc1ncc(Cn2ccc(-c3cc(Oc4c(F)cc5c(ccn5S(=O)(=O)c5ccc(C)cc5)c4C)ccc3F)n2)s1]

Step J: To a stirred solution of ethyl 3-(5-(chloromethyl)thiazol-2-yl)propanoate (93 mg, 0.4 mmol), 6-fluoro-5-(4-fluoro-3-(1H-pyrazol-3-yl)phenoxy)-4-methyl-1-tosyl-1H-indole (98 mg, 0.20 mmol) in DMF (8 mL) was added K₂CO₃ (110 mg, 0.8 mmol). The mixture was stirred and heated overnight at 80° C. After cooling to room temperature, the mixture was diluted with EtOAc (90 mL), washed with water (25 mL×2), brine (25 mL), dried over Na₂SO₄, filtered, and concentrated. The crude product was purified by silica gel column chromatography, eluting with 0-70% ethyl acetate in petroleum ether to give ethyl 3-(5-((3-(2-fluoro-5-((6-fluoro-4-methyl-1-tosyl-1H-indol-5-yl)oxy)phenyl)-1H-pyrazol-1-yl)methyl)thiazol-2-yl)propanoate (60 mg, 45%) as a solid. MS (ESI): 677.1 m/z (M+H)⁺. 3-(5-((3-(2-Fluoro-5-((6-fluoro-3-methyl-JH-indol-5-yl)oxy)phenyl)-JH-pyrazol-1-yl)methyl)thiazol-2-yl)propanoic acid

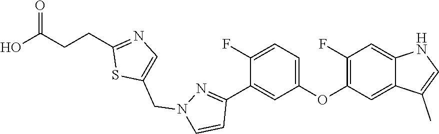 [SMILES: Cc1c[nH]c2cc(F)c(Oc3ccc(F)c(-c4ccn(Cc5cnc(CCC(=O)O)s5)n4)c3)cc12]

Step K: Ethyl 3-(5-((3-(2-fluoro-5-((6-fluoro-4-methyl-1-tosyl-1H-indol-5-yl)oxy)phenyl)-1H-pyrazol-1-yl)methyl)thiazol-2-yl)propanoate (54 mg, 0.08 mmol) was added to a solution of TBAF (1M in THF, 1.6 mL). The mixture was stirred at 75° C. for 3 hours and reaction progress monitored by LC-MS until reaction completion. The mixture was concentrated. The residue was dissolved in ethyl acetate (20 mL), washed with brine (20 mL×3), dried over Na₂SO₄, filtered, and concentrated. The crude product was purified by flash column chromatography, eluting with 0-12% MeOH in DCM. The product was dissolved in MeOH (1 mL) and diluted with water (10 mL), concentrated to remove the MeOH and lyophilized to give the desired product, 3-(5-((3-(2-fluoro-5-((6-fluoro-3-methyl-1H-indol-5-yl)oxy)phenyl)-1H-pyrazol-1-yl)methyl)thiazol-2-yl)propanoic acid (3.8 mg, 10%) as a white solid. MS (ESI): 495.1 m/z (M+H)⁺. ¹H NMR (400 MHz, DMSO-d6): δ 12.29 (s, 1H), 11.45 (s, 1H), 7.87 (d, 1H), 7.62 (s, 1H), 7.39 (t, 1H), 7.23-7.29 (m, 3H), 6.82-6.87 (m, 1H), 6.60 (t, 1H), 6.53 (s, 1H), 5.55 (s, 2H), 3.10 (t, 2H), 2.64 (t, 2H), 2.34 (s, 3H) ppm.

### Example 7. Synthesis of 3-(5-((3-(2-fluoro-5-((6-fluoro-3-methyl-1H-indol-5-yl)oxy)phenyl)-1H-pyrazol-1-yl)methyl)thiazol-2-yl)propanamide

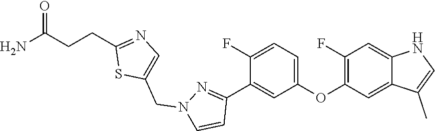 [SMILES: Cc1c[nH]c2cc(F)c(Oc3ccc(F)c(-c4ccn(Cc5cnc(CCC(N)=O)s5)n4)c3)cc12]

To a stirred solution of 3-(5-((3-(2-fluoro-5-((6-fluoro-3-methyl-1H-indol-5-yl)oxy)phenyl)-1H-pyrazol-1-yl)methyl)thiazol-2-yl)propanoic acid (30 mg, 0.064 mmol), NH₄Cl (64 mg, 1.2 mmol) and HATU (68.4 mg, 0.18 mmol) in DMF (4 mL) was added Et₃N (0.16 mL, 1.2 mmol). The mixture was stirred at room temperature for 2 hours. The solids were filtered off and the filtrate was concentrated. The residue was dissolved in ethyl acetate (60 mL), washed with brine (10 mL×3), dried over Na₂SO₄, filtered, and concentrated. The crude product was purified by flash column chromatography on silica, eluting with 0-10% MeOH in DCM to give the desired product, 3-(5-((3-(2-fluoro-5-((6-fluoro-3-methyl-1H-indol-5-yl)oxy)phenyl)-1H-pyrazol-1-yl)methyl)thiazol-2-yl)propanamide (4.7 mg, 15%) as a white solid. MS (ESI): 494.1 m/z (M+H)⁺. ¹H NMR (400 MHz, DMSO-d6): δ 11.27 (s, 1H), 7.87 (d, 1H), 7.62 (s, 1H), 7.22-7.41 (m, 5H), 6.82-6.87 (m, 2H), 6.60 (t, 1H), 6.54 (s, 1H), 5.55 (s, 2H), 3.09 (t, 2H), 2.54 (t, 2H), 2.34 (s, 3H), ¹HNMR (400 MHz, CD₃OD). 7.72 (d, 1H), 7.63 (s, 1H), 7.37-7.39 (m, 1H), 7.29 (d, 1H), 7.06-7.15 (m, 2H), 6.78-6.82 (m, 1H), 6.66-6.68 (m, 1H), 6.54 (d, 1H), 5.53 (s, 2H), 3.23 (t, 2H), 2.67 (t, 2H), 2.40 (s, 3H) ppm.

### Example 8. Synthesis of 5-(3-(5-((5-methylthiophen-2-yl)methyl)-4H-1,2,4-triazol-3-yl)phenoxy)-1H-pyrrolo[2,3-c]pyridine

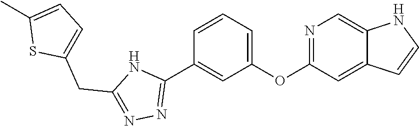 [SMILES: Cc1ccc(Cc2nnc(-c3cccc(Oc4cc5cc[nH]c5cn4)c3)[nH]2)s1]

### Methyl 3-((4-methyl-5-nitropyridin-2-yl)oxy)benzoate

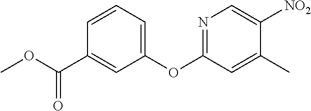 [SMILES: COC(=O)c1cccc(Oc2cc(C)c([N+](=O)[O-])cn2)c1]

Step A: A mixture of 2-chloro-4-methyl-5-nitropyridine (6.5 g, 37.8 mmol), methyl 3-hydroxybenzoate (6.3 g, 41.6 mmol) and K₂CO₃ (10.4 g, 75.6 mmol) in DMF (40 mL) was stirred at 100° C. for 3 hours. The mixture was cooled to room temperature, quenched with water (30 mL), and extracted with EtOAc (50 mL×3). The combined organic phase was washed with brine (50 mL), dried over anhydrous sodium sulfate, filtered, and concentrated in vacuo to afford the crude product, which was purified by silica gel column chromatography (eluting with 10/1 petroleum ether/ethyl acetate) to afford methyl 3-((4-methyl-5-nitropyridin-2-yl)oxy)benzoate (10.0 g, 93%) as a yellow solid. MS (ESI): 289.0 m/z (M+H)⁺.

### Methyl 3-((1H-pyrrolo[2,3-c]pyridin-5-yl)oxy)benzoate

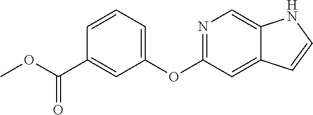 [SMILES: COC(=O)c1cccc(Oc2cc3cc[nH]c3cn2)c1]

Step B: A mixture of methyl 3-((4-methyl-5-nitropyridin-2-yl)oxy)benzoate (4.4 g, 15.3 mmol) and N,N-dimethylformamide dimethyl acetal (9.1 g, 76.4 mmol) in DMF (30 mL) was stirred at 130° C. for 1 hour under an N₂ atmosphere. The residue was dissolved in 80% aqueous acetic acid (50 mL), and Zn (9.9 g, 152.7 mmol) was added. The resulting mixture was stirred at 85° C. overnight. The mixture was filtered through Celite and concentrated in vacuo. The residue was dissolved in ethyl acetate (200 mL), and the organic phase was washed with water (60 mL) and brine (60 mL), dried over anhydrous sodium sulfate, filtered, and concentrated in vacuo. The residue was purified by silica gel column chromatography (eluting with 1/1 petroleum ether/ethyl acetate) to afford methyl 3-((1H-pyrrolo[2,3-c]pyridin-5-yl)oxy)benzoate (2.5 g, 63%) as a pale solid. MS (ESI): 269.1 m/z (M+H)⁺. ¹H NMR (500 MHz, DMSO-d₆): δ 11.6 (s, 1H), 8.46 (s, 1H), 7.70-7.68 (m, 2H), 7.53-7.50 (m, 1H), 7.43-7.42 (m, 1H), 7.32-7.29 (m, 1H), 7.23 (s, 1H), 6.51 (s, 1H), 3.82 (s, 3H) ppm.

### 3-((1H-Pyrrolo[2,3-c]pyridin-5-yl)oxy)benzohydrazide

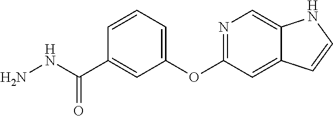 [SMILES: NNC(=O)c1cccc(Oc2cc3cc[nH]c3cn2)c1]

Step C: A mixture of methyl 3-((1H-pyrrolo[2,3-c]pyridin-5-yl)oxy)benzoate (1.0 g, 3.73 mmol) and hydrazine hydrate (0.9 mL, 18.6 mmol) in methanol (25 mL) was stirred at 70° C. for 2 hours. The mixture was concentrated in vacuo and water (20 mL) was added. The aqueous layer was extracted with ethyl acetate (80 mL×4), the combined organic phases were washed with brine (100 mL), dried over anhydrous sodium sulfate, filtered, and concentrated in vacuo to give 3-((1H-pyrrolo[2,3-c]pyridin-5-yl)oxy)benzohydrazide (1.0 g, 100%) as a yellow solid, which was used directly in the next step. MS (ESI): 269.1 m/z (M+H)⁺.

### (5-Methylthiophen-2-yl)methanol

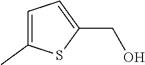

Step D: To a stirred solution of 5-methylthiophene-2-carbaldehyde (1.5 g, 11.9 mmol) in methanol (30 mL) was added NaBH₄ (904.8 mg, 23.8 mmol) at 0° C. The resulting mixture was stirred at room temperature for 3 hours, and the excess NaBH₄ was decomposed by adding a small amount of saturated aqueous NH₄Cl. The mixture was filtered through Celite, and the filtrate was concentrated in vacuo to give (5-methylthiophen-2-yl)methanol (1.4 g, 93%) as brown oil, which was used directly in the next step.

### 2-(Chloromethyl)-5-methylthiophene

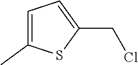

Step E: To a stirred solution of (5-methylthiophen-2-yl)methanol (1.4 g, 10.9 mmol) in dichloromethane (20 mL) was added SOCl₂ (1.6 mL, 21.9 mmol) at 0° C. The resulting mixture was stirred at room temperature for 2 hours, and then quenched with saturated aqueous NaHCO₃. The organic layer was separated, washed with brine, dried over anhydrous sodium sulfate, filtered, and concentrated in vacuo to give 2-(chloromethyl)-5-methylthiophene (1.0 g, 63%) as a brown oil, which was used directly in the next step.

### 2-(5-Methylthiophen-2-yl)acetonitrile

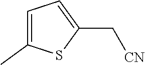

Step F: A mixture of 2-(chloromethyl)-5-methylthiophene (1.0 g, 6.85 mmol), trimethylsilyl cyanide (6.8 g, 68.5 mmol) and TBAF (17.8 g, 68.5 mmol) in DCM (40 mL) was stirred at 40° C. for 2 hours. The resulting solution was concentrated in vacuo. The residue was dissolved in EtOAc (50 mL). The organic phase was washed with water, brine, dried over anhydrous sodium sulfate, filtered, and concentrated in vacuo to give 2-(5-methylthiophen-2-yl)acetonitrile (835 mg, 89%) as a brown oil which was used directly in the next step.

### 5-(3-(5-((5-Methylthiophen-2-yl)methyl)-4H-1,2,4-triazol-3-yl)phenoxy)-1H-pyrrolo[2,3-c]pyridine

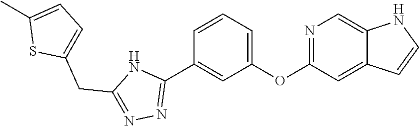 [SMILES: Cc1ccc(Cc2nnc(-c3cccc(Oc4cc5cc[nH]c5cn4)c3)[nH]2)s1]

Step G: A mixture of 3-((1H-pyrrolo[2,3-c]pyridin-5-yl)oxy)benzohydrazide (150 mg, 0.56 mmol), 2-(5-methylthiophen-2-yl)acetonitrile (92 mg, 0.67 mmol) and K₂CO₃ (154.5 mg, 1.12 mmol) in n-BuOH (10 mL) was stirred at 130° C. for 2 hours under an N₂ atmosphere. The mixture was concentrated in vacuo, and ethyl acetate (50 mL) was added. The organic layer was washed with water (50 mL), brine (50 mL), dried over anhydrous sodium sulfate, filtered, and concentrated in vacuo. The residue was purified by prep-HPLC to afford 5-(3-(5-((5-methylthiophen-2-yl)methyl)-4H-1,2,4-triazol-3-yl)phenoxy)-1H-pyrrolo[2,3-c]pyridine (40.3 mg, 19%) as a white solid. MS (ESI): 388.1 m/z (M+H)⁺. ¹H NMR (500 MHz, DMSO-d₆): d 14.05 (brs, 1H), 11.62 (s, 1H), 8.46 (s, 1H), 7.72-7.67 (m, 2H), 7.50 (s, 1H), 7.47-7.43 (m, 1H), 7.22 (s, 1H), 7.06 (dd, J=8.0 Hz & 1.5 Hz, 1H), 6.72 (d, J=3.0 Hz, 1H), 6.61 (d, J=2.0 Hz, 1H), 6.51 (s, 1H), 4.19 (s, 2H), 2.08 (s, 3H) ppm.

### Example 9. Synthesis of 5-(3-(5-((5-methylthiophen-3-yl)methyl)-4H-1,2,4-triazol-3-yl)phenoxy)-1H-pyrrolo[2,3-c]pyridine

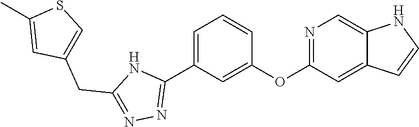 [SMILES: Cc1cc(Cc2nnc(-c3cccc(Oc4cc5cc[nH]c5cn4)c3)[nH]2)cs1]

### (5-Methylthiophen-3-yl))methanol

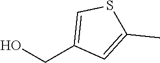

Step A: To a stirred solution of 5-methylthiophene-3-carboxylic acid (800 mg, 5.63 mmol) in THE (10 mL) was added lithium aluminum hydride (642 mg, 16.9 mmol) at −10° C. The resulting mixture was stirred at room temperature for 3 hours, and then quenched with Na₂SO₄·10H₂O at 0° C. The mixture was filtered through Celite, and the filtrate was concentrated in vacuo to give (5-methylthiophen-3-yl)methanol (680 mg, 94%) as colorless oil, which was used directly in the next step. MS (ESI): 129.2 m/z (M+H)⁺.

### 4-(Chloromethyl)-2-methylthiophene

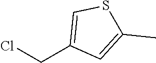

Step B: To a stirred solution of (5-methylthiophen-3-yl)methanol (680 mg, 5.31 mmol) in dichloromethane (20 mL) was added SOCl₂ (1.25 g, 10.6 mmol) at 0° C. The resulting mixture was stirred at room temperature for 2 hours, and then quenched with saturated aqueous NaHCO₃. The organic layer was separated, washed with brine (20 mL), dried over anhydrous sodium sulfate, filtered, and concentrated in vacuo to give 4-(chloromethyl)-2-methylthiophene (700 mg, crude) as brown oil, which was used directly in the next step. MS (ESI): 147.0 m/z (M+H)⁺.

### 2-(5-Methylthiophen-3-yl)acetonitrile

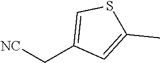

Step C: A mixture of 4-(chloromethyl)-2-methylthiophene (200 mg, 1.37 mmol) and NaCN (201 mg, 4.11 mmol) in DMSO (10 mL) was stirred at 60° C. for 2 hours. The reaction mixture was cooled to room temperature, quenched with water (20 mL) and extracted with ethyl acetate (20 mL×3). The combined organic phase was washed with brine, dried over anhydrous sodium sulfate, filtered, and concentrated in vacuo to give the crude product, which was purified by prep-TLC (eluting with 15/1 petroleum ether/ethyl acetate) to give 2-(5-methylthiophen-3-yl)acetonitrile (142 mg, 66% over 2 steps) as a yellow oil. MS (ESI): 138.2 m/z (M+H)⁺.

### 5-(3-(5-((5-Methylthiophen-3-yl)methyl)-4H-1,2,4-triazol-3-yl)phenoxy)-1H-pyrrolo[2,3-c]pyridine

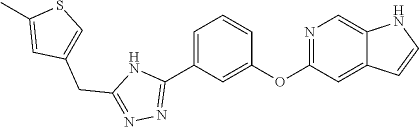 [SMILES: Cc1cc(Cc2nnc(-c3cccc(Oc4cc5cc[nH]c5cn4)c3)[nH]2)cs1]

Step D: A mixture of 2-(5-methylthiophen-3-yl)acetonitrile (142 mg, 1.04 mmol), 3-((1H-pyrrolo[2,3-c]pyridin-5-yl)oxy)benzohydrazide (167 mg, 0.62 mmol) and K₂CO₃ (286 mg, 2.08 mmol) in n-BuOH (10 mL) was stirred at 140° C. for 2 hours under an N₂ atmosphere. The mixture was concentrated, and ethyl acetate (50 mL) was added. The organic layer was washed with water (50 mL) and brine (50 mL), dried with anhydrous sodium sulfate, filtered and concentrated in vacuo. The residue was purified by prep-HPLC to afford 5-(3-(5-((5-methylthiophen-3-yl)methyl)-4H-1,2,4-triazol-3-yl)phenoxy)-1H-pyrrolo[2,3-c]pyridine (37 mg, 16%) as a white solid. MS (ESI): 388.0 m/z (M+H)⁺. ¹H NMR (500 MHz, DMSO-d₆): δ 13.86 (br s, 1H), 11.61 (s, 1H), 8.46 (s, 1H), 7.71-7.67 (m, 2H), 7.50-7.40 (m, 2H), 7.21 (s, 1H), 7.06-6.97 (m, 2H), 6.71 (s, 1), 6.50 (s, 1H), 3.98 (s, 2H), 2.38 (s, 3H) ppm.

### Example 10. Synthesis of 5-(3-(5-(thiophen-2-ylmethyl)-4H-1,2,4-triazol-3-yl)phenoxy)-1H-indole

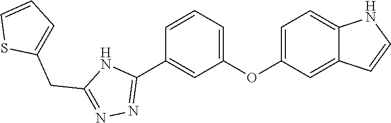 [SMILES: c1cc(Oc2ccc3[nH]ccc3c2)cc(-c2nnc(Cc3cccs3)[nH]2)c1]

A mixture of 3-((1H-indol-5-yl)oxy)benzohydrazide (Intermediate 5) (262 mg, 0.98 mmol), 2-(thiophen-2-yl)acetonitrile (181 mg, 1.47 mmol) and K₂CO₃ (406 mg, 2.94 mmol) in n-butanol (10 mL) was stirred at 130° C. for 12 hours under an N₂ atmosphere. The mixture was concentrated in vacuo, and ethyl acetate (50 mL) was added. The organic layer was washed with water (50 mL), brine (50 mL), dried with anhydrous sodium sulfate, filtered, and concentrated in vacuo. The residue was purified by prep-HPLC to afford 5-(3-(5-(thiophen-2-ylmethyl)-4H-1,2,4-triazol-3-yl)phenoxy)-1H-indole (69.2 mg, 19%) as a yellow solid. MS (ESI): 373.1 m/z (M+H)⁺. ¹H NMR (500 MHz, CDCl₃): δ 9.04 (s, 1H), 7.48 (s, 1H), 7.41-7.23 (m, 3H), 7.13-7.09 (m, 2H), 7.03-6.95 (m, 2H), 6.86-6.85 (m, 1), 6.80 (s, 1H), 6.73 (d, J=7.5 Hz, 1H), 6.34 (s, 1H), 4.16 (s, 2H) ppm.

### Example 11. Synthesis of 5-(3-(5-(thiophen-3-ylmethyl)-4H-1,2,4-triazol-3-yl)phenoxy)-1H-indole

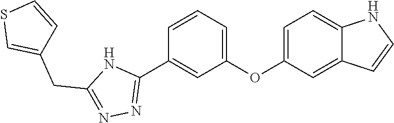 [SMILES: c1cc(Oc2ccc3[nH]ccc3c2)cc(-c2nnc(Cc3ccsc3)[nH]2)c1]

A mixture of 3-((1H-indol-5-yl)oxy)benzohydrazide (Intermediate 5) (250 mg, 0.94 mmol), 2-(thiophen-3-yl)acetonitrile (173 mg, 1.40 mmol) and K₂CO₃ (388 mg, 2.81 mmol) in n-butanol (10 mL) was stirred at 140° C. for 6 hours under a N₂ atmosphere. The mixture was diluted with ethyl acetate (80 mL), and the organic layer was washed with water (50 mL), brine (50 mL), dried over anhydrous sodium sulfate, filtered, and concentrated in vacuo. The residue was purified by prep-HPLC to afford 5-(3-(5-(thiophen-3-ylmethyl)-4H-1,2,4-triazol-3-yl)phenoxy)-1H-indole (130 mg, 37%) as a white solid. MS (ESI): 373.1 m/z (M+H)⁺. ¹H NMR (500 MHz, DMSO-d₆): δ 11.19 (br s, 1H), 7.66 (d, J=7.5 Hz, 1H), 7.47-7.40 (m, 5H), 7.27 (d, J=2.0 Hz, 2H), 7.04-7.00 (m, 2H), 6.88 (dd, J=8.5 Hz & 2.0 Hz, 1H), 6.43 (d, J=3.5 Hz, 1H), 4.07 (s, 2H) ppm.

### Example 12. Synthesis of 2-((5-(3-((1H-indol-5-yl)oxy)phenyl)-4H-1,2,4-triazol-3-yl)methyl)thiazole

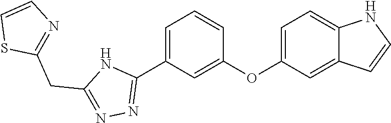 [SMILES: c1cc(Oc2ccc3[nH]ccc3c2)cc(-c2nnc(Cc3nccs3)[nH]2)c1]

### Thiazol-2-ylmethanol

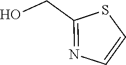

Step A: To a stirred solution of thiazole-2-carboxylic acid (7.9 g, 61.2 mmol) in THE (100 mL) was added lithium aluminum hydride (4.65 g, 0.12 mol) at 0° C. The resulting mixture was stirred at 0° C. for 0.5 hours, warmed to room temperature, and then quenched with Na₂SO₄·10H₂O at 0° C. The mixture was filtered through Celite, and the filtrate was concentrated in vacuo to give thiazol-2-ylmethanol (1.09 g, 15%) as a brown oil, which was used directly in the next step. MS (ESI): 116.1 m/z (M+H)⁺. 2-(Chloromethyl)thiazole ci s

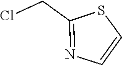

Step B: To a stirred solution of thiazol-2-ylmethanol (500 mg, 4.3 mmol) and CCl₄ (3.31 g, 21.5 mmol) in toluene (5 mL) was added PPh₃ (2.25 g, 8.6 mmol). The reaction mixture was stirred at 100° C. under N₂ for 2 hours, cooled to room temperature and filtered through celite. The filtrate was concentrated in vacuo to afford 2-(chloromethyl)thiazole as a brown oil, which was used directly in the next step. MS (ESI): 133.7 m/z (M+H)⁺.

### 2-(Thiazol-2-yl)acetonitrile

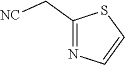

Ste C: To a stirred solution of 2-(chloromethyl)thiazole (4.3 mmol) in CH₂Cl₂ (5 mL) was added trimethylsilyl cyanide (1.856 g, 18.75 mmol) and TBAF (18.75 mL, 1 M in THF, 18.75 mmol). The reaction mixture was stirred at 40° C. for 2 hours. The resulting mixture was concentrated in vacuo. The residue was quenched with water (50 mL). The aqueous layer was extracted with ethyl acetate (50 mL×3). The combined organic layer was washed with brine, dried over anhydrous sodium sulfate, filtered, and concentrated in vacuo. The crude product was purified by prep-TLC (eluting with 1/1 petroleum ether/ethyl acetate) to afford 2-(thiazol-2-yl)acetonitrile (160 mg, 30%, 2 steps) as a light-yellow oil. MS (ESI): 124.7 m/z (M+H)⁺.

### 2-((5-(3-((1H-Indol-5-yl)oxy)phenyl)-4H-1,2,4-triazol-3-yl)methyl)thiazole

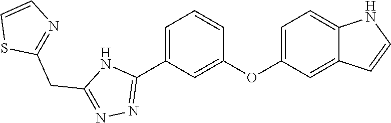 [SMILES: c1cc(Oc2ccc3[nH]ccc3c2)cc(-c2nnc(Cc3nccs3)[nH]2)c1]

Step D: A mixture of 2-(thiazol-2-yl)acetonitrile (160 mg, 1.29 mmol), 3-((1H-indol-5-yl)oxy)benzohydrazide (Intermediate 5) (344 mg, 1.29 mmol) and K₂CO₃ (534 mg, 3.87 mmol) in n-butanol (6 mL) was stirred at 130° C. overnight. The reaction mixture was cooled to room temperature, quenched with water (30 mL) and extracted with ethyl acetate (3×30 mL). The combined organic phase was dried over anhydrous Na₂SO₄ and concentrated in vacuo.. The crude product was purified by prep-HPLC to afford 2-((5-(3-((1H-indol-5-yl)oxy)phenyl)-4H-1,2,4-triazol-3-yl)methyl)thiazole (48 mg, 10%) as a yellow solid. MS (ESI): 374.1 m/z (M+H)⁺. ¹H NMR (500 MHz, DMSO-d₆): δ 11.20 (brs, 1H), 7.72 (d, J=4.0 Hz, 1H), 7.66-7.61 (m, 3H), 7.47-7.41 (m, 4H), 7.27 (d, J=2.0 Hz, 1H), 7.03 (dd, J=2.5, 8.5 Hz, 1H), 6.88 (dd, J=2.5, 8.5 Hz, 1H), 6.42 (dd, J=2.5 Hz & 2.5 Hz, 1H), 4.48 (s, 2H) ppm.

### Example 13. Synthesis of 4-((5-(3-((1H-indol-5-yl)oxy)phenyl)-4H-1,2,4-triazol-3-yl)methyl)thiazole

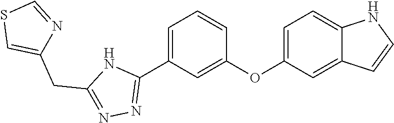 [SMILES: c1cc(Oc2ccc3[nH]ccc3c2)cc(-c2nnc(Cc3cscn3)[nH]2)c1]

### Thiazol-4-ylmnethanol

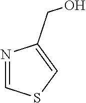

Step A: To a stirred solution of thiazole-4-carboxylic acid (5.0 g, 38.8 mmol) in THE (50 mL) was added lithium aluminum hydride (2.2 g, 58.2 mmol) at 0° C. The resulting mixture was stirred at 0° C. for 0.5 hours, warmed to room temperature, and then quenched with Na₂SO₄·10H₂O at 0° C. The mixture was filtered through Celite, and the filtrate was concentrated in vacuo. The residue was purified by silica gel column chromatography (eluting with 30/1-10/1 dichloromethane/methanol) to give thiazol-4-ylmethanol (500 mg, 10%) as a brown oil. MS (ESI): 116.1 m/z (M+H)⁺.

### 4-(Chloromethyl)thizaole

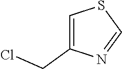

Step B: To a stirred solution of thiazol-4-ylmethanol (500 mg, 4.3 mmol) in dichloromethane (10 mL) was added SOCl₂ (1.54 g, 12.9 mmol) at 0° C. The resulting mixture was stirred at room temperature for 3 hours, and then quenched with saturated aqueous NaHCO₃. The organic layer was separated, washed with brine (20 mL), dried over anhydrous sodium sulfate, filtered, and concentrated in vacuo to give 4-(chloromethyl)thiazole (500 mg, 87%) as a yellow solid, which was used directly in the next step. MS (ESI): 133.7 m/z (M+H)⁺.

### 2-(Thiazol-4-yl)acetonitrile

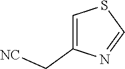

Step C: A mixture of 4-(chloromethyl)thiazole (500 mg, 3.8 mmol) and NaCN (411 mg, 8.4 mmol) in DMSO (5 mL) was stirred at 60° C. for 4 hours. The reaction mixture was cooled to room temperature, quenched with water (20 mL), and extracted with ethyl acetate (20 mL×3). The combined organic phase was washed with brine, dried over anhydrous sodium sulfate, filtered, and concentrated in vacuo. The crude product was purified by silicagel column chromatography (eluting with 2/1-1/1 petroleum ether/ethyl acetate) to give 2-(thiazol-4-yl)acetonitrile (400 mg, 86%) as a colorless liquid. MS (ESI): 125.2 m/z (M+H)⁺.

### 4-((5-(3-((1H-Indol-5-yl)oxy)phenyl)-4H-1,2,4-triazol-3-yl)methyl)thiazole

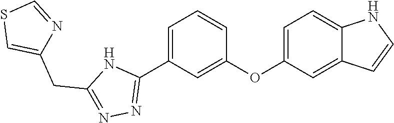 [SMILES: c1cc(Oc2ccc3[nH]ccc3c2)cc(-c2nnc(Cc3cscn3)[nH]2)c1]

Step D: A mixture of 2-(thiazol-4-yl)acetonitrile (248 mg, 2.0 mmol), 3-((1H-indol-5-yl)oxy)benzohydrazide (350 mg, 1.3 mmol) and K₂CO₃ (538 mg, 3.9 mmol) in n-BuOH (5 ml) was stirred at 140° C. for 2 hours under an N₂ atmosphere. The reaction mixture was cooled to room temperature, quenched with water (30 mL) and extracted with ethyl acetate (30 mL×3). The combined organic phase was washed with brine, dried over anhydrous sodium sulfate, filtered, and concentrated in vacuo. The crude product was purified by prep-HPLC to afford 4-((5-(3-((1H-indol-5-yl)oxy)phenyl)-4H-1,2,4-triazol-3-yl)methyl)thiazole (15.5 mg, 3%) as a white solid. MS (ESI): 374.2 m/z (M+H)⁺. ¹H NMR (500 MHz, CDCl₃): δ 8.68 (d, J=2.0 Hz, 1H), 7.58 (d, J=7.5 Hz, 1H), 7.48 (s, 1H), 7.27-7.22 (m, 3H), 7.16-7.13 (m, 2H), 6.95 (dd, J=8.0 Hz & 2.0 Hz, 1H), 6.83 (dd, J=8.5 Hz & 2.5 Hz, 1H), 6.38 (d, J=3.0 Hz, 1H), 4.24 (s, 2H) ppm.

### Example 14. Synthesis of 5-((5-(3-((1H-indol-5-yl)oxy)phenyl)-4H-1,2,4-triazol-3-yl)methyl)thiazole

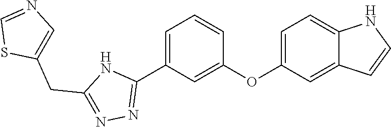 [SMILES: c1cc(Oc2ccc3[nH]ccc3c2)cc(-c2nnc(Cc3cncs3)[nH]2)c1]

### Thiazol-5-ylmethanol

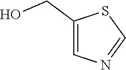

Step A: To a stirred solution of thiazole-5-carboxylic acid (1.9 g, 14.7 mmol) in THE (50 mL) was added lithium aluminum hydride (1.12 g, 29.4 mmol) at 0° C. The resulting mixture was stirred at 0° C. for 0.5 hours, warmed to room temperature, and then quenched with Na₂SO₄·10H₂O at 0° C. The mixture was filtered through Celite, and the filtrate was concentrated in vacuo to give thiazol-5-ylmethanol (635 mg, 35%) as a brown oil, which was used directly for the next step. MS (ESI): 116.1 m/z (M+H)⁺.

### 5-(Chloromethyl)thiazole

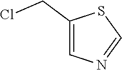

Step B: To a stirred solution of thiazol-5-ylmethanol (635 mg, 5.5 mmol) in dichloromethane (20 mL) was added SOCl₂ (1.963 g, 16.5 mmol) at 0° C. The resulting mixture was stirred at room temperature for 3 hours, and then quenched with saturated aqueous NaHCO₃. The organic layer was separated, washed with brine (20 mL), dried over anhydrous sodium sulfate, filtered and concentrated in vacuo to give 5-(chloromethyl)thiazole (470 mg, 65%) as a brown oil, which was used directly for the next step. MS (ESI): 134.1 m/z (M+H)⁺.

### 2-(Thiazol-5-yl)acetonitrile

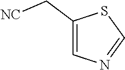

Step C: A mixture of 5-(chloromethyl)thiazole (470 mg, 3.5 mmol) and sodium cyanide (515 mg, 10.5 mmol) in DMSO (5 mL) was stirred at 60° C. for 3 hours. The reaction mixture was cooled to room temperature, quenched with water (20 mL) and extracted with ethyl acetate (20 mL×3). The combined organic phase was washed with brine, dried over anhydrous sodium sulfate, filtered and concentrated in vacuo. The crude product was purified by prep-TLC (eluting with 1/1 petroleum ether/ethyl acetate) to afford 2-(thiazol-5-yl)acetonitrile (150 mg, 45%) as a yellow oil. MS (ESI): 124.7 m/z (M+H)⁺.

### 5-((5-(3-((1H-Indol-5-yl)oxy)phenyl)-4H-1,2,4-triazol-3-yl)methyl)thiazole

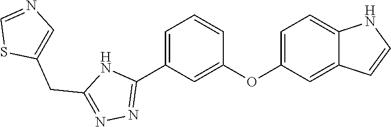 [SMILES: c1cc(Oc2ccc3[nH]ccc3c2)cc(-c2nnc(Cc3cncs3)[nH]2)c1]

Step D: A mixture of 2-(thiazol-5-yl)acetonitrile (120 mg, 0.9 mmol), 3-((1H-indol-5-yl)oxy)benzohydrazide (160 mg, 0.6 mmol) and K₂CO₃ (248 mg, 1.80 mmol) in n-BuOH (5 mL) was stirred at 140° C. for 2 hours under an N₂ atmosphere. The reaction mixture was cooled to room temperature, quenched with water (30 mL), and extracted with ethyl acetate (30 mL×3). The combined organic phase was washed with brine, dried over anhydrous sodium sulfate, filtered, and concentrated in vacuo. The crude product was purified by prep-HPLC to afford 5-((5-(3-((1H-indol-5-yl)oxy)phenyl)-4H-1,2,4-triazol-3-yl)methyl)thiazole (58 mg, 25%) as a white solid. MS (ESI): 374.1 n/z (M+H)⁺. ¹H NMR (500 MHz, DMSO-d₆): δ 14.07 (brs, 1H), 11.19 (s, 1H), 8.96 (s, 1H), 7.71 (s, 1H), 7.64 (d, J=7.5 Hz, 1H), 7.45-7.40 (m, 4H), 7.26 (d, J=1.5 Hz, 1H), 7.02 (d, J=8.0 Hz, 1H), 6.87 (dd, J=8.5 Hz & 2.0 Hz, 1H), 6.42 (s, 1H), 4.34 (s, 2H) ppm.

### Example 15. Synthesis of 2-((5-(3-((1H-indol-5-yl)oxy)phenyl)-4H-1,2,4-triazol-3-yl)methyl)-4-methylthiazole

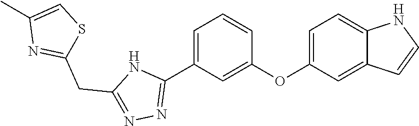 [SMILES: Cc1csc(Cc2nnc(-c3cccc(Oc4ccc5[nH]ccc5c4)c3)[nH]2)n1]

### (4-Methylthiazol-2-yl)methanol

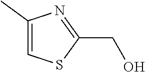

Step A: To a stirred solution of 4-methylthiazole-2-carboxylic acid (3.0 g, 20.9 mmol) in THE (50 mL) was added lithium aluminum hydride (1.6 g, 41.9 mmol) at 0° C. The resulting mixture was stirred at 0° C. for 0.5 hours, warmed to room temperature, and then quenched with Na₂SO₄·10H₂O at 0° C. The mixture was filtered through Celite, and the filtrate was concentrated in vacuo to give (4-methylthiazol-2-yl)methanol (980 mg, 36%) as a brown oil, which was used directly for the next step. MS (ESI): 130.1 m/z (M+H)⁺.

### 2-(Chloromethyl)-4-methylthiazole

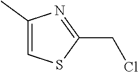

Step B: To a stirred solution of (4-methylthiazol-2-yl)methanol (980 mg, 7.59 mmol) in dichloromethane (20 mL) was added SOCl₂ (1.6 mL, 22.8 mmol) at 0° C. The resulting mixture was stirred at room temperature for 2 hours, and then quenched with saturated aqueous NaHCO₃. The organic layer was separated, washed with brine (20 mL), dried over anhydrous sodium sulfate, filtered and concentrated in vacuo to give the crude product, which was purified by silica gel column chromatography (eluting with 20/1 dichloromethane/methanol) to give 2-(chloromethyl)-4-methylthiazole (273 mg, 24%) as a brown oil. MS (ESI): 148.0 m/z (M+H)⁺.

### 2-(4-Methylthiazol-2-yl)acetonitrile

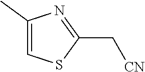

Step C: A mixture of 2-(chloromethyl)-4-methylthiazole (273 mg, 1.86 mmol) and NaCN (273 mg, 5.57 mmol) in DMSO (10 mL) was stirred at 60° C. for 2 hours. The reaction mixture was cooled to room temperature, quenched with water (20 mL) and extracted with ethyl acetate (20 mL×3). The combined organic phase was washed with brine, dried over anhydrous sodium sulfate, filtered and concentrated in vacuo to give 2-(4-methylthiazol-2-yl)acetonitrile (169 mg, 55%) as a brown solid, which was used directly in the next step. MS (ESI): 139.1 m/z (M+H)⁺.

### 2-((5-(3-((1H-Indol-5-yl)oxy)phenyl)-4H-1,2,4-triazol-3-yl)methyl)-4-methylthiazole

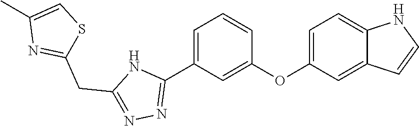 [SMILES: Cc1csc(Cc2nnc(-c3cccc(Oc4ccc5[nH]ccc5c4)c3)[nH]2)n1]

Step D: A mixture of 2-(4-methylthiazol-2-yl)acetonitrile (116.3 mg, 0.84 mmol), 3-((1H-indol-5-yl)oxy)benzohydrazide (Intermediate 5) (150 mg, 0.56 mmol) and K₂CO₃ (232.6 mg, 1.68 mmol) in n-BuOH (10 mL) was stirred at 130° C. for 12 hours under an N₂ atmosphere. The mixture was concentrated, and ethyl acetate (50 mL) was added. The organic layer was washed with water (50 mL) and brine (50 mL), dried over anhydrous sodium sulfate, filtered, and concentrated in vacuo. The residue was purified by prep-HPLC to afford 2-((5-(3-((1H-indol-5-yl)oxy)phenyl)-4H-1,2,4-triazol-3-yl)methyl)-4-methylthiazole (28.6 mg, 11%) as a yellow solid. MS (ESI): 388.1 m/z (M+H)⁺. ¹H NMR (500 MHz, MeOD-d₄): δ 7.63 (d, J=7.5 Hz, 1H), 7.57-7.56 (m, 1H), 7.44-7.39 (m, 2H), 7.30 (d, J=3.0 Hz, 1H), 7.27 (d, J=2.0 Hz, 1H), 7.06-7.02 (m, 2H), 6.89 (dd, J=8.5 Hz & 2.0 Hz, 1H), 6.45 (d, J=3.0 Hz, 1H), 4.48 (s, 2H), 2.40 (s, 3H) ppm.

### Example 16. Synthesis of 2-((5-(3-((1H-indol-5-yl)oxy)phenyl)-4H-1,2,4-triazol-3-yl)methyl)-5-methylthiazole

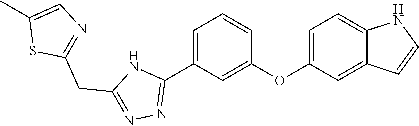 [SMILES: Cc1cnc(Cc2nnc(-c3cccc(Oc4ccc5[nH]ccc5c4)c3)[nH]2)s1]

### (5-Methylthiazol-2-yl)methanol

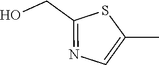

Step A: To a stirred solution of 5-methylthiazole-2-carboxylic acid (1.5 g, 10.5 mmol) in THE (60 mL) was added lithium aluminum hydride (797 mg, 20.9 mmol) at 0° C. The resulting mixture was warmed to room temperature and stirred at room temperature for 2 hours, and then quenched with Na₂SO₄·10H₂O at 0° C. The mixture was filtered through Celite, and the filtrate was concentrated in vacuo to give (5-methylthiazol-2-yl)methanol (780 mg, 58%) as a brown oil, which was used directly in the next step. MS (ESI): 130.2 m/z (M+H)⁺.

### 2-(Chloromethyl)-5-methylthiazole

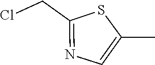

Step B: A mixture of (5-methylthiazol-2-yl)methanol (500 mg, 3.87 mmol), Ph₃P (2.3 g, 7.75 mmol) and toluene (15 mL) in CCl₃ (15 mL) was stirred at 100° C. for 2 hours. The resulting solution was concentrated in vacuo, and the residue was dissolved in EtOAc (50 mL). The organic phase was washed with water, brine, dried over anhydrous sodium sulfate, filtered, and concentrated in vacuo to give 2-(chloromethyl)-5-methylthiazole (554 mg, crude) as a brown oil, which was used directly in the next step. MS (ESI): 148.0 m/z (M+H)⁺.

### 2-(5-Methylthiazol-2-yl)acetonitrile

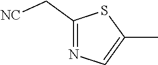

Step C: A mixture of 2-(chloromethyl)-5-methylthiazole (554 mg, 3.77 mmol), trimethylsilyl cyanide (3.7 g, 37.7 mmol) and TBAF (9.8 g, 37.7 mmol) in DCM (30 mL) was stirred at 40° C. for 2 hours. The resulting solution was concentrated in vacuo, and the residue was dissolved in EtOAc (50 mL). The organic phase was washed with water, brine, dried over anhydrous sodium sulfate, filtered and concentrated in vacuo. The residue was purified by prep-TLC (eluting with 1/1 petroleum ether/ethyl acetate) to afford 2-(5-methylthiazol-2-yl)acetonitrile (145 mg, 28% of 2 steps) as a yellow oil. MS (ESI): 139.1 m/z (M+H)⁺.

### 2-((5-(3-((1H-Indol-5-yl)oxy)phenyl)-4H-1,2,4-triazol-3-yl)methyl)-5-methylthiazole

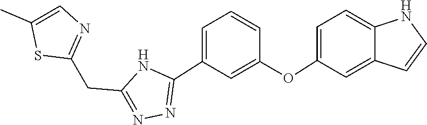 [SMILES: Cc1cnc(Cc2nnc(-c3cccc(Oc4ccc5[nH]ccc5c4)c3)[nH]2)s1]

Step D: A mixture of 2-(5-methylthiazol-2-yl)acetonitrile (155 mg, 1.12 mmol), 3-((1H-indol-5-yl)oxy)benzohydrazide (Intermediate 5) (200 mg, 0.75 mmol) and K₂CO₃ (310.1 mg, 2.25 mmol) in n-BuOH (10 mL) was stirred at 140° C. for 2 hours under an N₂ atmosphere. The mixture was concentrated in vacuo, and the residue was dissolved in EtOAc (50 mL). The organic layer was washed with water (50 mL), brine (50 mL), dried over anhydrous sodium sulfate, filtered and concentrated in vacuo. The residue was purified by prep-HPLC to afford 2-((5-(3-((1H-indol-5-yl)oxy)phenyl)-4H-1,2,4-triazol-3-yl)methyl)-5-methylthiazole (5.1 mg, 2%) as a yellow solid. MS (ESI): 388.1 m/z (M+H)⁺. ¹H NMR (500 MHz, MeOH-d₄): δ 7.63 (d, J=8.0 Hz, 1H), 7.56 (s, 1H), 7.43-7.36 (m, 3H), 7.30 (d, J=3.0 Hz, 1H), 7.27 (d, J=2.0 Hz, 1H), 7.05-7.03 (m, 1H), 6.89 (dd, J=8.5 Hz & 2.0 Hz, 1H), 6.44 (d, J=3.0 Hz, 1H), 4.45 (s, 2H), 2.45 (s, 3H) ppm.

### Example 17. Synthesis of 4-((5-(3-((1H-indol-5-yl)oxy)phenyl)-4H-1,2,4-triazol-3-yl)methyl)-2-methylthiazole

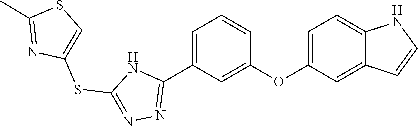 [SMILES: Cc1nc(Sc2nnc(-c3cccc(Oc4ccc5[nH]ccc5c4)c3)[nH]2)cs1]

### (2-Methylthiazol-4-yl)methanol

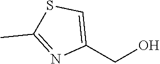

Step A: To a stirred solution of ethyl 2-methylthiazole-4-carboxylate (3.0 g, 17.5 mmol) in THE (50 mL) was added lithium aluminum hydride (1.0 g, 26.3 mmol) at −10° C. The resulting mixture was stirred at −10° C. for 0.5 hours, warmed to room temperature, and then quenched with Na₂SO₄·10H₂O at 0° C. The mixture was filtered through Celite, and the filtrate was concentrated in vacuo to give (2-methylthiazol-4-yl)methanol (2.1 g, 95%) as a brown oil, which was used directly in the next step. MS (ESI): 130.2 m/z (M+H)⁺.

### 4-(Chloromethyl)-2-methylthiazole

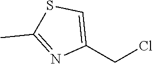

Step B: To a stirred solution of (2-methylthiazol-4-yl)methanol (2.1 g, 16.3 mmol) in dichloromethane (50 mL) was added SOCl₂ (3.5 mL, 48.8 mmol) at 0° C. The resulting mixture was stirred at room temperature for 2 hours, and then quenched with saturated aqueous NaHCO₃. The organic layer was separated, washed with brine (20 mL), dried over anhydrous sodium sulfate, filtered, and concentrated in vacuo. The crude product was purified by silica gel chromatography (eluting with 20/1 dichloromethane/methanol) to give 4-(chloromethyl)-2-methylthiazole (363 mg, 15%) as a brown oil. MS (ESI): 148.0 m/z (M+H)⁺. ¹H NMR (500 MHz, CDCl₃) δ 7.16 (s, 1H), 4.68 (s, 2H), 2.75 (s, 3H) ppm.

### 2-(2-Methylthiazol-4-yl)acetonitrile

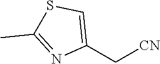

Step C: A mixture of 4-(chloromethyl)-2-methylthiazole (363 mg, 2.47 mmol) and NaCN (363 mg, 7.41 mmol) in DMSO (10 mL) was stirred at 60° C. for 2 hours. The reaction mixture was cooled to room temperature, quenched with water (20 mL) and extracted with ethyl acetate (20 mL×3). The combined organic phase was washed with brine, dried over anhydrous sodium sulfate, filtered and concentrated in vacuo to give 2-(2-methylthiazol-4-yl)acetonitrile (263 mg, 77%) as a brown solid, which was used directly in the next step. MS (ESI): 139.2 m/z (M+H)⁺.

### 4-((5-(3-((1H-Indol-5-yl)oxy)phenyl)-4H-1,2,4-triazol-3-yl)methyl)-2-methylthiazole

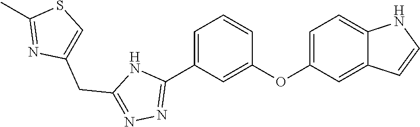 [SMILES: Cc1nc(Cc2nnc(-c3cccc(Oc4ccc5[nH]ccc5c4)c3)[nH]2)cs1]

Step D: A mixture of 2-(2-methylthiazol-4-yl)acetonitrile (116.3 mg, 0.84 mmol), 3-((1H-indol-5-yl)oxy)benzohydrazide (Intermediate 5) (150 mg, 0.56 mmol) and K₂CO₃ (232.6 mg, 1.68 mmol) in n-BuOH (10 mL) was stirred at 130° C. for 12 hours under an N₂ atmosphere. The mixture was concentrated, and ethyl acetate (50 mL) was added. The organic layer was washed with water (50 mL) and brine (50 mL), dried over anhydrous sodium sulfate, filtered, and concentrated in vacuo. The residue was purified by prep-HPLC to afford 4-((5-(3-((1H-indol-5-yl)oxy)phenyl)-4H-1,2,4-triazol-3-yl)methyl)-2-methylthiazole (36.8 mg, 17%) as a yellow solid. MS (ESI): 388.1 m/z (M+H)⁺. ¹H NMR (500 MHz, MeOH-d₄) δ 7.64 (d, J=7.0 Hz, 1H), 7.56 (s, 1H), 7.43-7.38 (m, 2H), 7.29 (d, J=3.0 Hz, 1H), 7.26 (d, J=2.0 Hz, 1H), 7.14 (s, 1H), 7.02 (d, J=7.0 Hz, 1H), 6.89 (dd, J=9.0 Hz & 2.5 Hz, 1H), 6.44 (d, J=3.5 Hz, 1H), 4.24 (s, 2H), 2.68 (s, 3H) ppm.

### Example 18. Synthesis of 5-((5-(3-((1H-indol-5-yl)oxy)phenyl)-4H-1,2,4-triazol-3-yl)methyl)-2-methylthiazole

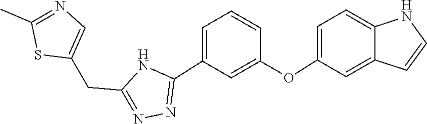 [SMILES: Cc1ncc(Cc2nnc(-c3cccc(Oc4ccc5[nH]ccc5c4)c3)[nH]2)s1]

### (2-Methylthiazol-5-yl)methanol

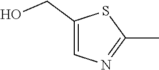

Step A: To a stirred solution of 2-methylthiazole-5-carboxylic acid (1.5 g, 10.5 mmol) in THF (30 mL) was added lithium aluminum hydride (598 mg, 26.3 mmol) at −10° C. The resulting mixture was stirred at −10° C. for 0.5 hours, warmed to room temperature, and then quenched with Na₂SO₄·10H₂O at 0° C. The mixture was filtered through Celite, and the filtrate was concentrated in vacuo to give (2-methylthiazol-5-yl)methanol (1.08 g, 80%) as a brown oil, which was used directly in the next step. MS (ESI): 130.2 m/z (M+H)⁺.

### 5-(Chloromethyl)-2-methylthiazole

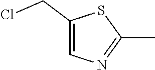

Step B: To a stirred solution of (2-methylthiazol-5-yl)methanol (1.6 g, 12.4 mmol) in dichloromethane (50 mL) was added SOCl₂ (4.39 mL, 37.2 mmol) at 0° C. The resulting mixture was stirred at room temperature for 2 hours, and then quenched with saturated aqueous NaHCO₃. The organic layer was separated, washed with brine (20 mL), dried over anhydrous sodium sulfate, filtered and concentrated in vacuo to give the crude product, which was purified by silica gel chromatography (eluting with 1/1-1/3 petroleum ether/ethyl acetate) to give 5-(chloromethyl)-2-methylthiazole (1.13 g, 62%) as a yellow oil. MS (ESI): 148.0 m/z (M+H)⁺.

### 2-(2-Methylthiazol-5-yl)acetonitrile

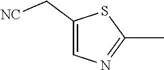

Step C: A mixture of 5-(chloromethyl)-2-methylthiazole (1.1 g, 2.47 mmol) and NaCN (363 mg, 7.41 mmol) in DMSO (10 mL) was stirred at 60° C. for 2 hours. The reaction mixture was cooled to room temperature, quenched with water (20 mL) and extracted with ethyl acetate (20 mL×3). The combined organic phase was washed with brine, dried over anhydrous sodium sulfate, filtered, and concentrated in vacuo. The crude product was purified by silica gel chromatography (eluting with 2/1-1/1 petroleum ether/ethyl acetate) to give 2-(2-methylthiazol-5-yl)acetonitrile (528 mg, 51%) as a yellow oil. MS (ESI): 139.2 m/z (M+H)⁺. 5-((5-(3-((1H-Indol-5-yl)oxy)phenyl)-4H-1,2,4-triazol-3-yl)methyl)-2-methylthiazole

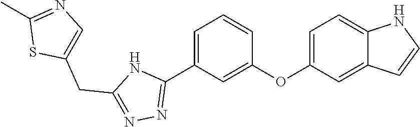 [SMILES: Cc1ncc(Cc2nnc(-c3cccc(Oc4ccc5[nH]ccc5c4)c3)[nH]2)s1]

Step D: A mixture of 2-(2-methylthiazol-5-yl)acetonitrile (150 mg, 1.09 mmol), 3-((1H-indol-5-yl)oxy)benzohydrazide (Intermediate 5) (203 mg, 0.76 mmol) and K₂CO₃ (450 mg, 3.26 mmol) in n-BuOH (10 mL) was stirred at 130° C. for 12 hours under an N₂ atmosphere. The mixture was concentrated, and ethyl acetate (50 mL) was added. The organic layer was washed with water (50 mL) and brine (50 mL), dried with anhydrous sodium sulfate, filtered, and concentrated in vacuo. The residue was purified by prep-HPLC to afford 5-((5-(3-((1H-indol-5-yl)oxy)phenyl)-4H-1,2,4-triazol-3-yl)methyl)-2-methylthiazole (40 mg, 14%) as a white solid. MS (ESI): 388.0 m/z (M+H)⁺. ¹H NMR (500 MHz, MeOH-d₄): δ 7.61 (d, J=7.0 Hz, 1H), 7.54 (s, 1H), 7.46 (s, 1H), 7.43-7.37 (m, 2H), 7.29 (d, J=3.0 Hz, 1H), 7.26 (d, J=2.0 Hz, 1H), 7.02 (dd, J=8.5 Hz & 2.5 Hz, 1H), 6.88 (dd, J=9.0 Hz & 2.0 Hz, 1H), 6.44 (d, J=3.5 Hz, 1H), 4.29 (s, 2H), 2.63 (s, 3H) ppm.

### Example 19. Synthesis of 5-(3-(3-((5-chlorothiophen-2-yl)methyl)-1H-1,2,4-triazol-5-yl)phenoxy)-1H-pyrrolo[2,3-c]pyridine

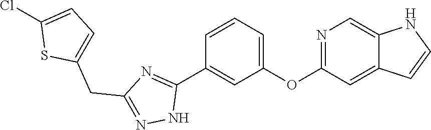 [SMILES: Clc1ccc(Cc2n[nH]c(-c3cccc(Oc4cc5cc[nH]c5cn4)c3)n2)s1]

### (5-Chlorothiophen-2-yl)methanol

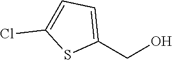

Step A: To a stirred solution of 5-chlorothiophen-2-carbaldehyde (2.2 g, 15.1 mmol) in methanol (30 mL) was added NaBH₄ (1.2 g, 31.2 mol) at 0° C. The resulting mixture was stirred at 0° C. for 3 hours, warmed to room temperature, and then quenched with saturated aqueous NH₄Cl at 0° C. The mixture was filtered through Celite, and the filtrate was concentrated in vacuo, purified by silica gel column chromatography (eluting with 10/1 petroleum ether/ethyl acetate) to give (5-chlorothiophen-2-yl)methanol (1.1 g, 49%) as a colorless oil. MS (ESI): 149.2 m/z (M+H)⁺.

### 2-Chloro-5-(chloromethyl)thiophene

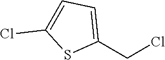

Step B: To a stirred solution of (5-chlorothiophen-2-yl)methanol (1.1 g, 7.4 mmol) in dichloromethane (15 mL) was added SOCl₂ (2.65 g, 22.3 mmol) at 0° C. The resulting mixture was stirred at room temperature for 3 hours, and then quenched with saturated aqueous NaHCO₃. The organic layer was separated, washed with brine (20 mL), dried over anhydrous sodium sulfate, filtered, and concentrated in vacuo to give 2-chloro-5-(chloromethyl)thiophene (1.1 g, 89%) as a brown oil, which was used directly in the next step. MS (ESI): 167.2 m/z (M+H)⁺.

### 2-(5-Chlorothiophen-2-yl)acetonitrile

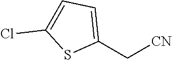

Step C: To a solution of 2-chloro-5-(chloromethyl)thiophene (700 mg, 4.2 mmol) in CH₂CL2 (5 mL) was added trimethylsilyl cyanide (1.856 g, 18.75 mmol) and TBAF (18.75 mL, 1 M in THF, 18.75 mmol). The reaction mixture was stirred at 40° C. for 2 hours. The resulting mixture was concentrated in vacuo. The residue was quenched with water (50 mL). The aqueous layer was extracted with ethyl acetate (50 mL×3). The combined organic layer was washed with brine, dried over anhydrous sodium sulfate, filtered, and concentrated in vacuo. The crude product was purified by silica gel column chromatography (eluting with 15/1 petroleum ether/ethyl acetate) to give 2-(5-chlorothiophen-2-yl)acetonitrile (835 mg, 89%) as a brown oil. MS (ESI): 158.2 m/z (M+H)⁺.

### 5-(3-(3-((5-Chlorothiophen-2-yl)methyl)-1H-1,2,4-triazol-5-yl)phenoxy)-1H-pyrrolo[2,3-c]pyridine

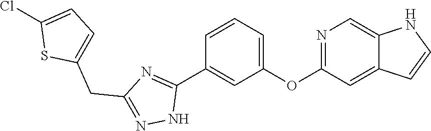 [SMILES: Clc1ccc(Cc2n[nH]c(-c3cccc(Oc4cc5cc[nH]c5cn4)c3)n2)s1]

Step D: A mixture of 3-((1H-pyrrolo[2,3-c]pyridin-5-yl)oxy)benzohydrazide (Step C, Example 8) (300 mg, 1.1 mmol), 2-(5-chlorothiophen-2-yl)acetonitrile (175 mg, 1.1 mmol) and K₂CO₃ (463 mg, 3.3 mmol) in n-BuOH (10 mL) was stirred at 130° C. for 3 hours under an N₂ atmosphere. The mixture was concentrated in vacuo, and ethyl acetate (50 mL) was added. The organic layer was washed with water (50 mL) and brine (50 mL), dried with anhydrous sodium sulfate, filtered and concentrated in vacuo to give a residue, which was purified by prep-HPLC to afford the title compound (12.7 mg, 3%) as a yellow solid. MS (ESI): 408.0 m/z (M+H)⁺. ¹H NMR (500 MHz, MeOH-d₄): δ 8.44 (s, 1H), 7.74 (d, J=8.0 Hz, 1H), 7.61 (d, J=3.0 Hz, 1H), 7.60-7.58 (m, 1H), 7.48 (dd, J=8.0 Hz & 8.0 Hz, 1H), 7.22 (s, 1H), 7.11 (dd, J=8.0 Hz & 1.5 Hz, 1H), 6.83-6.79 (m, 2H), 6.56 (d, J=3.0 Hz, 1H), 4.26 (s, 2H) ppm.

### Example 20. Synthesis of (2-(3-((1H-indol-5-yl)oxy)phenyl)-1H-imidazol-4-yl)(5-methylthiophen-2-yl)methanol

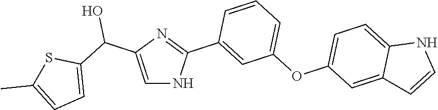 [SMILES: Cc1ccc(C(O)c2c[nH]c(-c3cccc(Oc4ccc5[nH]ccc5c4)c3)n2)s1]

To a stirred solution of 2-methy thiophene (324 mg, 3.30 mmol) in THF (10 mL) was added n-BuLi (1.37 mL, 2.4 N in hexane, 3.30 mmol) at −78° C. The resulting mixture was stirred at −78° C. for 1 hour to give a solution of (5-methylthiophen-2-yl)lithium. To this solution was added at −78° C. a solution of 2-(3-((1H-indol-5-yl)oxy)phenyl)-1H-imidazole-5-carbaldehyde (Intermediate 6) (100 mg, 0.33 mmol) in THE (1 mL). The resulting mixture was warmed to room temperature and stirred at room temperature overnight, and then quenched with water (30 mL). The aqueous phase was extracted by ethyl acetate (30 mL×3). The combined organic layers were washed with brine (30 mL), dried over anhydrous sodium sulfate, filtered and concentrated in vacuo. The crude product was purified by prep-HPLC to afford the title compound (15 mg, 11%) as a white solid. MS (ESI): 402.1 m/z (M+H)⁺. ¹H NMR (500 MHz, MeOH-d₄): δ 7.52 (d, J=8.0 Hz, 1H), 7.48 (dd, J=2.0 Hz & 2.0 Hz, 1H), 7.42 (d, J=9.0 Hz, 1H), 7.36 (dd, J=8.0 Hz & 8.0 Hz, 1H), 7.29 (d, J=3.5 Hz, 1H), 7.26 (d, J=2.5 Hz, 1H), 6.98 (br s, 1H), 6.93 (dd, J=8.0 Hz & 1.5 Hz, 1H), 6.89 (dd, J=9.0 Hz & 2.0 Hz, 1H), 6.79 (d, J=3.0 Hz, 1H), 6.62 (d, J=2.5 Hz, 1H), 6.44 (d, J=3.0 Hz, 1H), 5.95 (s, 1H), 2.45 (s, 3H) ppm.

### Example 21. Synthesis of (2-(3-((1H-indol-5-yl)oxy)phenyl)-1H-imidazol-4-yl)(thiazol-2-yl)methanol

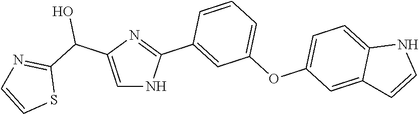 [SMILES: OC(c1c[nH]c(-c2cccc(Oc3ccc4[nH]ccc4c3)c2)n1)c1nccs1]

To a stirred solution of thiazole (2.0 g, 23.5 mmol) in THE (10 mL) was added n-BuLi (9.5 mL, 2.4 N in THF, 22.8 mmol) at −78° C. The resulting mixture was stirred at −78° C. for 1 hour. To 3 mL of this solution was then added at −78° C. a solution of 2-(3-((1H-indol-5-yl)oxy)phenyl)-1H-imidazole-5-carbaldehyde (Intermediate 6) (100 mg, 0.33 mmol) in THE (1 mL). The resulting mixture was warmed to room temperature and stirred at room temperature overnight, and then quenched with saturated aqueous NH₄Cl (10 mL). The aqueous phase was extracted with ethyl acetate (15 mL×3). The combined organic layers were washed with brine (30 mL), dried over anhydrous sodium sulfate, filtered, and concentrated in vacuo. The crude product was purified by prep-HPLC to afford (2-(3-((1H-indol-5-yl)oxy)phenyl)-1H-imidazol-4-yl)(thiazol-2-yl)methanol (30 mg, 23%) as a white solid. MS (ESI): 389.1 m/z (M+H)⁺. ¹H NMR (500 MHz, MeOH-d₄) δ 7.75 (d, J=3.0 Hz, TH), 7.57 (d, J=3.0 Hz, TH), 7.53 (dd, J=8.0 Hz & 1.0 Hz, 1H), 7.48 (dd, J=2.0 Hz & 2.0 Hz, 1H), 7.41 (d, J=8.5 Hz, 1H), 7.36 (dd, J=8.0 Hz & 8.0 Hz, 1H), 7.29 (d, J=2.5 Hz, 1H), 7.26 (d, J=2.0 Hz, 1H), 6.97-6.92 (m, 2H), 6.88 (dd, J=8.5 Hz & 2.0 Hz, 1H), 6.44 (dd, J=3.0 Hz & 0.5 Hz, 1H), 6.08 (s, 1H) ppm.

### Example 22. Synthesis of (2-(3-((1H-indol-5-yl)oxy)phenyl)-1H-imidazol-4-yl)(5-methylthiazol-2-yl)methanol

### Example 23. Synthesis of (S)-1-(2-(3-((1H-indol-5-yl)oxy)phenyl)-1H-imidazol-4-yl)-2-(thiazol-5-yl)ethan-1-ol

### Example 24. Synthesis of (R)-1-(2-(3-((1H-indol-5-yl)oxy)phenyl)-1H-imidazol-4-yl)-2-(thiazol-5-yl)ethan-1-ol

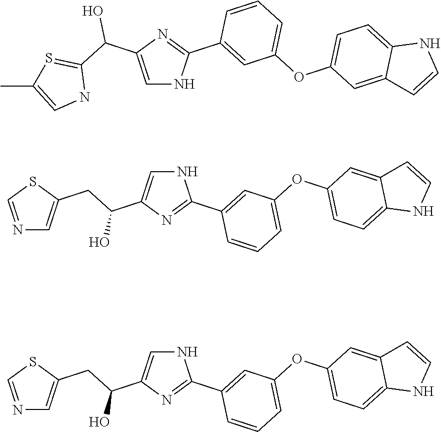

To a stirred solution of 2-methylthiazole (327 mg, 3.30 mmol) in THF (10 mL) was added n-BuLi (0.96 mL, 2.4 N in hexane, 2.30 mmol) at −78° C. The resulting mixture was stirred at −78° C. for 1 hour to afford a solution of (5-methylthiazol-2-yl)lithium and (thiazol-5-ylmethyl)lithium, which was used directly in the next step. To this solution was added at −78° C. a solution of 2-(3-((1H-indol-5-yl)oxy)phenyl)-1H-imidazole-5-carbaldehyde (Intermediate 6) (100 mg, 0.33 mmol) in THE (6 mL). The resulting mixture was warmed to room temperature and stirred at room temperature overnight, and then quenched with water (30 mL). The aqueous phase was extracted with ethyl acetate (30 mL×3). The combined organic layers were washed with brine (30 mL), dried over anhydrous sodium sulfate, filtered, and concentrated in vacuo. The crude product was purified by prep-HPLC and then Chiral-HPLC to afford (2-(3-((1H-indol-5-yl)oxy)phenyl)-1H-imidazol-4-yl)(5-methylthiazol-2-yl)methanol (Example 22) (9.0 mg, 6.7%) as white solid, (S)-1-(2-(3-((1H-indol-5-yl)oxy)phenyl)-1H-imidazol-4-yl)-2-(thiazol-5-yl)ethan-1-ol (Example 23) (retention time in chiral HPLC: 5.11 min) (7.5 mg, 5.6%) as white solid and (R)-1-(2-(3-((1H-indol-5-yl)oxy)phenyl)-1H-imidazol-4-yl)-2-(thiazol-5-yl)ethan-1-ol (Example 24) (retention time in chiral HPLC: 9.00 min) (10.6 mg, 7.9%) as white solid. The absolute configurations of the structures of Example 23 and Example 24 have been assigned arbitrarily.

(2-(3-((1H-indol-5-yl)oxy)phenyl)-1H-imidazol-4-yl)(5-methylthiazol-2-yl)methanol (Example 22) MS (ESI): 403.0 m/z (M+H)⁺. ¹H NMR (500 MHz, MeOD-d₄): δ 7.53-7.47 (m, 3H), 7.42 (d, J=8.5 Hz, 1H), 7.37 (dd, J=8.0 Hz & 8.0 Hz, 1H), 7.29 (d, J=3.0 Hz, 1H), 7.26 (d, J=2.0 Hz, 1H), 7.06 (br s, 1H), 6.96-6.93 (m, 1H), 6.89 (dd, J=8.5 Hz & 2.0 Hz, 1H), 6.44 (d, J=2.5 Hz, 1H), 6.03 (s, 1H), 2.67 (s, 3H).

(S)-1-(2-(3-((1H-indol-5-yl)oxy)phenyl)-1H-imidazol-4-yl)-2-(thiazol-5-yl)ethan-1-ol (Example 23) MS (ESI): 403.0 m/z (M+H)⁺. ¹H NMR (500 MHz, MeOD-d₄) δ 7.67 (d, J=3.5 Hz, 1H), 7.52 (d, J=8.0 Hz, 1H), 7.47 (dd, J=2.0 Hz & 2.0 Hz, 1H), 7.43-7.41 (m, 2H), 7.37 (dd, J=7.5 Hz & 7.5 Hz, 1H), 7.30 (d, J=3.0 Hz, 1H), 7.27 (d, J=2.0 Hz, 1H), 7.01 (br s, TH), 6.94 (dd, J=8.0 Hz & 1.5 Hz, 1H), 6.89 (dd, J=7.0 Hz & 2.0 Hz, 1H), 6.45 (d, J=2.0 Hz, 1H), 5.09 (t, J=6.0 Hz, 1H), 3.62-3.49 (m, 2H).

(R)-1-(2-(3-((1H-indol-5-yl)oxy)phenyl)-1H-imidazol-4-yl)-2-(thiazol-5-yl)ethan-1-ol (Example 24) MS (ESI): 403.0 n/z (M+H)⁺. ¹H NMR (500 MHz, MeOD-d₄): δ 7.67 (d, J=3.5 Hz, 1H), 7.52 (d, J=8.0 Hz, 1H), 7.47 (dd, J=2.0 Hz & 2.0 Hz, 1H), 7.43-7.41 (m, 2H), 7.37 (dd, J=7.5 Hz & 7.5 Hz, 1H), 7.30 (d, J=3.0 Hz, 1H), 7.26 (d, J=2.0 Hz, TH), 7.01 (br s, 1H), 6.94 (d, J=8.0 Hz, 1H), 6.89 (dd, J=7.0 Hz & 2.0 Hz, 1H), 6.45 (dd, J=3.5 Hz & 1.0 Hz, 1H), 5.11-5.07 (m, 1H), 3.62-3.49 (m, 2H).

### Example 25. Synthesis of 5-(3-(5-((1-methyl-1H-pyrazol-4-yl)methyl)-4H-1,2,4-triazol-3-yl)phenoxy)-1H-indole

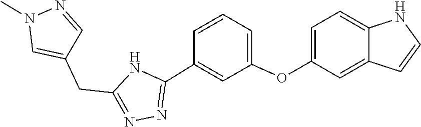 [SMILES: Cn1cc(Cc2nnc(-c3cccc(Oc4ccc5[nH]ccc5c4)c3)[nH]2)cn1]

### 2-(1-Methyl-1H-pyrazol-4-yl)acetonitrile

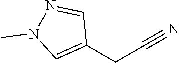

Step A: To a stirred suspension of t-BuOK (2.8 g, 25 mmol) in anhydrous 1,2-dimethoxyethane (20 mL) at −50° C. was added dropwise a solution of TsCH2NC (2.34 g, 12 mmol) in anhydrous 1,2-dimethoxyethane (20 mL) under an N₂ atmosphere, followed by dropwise addition of a solution of 1-methyl-1H-pyrazole-4-carbaldehyde (1.10 g, 10 mmol) in anhydrous 1,2-dimethoxyethane (20 mL) at −50° C. The resulting mixture was stirred at −50° C. for 0.5 hours, and then methanol (20 mL) was added. The mixture was refluxed for 0.5 hours and then concentrated in vacuo. The residue was diluted with water (20 mL) and acetic acid (1.0 mL). The aqueous phase was extracted with dichloromethane (50 mL×3). The combined organic layer was washed with saturated aqueous Na₂CO₃ (50 mL), dried over anhydrous sodium sulfate, filtered, and concentrated in vacuo to give 2-(1-methyl-1H-pyrazol-4-yl)acetonitrile (1.0 g, 82%) as a yellow oil, which was used directly for next step. MS (ESI): 122.1 m/z (M+H)⁺.

### Methyl 2-(1-methyl-1H-pyrazol-4-yl)acetate

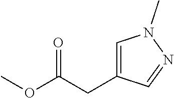

Step B: To a stirred solution of 2-(1-methyl-1H-pyrazol-4-yl)acetonitrile (850 mg, 7.02 mmol) in methanol (20 mL) was added dropwise acetyl chloride (5.54 g, 70.2 mmol) at 0° C. The resulting mixture was stirred at room temperature for 1 hour, and then refluxed overnight and concentrated in vacuo. The residue was dissolved in dichloromethane (50 mL), and the organic phase was washed with saturated aqueous Na₂CO₃ (50 mL) and brine (50 mL), dried over anhydrous sodium sulfate, filtered and concentrated in vacuo. The residue was purified by silica gel column chromatography (eluting with 3/1-1/1 petroleum ether/ethyl acetate) to give methyl 2-(1-methyl-1H-pyrazol-4-yl)acetate (810 mg, 75%) as a yellow solid. MS m/z: 155.1 (M+H⁺).

### 2-(1-Methyl-1H-pyrazol-4-yl)acetohydrazide

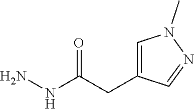

Step C: To a solution of methyl 2-(1-methyl-1H-pyrazol-4-yl)acetate (930 mg, 6.0 mmol) in methanol (20 mL) was added hydrazine hydrate (85%, 5 mL). The resulting reaction mixture was refluxed overnight and concentrated in vacuo to give 2-(1-methyl-1H-pyrazol-4-yl)acetohydrazide (900 mg, 96%) as a light-yellow solid, which was used directly in the next step. MS m/z: 155.1 (M+H⁺).

### 5-(3-(5-((1-Methyl-1H-pyrazol-4-yl)methyl)-4H-1,2,4-triazol-3-yl)phenoxy)-1H-indole

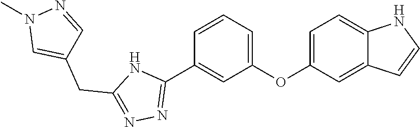 [SMILES: Cn1cc(Cc2nnc(-c3cccc(Oc4ccc5[nH]ccc5c4)c3)[nH]2)cn1]

Step D: A solution of 2-(1-methyl-1H-pyrazol-4-yl)acetohydrazide (123.2 mg, 0.8 mmol), 3-((1H-indol-5-yl)oxy)benzonitrile (Intermediate 6C) (93.6 mg, 0.4 mmol) and K₂CO₃ (110.4 mg, 0.8 mmol) in n-BuOH (4.0 mL) was irradiated at 170° C. for 3 hours in a nitrogen filled microwave tube in a microwave reactor. The reaction mixture was cooled to room temperature, quenched with water (20 mL), and extracted with ethyl acetate (20 mL×3). The combined organic phase was washed with brine (30 mL), dried over anhydrous sodium sulfate, filtered and concentrated in vacuo. The residue was purified by prep-HPLC to afford the title compound (16 mg, 11%) as a white solid. MS m/z: 371.2 (M+H⁺). ¹H NMR (400 MHz, MeOD-d₄): d 7.63 (d, J=8.0 Hz, 1H), 7.55 (d, J=2.0 Hz, 1H), 7.51 (s, 1H), 7.43-7.37 (m, 3H), 7.29 (d, J=3.2 Hz, 1H), 7.26 (d, J=2.4 Hz, 1H), 7.02 (dd, J=8.0 Hz & 2.0 Hz, 1H), 6.89 (dd, J=8.8 Hz & 2.4 Hz, 1H), 6.44 (d, J=3.2 Hz, 1H), 3.99 (s, 2H), 3.85 (s, 3H) ppm.

### Example 26. Synthesis of (2-(3-((1H-indol-5-yl)oxy)phenyl)-1H-imidazol-5-yl)(1-methyl-1H-pyrazol-5-yl)methanol

### Example 27. Synthesis of (2-(3-((1H-indol-5-yl)oxy)phenyl)-1H-imidazol-5-yl)(1-methyl-1H-pyrazol-4-yl)methanol

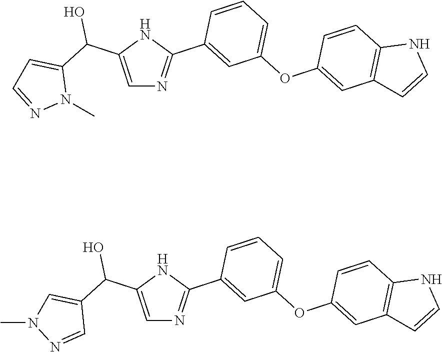 [SMILES: Cn1nccc1C(O)c1cnc(-c2cccc(Oc3ccc4[nH]ccc4c3)c2)[nH]1 ; Cn1cc(C(O)c2cnc(-c3cccc(Oc4ccc5[nH]ccc5c4)c3)[nH]2)cn1]

To a stirred solution of 4-bromo-1-methyl-1H-pyrazole (1.8 g, 11.35 mmol) in THE (50 mL) was added dropwise n-BuLi (4.3 mL, 2.5 N in THF, 10.6 mmol) at −78° C., the mixture was stirred at −78° C. for 0.5 hours, then a solution of 2-(3-((1H-indol-5-yl)oxy)phenyl)-1H-imidazole-5-carbaldehyde (Intermediate 6) (430 mg, 1.42 mmol) in THE (10 mL) was added. The mixture was warmed to room temperature, stirred at room temperature for 1.5 hours and quenched with saturated aqueous NH₄Cl (10 mL). The organic layer was separated, and the aqueous phase was extracted by dichloromethane (15 mL×3). The combined organic layers were washed with brine (10 mL), dried over anhydrous sodium sulfate, filtered and concentrated in vacuo to give a residue. The residue was purified by prep-HPLC to afford (2-(3-((1H-indol-5-yl)oxy)phenyl)-1H-imidazol-5-yl)(1-methyl-1H-pyrazol-5-yl)methanol (38 mg, 7%) and (2-(3-((1H-indol-5-yl)oxy)phenyl)-1H-imidazol-5-yl)(1-methyl-1H-pyrazol-4-yl)methanol (157 mg, 29%) as a white solid.

(2-(3-((1H-indol-5-yl)oxy)phenyl)-1H-imidazol-5-yl)(1-methyl-1H-pyrazol-4-yl)methanol (Example 27): MS m/z: 386.2 (M+H⁺). ¹H NMR (400 MHz, MeOD-d₄): δ 7.54-7.50 (m, 2H), 7.48-7.46 (m, 2H), 7.41 (d, J=8.4 Hz, 1H), 7.36 (dd, J=8.0 Hz & 8.0 Hz, 1H), 7.29 (d, J=3.2 Hz, 1H), 7.25 (d, J=2.4 Hz, 1H), 6.94-6.87 (m, 3H), 6.44 (d, J=3.2 Hz, 1H), 5.81 (s, 1H), 3.86 (s, 3H) ppm.

(2-(3-((1H-indol-5-yl)oxy)phenyl)-1H-imidazol-5-yl)(1-methyl-1H-pyrazol-5-yl)methanol (Example 26): MS m/z: 386.2 (M+H⁺). ¹H NMR (400 MHz, MeOD-d₄): δ 7.52 (d, J=7.6 Hz, 1H), 7.47 (s, 1H), 7.43-7.34 (m, 3H), 7.29 (d, J=3.2 Hz, 1H), 7.25 (d, J=2.0 Hz, 1H), 7.00-6.87 (m, 3H), 6.44 (d, J=2.8 Hz, 1H), 6.18 (s, 1H), 5.94 (s, 1H), 3.87 (s, 3H) ppm.

### Example 28. Synthesis of 5-(3-(5-((1-methyl-1H-pyrazol-4-yl)methyl)-1H-imidazol-2-yl)phenoxy)-1H-indole

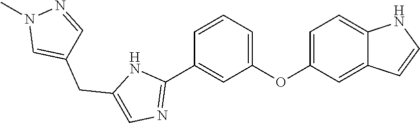 [SMILES: Cn1cc(Cc2cnc(-c3cccc(Oc4ccc5[nH]ccc5c4)c3)[nH]2)cn1]

To a stirred solution of (2-(3-((1H-indol-5-yl)oxy)phenyl)-1H-imidazol-5-yl)(1-methyl-1H-pyrazol-4-yl)methanol (Example 27) (70 mg, 0.18 mmol) in methanol (50 mL) and concentrated HCl (2.5 mL) was added Pd/C (10% wt, 50 mg, 0.34 mmol), the resulting mixture was stirred at room temperature for 16 hours under a H₂ atmosphere. The resulting mixture was filtered through Celite, and the filtrate was concentrated in vacuo. The residue was purified by prep-HPLC to afford 5-(3-(5-((1-methyl-1H-pyrazol-4-yl)methyl)-1H-imidazol-2-yl)phenoxy)-1H-indole (5 mg, 7.5%) as a white solid. MS m/z: 370.2 (M+H⁺). ¹H NMR (400 MHz, MeOD-d₄): δ 7.49 (d, J=7.6 Hz, 1H), 7.46-7.40 (m, 3H), 7.37-7.33 (m, 2H), 7.29 (d, J=3.2 Hz, 1H), 7.25 (d, J=2.0 Hz, 1H), 6.93-6.87 (m, 2H), 6.77 (s, 1H), 6.44 (d, J=2.8 Hz, 1H), 3.84 (s, 3H), 3.79 (s, 2H) ppm.

### Example 29. Synthesis of (2-(3-((1H-benzo[d]imidazol-5-yl)oxy)phenyl)-1H-imidazol-5-yl)(1-methyl-1H-pyrazol-5-yl)methanol

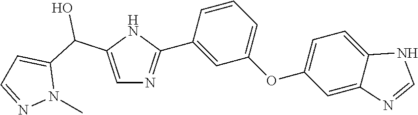 [SMILES: Cn1nccc1C(O)c1cnc(-c2cccc(Oc3ccc4[nH]cnc4c3)c2)[nH]1]

To a stirred solution of 4-bromo-1-methyl-1H-pyrazole (966 mg, 6.0 mmol) in anhydrous THE (20 mL) was added n-BuLi (2.5 mL, 2.5M, 6.0 mmol) at −78° C. under a nitrogen atmosphere. The resulting mixture was stirred at −78° C. for 1 hour, and a solution of compound 2-(3-((1H-benzo[d]imidazol-5-yl)oxy)phenyl)-1H-imidazole-5-carbaldehyde (Intermediate 7) (182.4 mg, 0.6 mmol) in THE (10 mL) was added dropwise at −78° C. The resulting mixture was stirred at −78° C. for 0.5 hours, warmed up to room temperature for 1.5 hours, quenched with methanol (5.0 mL) at 0° C. and then concentrated in vacuo. The residue was purified by prep-HPLC to give (2-(3-((1H-benzo[d]imidazol-5-yl)oxy)phenyl)-1H-imidazol-5-yl)(1-methyl-1H-pyrazol-5-yl)methanol (24 mg, 10%) as a white solid. MS m/z: 387.2 (M+H)⁺. ¹H NMR (400 MHz, MeOD-d₄): δ 8.19 (s, 1H), 7.64 (d, J=8.4 Hz, 1H), 7.60 (d, J=7.6 Hz, 1H), 7.53 (s, 1H), 7.43 (dd, J=8.0 Hz & 8.0 Hz, 1H), 7.38 (d, J=2.0 Hz, 1H), 7.29 (s, 1H), 7.08-7.01 (m, 3H), 6.17 (s, 1H), 5.94 (s, 1H), 3.87 (s, 3H) ppm.

### Example 30. Synthesis of (5-(3-((1H-indol-5-yl)oxy)phenyl)-4H-1,2,4-triazol-3-yl)(1-methyl-1H-pyrazol-4-yl)methanol

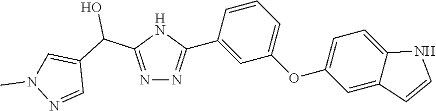 [SMILES: Cn1cc(C(O)c2nnc(-c3cccc(Oc4ccc5[nH]ccc5c4)c3)[nH]2)cn1]

### 2-(1-Methyl-1H-pyrazol-4-yl)-2-((trimethylsilyl)oxy)acetonitrile

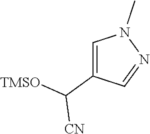

Step A: To a stirred solution of compound 1-methyl-1H-pyrazole-4-carbaldehyde (4.40 g, 40.0 mmol) in anhydrous DCM (100 mL) were added zinc iodide (1.27 g, 4.0 mmol) and trimethylsilyl cyanide (6.0 g, 60.0 mmol) at 0° C. under a nitrogen atmosphere and with good stirring. The resulting mixture was then stirred at room temperature overnight. A saturated aqueous solution of sodium hydrogen carbonate (15 mL) was added. Layers were separated and the aqueous layer was extracted with DCM (2×20 mL). The combined organic layers were dried over sodium sulfate and concentrated in vacuo to provide 2-(1-methyl-1H-pyrazol-4-yl)-2-((trimethylsilyl)oxy)acetonitrile (8.1 g, 96%) as a yellow oil which was used directly in the next step. ¹H NMR (400 MHz, CDCl₃) d 7.30 (s, 1H), 7.26 (s, 1H), 5.26 (s, 1H), 3.68 (s, 3H), 0.00 (s, 9H) ppm.

### Methyl 2-hydroxy-2-(1-methyl-1H-pyrazol-4-yl)acetate

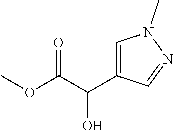

Step B: Acetyl chloride (31.6 g, 400 mmol) was added into methanol (100 mL) at 0° C. under a nitrogen atmosphere with good stirring. The solution was stirred at 0° C. for 10 minutes. 2-(1-Methyl-1H-pyrazol-4-yl)-2-((trimethylsilyl)oxy)acetonitrile (4.18 g, 20.0 mmol) in methanol (10 mL) was added dropwise into the solution. After stirring at room temperature for 1 hour, the reaction mixture was heated at 60° C. for 7 hours, and concentrated. The residue was dissolved in EtOAc (100 mL). The organic phase was washed with saturated sodium hydrogen carbonate solution (20 mL), brine, dried over Na₂SO₄ and concentrated. The crude product was purified by silica gel column chromatography (3:1 to 1:1 petroleum ether:EtOAc) to give methyl 2-hydroxy-2-(1-methyl-1H-pyrazol-4-yl)acetate (2.2 g, 64%) as a pale-yellow solid. ¹H NMR (400 MHz, MeOH-d₄): δ 7.70 (s, 1H), 7.37 (s, 1H), 4.35 (s, 1H), 3.80 (s, 3H), 3.65 (s, 3H) ppm

### 2-Hydroxy-2-(1-methyl-1H-pyrazol-4-yl)acetohydrazide

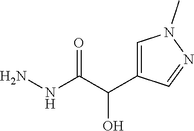

Step C: To a solution of methyl 2-hydroxy-2-(1-methyl-1H-pyrazol-4-yl)acetate (850 mg, 5.0 mmol) in methanol (20 mL) was added hydrazine hydrate (85%, 5 mL). The resulting reaction mixture was heated overnight. The reaction was monitored by TLC. Upon reaction completion, all volatiles were removed in vacuo to give the crude product 2-hydroxy-2-(1-methyl-1H-pyrazol-4-yl)acetohydrazide (860 mg, 100%) as a white solid, which was used directly in the next step. MS m/z: 171.1 (M+H⁺)

### (1-Methyl-1H-pyrazol-4-yl)(5-(3-((I-tosyl-1H-indol-5-yl)oxy)phenyl)-4H-1,2,4-triazol-3-yl)methanol

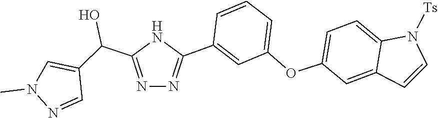 [SMILES: Cc1ccc(S(=O)(=O)n2ccc3cc(Oc4cccc(-c5nnc(C(O)c6cnn(C)c6)[nH]5)c4)ccc32)cc1]

Step D: To a solution of ethyl 3-((1-tosyl-1H-indol-5-yl)oxy)benzimidate (Intermediate 8) (200 mg, crude) in anhydrous ethanol (10 mL) was added triethylamine (202 mg, 2.0 mmol) at room temperature under a nitrogen atmosphere and with good stirring. Ten minutes later, 2-hydroxy-2-(1-methyl-1H-pyrazol-4-yl)acetohydrazide (170 mg, 1.0 mmol) was added. The resulting mixture was heated at 60° C. for 20 hours. All volatiles were removed under reduced pressure. The crude compound was purified by silica gel column chromatography (20:1 to 10:1 DCM:MeOH) to give (1-methyl-1H-pyrazol-4-yl)(5-(3-((1-tosyl-1H-indol-5-yl)oxy)phenyl)-4H-1,2,4-triazol-3-yl)methanol (95 mg, 35%) as a white solid. MS m/z: 541.2 (M+H)⁺. ¹H NMR (400 MHz, MeOD-d₄): δ 8.00 (d, J=8.8 Hz, 1H), 7.84 (s, 1H), 7.82 (s, 1H), 7.74-7.68 (m, 2H), 7.60 (s, 2H), 7.50 (s, 1H), 7.42 (bs, 1H), 7.35 (s, 1H), 7.33 (s, 1H), 7.20 (s, 1H), 7.07 (d, J=9.2 Hz, 1H), 7.02 (bs, 1H), 6.69 (d, J=3.2 Hz, 1H), 5.95 (s, 1H), 3.86 (d, J=6.4 Hz, 3H), 2.37 (s, 3H) ppm.

### (5-(3-((1H-Indol-5-yl)oxy)phenyl)-4H-1,2,4-triazol-3-yl)(1-methyl-1H-pyrazol-4-yl)methanol

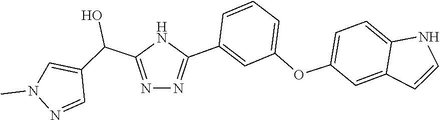 [SMILES: Cn1cc(C(O)c2nnc(-c3cccc(Oc4ccc5[nH]ccc5c4)c3)[nH]2)cn1]

Step E: To a stirred solution of (1-methyl-1H-pyrazol-4-yl)(5-(3-((1-tosyl-1H-indol-5-yl)oxy)phenyl)-4H-1,2,4-triazol-3-yl)methanol (95 mg, 0.18 mmol) in anhydrous methanol (3 mL) was added potassium carbonate (48.6 mg, 0.36 mmol) at room temperature under a nitrogen atmosphere and with good stirring. The resulting mixture was heated at 130° C. for 30 minutes. The mixture was filtered and concentrated in vacuo, and the residue was purified by preparative HPLC to give (5-(3-((1H-indol-5-yl)oxy)phenyl)-4H-1,2,4-triazol-3-yl)(1-methyl-1H-pyrazol-4-yl)methanol (35 mg, 50%) as a white solid. MS m/z: 387.2 (M+H)⁺. ¹H NMR (400 MHz, MeOD-d₄): δ 7.65 (d, J=7.6 Hz, 1H), 7.60 (s, 1H), 7.58 (s, 1H), 7.51 (s, 1H), 7.43-7.37 (m, 2H), 7.02-7.00 (m, 2H), 7.01 (dd, J₁=8.0 Hz & J₂=1.6 Hz, 1H), 6.99 (dd, J₁=8.8 Hz & J₂=2.4 Hz, 1H), 6.44 (d, J=3.2 Hz, 1H), 5.93 (s, 1H), 3.85 (s, 3H) ppm.

### Example 31. Synthesis of 5-(3-(5-((1-methyl-1H-pyrazol-4-yl)methyl)-4H-1,2,4-triazol-3-yl)phenoxy)-1H-pyrrolo[3,2-b]pyridine

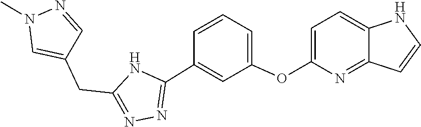 [SMILES: Cn1cc(Cc2nnc(-c3cccc(Oc4ccc5[nH]ccc5n4)c3)[nH]2)cn1]

To a suspension of 2-(1-methyl-1H-pyrazol-4-yl)acetohydrazide (Step C, Example 25) (308 mg, 2.0 mmol) in n-BuOH (4.0 mL) were added 3-((1H-pyrrolo[3,2-b]pyridin-5-yl)oxy)benzonitrile (Intermediate 10) (235 mg, 1.0 mmol) and potassium carbonate (276 mg, 2.0 mmol). The resulting mixture was heated at 170° C. for 3 hours in a microwave reactor. After reaction completion, the mixture was purified by preparative HPLC to afford 5-(3-(5-((1-methyl-1H-pyrazol-4-yl)methyl)-4H-1,2,4-triazol-3-yl)phenoxy)-1H-pyrrolo[3,2-b]pyridine (40 mg, 11%) as a white solid. MS-ESI: [M+H]⁺ 372.2; ¹H NMR (400 MHz, MeOH-d₄): δ 7.90 (d, J=8.4 Hz, 1H), 7.79 (d, J=7.6 Hz, 1H), 7.68 (s, 1H), 7.55-7.48 (m, 3H), 7.41 (s, 1H), 7.16 (m, 1H), 6.84 (d, J=8.8 Hz, 1H), 6.48 (d, J=2.8 Hz, 1H), 4.00 (s, 2H), 3.85 (s, 3H) ppm.

### Example 32. Synthesis of (2-(3-((1H-pyrrolo[3,2-b]pyridin-5-yl)oxy)phenyl)-1H-imidazol-5-yl)(1-methyl-1H-pyrazol-4-yl)methanol

### Example 33. Synthesis of (2-(3-((1H-pyrrolo[3,2-b]pyridin-5-yl)oxy)phenyl)-1H-imidazol-5-yl)(1-methyl-1H-pyrazol-5-yl)methanol

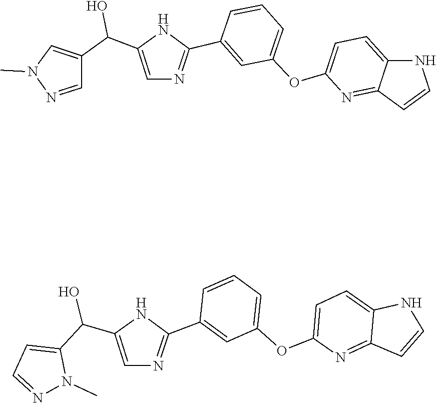 [SMILES: Cn1cc(C(O)c2cnc(-c3cccc(Oc4ccc5[nH]ccc5n4)c3)[nH]2)cn1 ; Cn1nccc1C(O)c1cnc(-c2cccc(Oc3ccc4[nH]ccc4n3)c2)[nH]1]

To a stirred solution of 4-bromo-1-methyl-1H-pyrazole (1.05 g, 6.6 mmol) in THE (80 mL) was added dropwise n-BuLi (2.4 mL, 2.5 N in THF, 5.9 mmol)) at −78° C., the mixture was stirred at −78° C. for 30 minutes and then a solution of 2-(3-((1H-pyrrolo[3,2-b]pyridin-5-yl)oxy)phenyl)-1H-imidazole-5-carbaldehyde (prepared from Intermediate 10 via a sequence identical to the one for Intermediate 6 from Intermediate 6C) (200 mg, 0.66 mmol) in THE (10 mL) was added to the mixture. The resulting mixture was stirred at room temperature for 16 hours and quenched with MeOH (10 mL). The mixture was concentrated in vacuo. The residue was purified by prep-HPLC to afford (2-(3-((1H-pyrrolo[3,2-b]pyridin-5-yl)oxy)phenyl)-1H-imidazol-5-yl)(1-methyl-1H-pyrazol-4-yl)methanol (27 mg, 11%) and (2-(3-((1H-pyrrolo[3,2-b]pyridin-5-yl)oxy)phenyl)-1H-imidazol-5-yl)(1-methyl-1H-pyrazol-5-yl)methanol (15 mg, 6%) as a white solid.

(2-(3-((1H-pyrrolo[3,2-b]pyridin-5-yl)oxy)phenyl)-1H-imidazol-5-yl)(1-methyl-1H-pyrazol-4-yl)methanol (Example 32): MS m/z: 387.2 (M+H); ¹H NMR (400 MHz, MeOD-d₄): δ 7.89 (d, J=8.4 Hz, 1H), 7.67 (d, J=7.6 Hz, 1H), 7.60 (s, 1H), 7.54-7.53 (m, 2H), 7.47 (t, J=8.4 Hz, 2H), 7.08 (dd, J=2.0 Hz & 8.0 Hz, 1H), 6.99 (s, 1H), 6.82 (d, J=8.4 Hz, 1H), 6.48 (d, J=2.8 Hz, 1H), 5.81 (s, 1H), 3.87 (s, 3H) ppm.

(2-(3-((1H-pyrrolo[3,2-b]pyridin-5-yl)oxy)phenyl)-1H-imidazol-5-yl)(1-methyl-1H-pyrazol-5-yl)methanol (Example 33): MS m/z: 387.2 (M+H)+; ¹H NMR (400 MHz, MeOD-d₄): δ 7.89 (d, J=8.4 Hz, 1H), 7.67 (d, J=8.0 Hz, 1H), 7.60 (s, 1H), 7.54-7.53 (d, J=3.2 Hz, 1H), 7.47 (t, J=8.0 Hz, 1H), 7.37 (s, 1H), 7.08 (dd, J=1.6 Hz & 8.0 Hz, 1H), 6.99 (s, 1H), 6.83 (d, J=8.8 Hz, 1H), 6.48 (d, J=2.8 Hz, 1H), 6.18 (s, 1H), 5.81 (s, 1H), 3.87 (s, 3H) ppm.

### Example 34. Synthesis of 1-(2-(3-((1H-benzo[d]imidazol-5-yl)oxy)phenyl)-1H-imidazol-5-yl)-2-(thiazol-2-yl)ethan-1-ol

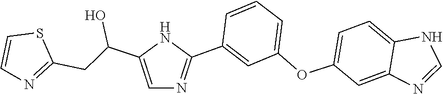 [SMILES: OC(Cc1nccs1)c1cnc(-c2cccc(Oc3ccc4[nH]cnc4c3)c2)[nH]1]

To a stirred solution of 2-methylthiazole (696 mg, 7.03 mmol) in THF (10 mL) was added n-BuLi (2.5 mL, 6.25 mmol) at −78° C. The resulting mixture was stirred at −78° C. for 1 hour. Then 2-(3-((1H-benzo[d]pyridine-5-yl)oxy)phenyl)-1H-imidazole-5-carbaldehyde (Intermediate 7) (356 mg, 1.17 mmol) in THE (5 mL) was added dropwise, the mixture was stirred at room temperature for 16 hours. The resulting mixture was quenched with MeOH (10 ml) and concentrated in vacuo. The residue was purified by prep-HPLC to give 1-(2-(3-((1H-benzo[d]imidazol-5-yl)oxy)phenyl)-1H-imidazol-5-yl)-2-(thiazol-2-yl)ethan-1-ol (90 mg, 19%) as a white solid. MS m/z: 403.9 (M+). ¹H NMR (400 MHz, MeOD-d₄): δ 8.19 (s, 1H), 7.68-7.59 (m, 3H), 7.54-7.53 (m, 1H), 7.45-7.41 (m, 2H), 7.29 (d, J=1.6 Hz, 1H), 7.08-7.00 (m, 3H), 5.11-5.08 (m, 1H), 3.63-3.49 (m, 2H) ppm.

### Example 35. Synthesis of (5-(3-((1H-benzo[d]imidazol-5-yl)oxy)phenyl)-4H-1,2,4-triazol-3-yl)(2-methylthiazol-5-yl)methanol

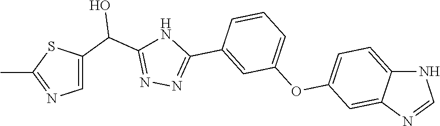 [SMILES: Cc1ncc(C(O)c2nnc(-c3cccc(Oc4ccc5[nH]cnc5c4)c3)[nH]2)s1]

### 2-(2-Methylthiazol-5-yl)-2-((trimethylsilyl)oxy)acetonitrile

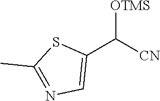

Step A: To a stirred solution of 2-methylthiazole-5-carbaldehyde (3 g, 23.6 mmol) in DCM (30 mL) was added trimethylsilyl cyanide (3.55 g, 35.5 mmol) and ZnI₂ (766 mg, 2.4 mmol) at room temperature under an N₂ atmosphere. The resulting mixture was stirred at room temperature overnight. The reaction was neutralized by the addition of saturated aqueous NaHCO₃. The organic phase was washed with brine, dried over Na₂SO₄, filtered, and concentrated in vacuo to give 2-(2-methylthiazol-5-yl)-2-((trimethylsilyl)oxy)acetonitrile (5.3 g, crude) as brown oil. This was used in the next step directly without further purification. MS-ESI: [M+H]⁺ 227.0

### Methyl 2-hydroxy-2-(2-methylthiazol-5-yl)acetate

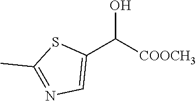

Step B: To a stirred solution of 2-(2-methylthiazol-5-yl)-2-((trimethylsilyl)oxy)acetonitrile (5.3 g, 23.6 mmol) in MeOH (100 mL) was added acetyl chloride (34 mL, 472 mmol) dropwise at 0° C. The resulting mixture was stirred at room temperature overnight. The reaction was neutralized by the addition of saturated aqueous NaHCO₃ at 0° C., then extracted with 10% MeOH in DCM (150 mL×5). The organic phases were combined, dried over anhydrous Na₂SO₄, filtered, and concentrated. The residue was purified by flash column chromatography on silica gel, eluting with 0-75% EtOAc in petroleum ether to give methyl 2-hydroxy-2-(2-methylthiazol-5-yl)acetate (930 mg, 21% over two steps) as a yellow oil. MS-ESI: [M+H]⁺ 188.1

### Tert-Butyl 2-(2-hydroxy-2-(2-methylthiazol-5-yl)acetyl)hydrazine-1-carboxylate

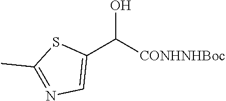

Step C: To a stirred solution of methyl 2-hydroxy-2-(2-methylthiazol-5-yl)acetate (930 mg, 4.9 mmol) in MeOH (30 mL) was added tert-butyl hydrazinecarboxylate (13 g, 98 mmol). The resulting mixture was stirred at 70° C. overnight, concentrated in vacuo. The residue was purified by flash column chromatography on silica gel, eluting with 0-10% MeOH in DCM to give tert-butyl 2-(2-hydroxy-2-(2-methylthiazol-5-yl)acetyl)hydrazine-1-carboxylate (360 mg). 145 mg of 2-hydroxy-2-(2-methylthiazol-5-yl)acetohydrazide was also recovered. MS-ESI: [M+H]⁺ 288.1

### 2-Hydroxy-2-(2-methylthiazol-5-yl)acetohydrazide

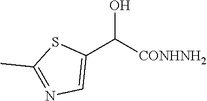

Step D: A solution of tert-butyl 2-(2-hydroxy-2-(2-methylthiazol-5-yl)acetyl)hydrazine-1-carboxylate (360 mg, 1.25 mmol) in 27 mL of 4M HCl in 1,4-dioxane was stirred at room temperature for 1 hour. The mixture was concentrated in vacuo to give 2-hydroxy-2-(2-methylthiazol-5-yl)acetohydrazide (235 mg, crude) as a light-yellow solid. MS-ESI: [M+H]⁺ 188.1

### (5-(3-((1H-Benzo[d]imidazol-5-yl)oxy)phenyl)-4H-1,2,4-triazol-3-yl)(2-methylthiazol-5-yl)methanol

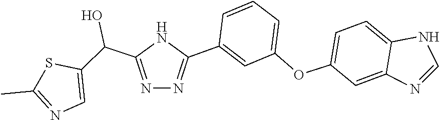 [SMILES: Cc1ncc(C(O)c2nnc(-c3cccc(Oc4ccc5[nH]cnc5c4)c3)[nH]2)s1]

Step E: To a stirred solution of 2-hydroxy-2-(2-methylthiazol-5-yl)acetohydrazide (130 mg, 0.7 mmol) and ethyl 3-((1H-benzo[d]imidazol-5-yl)oxy)benzimidate (Intermediate 9) (130 mg, 0.46 mmol) in EtOH (30 mL) was added Et₃N (460 mg, 4.6 mmol). The resulting mixture was stirred at 90° C. overnight. The reaction mixture was concentrated in vacuo and purified by flash column chromatography on silica gel, eluting with 0-10% MeOH in DCM containing 1 mol-% NH₄OH to give (5-(3-((1H-benzo[d]imidazol-5-yl)oxy)phenyl)-4H-1,2,4-triazol-3-yl)(2-methylthiazol-5-yl)methanol (186 mg, 100%) as a brown oil. MS-ESI: [M+H]+ 405.1; ¹H NMR (400 MHz, MeOH-d₄): δ 8.18 (s, 1H), 7.71 (d, J=7.6 Hz, 1H), 7.64-7.56 (m, 3H), 7.44 (t, J=8.0 Hz, 1H), 7.27 (s, 1H), 7.06 (t, J=8.8 Hz, 2H), 6.17 (s, 1H), 2.65 (s, 3H) ppm.

### Example 36. Synthesis of (5-(3-((1H-benzo[d]imidazol-5-yl)oxy)phenyl)-4H-1,2,4-triazol-3-yl)(2-methylthiazol-5-yl)methanamine

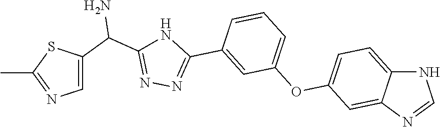 [SMILES: Cc1ncc(C(N)c2nnc(-c3cccc(Oc4ccc5[nH]cnc5c4)c3)[nH]2)s1]

### (5-(3-((1H-Benzo[d]imidazol-5-yl)oxy)phenyl)-4H-1,2,4-triazol-3-yl)(2-methylthiazol-5-yl)methanone

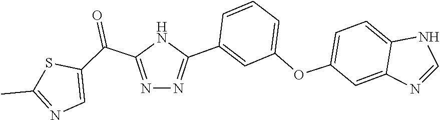 [SMILES: Cc1ncc(C(=O)c2nnc(-c3cccc(Oc4ccc5[nH]cnc5c4)c3)[nH]2)s1]

Step A: To a stirred solution of (5-(3-((1H-benzo[d]imidazol-5-yl)oxy)phenyl)-4H-1,2,4-triazol-3-yl)(2-methylthiazol-5-yl)methanol (Example 36) (85 mg, 0.21 mmol) in THE (150 mL) was added MnO₂ (183 mg, 2.1 mmol). The resulting mixture was stirred at room temperature overnight, filtered, and concentrated in vacuo. The residue was purified by flash column chromatography on silica gel, eluting with 0-10% MeOH in DCM to give (5-(3-((1H-benzo[d]imidazol-5-yl)oxy)phenyl)-4H-1,2,4-triazol-3-yl)(2-methylthiazol-5-yl)methanone (66 mg, 78%) as a brown solid. MS-ESI: [M+H]⁺ 403.1

### (5-(3-((1H-Benzo[d]imidazol-5-yl)oxy)phenyl)-4H-1,2,4-triazol-3-yl)(2-methylthiazol-5-yl)methanamine

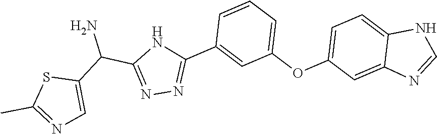 [SMILES: Cc1ncc(C(N)c2nnc(-c3cccc(Oc4ccc5[nH]cnc5c4)c3)[nH]2)s1]

Step B: To a 25 mL microwave tube was added (5-(3-((1H-benzo[d]imidazol-5-yl)oxy)phenyl)-4H-1,2,4-triazol-3-yl)(2-methylthiazol-5-yl)methanone (66 mg, 0.164 mmol), Ti(i-PrO)₄ (466 mg, 1.64 mmol) and NH₃ (15 mL, 2 M MeOH solution) under an N₂ atmosphere. The sealed tube was heated and stirred at 50° C. overnight. NaBH₄ (62 mg, 1.64 mmol) was added to the mixture after cooling to room temperature and stirring continued at room temperature overnight. The mixture was filtered, and the filtrate was stirred with an acidic cation exchange resin for 1 hour, then the solids were isolated. The filtrate was discarded, the resin was washed with a NH₃ solution in methanol, the filtrate was concentrated and purified by prep-HPLC to give (5-(3-((1H-benzo[d]imidazol-5-yl)oxy)phenyl)-4H-1,2,4-triazol-3-yl)(2-methylthiazol-5-yl)methanamine (20 mg, 30%) as a white solid. MS-ESI: [M+H]⁺ 404.1; ¹H NMR (400 MHz, MeOH-d₄): δ 8.18 (s, 1H), 7.71-7.60 (m, 3H), 7.56-7.45 (m, 2H), 7.27 (d, J=8.4 Hz, 1H), 7.12-7.05 (m, 2H), 5.52 (s, 1H), 2.64 (s, 3H) ppm.

### Example 37. Synthesis of (5-(3-((4-(hydroxymethyl)-1H-indol-5-yl)oxy)phenyl)-4H-1,2,4-triazol-3-yl)(2-methylthiazol-5-yl)methanol (1-c-80)—Intermediate 11

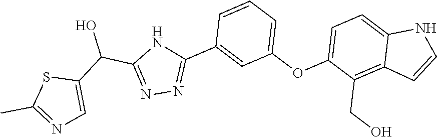 [SMILES: Cc1ncc(C(O)c2nnc(-c3cccc(Oc4ccc5[nH]ccc5c4CO)c3)[nH]2)s1]

### Methyl 5-(3-(5-(hydroxy(2-methylthiazol-5-yl)methyl)-4H-1,2,4-triazol-3-yl)phenoxy)-1H-indole-4-carboxylate

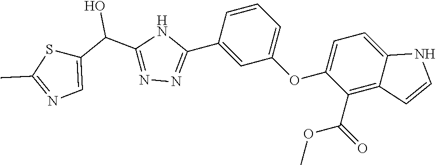 [SMILES: COC(=O)c1c(Oc2cccc(-c3nnc(C(O)c4cnc(C)s4)[nH]3)c2)ccc2[nH]ccc12]

To a solution of 2-hydroxy-2-(2-methylthiazol-5-yl)acetohydrazide (Step D, Example 36) (490 mg, 2.6 mmol) and 5-(3-(ethoxy(imino)methyl)phenoxy)-1H-indole-4-carboxylate (Intermediate 11) (586 mg, 1.7 mmol) in EtOH (30 mL) was added Et₃N (2.6 g, 26 mmol). The resulting mixture was stirred at 90° C. overnight and concentrated in vacuo. The residue was purified by flash column chromatography on silica gel, eluting with 0-10% MeOH in DCM containing 1% NH₄OH) to give methyl 5-(3-(5-(hydroxy(2-methylthiazol-5-yl)methyl)-4H-1,2,4-triazol-3-yl)phenoxy)-1H-indole-4-carboxylate (438 mg, 36.6%) as a brown oil. MS-ESI: [M+H]⁺ 462.1

### (5-(3-((4-(Hydroxymethyl)-1H-indol-5-yl)oxy)phenyl)-4H-1,2,4-triazol-3-yl)(2-methylthiazol-5-yl)methanol

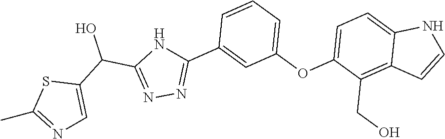 [SMILES: Cc1ncc(C(O)c2nnc(-c3cccc(Oc4ccc5[nH]ccc5c4CO)c3)[nH]2)s1]

To a stirred solution of methyl 5-(3-(5-(hydroxy(2-methylthiazol-5-yl)methyl)-4H-1,2,4-triazol-3-yl)phenoxy)-1H-indole-4-carboxylate (60 mg, 0.13 mmol) in THE (5 mL) was added LiAlH₄ (0.39 mL, 1M in THF, 0.39 mmol) at room temperature under an N₂ atmosphere. The mixture was stirred at room temperature overnight, quenched by the dropwise addition of saturated aqueous NH4Cl. The resulting mixture was filtered through a pad of Celite. The filtrate was washed with a 10% solution of MeOH in DCM. The organic phase was washed with brine, dried over anhydrous Na₂SO₄, filtered, and concentrated. The residue was purified by prep-HPLC to give (5-(3-((4-(hydroxymethyl)-1H-indol-5-yl)oxy)phenyl)-4H-1,2,4-triazol-3-yl)(2-methylthiazol-5-yl)methanol (30 mg, 53%) as a white solid. MS-ESI: [M+H]⁺ 434.1; ¹H NMR (400 MHz, MeOH-d₄): δ 7.61 (d, J=8 Hz, 1H), 7.54 (s, 1H), 7.46 (m, 1H), 7.40-7.36 (m, 2H), 7.33 (d, J=3.2 Hz, 1H), 7.01 (dd, J=1.6, 8.0 Hz, 1H), 6.81 (d, J=8.8 Hz, 1H), 6.72 (d, J=2.8 Hz, 1H), 6.14 (s, 1H), 4.88 (s, 2H), 2.65 (s, 3H) ppm.

### Example 38. Synthesis of 5-(3-(5-(hydroxy(2-methylthiazol-5-yl)methyl)-4H-1,2,4-triazol-3-yl)phenoxy)-1H-indole-4-carboxylic acid

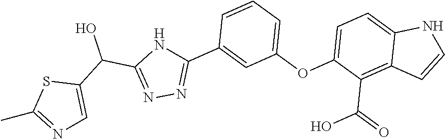 [SMILES: Cc1ncc(C(O)c2nnc(-c3cccc(Oc4ccc5[nH]ccc5c4C(=O)O)c3)[nH]2)s1]

To a stirred solution of methyl 5-(3-(5-(hydroxy(2-methylthiazol-5-yl)methyl)-4H-1,2,4-triazol-3-yl)phenoxy)-1H-indole-4-carboxylate (120 mg, 0.26 mmol) in THE (10 mL) and H₂O (5 mL) was added LiOH·H₂O (218 mg, 5.2 mmol). The resulting mixture was stirred at 50° C. for 5 hours. The mixture was neutralized by the addition of 1M hydrochloric acid and the aqueous phase was extracted with a 1:1 THF:EtOAc solution. The organic phases were combined and concentrated. The residue was purified by prep-HPLC to give 5-(3-(5-(hydroxy(2-methylthiazol-5-yl)methyl)-4H-1,2,4-triazol-3-yl)phenoxy)-1H-indole-4-carboxylic acid (85 mg, 73%) as a white solid. MS-ESI: [M+H]⁺ 448.1; ¹H NMR (400 MHz, MeOH-d₄): δ 7.58 (d, J=8 Hz, 1H), 7.55 (s, 1H), 7.44-7.32 (m, 4H), 7.07 (d, J=7.2 Hz, 1H), 6.83 (d, J=8.8 Hz, 1H), 6.73 (d, J=3.2 Hz, 1H), 6.13 (s, 1H), 2.65 (s, 3H) ppm.

### Example 39. Synthesis of 5-(3-(5-(hydroxy(2-methylthiazol-5-yl)methyl)-4H-1,2,4-triazol-3-yl)phenoxy)-1H-indole-4-carboxamide

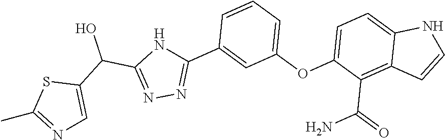 [SMILES: Cc1ncc(C(O)c2nnc(-c3cccc(Oc4ccc5[nH]ccc5c4C(N)=O)c3)[nH]2)s1]

To a stirred solution of 5-(3-(5-(hydroxy(2-methylthiazol-5-yl)methyl)-4H-1,2,4-triazol-3-yl)phenoxy)-1H-indole-4-carboxylic acid (35 mg, 0.078 mmol) in DMF (3 mL) was added NH₄Cl (8 mg, 0.15 mmol), HATU (60 mg, 0.16 mmol), and Et₃N (16 mg, 0.16 mmol) at room temperature under an N₂ atmosphere. The mixture was stirred at room temperature overnight and concentrated. The residue was purified by prep-HPLC to give 5-(3-(5-(hydroxy(2-methylthiazol-5-yl)methyl)-4H-1,2,4-triazol-3-yl)phenoxy)-1H-indole-4-carboxamide (21 mg, 61%) as a white solid. MS-ESI: [M+H]⁺ 447.1; ¹H NMR (400 MHz, MeOH-d₄): δ 7.59 (d, J=8 Hz, 1H), 7.53-7.50 (m, 2H), 7.44-7.37 (m, 3H), 6.98 (dd, J=2.0, 8.0 Hz, 1H), 6.81 (d, J=8.8 Hz, 1H), 6.63 (d, J=2.8 Hz, 1H), 6.07 (s, 1H), 2.55 (s, 3H) ppm.

### Example 40. Synthesis of (5-(3-((4-(hydroxymethyl)-1H-indol-5-yl)oxy)phenyl)-4H-1,2,4-triazol-3-yl)(thiazol-5-yl)methanol

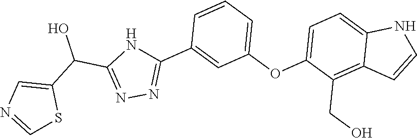 [SMILES: OCc1c(Oc2cccc(-c3nnc(C(O)c4cncs4)[nH]3)c2)ccc2[nH]ccc12]

### 2-(Thiazol-5-yl)-2-((trimethylsilyl)oxy)acetonitrile

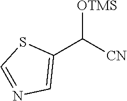

To a stirred solution of thiazole-5-carbaldehyde (5 g, 44 mmol) in DCM (50 mL) was added trimethylsilyl cyanide (6.6 g, 66 mmol) and ZnI₂ (1.4 g, 4.4 mmol) at room temperature under an N₂ atmosphere. The resulting mixture was stirred at room temperature for 48 hours, then neutralized by addition of saturated aqueous NaHCO₃. The organic phase was washed with brine, dried over Na₂SO₄, filtered, and concentrated in vacuo to give 2-(thiazol-5-yl)-2-((trimethylsilyl)oxy)acetonitrile (9.3 g, crude) as a brown oil. It was used directly in the next step without further purification. MS-ESI: [M+H]⁺ 213.1

### Methyl 2-hydroxy-2-(thiazol-5-yl)acetate

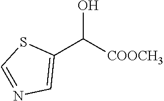 [SMILES: COC(=O)C(O)c1cncs1]

To a stirred solution of 2-(thiazol-5-yl)-2-((trimethylsilyl)oxy)acetonitrile (9.3 g, 44 mmol) in MeOH (120 mL) was added acetyl chloride (61 mL, 880 mmol) dropwise at 0° C. The resulting mixture was stirred overnight at room temperature. The mixture was neutralized by the addition of saturated aqueous NaHCO₃ at 0° C., then extracted with 10% MeOH in DCM. The organic phases were combined, dried over anhydrous Na₂SO₄, filtered, and concentrated. The residue was purified by flash column chromatography on silica gel, eluting with 0-75% EtOAc in petroleum ether to give methyl 2-hydroxy-2-(thiazol-5-yl)acetate (3.47 g, 46% over two steps) as a brown syrup. MS-ESI: [M+H]⁺ 174.1

### Tert-Butyl 2-(2-hydroxy-2-(thiazol-5-yl)acetyl)hydrazine-1-carboxylate

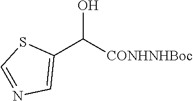

To a stirred solution of methyl 2-hydroxy-2-(thiazol-5-yl)acetate (692 mg, 4 mmol) in MeOH (30 mL) was added tert-butyl hydrazinecarboxylate (10 g, 80 mmol). The resulting mixture was stirred at 70° C. overnight and concentrated in vacuo. The residue was purified by flash column chromatography on silica gel, eluting with 0-10% MeOH in DCM to give tert-butyl 2-(2-hydroxy-2-(thiazol-5-yl)acetyl)hydrazine-1-carboxylate (230 mg). 85 mg of 2-hydroxy-2-(thiazol-5-yl)acetohydrazide were also recovered. MS-ESI: [M+H]⁺ 274.1

### 2-Hydroxy-2-(thiazol-5-yl)acetohydrazide

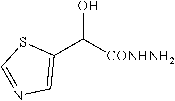

A solution of tert-butyl 2-(2-hydroxy-2-(thiazol-5-yl)acetyl)hydrazine-1-carboxylate (230 mg, 0.84 mmol) in 30 mL of 4M HCl in 1,4-dioxane was stirred at room temperature for 1 hour. The mixture was concentrated in vacuo to give 2-hydroxy-2-(thiazol-5-yl)acetohydrazide (145 mg, crude) as an off-white solid. MS-ESI: [M+H]+ 174.1

### Methyl 5-(3-(5-(hydroxy(thiazol-5-yl)methyl)-4H-1,2,4-triazol-3-yl)phenoxy)-1H-indole-4-carboxylate

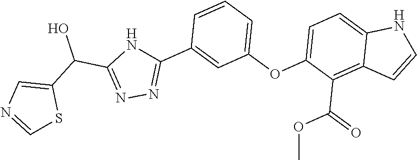 [SMILES: COC(=O)c1c(Oc2cccc(-c3nnc(C(O)c4cncs4)[nH]3)c2)ccc2[nH]ccc12]

To a stirred solution of 2-hydroxy-2-(thiazol-5-yl)acetohydrazide (145 mg, 0.84 mmol) and ethyl 3-((1H-benzo[d]imidazol-5-yl)oxy)benzimidate (Intermediate 9) (426 mg, 1.26 mmol) in EtOH (30 mL) was added Et₃N (840 mg, 8.4 mmol). The resulting mixture was stirred at 90° C. overnight. The mixture was concentrated in vacuo. The residue was purified by flash column chromatography on silica gel, eluting with 0-10% MeOH in DCM containing 1% NH₄OH) to give methyl 5-(3-(5-(hydroxy(thiazol-5-yl)methyl)-4H-1,2,4-triazol-3-yl)phenoxy)-1H-indole-4-carboxylate (505 mg, 85%) as a brown oil. MS-ESI: [M+H]⁺ 448.1

### (5-(3-((4-(Hydroxymethyl)-1H-indol-5-yl)oxy)phenyl)-4H-1,2,4-triazol-3-yl)(thiazol-5-yl)methanol

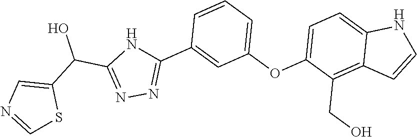 [SMILES: OCc1c(Oc2cccc(-c3nnc(C(O)c4cncs4)[nH]3)c2)ccc2[nH]ccc12]

To a stirred solution of methyl 5-(3-(5-(hydroxy(thiazol-5-yl)methyl)-4H-1,2,4-triazol-3-yl)phenoxy)-1H-indole-4-carboxylate (200 mg, 0.447 mmol) in THE (10 mL) was added LiAlH₄ (1.8 mL, 1M in THF, 1.8 mmol) at room temperature under an N₂ atmosphere. The mixture was stirred at room temperature overnight, quenched by the dropwise addition of saturated aqueous NH₄Cl, filtered through a pad of Celite, and washed with a solution of 10% MeOH in DCM. The organic phase was washed with brine, dried over anhydrous Na₂SO₄, filtered, and concentrated. The residue was purified by prep-HPLC to give (5-(3-((4-(hydroxymethyl)-1H-indol-5-yl)oxy)phenyl)-4H-1,2,4-triazol-3-yl)(thiazol-5-yl)methanol (19.8 mg, 16%) as a white solid. MS-ESI: [M+H]+ 420.1; ¹H NMR (400 MHz, MeOH-d₄): δ 8.96 (d, J=0.4 Hz, 1H), 7.85 (s, 1H), 7.61 (d, J=8.0 Hz, 1H), 7.47 (t, J=2.0 Hz, 1H), 7.40-7.37 (m, 2H), 7.33 (d, J=3.2 Hz, 1H), 7.01 (dd, J=2.4, 8.0 Hz, 1H), 6.81 (d, J=8.0 Hz, 1H), 6.72 (dd, J=0.8, 3.2 Hz, 1H), 6.25 (s, 1H) ppm.

### Example 41. Synthesis of N-(2-(dimethylamino)ethyl)-5-(3-(5-(hydroxy(thiazol-5-yl)methyl)-4H-1,2,4-triazol-3-yl)phenoxy)-1H-indole-4-carboxamide

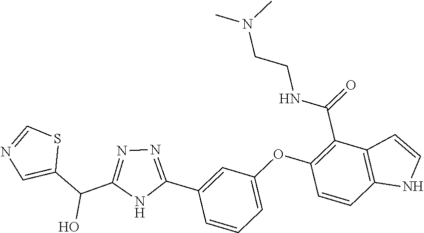 [SMILES: CN(C)CCNC(=O)c1c(Oc2cccc(-c3nnc(C(O)c4cncs4)[nH]3)c2)ccc2[nH]ccc12]

### 5-(3-(5-(Hydroxy(thiazol-5-yl)methyl)-4H-1,2,4-triazol-3-yl)phenoxy)-1H-indole-4-carboxylic acid

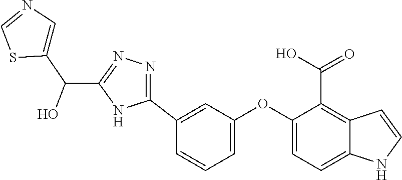 [SMILES: O=C(O)c1c(Oc2cccc(-c3nnc(C(O)c4cncs4)[nH]3)c2)ccc2[nH]ccc12]

Step A: To a stirred solution of methyl 5-(3-(5-(hydroxy(thiazol-5-yl)methyl)-4H-1,2,4-triazol-3-yl)phenoxy)-1H-indole-4-carboxylate (250 mg, 0.56 mmol) in THE (20 mL) and H₂O (10 mL) was added LiOH·H₂O (235 mg, 5.6 mmol). It was stirred at room temperature overnight. The mixture was neutralized by addition of 0.5M aqueous HCl and then the aqueous phase was extracted with a 1:1 THF:ethyl acetate solution. The organic phase was combined, dried over anhydrous Na₂SO₄, filtered, and concentrated to give 5-(3-(5-(hydroxy(thiazol-5-yl)methyl)-4H-1,2,4-triazol-3-yl)phenoxy)-1H-indole-4-carboxylic acid (175 mg, 72%, crude) as a brown solid. MS-ESI: [M+H]⁺ 434.1

### N-(2-(Dimethylamino)ethyl)-5-(3-(5-(hydroxy(thiazol-5-yl)methyl)-4H-1,2,4-triazol-3-yl)phenoxy)-1H-indole-4-carboxamide

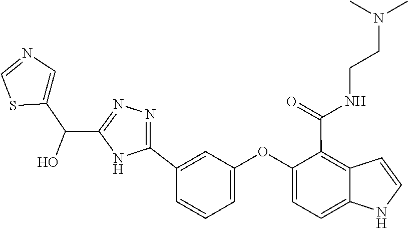 [SMILES: CN(C)CCNC(=O)c1c(Oc2cccc(-c3nnc(C(O)c4cncs4)[nH]3)c2)ccc2[nH]ccc12]

Step B: To a stirred solution of 5-(3-(5-(hydroxy(thiazol-5-yl)methyl)-4H-1,2,4-triazol-3-yl)phenoxy)-1H-indole-4-carboxylic acid (Example 40) (50 mg, 0.115 mmol) in THE (5 mL) was added N₁,N₁-dimethylethane-1,2-diamine (20 mg, 0.23 mmol), HATU (87.8 mg, 0.23 mmol), and Et₃N (23 mg, 0.23 mmol) at room temperature under N₂ atmosphere. The mixture was stirred at room temperature for 0.5 h and concentrated. The residue was dissolved in 50 mL of 10% MeOH in DCM solution, washed with brine (10 mL), dried over anhydrous Na₂SO₄, filtered, and concentrated. The residue was purified by prep-HPLC to give N-(2-(dimethylamino)ethyl)-5-(3-(5-(hydroxy(thiazol-5-yl)methyl)-4H-1,2,4-triazol-3-yl)phenoxy)-1H-indole-4-carboxamide (23 mg, 40%) as a white solid. MS-ESI: [M+H]+ 504.1 ; ¹H NMR (400 MHz, MeOH-d₄): δ 8.96 (s, 1H), 7.86 (s, 1H), 7.65 (d, J=8 Hz, 1H), 7.54 (d, J=8.8 Hz, 1H), 7.50 (t, J=2.0 Hz, 1H), 7.42-7.37 (m, 2H), 7.01 (dd, J=2.0, 8.0 Hz, 1H), 6.91 (d, J=8.8 Hz, 1H), 6.73 (d, J=3.2 Hz, 1H), 6.26 (s, 1H), 3.42 (t, J=7.2 Hz, 2H), 2.35 (t, J=7.2 Hz, 2H), 2.18 (s, 6H) ppm.

### Example 42. Synthesis of 5-(3-(5-(hydroxy(thiazol-5-yl)methyl)-4H-1,2,4-triazol-3-yl)phenoxy)-N-(3,3,3-trifluoro-2-hydroxypropyl)-1H-indole-4-carboxamide

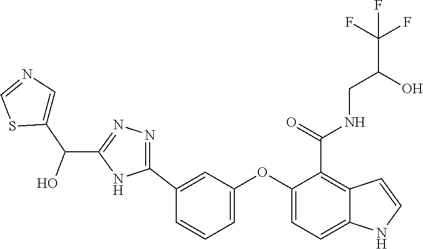 [SMILES: O=C(NCC(O)C(F)(F)F)c1c(Oc2cccc(-c3nnc(C(O)c4cncs4)[nH]3)c2)ccc2[nH]ccc12]

To a stirred solution of 5-(3-(5-(hydroxy(thiazol-5-yl)methyl)-4H-1,2,4-triazol-3-yl)phenoxy)-1H-indole-4-carboxylic acid (35 mg, 0.081 mmol) in THE (4 mL) was added 3-amino-1,1,1-trifluoropropan-2-ol (21 mg, 0.16 mmol), HATU (61 mg, 0.16 mmol), and Et₃N (16 mg, 0.16 mmol) at room temperature under N₂ atmosphere. The mixture was stirred at room temperature for 0.5 h and concentrated. The residue was dissolved in 50 mL of 10% MeOH in DCM. washed with brine (10 mL), dried over anhydrous Na₂SO₄, filtered, and concentrated. The residue was purified by prep-HPLC to give 5-(3-(5-(hydroxy(thiazol-5-yl)methyl)-4H-1,2,4-triazol-3-yl)phenoxy)-N-(3,3,3-trifluoro-2-hydroxypropyl)-1H-indole-4-carboxamide (4.1 mg, 9.3%) as a white solid. MS-ESI: [M+H]+ 545.1; ¹H NMR (400 MHz, MeOH-d₄): δ 8.96 (s, 1H), 7.85 (s, 1H), 7.66 (d, J=7.2 Hz, 1H), 7.54-7.52 (m, 2H), 7.43-7.39 (m, 2H), 7.06 (d, J=7.20 Hz, 1H), 6.89 (d, J=8.4 Hz, 1H), 6.79 (d, J=2.8 Hz, 1H), 6.26 (s, 1H), 4.10-4.05 (m, 1H), 3.76-3.72 (m, 1H), 3.38-3.35 (m, 1H) ppm.

### Example 43. Synthesis of 3-(5-((2-(5-((4,6-difluoro-1H-indol-5-yl)oxy)-2-fluorophenyl)-1H-imidazol-5-yl)methyl)thiophen-2-yl)propanoic acid

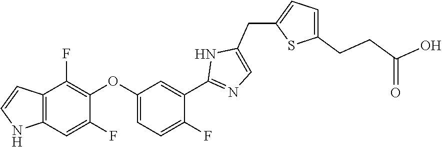 [SMILES: O=C(O)CCc1ccc(Cc2cnc(-c3cc(Oc4c(F)cc5[nH]ccc5c4F)ccc3F)[nH]2)s1]

### (E)-2-(5-(3-ethoxy-3-oxoprop-1-en-1-yl)thiophen-2-yl)acetic acid

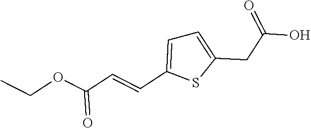 [SMILES: CCOC(=O)/C=C/c1ccc(CC(=O)O)s1]

Step A: To a solution of 2-(5-bromo-2-thienyl)acetic acid (1.00 eq, 1.10 g, 4.98 mmol) in 1,4-dioxane (20 mL) and Water (2 mL) was added ethyl (E)-3-(4,4,5,5-tetramethyl-1,3,2-dioxaborolan-2-yl)prop-2-enoate (1.50 eq, 1687 mg, 7.46 mmol), potassium carbonate (2.00 eq, 1375 mg, 9.95 mmol) and 1,1′-Bis-(di-tert.-butylphosphino-)ferrocen-palladiumdichloride (0.050 eq, 162 mg, 0.249 mmol). The resulting mixture was stirred for 2 hours at 80° C. under an atmosphere of argon. The mixture was concentrated and purified by flash column chromatography on silica gel, eluting with 20-35% MeOH in DCM, to give 2-[5-[(E)-3-ethoxy-3-oxo-prop-1-enyl]-2-thienyl]acetic acid (868 mg, 3.61 mmol, 72.6%) as a brown oil. MS (ESI): 241.1 m/z (M+H)⁺.

### 2-(5-(3-Ethoxy-3-oxopropyl)thiophen-2-yl)acetic acid

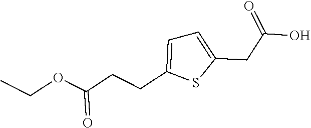 [SMILES: CCOC(=O)CCc1ccc(CC(=O)O)s1]

Step B: A mixture of 2-[5-[(E)-3-ethoxy-3-oxo-prop-1-enyl]-2-thienyl]acetic acid (1.00 eq, 868 mg, 3.61 mmol) and Pd/C (10 wt. %, 0.300 eq, 115 mg, 1.08 mmol) in ethyl acetate (100 mL) was stirred for 5 hours at 50° C. under a hydrogen atmosphere. The mixture was filtered and concentrated to give 2-[5-(3-ethoxy-3-oxo-propyl)-2-thienyl]acetic acid (650 mg, 2.68 mmol, 74.3%) as a yellow liquid. MS (ESI): 243.1 m/z (M+H)⁺.

### Ethyl 3-(5-(3-chloro-2-oxopropyl)thiophen-2-yl)propanoate

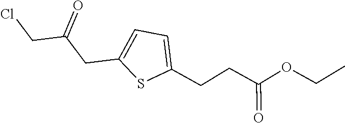 [SMILES: CCOC(=O)CCc1ccc(CC(=O)CCl)s1]

Step C: A solution of (2-[5-(3-ethoxy-3-oxo-propyl)-2-thienyl]acetic acid (1.00 eq, 600 mg, 2.48 mmol) in thionyl chloride (182 eq, 33 mL, 452 mmol) was stirred at 70° C. for 1 hour. The mixture was concentrated under reduced pressure. The residue was dissolved in acetonitrile (20 mL), and trimethylsilyldiazomethane (4.00 eq, 1131 mg, 9.91 mmol) was added slowly at 0° C. The solution was allowed to warm to room temperature and stirred for 16 hours. Hydrogen chloride (4.50 eq, 406 mg, 11.1 mmol) was added and the mixture was stirred for 1 hour. Water (50 mL) was added, and the mixture was extracted with ethyl acetate (100 mL×3). The organic extracts were combined, washed with water (100 mL) and brine (100 mL×2), dried over anhydrous sodium sulfate, filtered and concentrated to give the product, ethyl 3-[5-(3-chloro-2-oxo-propyl)-2-thienyl]propanoate (500 mg, 1.82 mmol, 73.5%) as a yellow liquid. MS (ESI): 275.1 m/z (M+H)⁺.

### Ethyl 3-(5-((2-(5-((4,6-difluoro-1H-indol-5-yl)oxy)-2-fluorophenyl)-1H-imidazol-5-yl)methyl)thiophen-2-yl)propanoate

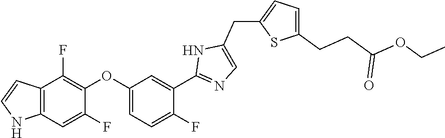 [SMILES: CCOC(=O)CCc1ccc(Cc2cnc(-c3cc(Oc4c(F)cc5[nH]ccc5c4F)ccc3F)[nH]2)s1]

Step D: To a solution of ethyl 3-[5-(3-chloro-2-oxo-propyl)-2-thienyl]propanoate (1.00 eq, 450 mg, 1.64 mmol) in THE (50 mL) were added sodium bicarbonate (3.00 eq, 413 mg, 4.91 mmol) and 5-[(4,6-difluoro-1H-indol-5-yl)oxy]-2-fluoro-benzamidine (Intermediate 4, 500 mg, 1.64 mmol). The mixture was stirred at 70° C. for 2 days, cooled to room temperature, quenched with water (50 mL), and extracted with ethyl acetate (50 mL×3). The organic layers were combined, washed with water (50 mL) and brine (50 mL×2), dried over anhydrous sodium sulfate, filtered, and concentrated. The residue was purified by prep-HPLC to give the product, ethyl 3-(5-((2-(5-((4,6-difluoro-1H-indol-5-yl)oxy)-2-fluorophenyl)-1H-imidazol-5-yl)methyl)thiophen-2-yl)propanoate (30 mg, 0.057 mmol, 3.5%) as a white solid. MS (ESI): 526.1 m/z (M+H)⁺.

### 3-(5-((2-(5-((4,6-Difluoro-1H-indol-5-yl)oxy)-2-fluorophenyl)-1H-imidazol-5-yl)methyl)thiophen-2-yl)propanoic acid

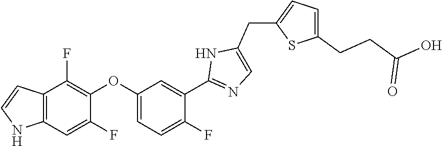 [SMILES: O=C(O)CCc1ccc(Cc2cnc(-c3cc(Oc4c(F)cc5[nH]ccc5c4F)ccc3F)[nH]2)s1]

Step E: A mixture of ethyl 3-[5-[[2-[5-[(4,6-difluoro-1H-indol-5-yl)oxy]-2-fluoro-phenyl]-1H-imidazol-5-yl]methyl]-2-thienyl]propanoate (1.00 eq, 30 mg, 0.057 mmol) and lithium hydroxide (2.00 eq, 2.7 mg, 0.114 mmol) in THE (10 mL) and Water (1 mL) was stirred overnight at room temperature. The mixture was acidified with 1.0 M hydrochloric acid to pH=6, and extracted with ethyl acetate (3×20 mL). The combined organic extracts were washed with brine (20 mL×2), dried over anhydrous sodium sulfate, filtered and concentrated to give the title compound, 3-[5-[[2-[5-[(4,6-difluoro-1H-indol-5-yl)oxy]-2-fluoro-phenyl]-1H-imidazol-5-yl]methyl]-2-thienyl]propanoic acid (7.0 mg, 0.014 mmol, 25% yield). MS (ESI): 498.1 m/z (M+H)⁺. ¹H NMR (400 MHz, MeOH-d₄): δ 7.49 (q, 1H), 7.29 (d, 1H), 7.21-7.11 (m, 2H), 6.98-6.96 (m, 1H), 6.89 (s, 1H), 6.64 (s, 2H), 6.54 (d, 1H), 4.05 (s, 2H), 3.04 (bs, 2H), 2.62 (bs, 2H) ppm.

### Example 44. Synthesis of (S)-3-(5-(1-(5-(5-((4,6-difluoro-1H-indol-5-yl)oxy)-2-fluorophenyl)-1-methyl-1H-1,2,4-triazol-3-yl)ethyl)thiophen-2-yl)propanoic acid

### Example 45. Synthesis of (R)-3-(5-(1-(5-(5-((4,6-difluoro-1H-indol-5-yl)oxy)-2-fluorophenyl)-1-methyl-1H-1,2,4-triazol-3-yl)ethyl)thiophen-2-yl)propanoic acid

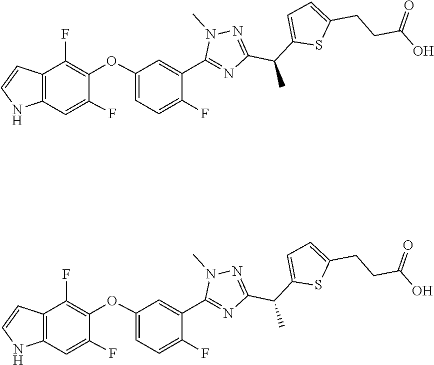 [SMILES: C[C@@H](c1nc(-c2cc(Oc3c(F)cc4[nH]ccc4c3F)ccc2F)n(C)n1)c1ccc(CCC(=O)O)s1 ; C[C@H](c1nc(-c2cc(Oc3c(F)cc4[nH]ccc4c3F)ccc2F)n(C)n1)c1ccc(CCC(=O)O)s1]

### Methyl 2-(5-bromothiophen-2-yl)acetate

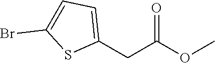

Step A: To a solution of methyl 2-(2-thienyl)acetate (1.00 eq, 1.00 g, 6.40 mmol) in methanol (20 mL) and THE (20 mL) was added N-bromosuccinimide (1.50 eq, 1709 mg, 9.60 mmol). The mixture was stirred at room temperature overnight, diluted with 200 mL of water, extracted with ethyl acetate (200 mL×3). The combined organic phase was washed with brine, dried over Na₂SO₄ and concentrated. The crude was purified by silica gel column chromatography, eluting with 0-20% of ethyl acetate in petroleum ether, to give methyl 2-(5-bromo-2-thienyl)acetate (1.20 g, 5.10 mmol, 79.7%) as a yellow liquid. MS (ESI): 235.1, 237.1 m/z (M+H)⁺.

### Methyl 2-(5-bromothiophen-2-yl)propanoate

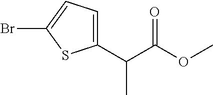

Step B: To a solution of methyl 2-(5-bromo-2-thienyl)acetate (1.00 eq, 1.20 g, 5.10 mmol) in THE (30 mL) was added LDA (2.0 M in THF/heptane/ethylbenzene) (1.30 eq, 3.3 mL, 6.64 mmol) at −78° C. under an Ar atmosphere and stirred for 30 min. Methyl iodide (1.20 eq, 0.38 mL, 6.13 mmol) was added and the mixture was stirred at room temperature for 1 hour, diluted with 100 mL of water, extracted with ethyl acetate (100 mL×3). The combined organic phase was washed with brine, dried over Na₂SO₄ and concentrated. The crude was purified by silica gel column chromatography (eluting with 0-10% of Ethyl acetate in Petroleum ether) to give methyl 2-(5-bromo-2-thienyl)propanoate (820 mg, 3.29 mmol, 64.5%) as a yellow liquid. MS (ESI): 249.0, 251.0 m/z (M+H)⁺.

### 2-(5-Bromothiophen-2-yl)propanoic acid

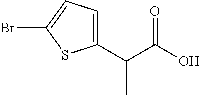

Step C: To a solution of methyl 2-(5-bromo-2-thienyl)propanoate (1.00 eq, 820 mg, 3.29 mmol) in Methanol (10 mL), THE (10 mL) and Water (5 mL) was added Lithium hydroxide monohydrate (5.00 eq, 691 mg, 16.5 mmol), the mixture was stirred at room temperature for 2 hours, diluted with 30 mL of water, acidified with HCl solution to pH=5, extracted with Ethyl acetate (100 mL×3). The combined organic phase was washed with brine, dried over Na₂SO₄ and concentrated to give 2-(5-bromo-2-thienyl)propanoic acid (780 mg, 3.32 mmol, 100.79% yield) as a yellow liquid. MS (ESI): 235.1, 237.1 m/z (M+H)⁺.

### Tert-butyl 2-(2-(5-bromothiophen-2-yl)propanoyl)-1-methylhydrazine-1-carboxylate

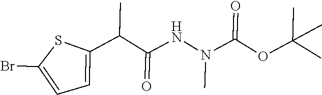 [SMILES: CC(C(=O)NN(C)C(=O)OC(C)(C)C)c1ccc(Br)s1]

Step D: To a solution of 2-(5-bromo-2-thienyl)propanoic acid (1.00 eq, 780 mg, 3.32 mmol) in acetonitrile (20 mL) was added 1-Boc-1-methylhydrazine (1.50 eq, 728 mg, 4.98 mmol), N,N,N′,N′-Tetramethylchloroformamidinium hexafluorophosphate (1.00 eq, 931 mg, 3.32 mmol) and 1-methylimidazole (3.50 eq, 0.93 mL, 11.6 mmol). The resulting mixture was stirred at room temperature for 1 hour, diluted with 100 mL of water, and extracted with ethyl acetate (100 mL×3). The combined organic phase was washed with brine, dried over Na₂SO₄ and concentrated. The crude was purified by silica gel column chromatography, eluting with 0-35% of ethyl acetate in petroleum ether, to afford tert-butyl N-[2-(5-bromo-2-thienyl)propanoylamino]-N-methyl-carbamate (1.05 g, 2.89 mmol, 87.12%) as a colorless liquid. MS (ESI): 385.0, 387.0 m/z (M+Na)⁺.

### 2-(5-Bromothiophen-2-yl)-N′-methylpropanehydrazide

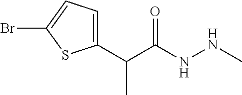

Step E: To a solution of tert-butyl N-[2-(5-bromo-2-thienyl)propanoylamino]-N-methyl-carbamate (1.00 eq, 1.05 g, 2.89 mmol) in DCM (20 mL) was added trifluoroacetic acid (2.00 eq, 0.45 mL, 5.78 mmol), and the mixture was stirred overnight at room temperature. The solvent was removed under reduced pressure to give 2-(5-bromo-2-thienyl)-N′-methyl-propanehydrazide (750 mg, 2.85 mmol, 99%) as a yellow liquid. MS (ESI): 263.0, 265.0 m/z (M+H)⁺.

### 5-(3-(3-(1-(5-Bromothiophen-2-yl)ethyl)-1-methyl-1H-1,2,4-triazol-5-yl)-4-fluorophenoxy)-4,6-difluoro-1H-indole

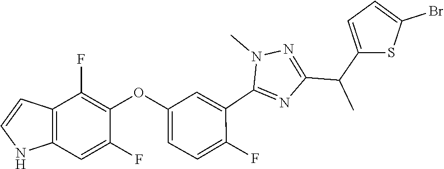 [SMILES: CC(c1nc(-c2cc(Oc3c(F)cc4[nH]ccc4c3F)ccc2F)n(C)n1)c1ccc(Br)s1]

Step F: To a solution of 2-(5-bromo-2-thienyl)-N′-methyl-propanehydrazide (1.00 eq, 750 mg, 2.85 mmol) in pyridine (30 mL) was added methyl 5-[(4,6-difluoro-1H-indol-5-yl)oxy]-2-fluoro-benzenecarboximidothioate; hydroiodide (1.00 eq, 1323 mg, 2.85 mmol) and magnesium sulfate (10.0 eq, 3431 mg, 28.5 mmol). The reaction mixture was stirred at 80° C. overnight. The solvent was removed under reduced pressure, the mixture was diluted with 100 mL of water, and extracted with ethyl acetate (100 mL×3). The combined organic phase was washed with brine, dried over Na₂SO₄ and concentrated. The crude was purified by silica gel column chromatography, eluting with 0-35% of ethyl acetate in petroleum ether, to give 5-[3-[5-[1-(5-bromo-2-thienyl)ethyl]-2-methyl-1,2,4-triazol-3-yl]-4-fluoro-phenoxy]-4,6-difluoro-1H-indole (650 mg, 1.22 mmol, 43%) as a white solid. MS (ESI): 533.0, 535.0 m/z (M+H)⁺.

### Ethyl (E)-3-(5-(1-(5-(5-((4,6-difluoro-1H-indol-5-yl)oxy)-2-fluorophenyl)-1-methyl-1H-1,2,4-triazol-3-yl)ethyl)thiophen-2-yl)acrylate

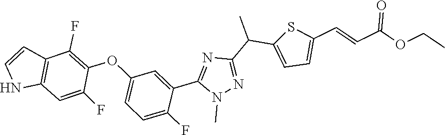 [SMILES: CCOC(=O)/C=C/c1ccc(C(C)c2nc(-c3cc(Oc4c(F)cc5[nH]ccc5c4F)ccc3F)n(C)n2)s1]

Step G: To a solution of 5-[3-[5-[1-(5-bromo-2-thienyl)ethyl]-2-methyl-1,2,4-triazol-3-yl]-4-fluoro-phenoxy]-4,6-difluoro-1H-indole (1.00 eq, 650 mg, 1.22 mmol) in 1,4-dioxane (20 ml) and water (4 mL) was added Pd(dppf)Cl₂ dichloromethane complex (1.00 eq, 995 mg, 1.22 mmol), ethyl (E)-3-(4,4,5,5-tetramethyl-1,3,2-dioxaborolan-2-yl)prop-2-enoate (1.00 eq, 276 mg, 1.22 mmol) and potassium carbonate (1.00 eq, 168 mg, 1.22 mmol). The reaction mixture was stirred overnight at 90° C. under an argon atmosphere, cooled to room temperature, diluted with 100 mL of water, and extracted with ethyl acetate (100 mL×3). The combined organic extracts were washed with brine, dried over Na₂SO₄ and concentrated. The crude was purified by silica gel column chromatography (eluting with 0-40% of ethyl acetate in petroleum ether) to give ethyl (E)-3-[5-[1-[5-[5-[(4,6-difluoro-1H-indol-5-yl)oxy]-2-fluoro-phenyl]-1-methyl-1,2,4-triazol-3-yl]ethyl]-2-thienyl]prop-2-enoate (615 mg, 1.11 mmol, 91.3%) as a white solid. MS (ESI): 553.2 m/z (M+H)⁺.

### Ethyl 3-(5-(1-(5-(5-((4,6-difluoro-1H-indol-5-yl)oxy)-2-fluorophenyl)-1-methyl-1H-1,2,4-triazol-3-yl)ethyl)thiophen-2-yl)propanoate

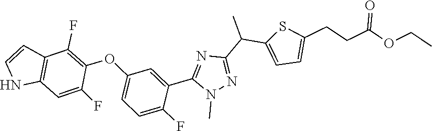 [SMILES: CCOC(=O)CCc1ccc(C(C)c2nc(-c3cc(Oc4c(F)cc5[nH]ccc5c4F)ccc3F)n(C)n2)s1]

Step H: To a solution of ethyl (E)-3-[5-[1-[5-[5-[(4,6-difluoro-1H-indol-5-yl)oxy]-2-fluoro-phenyl]-1-methyl-1,2,4-triazol-3-yl]ethyl]-2-thienyl]prop-2-enoate (1.00 eq, 615 mg, 1.11 mmol) in ethanol (20 mL) was added Pd/C (10 wt. %, 0.100 eq, 118 mg, 0.111 mmol) and the resulting suspension was stirred for 2 hours at 50° C. under a H₂ atmosphere. The reaction mixture was filtered and the filtrate was concentrated. The residue was purified by silica gel column chromatography (eluting with 0-40% of ethyl acetate in petroleum ether) to give ethyl 3-[5-[1-[5-[5-[(4,6-difluoro-1H-indol-5-yl)oxy]-2-fluoro-phenyl]-1-methyl-1,2,4-triazol-3-yl]ethyl]-2-thienyl]propanoate (505 mg, 0.911 mmol, 81.8%) as a white solid. MS (ESI): 555.2 m/z (M+H)⁺.

### Ethyl (S)-3-(5-(1-(5-(5-((4,6-difluoro-1H-indol-5-yl)oxy)-2 fluorophenyl)-1-methyl-1H-1,2,4-triazol-3-yl)ethyl)thiophen-2-yl)propanoate and ethyl (R)-3-(5-(1-(5-(5-((4,6-difluoro-1H-indol-5-yl)oxy)-2-fluorophenyl)-1-methyl-1H-1,2,4-triazol-3-yl)ethyl)thiophen-2-yl)propanoate

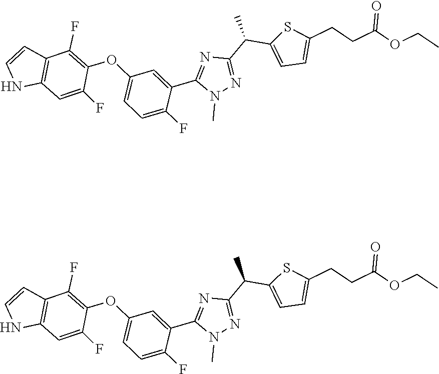 [SMILES: CCOC(=O)CCc1ccc([C@@H](C)c2nc(-c3cc(Oc4c(F)cc5[nH]ccc5c4F)ccc3F)n(C)n2)s1 ; CCOC(=O)CCc1ccc([C@H](C)c2nc(-c3cc(Oc4c(F)cc5[nH]ccc5c4F)ccc3F)n(C)n2)s1]

Step I: Ethyl 3-[5-[1-[5-[5-[(4,6-difluoro-1H-indol-5-yl)oxy]-2-fluoro-phenyl]-1-methyl-1,2,4-triazol-3-yl]ethyl]-2-thienyl]propanoate (1.00 eq, 450 mg, 0.811 mmol) was resolved by chiral SFC into its constituent enantiomers. The absolute configuration of the faster eluting enantiomer was arbitrarily assigned as ethyl 3-[5-[(1S)-1-[5-[5-[(4,6-difluoro-1H-indol-5-yl)oxy]-2-fluoro-phenyl]-1-methyl-1,2,4-triazol-3-yl]ethyl]-2-thienyl]propanoate (150 mg, 0.270 mmol, 33.3%. a white solid) and the absolute configuration of the slower eluting enantiomer was respectively assigned as ethyl 3-[5-[(R)-1-[5-[5-[(4,6-difluoro-1H-indol-5-yl)oxy]-2-fluoro-phenyl]-1-methyl-1,2,4-triazol-3-yl]ethyl]-2-thienyl]propanoate (210 mg, 0.379 mmol, 46.7%, a white solid). MS (ESI): 555.2 m/z (M+H)⁺ observed for both enantiomers.

### Chiral SFC Separation Conditions:

Instrument: SFC-150 (Waters)
Column: OJ 20*250 mm, 10 um (REGIS)
Column temperature: 35° C.
Mobile phase: 80/20 CO₂/MeOH (7M ammonia in MeOH)
Flow rate: 100 mL/min; Back pressure: 100 bar
Detection wavelength: 214 nm; Cycle time: 5.1 minutes
Sample solution: 450 mg dissolved in 40 mL methanol; Injection volume: 2.0 mL

### (S)-3-(5-(1-(5-(5-((4,6-difluoro-1H-indol-5-yl)oxy)-2-fluorophenyl)-1-methyl-1H-1,2,4-triazol-3-yl)ethyl)thiophen-2-yl)propanoic acid

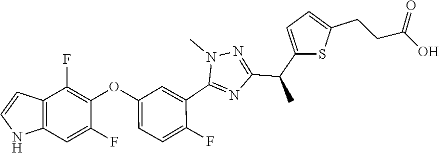 [SMILES: C[C@@H](c1nc(-c2cc(Oc3c(F)cc4[nH]ccc4c3F)ccc2F)n(C)n1)c1ccc(CCC(=O)O)s1]

Step J: To a solution of ethyl 3-[5-[(1S)-1-[5-[5-[(4,6-difluoro-1H-indol-5-yl)oxy]-2-fluoro-phenyl]-1-methyl-1,2,4-triazol-3-yl]ethyl]-2-thienyl]propanoate (1.00 eq, 150 mg, 0.270 mmol) in a mixture of methanol (10 mL), THE (10 mL) and water (5 mL) was added lithium hydroxide monohydrate (5.00 eq, 57 mg, 1.35 mmol). The reaction mixture was stirred at room temperature overnight, diluted with 100 mL of water, acidified with hydrochloric acid to pH=5, and extracted with ethyl acetate (100 mL×3). The combined organic extracts were washed with brine, dried over Na₂SO₄ and concentrated. The residue was purified by silica gel column chromatography (eluting with 0-6% MeOH in DCM) to give 3-[5-[(1S)-1-[5-[5-[(4,6-difluoro-1H-indol-5-yl)oxy]-2-fluoro-phenyl]-1-methyl-1,2,4-triazol-3-yl]ethyl]-2-thienyl]propanoic acid (Example 44, 118 mg, 0.224 mmol, 83%) as a white solid. MS (ESI): 527.1 m/z (M+H)⁺. ¹H NMR (400 MHz, MeOH-d₄): δ 7.36-7.29 (m, 2H), 7.20-7.11 (m, 3H), 6.74 (d, 1H), 6.64 (d, 1H), 6.56 (dd, 1H), 4.44 (q, 1H), 3.81 (d, 3H), 3.03 (t, 2H), 2.60 (t, 2H) ppm.

### (R)-3-(5-(1-(5-(5-((4,6-difluoro-1H-indol-5-yl)oxy,)-2-fluorophenyl)-1-methyl-1H-1,2,4-triazol-3-yl)ethyl)thiophen-2-yl)propanoic acid

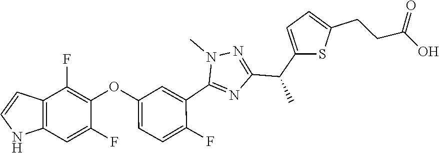 [SMILES: C[C@H](c1nc(-c2cc(Oc3c(F)cc4[nH]ccc4c3F)ccc2F)n(C)n1)c1ccc(CCC(=O)O)s1]

Step K: To a solution of ethyl 3-[5-[(1R)-1-[5-[5-[(4,6-difluoro-1H-indol-5-yl)oxy]-2-fluoro-phenyl]-1-methyl-1,2,4-triazol-3-yl]ethyl]-2-thienyl]propanoate (1.00 eq, 210 mg, 0.379 mmol) in a mixture of methanol (10 mL), THE (10 mL) and water (5 mL) was added lithium hydroxide monohydrate (5.00 eq, 79 mg, 1.89 mmol). The reaction mixture was stirred at room temperature overnight, diluted with 100 mL of water, acidified with hydrochloric acid to pH=5, and extracted with ethyl acetate (100 mL×3). The combined organic extracts were washed with brine, dried over Na₂SO₄ and concentrated. The residue was purified by silica gel column chromatography (eluting with 0-6% of MeOH in DCM) to give 3-[5-[(1R)-1-[5-[5-[(4,6-difluoro-1H-indol-5-yl)oxy]-2-fluoro-phenyl]-1-methyl-1,2,4-triazol-3-yl]ethyl]-2-thienyl]propanoic acid (Example 45, 182 mg, 0.346 mmol, 91%) as a white solid. MS (ESI): 527.2 m/z (M+H)⁺. ¹H NMR (400 MHz, MeOH-d₄): δ 7.34-7.29 (m, 2H), 7.20-7.11 (m, 3H), 6.71 (d, 1H), 6.64 (d, 1H), 6.56 (dd, 1H), 4.43 (q, 1H), 3.81 (d, 3H), 3.03 (t, 2H), 2.60 (t, 2H) ppm.

### Example 46. Synthesis of 3-(3-(1-(3-(5-((4,6-difluoro-1H-indol-5-yl)oxy)-2-fluorophenyl)imidazo[1,5-a]pyridin-1-yl)ethyl)-2-fluorophenyl)propanoic acid

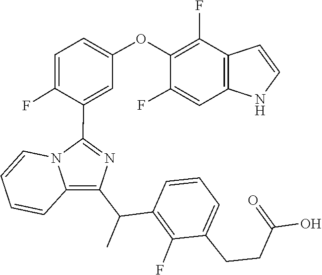 [SMILES: CC(c1cccc(CCC(=O)O)c1F)c1nc(-c2cc(Oc3c(F)cc4[nH]ccc4c3F)ccc2F)n2ccccc12]

### 2-(3-Bromo-2-fluorophenyl)-N-methoxy-N-methylpropanamide

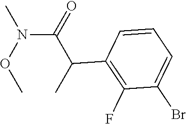 [SMILES: CON(C)C(=O)C(C)c1cccc(Br)c1F]

Step A: To a solution of 2-(3-bromo-2-fluoro-phenyl)propanoic acid (1.00 eq, 2.00 g, 8.10 mmol) in DCM (40 mL) was added N,N′-carbonyldiimadozole (2.00 eq, 2625 mg, 16.2 mmol) and N-methoxymethanamine (2.00 eq, 989 mg, 16.2 mmol). The resulting mixture was stirred at room temperature overnight, diluted with 200 mL of water, and extracted with DCM (200 mL×3). The combined organic extracts were washed with brine, dried over Na₂SO₄ and concentrated. The crude was purified by silica gel column chromatography (eluting with 0-50% ethyl acetate in petroleum ether) to give 2-(3-bromo-2-fluoro-phenyl)-N-methoxy-N-methyl-propanamide (2.10 g, 7.24 mmol, 89.4%) as a yellow liquid. MS (ESI): 290.2, 292.2 m/z (M+H)⁺.

### 2-(3-Bromo-2-fluorophenyl)-1-(pyridin-2-yl)propan-1-one

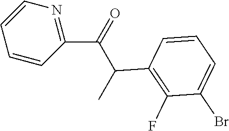 [SMILES: CC(C(=O)c1ccccn1)c1cccc(Br)c1F]

Step B: To a solution of 2-bromopyridine (1.50 eq, 1.1 mL, 10.9 mmol) in THE (50 mL) was added n-butyllithium (1.50 eq, 4.3 mL, 10.9 mmol) at −78° C. under an Ar atmosphere and stirring at −78° C. was continued for 1 hour. 2-(3-Bromo-2-fluoro-phenyl)-N-methoxy-N-methyl-propanamide (1.00 eq, 2.10 g, 7.24 mmol) was added and the mixture was stirred at room temperature overnight. The reaction was diluted with 100 mL of water and extracted with ethyl acetate (100 mL×3). The combined organic extracts were washed with brine, dried over Na₂SO₄ and concentrated. The crude was purified by silica gel column chromatography (eluting with 0-20% ethyl acetate in petroleum ether) to give 2-(3-bromo-2-fluoro-phenyl)-1-(2-pyridyl)propan-1-one (480 mg, 1.56 mmol, 21.5%) as a yellow liquid. MS (ESI): 308.2 m/z (M+H)⁺.

### 2-(3-Bromo-2-fluorophenyl)-1-(pyridin-2-yl)propan-1-amine

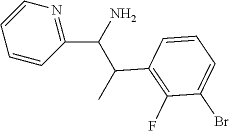 [SMILES: CC(c1cccc(Br)c1F)C(N)c1ccccn1]

Step C: To a stirred solution of 2-(3-bromo-2-fluoro-phenyl)-1-(2-pyridyl)propan-1-one (1.00 eq, 480 mg, 1.56 mmol) in methanol (20 mL) was added hydroxylamine hydrochloride (2.50 eq, 271 mg, 3.89 mmol), and stirring continued overnight at room temperature. The solvent was removed under reduced pressure, the residue diluted with 100 mL of water, and the resulting mixture extracted with ethyl acetate (100 mL×3). The combined organic extracts were washed with brine, dried over Na₂SO₄ and concentrated to afford the oxime as a yellow solid. To a solution of the oxime in trifluoroacetic acid (125 eq, 15 mL, 195 mmol) was added zinc powder (5.00 eq, 509 mg, 7.79 mmol), and the mixture stirred at room temperature for 2 hours. The reaction was diluted with 100 mL of water, and the pH adjusted to ~8 with aqueous NaOH. The aqueous mixture was extracted with ethyl acetate (100 mL×3). The combined organic extracts were washed with brine, dried over Na₂SO₄, and concentrated. The crude was purified by silica gel column chromatography (eluting with 0-5% methanol in DCM, spiked with 0.5% ammonium hydroxide) to give 2-(3-bromo-2-fluoro-phenyl)-1-(2-pyridyl)propan-1-amine (140 mg, 0.453 mmol, 29%) as a white solid. MS (ESI): 309.2 m/z (M+H)⁺.

### 1-(1-(3-Bromo-2-fluorophenyl)ethyl)-3-(5-((4,6-difluoro-1H-indol-5-yl)oxy)-2-fluorophenyl)imidazo[1,5-a]pyridine

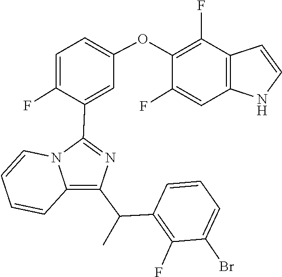 [SMILES: CC(c1cccc(Br)c1F)c1nc(-c2cc(Oc3c(F)cc4[nH]ccc4c3F)ccc2F)n2ccccc12]

Step D: To a solution of 2-(3-bromo-2-fluoro-phenyl)-1-(2-pyridyl)propan-1-amine (1.00 eq, 0.14 g, 0.45 mmol) in butyl acetate (15 mL) was added 5-[(4,6-difluoro-1H-indol-5-yl)oxy]-2-fluoro-benzoic acid (Intermediate 12, 1.00 eq, 0.14 g, 0.453 mmol) and propylphosphonic anhydride (5.00 eq, 1441 mg, 2.26 mmol). The reaction mixture was stirred at room temperature for 1 hour, then heated overnight at 140° C., cooled to room temperature, diluted with 100 mL of water, and extracted with ethyl acetate (100 mL×3). The combined organic extracts were washed with brine, dried over Na₂SO₄, and concentrated. The crude was purified by silica gel column chromatography (eluting with 0-30% ethyl acetate in petroleum ether) to give 1-[1-(3-bromo-2-fluoro-phenyl)ethyl]-3-[5-[(4,6-difluoro-1H-indol-5-yl)oxy]-2-fluoro-phenyl]imidazo[1,5-a]pyridine (75 mg, 0.13 mmol, 29%) as a white solid. MS (ESI): 580.2, 582.2 m/z (M+H)⁺.

### Ethyl (E)-3-(3-(1-(3-(5-((4,6-difluoro-1H-indol-5-yl)oxy)-2-fluorophenyl)imidazo[1,5-a]pyridin-1-yl)ethyl)-2-fluorophenyl)acrylate

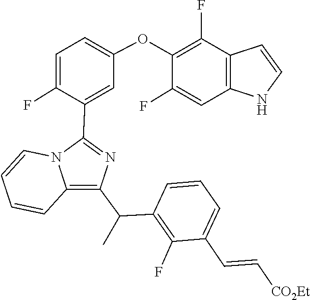 [SMILES: CCOC(=O)/C=C/c1cccc(C(C)c2nc(-c3cc(Oc4c(F)cc5[nH]ccc5c4F)ccc3F)n3ccccc23)c1F]

Step E: To a solution of 1-[1-(3-bromo-2-fluoro-phenyl)ethyl]-3-[5-[(4,6-difluoro-1H-indol-5-yl)oxy]-2-fluoro-phenyl]imidazo[1,5-a]pyridine (1.00 eq, 120 mg, 0.207 mmol) in 1,4-dioxane (5 mL) and water (1 mL) was added Pd(ddpf)Cl₂ dichloromethane complex (0.20 eq, 34 mg, 0.041 mmol), ethyl (E)-3-(4,4,5,5-tetramethyl-1,3,2-dioxaborolan-2-yl)prop-2-enoate (2.00 eq, 93 mg, 0.414 mmol) and potassium carbonate (2.00 eq, 57 mg, 0.41 mmol). The reaction mixture was stirred overnight at 90° C., diluted with 100 mL of water, and extracted with ethyl acetate (100 mL×3). The combined organic extracts were washed with brine, dried over Na₂SO₄, and concentrated. The crude was purified by silica gel column chromatography (eluting with 0-60% ethyl acetate in petroleum ether) to give ethyl (Z)-3-[3-[1-[3-[5-[(4,6-difluoro-1H-indol-5-yl)oxy]-2-fluoro-phenyl]imidazo[1,5-a]pyridin-1-yl]ethyl]-2-fluoro-phenyl]prop-2-enoate (90 mg, 0.15 mmol, 72.6%) as a white solid. MS (ESI): 600.3 m/z (M+H)⁺.

### Ethyl 3-(3-(1-(3-(5-((4,6-difluoro-1H-indol-5-yl)oxy)-2-fluorophenyl)imidazo[1,5-a]pyridin-1-yl)ethyl)-2-fluorophenyl)propanoate

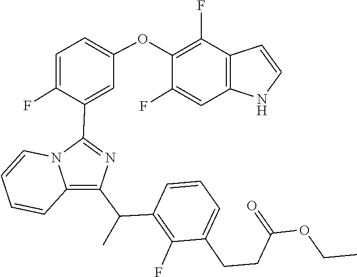 [SMILES: CCOC(=O)CCc1cccc(C(C)c2nc(-c3cc(Oc4c(F)cc5[nH]ccc5c4F)ccc3F)n3ccccc23)c1F]

Step F: To a solution of ethyl (Z)-3-[3-[1-[3-[5-[(4,6-difluoro-1H-indol-5-yl)oxy]-2-fluoro-phenyl]imidazo[1,5-a]pyridin-1-yl]ethyl]-2-fluoro-phenyl]prop-2-enoate (1.00 eq, 70 mg, 0.117 mmol) in Toluene (10 mL) was added p-toluenesulfonylhydrazide (217 mg, 1.17 mmol, 10.0 eq) and stirred at room temperature under a H₂ atmosphere for 2 hours. The reaction mixture was filtered and the filtrate was concentrated to give ethyl 3-[3-[1-[3-[5-[(4,6-difluoro-1H-indol-5-yl)oxy]-2-fluoro-phenyl]imidazo[1,5-a]pyridin-1-yl]ethyl]-2-fluoro-phenyl]propanoate (10 mg, 0.017 mmol, 14.2%) as a white solid. MS (ESI): 602.3 m/z (M+H)⁺.

### 3-(3-(1-(3-(5-((4,6-Difluoro-1H-indol-5-yl)oxy)-2-fluorophenyl)imidazo[1,5-a]pyridin-1-yl)ethyl)-2-fluorophenyl)propanoic acid

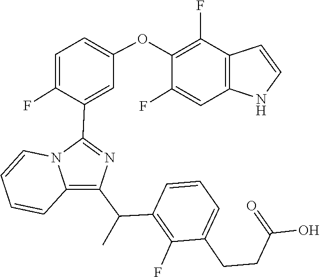 [SMILES: CC(c1cccc(CCC(=O)O)c1F)c1nc(-c2cc(Oc3c(F)cc4[nH]ccc4c3F)ccc2F)n2ccccc12]

Step G: To a solution of ethyl 3-[3-[1-[3-[5-[(4,6-difluoro-1H-indol-5-yl)oxy]-2-fluoro-phenyl]imidazo[1,5-a]pyridin-1-yl]ethyl]-2-fluoro-phenyl]propanoate (1.00 eq, 12 mg, 0.02 mmol) in a 1/1/1 H₂O/THF/MeOH mixture (3 mL) was added lithium hydroxide monohydrate (5.00 eq, 4.2 mg, 0.10 mmol). The mixture was allowed to stir overnight at room temperature. The mixture was quenched with a 1 M aqueous HCl solution (2 mL) to pH=2 and extracted with ethyl acetate (25 mL×3). The combined organic extracts were washed with brine (15 mL), dried over Na₂SO₄, filtered, and concentrated. The residue was purified by prep-HPLC to give the product, 3-[3-[1-[3-[5-[(4,6-difluoro-1H-indol-5-yl)oxy]-2-fluoro-phenyl]imidazo[1,5-a]pyridin-1-yl]ethyl]-2-fluoro-phenyl]propanoic acid (9.0 mg, 0.0157 mmol, 78.7%) as a white solid. MS (ESI): 574.3 m/z (M+H)⁺. ¹H NMR (400 MHz, MeOH-d₄): d 7.78 (dd, 1H), 7.34-7.06 (m, 8H), 6.97 (t, 1H), 6.75-6.72 (m, 1H), 6.66 (t, 1H), 6.55 (d, 1H), 4.86 (t, 1H), 2.91 (t, 2H), 2.54 (t, 2H), 1.74 (d, 3H) ppm.

### Example 47. Synthesis of 3-(3-(1-(3-(5-((4,6-difluoro-1H-indol-5-yl)oxy)-2-fluorophenyl)-5,6,7,8-tetrahydroimidazo[1,5-a]pyridin-1-yl)ethyl)-2-fluorophenyl)propanoic acid

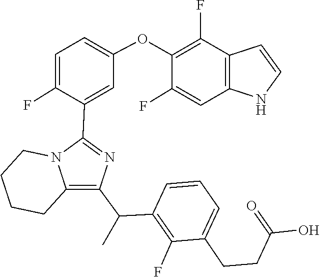 [SMILES: CC(c1cccc(CCC(=O)O)c1F)c1nc(-c2cc(Oc3c(F)cc4[nH]ccc4c3F)ccc2F)n2c1CCCC2]

### Ethyl 3-(3-(1-(3-(5-((4,6-difluoro-1H-indol-5-yl)oxy)-2-fluorophenyl)-5,6,7,8-tetrahydroimidazo[1,5-a]pyridin-1-yl)ethyl)-2-fluorophenyl)propanoate

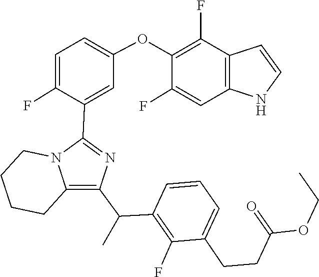 [SMILES: CCOC(=O)CCc1cccc(C(C)c2nc(-c3cc(Oc4c(F)cc5[nH]ccc5c4F)ccc3F)n3c2CCCC3)c1F]

Step A: To a solution of ethyl (E)-3-[3-[1-[3-[5-[(4,6-difluoro-1H-indol-5-yl)oxy]-2-fluoro-phenyl]imidazo[1,5-a]pyridin-1-yl]ethyl]-2-fluoro-phenyl]prop-2-enoate (1.00 eq, 55 mg, 0.0917 mmol) in methanol (10 mL) was added Pd/C (10 wt. %, 9.8 mg, 0.0092 mmol) and the resulting mixture stirred at room temperature under a H₂ atmosphere for 2 hours. The reaction mixture was filtered and the filtrate was concentrated to give ethyl 3-[3-[1-[3-[5-[(4,6-difluoro-1H-indol-5-yl)oxy]-2-fluoro-phenyl]-5,6,7,8-tetrahydroimidazo[1,5-a]pyridin-1-yl]ethyl]-2-fluoro-phenyl]propanoate (42 mg, 0.069 mmol, 76%) as a white solid. MS (ESI): 606.3 m/z (M+H)⁺.

### 3-(3-(1-(3-(5-((4,6-Difluoro-1H-indol-5-yl)oxy)-2-fluorophenyl)-5,6,7,8-tetrahydroimidazo[1,5-a]pyridin-1-yl)ethyl)-2-fluorophenyl)propanoic acid

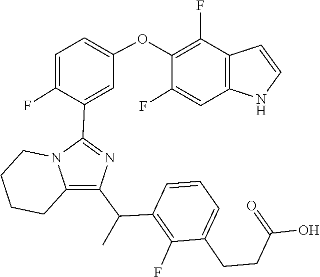 [SMILES: CC(c1cccc(CCC(=O)O)c1F)c1nc(-c2cc(Oc3c(F)cc4[nH]ccc4c3F)ccc2F)n2c1CCCC2]

Step B: To a solution of ethyl 3-[3-[1-[3-[5-[(4,6-difluoro-1H-indol-5-yl)oxy]-2-fluoro-phenyl]-5,6,7,8-tetrahydroimidazo[1,5-a]pyridin-1-yl]ethyl]-2-fluoro-phenyl]propanoate (1.00 eq, 42 mg, 0.0694 mmol) in a mixture of methanol (3 mL), THE (3 mL) and water (1 mL) was added lithium hydroxide monohydrate (5.00 eq, 15 mg, 0.35 mmol). The reaction mixture was stirred at room temperature overnight, diluted with 10 mL of water, acidified with hydrochloric acid to pH=5, and extracted with ethyl acetate (20 mL×3). The combined organic extracts were washed with brine, dried over Na₂SO₄, and concentrated. The residue was purified by prep-HPLC to give 3-[3-[1-[3-[5-[(4,6-difluoro-1H-indol-5-yl)oxy]-2-fluoro-phenyl]-5,6,7,8-tetrahydroimidazo[1,5-a]pyridin-1-yl]ethyl]-2-fluoro-phenyl]propanoic acid (11 mg, 0.019 mmol, 27%) as a white solid. MS (ESI): 578.3 m/z (M+H)⁺.

### Example 48. Synthesis of 3-(3-(1-(5-amino-1-(5-((4,6-difluoro-1H-indol-5-yl)oxy)-2-fluorophenyl)-1H-pyrazol-3-yl)ethyl)-2-fluorophenyl)propanoic acid

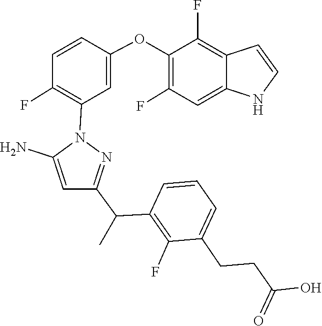 [SMILES: CC(c1cc(N)n(-c2cc(Oc3c(F)cc4[nH]ccc4c3F)ccc2F)n1)c1cccc(CCC(=O)O)c1F]

### 1-Bromo-3-(3-bromo-2-fluorophenyl)butan-2-one

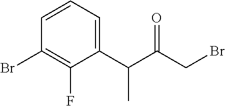

Step A: To a cooled (0° C.) solution of 2-(3-bromo-2-fluoro-phenyl)propanoyl chloride (5 g, 18.8 mmol) in 50 mL of acetonitrile was added dropwise trimethylsilyl diazomethane (38 mL, 76 mmol), and the reaction mixture was allowed to slowly warm to room temperature, and stirred for 3 hours. 33% aqueous HBr (18.3 g, 76 mmol) was added dropwise and the mixture was stirred for 30 min until gas evolution stopped. The solvent was evaporated and the residue was chromatographed on silica, eluting with 0-20% EtOAc in heptanes, to give 1-bromo-3-(3-bromo-2-fluoro-phenyl)butan-2-one (3 g, 47%) as a colorless oil. MS (ESI): 324 m/z (M+H)⁺. 4-(3-Bromo-2-fluorophenyl)-3-oxopentanenitrile

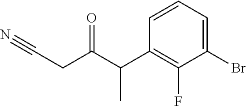

Step B: To a solution of 1-bromo-3-(3-bromo-2-fluoro-phenyl)butan-2-one (500 mg, 1.5433 mmol) in 4 mL of EtOH and 1 mL water, was added sodium cyanide (114 mg, 2.315 mmol), and the resulting mixture was stirred at room temperature for 16 hours. The reaction solution was diluted with 10 mL of water, extracted with EtOAc (3×10 mL), and the organic phase was dried and concentrated. The residue was purified by flash column chromatography on silica eluting with 0-20% EtOAc in hexane to give the title compound, 4-(3-bromo-2-fluoro-phenyl)-3-oxo-pentanenitrile (283 mg, 64.5%) as a yellow oil. MS (ESI): 270, 272 m/z (M+H)⁺.

### 3-(1-(3-Bromo-2-fluorophenyl)ethyl)-1-(5-((4,6-difluoro-1-tosyl-1H-indol-5-yl)oxy)-2-fluorophenyl)-1H-pyrazol-5-amine

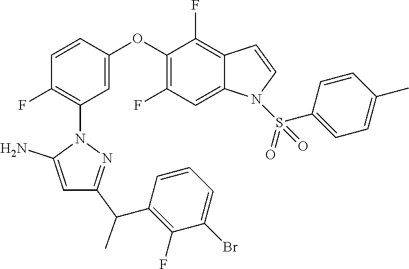 [SMILES: Cc1ccc(S(=O)(=O)n2ccc3c(F)c(Oc4ccc(F)c(-n5nc(C(C)c6cccc(Br)c6F)cc5N)c4)c(F)cc32)cc1]

Step C: To a solution of 4-(3-bromo-2-fluoro-phenyl)-3-oxo-pentanenitrile (54 mg, 0.2 mmol) in 2 mL of EtOH and 0.02 mL acetic acid, was added [5-[4,6-difluoro-1-(p-tolylsulfonyl)indol-5-yl]oxy-2-fluoro-phenyl]hydrazine (Intermediate 13, 90 mg, 0.2 mmol). The solution was stirred at 80° C. for 16 hours. After cooling the solution to room temperature, 5 mL of water were added, the resulting mixture was extracted with EtOAc (3×5 mL), and the organic phase was dried and concentrated. The residue was purified by flash column chromatography on silica, eluting with 0-20% EtOAc in hexane to give the title compound, 5-[1-(3-bromo-2-fluoro-phenyl)ethyl]-2-[5-[4,6-difluoro-1-(p-tolylsulfonyl)indol-5-yl]oxy-2-fluoro-phenyl]pyrazol-3-amine (70 mg, 50%) as a yellow oil. MS (ESI): 699, 701 m/z (M+H)⁺.

### Ethyl (E)-3-(3-(1-(5-amino-1-(5-((4,6-difluoro-1-tosyl-1H-indol-5-yl)oxy)-2-fluorophenyl)-1H-pyrazol-3-yl)ethyl)-2-fluorophenyl)acrylate

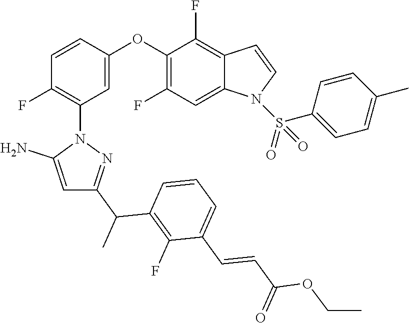 [SMILES: CCOC(=O)/C=C/c1cccc(C(C)c2cc(N)n(-c3cc(Oc4c(F)cc5c(ccn5S(=O)(=O)c5ccc(C)cc5)c4F)ccc3F)n2)c1F]

Step D: To a solution of 5-[1-(3-bromo-2-fluoro-phenyl)ethyl]-2-[5-[4,6-difluoro-1-(p-tolylsulfonyl)indol-5-yl]oxy-2-fluoro-phenyl]pyrazol-3-amine (70 mg, 0.1 mmol) in 5 mL of 1,4-dioxane and 0.5 mL water, were added ethyl (E)-3-(4,4,5,5-tetramethyl-1,3,2-dioxaborolan-2-yl)prop-2-enoate (45.2 mg, 0.2 mmol), Pd(dppf)Cl₂ dichloromethane complex (8.2 mg), and potassium carbonate (28 mg), and the solution was stirred at 80° C. for 16 hours under an argon atmosphere. 5 mL of water were added to the cooled reaction mixture, which was then extracted with EtOAc (3×5 mL). The organic extracts were dried and concentrated. The residue was purified by flash column chromatography on silica, eluting with 0-20% EtOAc in hexane to give the title compound, ethyl (E)-3-[3-[1-[5-amino-1-[5-[4,6-difluoro-1-(p-tolylsulfonyl)indol-5-yl]oxy-2-fluoro-phenyl]pyrazol-3-yl]ethyl]-2-fluoro-phenyl]prop-2-enoate (50 mg, 70%) as a yellow oil. MS (ESI): 719 m/z (M+H)⁺.

### Ethyl 3-(3-(1-(5-amino-1-(5-((4,6-difluoro-1-tosyl-1H-indol-5-yl)oxy)-2-fluorophenyl)-1H-pyrazol-3-yl)ethyl)-2-fluorophenyl)propanoate

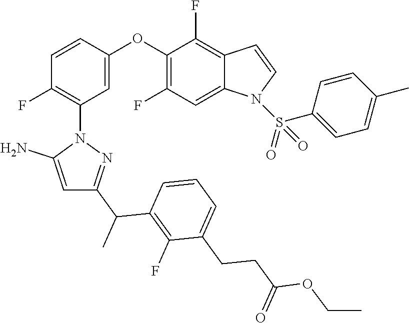 [SMILES: CCOC(=O)CCc1cccc(C(C)c2cc(N)n(-c3cc(Oc4c(F)cc5c(ccn5S(=O)(=O)c5ccc(C)cc5)c4F)ccc3F)n2)c1F]

Step E: To a solution of ethyl (E)-3-[3-[1-[5-amino-1-[5-[4,6-difluoro-1-(p-tolylsulfonyl)indol-5-yl]oxy-2-fluoro-phenyl]pyrazol-3-yl]ethyl]-2-fluoro-phenyl]prop-2-enoate (50 mg, 0.07 mmol) in 5 mL of EtOH, was added Pd/C (10 wt. %, 25 mg) and the resulting mixture was stirred at room temperature for 16 hours under a hydrogen atmosphere. The solids were filtered off and the filtrate was concentrated to afford the desired compound, ethyl 3-[3-[1-[5-amino-1-[5-[4,6-difluoro-1-(p-tolylsulfonyl)indol-5-yl]oxy-2-fluoro-phenyl]pyrazol-3-yl]ethyl]-2-fluoro-phenyl]propanoate (40 mg, 80%) as a yellow oil. MS (ESI): 721 m/z (M+H)⁺.

### 3-(3-(1-(5-amino-1-(5-((4,6-difluoro-1H-indol-5-yl)oxy)-2-fluorophenyl)-1H-pyrazol-3-yl)ethyl)-2-fluorophenyl)propanoic acid

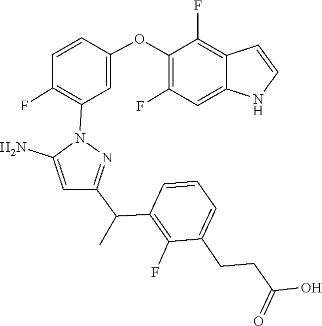 [SMILES: CC(c1cc(N)n(-c2cc(Oc3c(F)cc4[nH]ccc4c3F)ccc2F)n1)c1cccc(CCC(=O)O)c1F]

Step F: To a solution of ethyl 3-[3-[1-[5-amino-1-[5-[4,6-difluoro-1-(p-tolylsulfonyl)indol-5-yl]oxy-2-fluoro-phenyl]pyrazol-3-yl]ethyl]-2-fluoro-phenyl]propanoate (10 mg, 0.014 mmol) in THE (3 mL) and MeOH (1 mL) was added dropwise at 0° C. aqueous LiOH (0.5M, 0.14 mL, 0.07 mmol). The solution was warmed to room temperature and stirred for 3 hours. After removing most of the organics under reduced pressure, the pH of the mixture was adjusted to ~6 with 1M hydrochloric acid. The precipitate was isolated by suction filtration and air dried to give the title compound as a white solid (1.7 mg, 17%). MS (ESI): 539 m/z (M+H)⁺.

### Example 49. Synthesis of 3-(3-(1-(1-(5-((4,6-difluoro-1H-indol-5-yl)oxy)-2-fluorophenyl)-5-ethoxy-1H-pyrazol-3-yl)ethyl)-2-fluorophenyl)propanoic acid

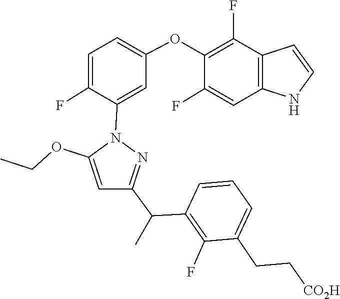 [SMILES: CCOc1cc(C(C)c2cccc(CCC(=O)O)c2F)nn1-c1cc(Oc2c(F)cc3[nH]ccc3c2F)ccc1F]

### Ethyl 4-(3-bromo-2-fluorophenyl)-3-oxopentanoate

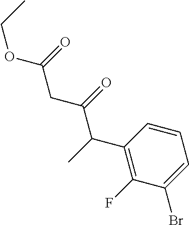 [SMILES: CCOC(=O)CC(=O)C(C)c1cccc(Br)c1F]

Step A: Triethylamine (3.10 eq, 9.6 mL, 69.0 mmol) and magnesium dichloride (2.50 eq, 5.30 g, 55.7 mmol) were added to a stirred suspension of (3-ethoxy-3-oxo-propanoyl)oxypotassium (2.00 eq, 7.58 g, 44.5 mmol) in dry acetonitrile (100 mL). Stirring at room temperature was continued for 2 hours. To the reaction mixture was then added a solution of 2-(3-bromo-2-fluoro-phenyl)propanoic acid (1.00 eq, 5.50 g, 22.3 mmol) and N,N′-carbonyldiimidazole (1.20 eq, 4.33 g, 26.7 mmol) in acetonitrile (20 mL) prepared 15 minutes prior to the start of the addition. The reaction mixture was stirred at room temperature for 16 hours, then heated at reflux for 2 hours, followed by cooling to room temperature. 13% hydrochloric acid (100 mL) were added to the reaction, and the resulting clear mixture was stirred for a further 15 minutes. The mixture was diluted with water (100 mL) and extracted with ethyl acetate (150 mL×2). The combined organic extracts were washed with brine (150 mL), dried over anhydrous sodium sulfate, filtered, and concentrated. The residue was purified by flash column chromatography on silica gel, eluting with 0-60% ethyl acetate in petroleum ether, to give the product, ethyl 4-(3-bromo-2-fluoro-phenyl)-3-oxo-pentanoate (6.30 g, 19.7 mmol, 88%) as an oil. MS (ESI): 317.0, 319.0 m/z (M+H)⁺.

### 5-(3-(3-(1-(3-Bromo-2-fluorophenyl)ethyl)-5-ethoxy-1H-pyrazol-1-yl)-4-fluorophenoxy)-4,6-difluoro-1-tosyl-1H-indole and 5-(1-(3-bromo-2-fluorophenyl)ethyl)-2-(5-((4,6-difluoro-1-tosyl-1H-indol-5-yl)oxy)-2-fluorophenyl)-2,4-dihydro-3H-pyrazol-3-one

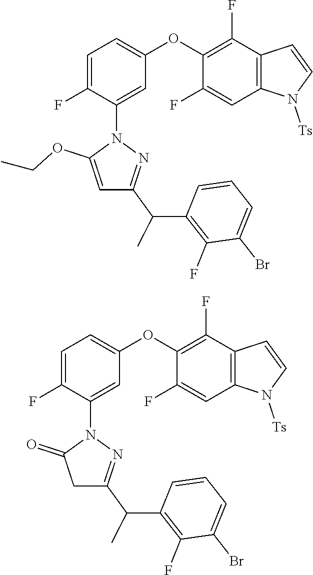

Step B: To a stirred solution of [5-[4,6-difluoro-1-(p-tolylsulfonyl)indol-5-yl]oxy-2-fluoro-phenyl]hydrazine hydrochloride (1.00 eq, 763 mg, 1.58 mmol) and ethyl 4-(3-bromo-2-fluoro-phenyl)-3-oxo-pentanoate (1.00 eq, 500 mg, 1.58 mmol) in ethanol (12 mL) was added acetic acid (0.08 mL). The mixture was stirred at 80° C. for 2 hours. The reaction mixture was quenched with water (30 mL) and extracted with ethyl acetate (20 mL×3). The combined organic extracts were washed with water (20 mL) and brine (20 mL), dried over anhydrate sodium sulfate, filtered, and concentrated. The residue was purified by flash chromatography on silica gel, eluting with 0-50% ethyl acetate in petroleum ether, to give the product, 5-[1-(3-bromo-2-fluoro-phenyl)ethyl]-2-[5-[4,6-difluoro-1-(p-tolylsulfonyl)indol-5-yl]oxy-2-fluoro-phenyl]-4H-pyrazol-3-one (400 mg, 0.568 mmol, 36%) as solid and 5-[3-[3-[l-(3-bromo-2-fluoro-phenyl)ethyl]-5-ethoxy-pyrazol-1-yl]-4-fluoro-phenoxy]-4,6-difluoro-1-(p-tolylsulfonyl)indole (205 mg, 0.263 mmol, 17%) as a solid.

### LC-MS Method:

Column: SunFire C18 (4.6×50 mm, 3.5 um);
Mobile phase: H₂O (0.01% TFA) (A)/acetonitrile (0.01% TFA) (B);
Elution program: Gradient from 5 to 95% of B in 1.4 minutes at 2.0 mL/min.
5-[3-[3-[1-(3-bromo-2-fluoro-phenyl)ethyl]-5-ethoxy-pyrazol-1-yl]-4-fluoro-phenoxy]-4,6-difluoro-1-(p-tolylsulfonyl)indole, MS (ESI): 728.1, 730.1 m/z (M+H)⁺ at 2.48 minutes.
5-[1-(3-bromo-2-fluoro-phenyl)ethyl]-2-[5-[4,6-difluoro-1-(p-tolylsulfonyl)indol-5-yl]oxy-2-fluoro-phenyl]-4H-pyrazol-3-one, MS (ESI): 700.0, 702.0 m/z (M+H)⁺ at 2.26 minutes.

### Ethyl (E)-3-(3-(1-(1-(5-((4,6-difluoro-1-tosyl-1H-indol-5-yl)oxy)-2-fluorophenyl)-5-ethoxy-1H-pyrazol-3-yl)ethyl)-2-fluorophenyl)acrylate

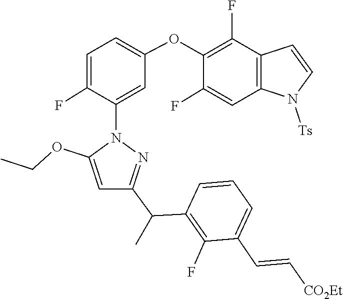 [SMILES: CCOC(=O)/C=C/c1cccc(C(C)c2cc(OCC)n(-c3cc(Oc4c(F)cc5c(ccn5S(=O)(=O)c5ccc(C)cc5)c4F)ccc3F)n2)c1F]

Step C: To a solution of 5-[3-[3-[1-(3-bromo-2-fluoro-phenyl)ethyl]-5-ethoxy-pyrazol-1-yl]-4-fluoro-phenoxy]-4,6-difluoro-1-(p-tolylsulfonyl)indole (1.00 eq, 160 mg, 0.220 mmol) in 1,4-dioxane (8 mL) and water (2 mL) was added ethyl (E)-3-(4,4,5,5-tetramethyl-1,3,2-dioxaborolan-2-yl)prop-2-enoate (2.00 eq, 99 mg, 0.439 mmol), Pd(dppf)Cl₂ dichloromethane complex (0.10 eq, 16 mg, 0.022 mmol) and potassium carbonate (3.00 eq, 91 mg, 0.66 mmol). The mixture was stirred at 100° C. for 16 hours, cooled to room temperature and concentrated. The residue was diluted with ethyl acetate (40 mL), washed with water (10 mL) and brine (10 mL), dried over sodium sulphate, filtered, and concentrated. The residue was purified by flash chromatography on silica gel, eluting with 0-30% ethyl acetate in petroleum ether, to give the product, ethyl (E)-3-[3-[1-[1-[5-[4,6-difluoro-1-(p-tolylsulfonyl)indol-5-yl]oxy-2-fluoro-phenyl]-5-ethoxy-pyrazol-3-yl]ethyl]-2-fluoro-phenyl]prop-2-enoate (125 mg, 0.17 mmol, 75.1%) as a solid. MS (ESI): 748.2 m/z (M+H)⁺.

### Ethyl 3-(3-(1-(1-(5-((4,6-difluoro-1-tosyl-JH-indol-5-yl)oxy)-2-fluorophenyl)-5-ethoxy-1H-pyrazol-3-yl)ethyl)-2-fluorophenyl)propanoate

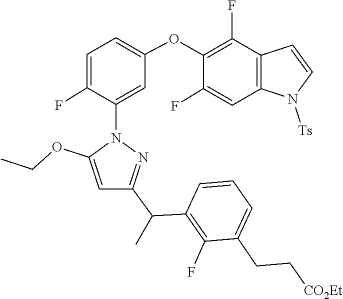 [SMILES: CCOC(=O)CCc1cccc(C(C)c2cc(OCC)n(-c3cc(Oc4c(F)cc5c(ccn5S(=O)(=O)c5ccc(C)cc5)c4F)ccc3F)n2)c1F]

Step D: To a solution of ethyl 3-[3-[1-[1-[5-[4,6-difluoro-1-(p-tolylsulfonyl)indol-5-yl]oxy-2-fluoro-phenyl]-5-ethoxy-pyrazol-3-yl]ethyl]-2-fluoro-phenyl]prop-2-enoate (1.00 eq, 125 mg, 0.167 mmol) in toluene (5 mL) was added p-toluenesulfonylhydrazide (10.0 eq, 311 mg, 1.67 mmol). The reaction was stirred at 110° C. for 2 hours. The reaction was cooled room temperature, filtered, and the solids washed with toluene (3 mL×2). The combined filtrates were concentrated. The residue was diluted with ethyl acetate (20 mL), washed with 1.0 M hydrochloric acid (10 mL×2) and brine (10 mL), dried over anhydrous sodium sulfate, filtered, and concentrated. The residue was purified by flash column chromatography on silica gel, eluting with 0-30% ethyl acetate in petroleum ether, to give the product, ethyl 3-[3-[1-[1-[5-[4,6-difluoro-1-(p-tolylsulfonyl)indol-5-yl]oxy-2-fluoro-phenyl]-5-ethoxy-pyrazol-3-yl]ethyl]-2-fluoro-phenyl]propanoate (75 mg, 0.098 mmol, 59%) as a solid. MS (ESI): 750.2 m/z (M+H)⁺.

### 3-(3-(1-(1-(5-((4,6-Difluoro-1H-indol-5-yl)oxy)-2-fluorophenyl)-5-ethoxy-1H-pyrazol-3-yl)ethyl)-2-fluorophenyl)propanoic acid

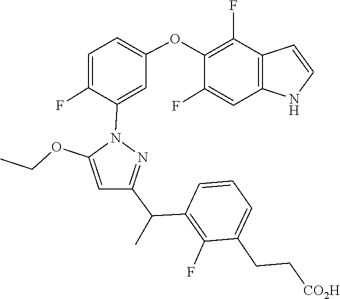 [SMILES: CCOc1cc(C(C)c2cccc(CCC(=O)O)c2F)nn1-c1cc(Oc2c(F)cc3[nH]ccc3c2F)ccc1F]

Step E: To a solution of ethyl 3-[3-[1-[1-[[5-[4,6-difluoro-1-(p-tolylsulfonyl)indol-5-yl]oxy-2-fluoro-phenyl]-5-ethoxy-pyrazol-3-yl]ethyl]-2-fluoro-phenyl]propanoate (1.00 eq, 72 mg, 0.096 mmol) in THE (3.9 mL) was added 1N aqueous lithium hydroxide (10.0 eq, 0.96 mL, 0.960 mmol). The reaction was stirred at room temperature for 72 hours. The mixture was acidified with 1N hydrochloric acid to pH=4, diluted with water (10 mL) and extracted with ethyl acetate (15 mL×2). The combined organic extracts were washed with water (5 mL) and brine (5 mL), dried over sodium sulphate, filtered, and concentrated. The residue was purified by prep-HPLC to give the product, 3-[3-[1-[1-[5-[(4,6-difluoro-1H-indol-5-yl)oxy]-2-fluoro-phenyl]-5-ethoxy-pyrazol-3-yl]ethyl]-2-fluoro-phenyl]propanoic acid (29 mg, 0.05 mmol, 52%) as a white solid. MS (ESI): 568.2 m/z (M+H)⁺. ¹H NMR (400 MHz, CD₃OD) δ 7.29 (d, J=3.2 Hz, 1H), 7.20 (t, J=9.2 Hz, 1H), 7.14-7.10 (m, 3H), 7.01-6.97 (m, 2H), 6.93-6.91 (m, 1H), 6.54 (d, J=3.2 Hz, 1H), 5.59 (s, 1H), 4.41-4.36 (m, 1H), 4.12-4.07 (m, 2H), 2.93 (t, J=7.6 Hz, 2H), 2.52 (t, J=7.6 Hz, 2H), 1.56 (d, J=7.2 Hz, 3H), 1.28 (t, J=7.2 Hz, 3H) ppm.

### Example 50. Synthesis of 3-(3-(1-(1-(5-((4,6-difluoro-1H-indol-5-yl)oxy)-2-fluorophenyl)-5-methoxy-1H-pyrazol-3-yl)ethyl)-2-fluorophenyl)propanoic acid

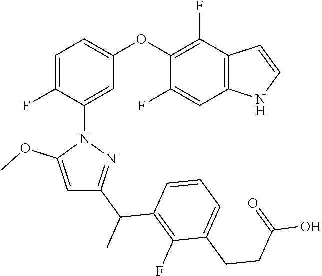 [SMILES: COc1cc(C(C)c2cccc(CCC(=O)O)c2F)nn1-c1cc(Oc2c(F)cc3[nH]ccc3c2F)ccc1F]

### (E)-2-(3-(3-ethoxy-3-oxoprop-1-en-1-yl)-2-fluorophenyl)propanoic acid

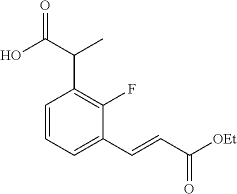 [SMILES: CCOC(=O)/C=C/c1cccc(C(C)C(=O)O)c1F]

Step A: To a solution of 2-(3-bromo-2-fluoro-phenyl)propanoic acid (1.00 eq, 1.96 g, 7.93 mmol) in 1,4-dioxane (24 mL) and water (6 mL) was added ethyl (E)-3-(4,4,5,5-tetramethyl-1,3,2-dioxaborolan-2-yl)prop-2-enoate (2.00 eq, 3.59 g, 15.9 mmol), Pd(dppf)Cl₂ dichloromethane complex (0.100 eq, 580 mg, 0.793 mmol) and cesium carbonate (3.00 eq, 7.75 g, 23.8 mmol). The mixture was stirred at 90° C. for 16 hours. The mixture was concentrated. The residue was dissolved in ethyl acetate (120 mL), washed with water (40 mL) and brine (30 mL), dried over sodium sulphate, filtered, and concentrated. The residue was purified by flash column chromatography on silica gel, eluting with 0-50% ethyl acetate in petroleum ether, to give the product, 2-[3-(3-ethoxy-3-oxo-prop-1-enyl)-2-fluoro-phenyl]propanoic acid (1.60 g, 5.91 mmol, 74.5%) as a solid. MS (ESI): 267.1 n/z (M+H)⁺.

### 2-(3-(3-ethoxy-3-oxopropyl)-2-fluorophenyl)propanoic acid

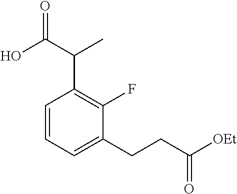 [SMILES: CCOC(=O)CCc1cccc(C(C)C(=O)O)c1F]

Step B: To a solution of 2-[3-(3-ethoxy-3-oxo-prop-1-enyl)-2-fluoro-phenyl]propanoic acid (1.00 eq, 1.60 g, 6.01 mmol) in methanol (15 mL) was added Pd/C (10 wt. % c, 0.089 eq, 569 mg, 0.535 mmol). The reaction was stirred at room temperature under a H₂ atmosphere for 2 hours. The catalyst was filtered off and rinsed with methanol (15 mL×2). The combined filtrates were concentrated to afford the desired product, 2-[3-(3-ethoxy-3-oxo-propyl)-2-fluoro-phenyl]propanoic acid (1.36 g, 4.95 mmol, 82.4%) as a solid. MS (ESI): 269.1 n/z (M+H)⁺.

### Methyl 4-(3-(3-ethoxy-3-oxopropyl)-2-fluorophenyl)-3-oxopentanoate

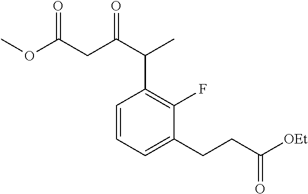 [SMILES: CCOC(=O)CCc1cccc(C(C)C(=O)CC(=O)OC)c1F]

Step C: Triethylamine (3.10 eq, 0.86 mL, 6.19 mmol) and magnesium dichloride (2.50 eq, 476 mg, 4.99 mmol) were added to a stirred suspension of (3-methoxy-3-oxo-propanoyl)oxypotassium (2.00 eq, 624 mg, 4.00 mmol) in dry acetonitrile (10 mL). Stirring continued at room temperature for 2 hours. To the reaction mixture was added a solution of 2-[3-(3-ethoxy-3-oxo-propyl)-2-fluoro-phenyl]propanoic acid (1.00 eq, 0.54 g, 2.00 mmol) and N,N′-carbonyldiimidazole (1.20 eq, 389 mg, 2.40 mmol) in acetonitrile (10 mL) prepared 15 minutes prior to the start of the addition. The reaction mixture was stirred at 80° C. for 16 hours, cooled to room temperature, and 13% hydrochloric acid (20 mL) added. The resulting clear mixture was stirred for a further 15 minutes, diluted with water (50 mL), and extracted with ethyl acetate (70 mL×2). The combined organic extracts were washed with brine (50 mL), dried over anhydrous sodium sulfate, filtered and concentrated. The residue was purified by flash column chromatography on silica gel, eluting with 0-40% ethyl acetate in petroleum ether, to give the product, methyl 4-[3-(3-ethoxy-3-oxo-propyl)-2-fluoro-phenyl]-3-oxo-pentanoate (0.40 g, 1.10 mmol, 54.9%) as an oil. MS (ESI): 325.1 m/z (M+H)⁺.

### Ethyl 3-(3-(1-(1-(5-((4,6-difluoro-1-tosyl-JH-indol-5-yl)oxy)-2-fluorophenyl)-5-methoxy-1H-pyrazol-3-yl)ethyl)-2-fluorophenyl)propanoate and ethyl 3-(3-(1-(1-(5-((4,6-difluoro-1-tosyl-1H-indol-5-yl)oxy)-2-fluorophenyl)-5-oxo-4,5-dihydro-JH-pyrazol-3-yl)ethyl)-2-fluorophenyl)propanoate

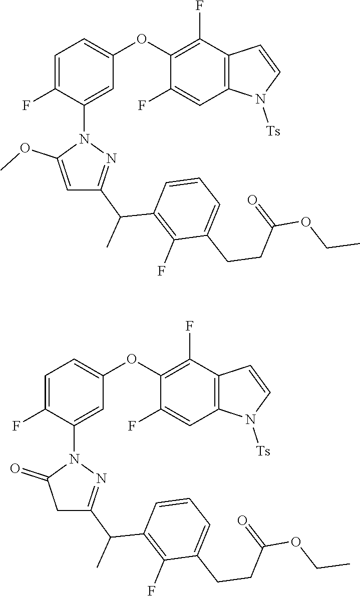 [SMILES: CCOC(=O)CCc1cccc(C(C)c2cc(OC)n(-c3cc(Oc4c(F)cc5c(ccn5S(=O)(=O)c5ccc(C)cc5)c4F)ccc3F)n2)c1F ; CCOC(=O)CCc1cccc(C(C)C2=NN(c3cc(Oc4c(F)cc5c(ccn5S(=O)(=O)c5ccc(C)cc5)c4F)ccc3F)C(=O)C2)c1F]

Step D: To a stirred solution of [5-[4,6-difluoro-1-(p-tolylsulfonyl)indol-5-yl]oxy-2-fluoro-phenyl]hydrazine hydrochloride (1.00 eq, 143 mg, 0.296 mmol) and methyl 4-[3-(3-ethoxy-3-oxo-propyl)-2-fluoro-phenyl]-3-oxo-pentanoate (1.00 eq, 96 mg, 0.296 mmol) in ethanol (8 mL) was added acetic acid (0.08 mL). The mixture was stirred at 80° C. for 2 hours. The reaction mixture was quenched with water (20 mL) and extracted with ethyl acetate (10 mL×3). The combined organic extracts were washed with water (10 mL×3) and brine (20 mL), dried over anhydrate sodium sulfate, filtered, and concentrated. The residue was purified by flash column chromatography on silica gel, eluting with 0-50% ethyl acetate in petroleum ether, to give the product, ethyl 3-[3-[1-[1-[5-[4,6-difluoro-1-(p-tolylsulfonyl)indol-5-yl]oxy-2-fluoro-phenyl]-5-oxo-4H-pyrazol-3-yl]ethyl]-2-fluoro-phenyl]propanoate (90 mg, 0.12 mmol, 42%) as a solid and ethyl 3-[3-[1-[1-[5-[4,6-difluoro-1-(p-tolylsulfonyl)indol-5-yl]oxy-2-fluoro-phenyl]-5-methoxy-pyrazol-3-yl]ethyl]-2-fluoro-phenyl]propanoate (16 mg, 0.019 mmol, 6.6%) as a solid.

An LC-MS method identical to the one used in Step B, Example 49 was used to characterize the products of this step.

3-[3-[1-[1-[5-[4,6-difluoro-1-(p-tolylsulfonyl)indol-5-yl]oxy-2-fluoro-phenyl]-5-oxo-4H-pyrazol-3-yl]ethyl]-2-fluoro-phenyl]propanoate, MS (ESI): 722.2 m/z (M+H)⁺ at 2.23 minutes.

3-(3-[1-[1[-[5-[4,6-difluoro-1-(p-tolylsulfonyl)indol-5-yl]oxy-2-fluoro-phenyl]-5-methoxy-pyrazol-3-yl]ethyl]-2-fluoro-phenyl]propanoate, MS (ESI): 722.2 m/z (M+H)⁺ at 2.35 minutes.

### 3-(3-(1-(1-(5-((4,6-Difluoro-H-indol-5-yl)oxy)-2-fluorophenyl)-5-methoxy-1H-pyrazol-3-yl)ethyl)-2-fluorophenyl)propanoic acid

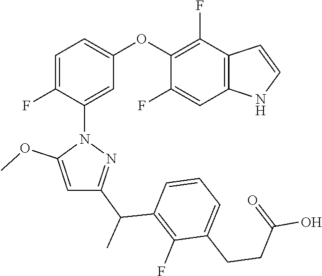 [SMILES: COc1cc(C(C)c2cccc(CCC(=O)O)c2F)nn1-c1cc(Oc2c(F)cc3[nH]ccc3c2F)ccc1F]

Step E: To a solution of ethyl 3-[3-[1-[1-[5-[4,6-difluoro-1-(p-tolylsulfonyl)indol-5-yl]oxy-2-fluoro-phenyl]-5-methoxy-pyrazol-3-yl]ethyl]-2-fluoro-phenyl]propanoate (1.00 eq, 15 mg, 0.02 mmol) in THE (1 mL) was added 1N aqueous lithium hydroxide (10.0 eq, 0.20 mL, 0.20 mmol). The reaction was stirred at room temperature for 72 hours. The mixture was acidified with 1N hydrochloric acid to pH=4, diluted with water (10 mL) and extracted with ethyl acetate (5 mL×2). The combined organic extracts were washed with water (3 mL) and brine (3 mL), dried over sodium sulphate, filtered, and concentrated. The residue was purified by prep-HPLC to give the product, 3-[3-[1-[1-[5-[(4,6-difluoro-1H-indol-5-yl)oxy]-2-fluoro-phenyl]-5-methoxy-pyrazol-3-yl]ethyl]-2-fluoro-phenyl]propanoic acid (6.7 mg, 0.012 mmol, 59%) as a white solid. MS (ESI): 722.2 m/z (M+H)⁺. ¹H NMR (400 MHz, CD₃OD) δ 7.29 (d, J=3.2 Hz, 1H), 7.20 (t, J=9.6 Hz, 1H), 7.14-7.10 (m, 3H), 6.99-6.91 (m, 3H), 6.54 (d, J=3.6 Hz, 1H), 5.61 (s, 1H), 4.41-4.36 (m, 1H), 3.85 (s, 3H), 2.92 (t, J=8.0 Hz, 2H), 2.52 (t, J=8.0 Hz, 2H), 1.56 (d, J=7.2 Hz, 3H) ppm.

### Example 51. Synthesis of 3-(3-(1-(1-(5-((4,6-difluoro-1H-indol-5-yl)oxy)-2-fluorophenyl)-5-oxo-4,5-dihydro-1H-pyrazol-3-yl)ethyl)-2-fluorophenyl)propanoic acid

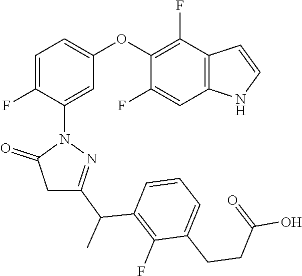 [SMILES: CC(C1=NN(c2cc(Oc3c(F)cc4[nH]ccc4c3F)ccc2F)C(=O)C1)c1cccc(CCC(=O)O)c1F]

Prepared from ethyl 3-[3-[1-[1-[5-[4,6-difluoro-1-(p-tolylsulfonyl)indol-5-yl]oxy-2-fluoro-phenyl]-5-oxo-4H-pyrazol-3-yl]ethyl]-2-fluoro-phenyl]propanoate following the method of Step F, Example 48. MS (ESI): 540.2 m/z (M+H)⁺.

### Example 52. Synthesis of 3-(3-(1-(1-(5-((4,6-difluoro-1H-indol-5-yl)oxy)-2-fluorophenyl)-5-(methylthio)-1H-pyrazol-3-yl)ethyl)-2-fluorophenyl)propanoic acid

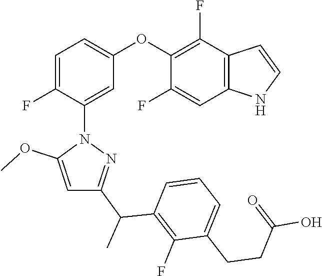 [SMILES: COc1cc(C(C)c2cccc(CCC(=O)O)c2F)nn1-c1cc(Oc2c(F)cc3[nH]ccc3c2F)ccc1F]

### Ethyl 3-(3-(1-(1-(5-((4,6-difluoro-1-tosyl-1H-indol-5-yl)oxy)-2-fluorophenyl)-5-thioxo-4,5-dihydro-1H-pyrazol-3-yl)ethyl)-2-fluorophenyl)propanoate

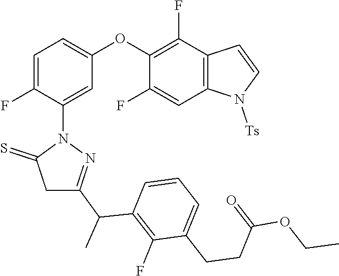 [SMILES: CCOC(=O)CCc1cccc(C(C)C2=NN(c3cc(Oc4c(F)cc5c(ccn5S(=O)(=O)c5ccc(C)cc5)c4F)ccc3F)C(=S)C2)c1F]

Step A: To a solution of ethyl 3-[3-[1-[1-[5-[4,6-difluoro-1-(p-tolylsulfonyl)indol-5-yl]oxy-2-fluoro-phenyl]-5-oxo-4H-pyrazol-3-yl]ethyl]-2-fluoro-phenyl]propanoate (1.00 eq, 80 mg, 0.11 mmol) in toluene (3 mL) was added 2,4-bis(4-methoxyphenyl)-1,3,2,4-dithiadiphosphetane 2,4-disulfide (0.50 eq, 22 mg, 0.055 mmol). The reaction mixture was stirred at 110° C. for 2 hours. The mixture was quenched with water (15 mL), extracted with ethyl acetate (15 mL×2), washed with brine (5 mL), dried over anhydrous sodium sulfate, filtered, and concentrated. The residue was purified by flash column chromatography on silica gel, eluting with 0-40% ethyl acetate in petroleum ether, to give the desired compound, ethyl 3-[3-[1-[1-[5-[4,6-difluoro-1-(p-tolylsulfonyl)indol-5-yl]oxy-2-fluoro-phenyl]-5-thioxo-4H-pyrazol-3-yl]ethyl]-2-fluoro-phenyl]propanoate (40 mg, 0.032 mmol, 29%) as a solid. MS (ESI): 738.1 m/z (M+H)⁺.

### Ethyl 3-(3-(1-(1-(5-((4,6-difluoro-1-tosyl-H-indol-5-yl)oxy)-2-fluorophenyl)-5-(methylthio)-1H-pyrazol-3-yl)ethyl)-2-fluorophenyl)propanoate

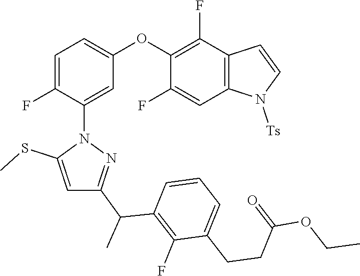 [SMILES: CCOC(=O)CCc1cccc(C(C)c2cc(SC)n(-c3cc(Oc4c(F)cc5c(ccn5S(=O)(=O)c5ccc(C)cc5)c4F)ccc3F)n2)c1F]

Step B: To a solution of ethyl 3-[3-[1-[1-[5-[4,6-difluoro-1-(p-tolylsulfonyl)indol-5-yl]oxy-2-fluoro-phenyl]-5-thioxo-4H-pyrazol-3-yl]ethyl]-2-fluoro-phenyl]propanoate (1.00 eq, 65 mg, 0.088 mmol) in acetonitrile (2 mL) was added potassium carbonate (2.00 eq, 24 mg, 0.18 mmol) and methyl iodide (2.00 eq, 0.011 mL, 0.18 mmol). The mixture was stirred at room temperature for 16 hours. The mixture was quenched with water (10 mL), extracted with ethyl acetate (10 mL×2), washed with brine (5 mL), dried over anhydrous sodium sulfate, filtered and concentrated to give the desired product, ethyl 3-[3-[1-[1-[5-[4,6-difluoro-1-(p-tolylsulfonyl)indol-5-yl]oxy-2-fluoro-phenyl]-5-methylsulfanyl-pyrazol-3-yl]ethyl]-2-fluoro-phenyl]propanoate (50 mg, 0.038 mmol, 43%) as a solid. MS (ESI): 752.2 m/z (M+H)⁺.

### 3-(3-(1-(1-(5-((4,6-Difluoro-1H-indol-5-yl)oxv)-2-fluorophenyl)-5-(methylthio)-1H-pyrazol-3-yl)ethyl)-2-fluorophenyl)propanoic acid

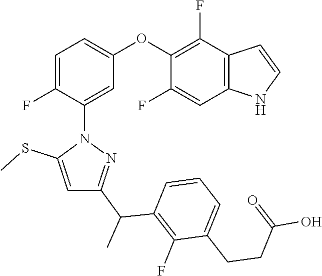 [SMILES: CSc1cc(C(C)c2cccc(CCC(=O)O)c2F)nn1-c1cc(Oc2c(F)cc3[nH]ccc3c2F)ccc1F]

Step C: To a solution of ethyl 3-[3-[1-[1-[5-[4,6-difluoro-1-(p-tolylsulfonyl)indol-5-yl]oxy-2-fluoro-phenyl]-5-methylsulfanyl-pyrazol-3-yl]ethyl]-2-fluoro-phenyl]propanoate (1.00 eq, 50 mg, 0.067 mmol) in THE (2 mL) was added 1N aqueous lithium hydroxide (10.0 eq, 0.67 mL, 0.67 mmol). The reaction was stirred at room temperature for 72 hours. The mixture was acidified with 1N hydrochloric acid to pH=4, diluted with water (10 mL) and extracted with ethyl acetate (5 mL×2). The combined organic extracts were washed with water (3 mL) and brine (3 mL), dried over sodium sulphate, filtered and concentrated. The residue was purified by prep-HPLC to give the product, 3-[3-[1-[1-[5-[(4,6-difluoro-1H-indol-5-yl)oxy]-2-fluoro-phenyl]-5-methylsulfanyl-pyrazol-3-yl]ethyl]-2-fluoro-phenyl]propanoic acid (19 mg, 0.033 mmol, 49%) as a white solid. MS (ESI): 570.1 m/z (M+H)⁺. ¹H NMR (400 MHz, CD₃OD) δ 7.30-7.24 (m, 2H), 7.15-7.06 (m, 4H), 6.99 (t, J=7.6 Hz, 1H), 6.95-6.93 (m, 1H), 6.54 (d, J=2.8 Hz, 1H), 6.27 (s, 1H), 4.50-4.45 (m, 1H), 2.93 (t, J=7.6 Hz, 2H), 2.55 (t, J=7.6 Hz, 2H), 2.33 (s, 3H), 1.59 (d, J=7.2 Hz, 3H) ppm.

### Example 53. Synthesis of 3-(3-(1-(5-(5-((4,6-difluoro-1H-indol-5-yl)oxy)-2-fluorophenyl)-1,2,4-oxadiazol-3-yl)ethyl)-2-fluorophenyl)propanoic acid

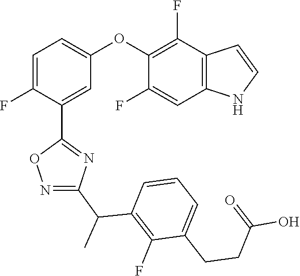 [SMILES: CC(c1noc(-c2cc(Oc3c(F)cc4[nH]ccc4c3F)ccc2F)n1)c1cccc(CCC(=O)O)c1F]

### 2-(3-Bromo-2-fluorophenyl)-N-hydroxypropanimidamide

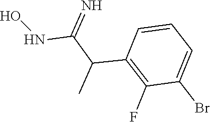

Step A: To a solution of 2-(3-bromo-2-fluoro-phenyl)propanenitrile (1.00 eq, 1.10 g, 4.82 mmol) and hydroxylamine hydrochloride (3.00 eq, 1.01 g, 14.5 mmol) in methanol (20 mL) was added potassium carbonate (2.00 eq, 1.33 g, 9.65 mmol). The mixture was stirred overnight at 60° C. Water (80 mL) was added, and the resulting mixture was extracted with ethyl acetate (40 mL×2). The combined organic extracts were washed with brine (80 mL), dried over anhydrous sodium sulfate, filtered, and concentrated to give 2-(3-bromo-2-fluoro-phenyl)-N-hydroxy-propenamidine (1.20 g, 4.60 mmol, 95.3%) as a solid. MS (ESI): 261, 263 m/z (M+H)⁺.

### 3-(1-(3-Bromo-2-fluorophenyl)ethyl)-5-(5-((4,6-difluoro-1H-indol-5-yl)oxy)-2-fluorophenyl)-1,2,4-oxadiazole

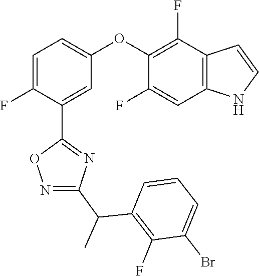 [SMILES: CC(c1noc(-c2cc(Oc3c(F)cc4[nH]ccc4c3F)ccc2F)n1)c1cccc(Br)c1F]

Step B: To a solution of 2-(3-bromo-2-fluoro-phenyl)-N-hydroxy-propenamidine (1.00 eq, 1.20 g, 4.60 mmol), 5-[(4,6-difluoro-1H-indol-5-yl)oxy]-2-fluoro-benzoic acid (Intermediate 12, 1.00 eq, 1.41 g, 4.60 mmol) and triethylamine (2.00 eq, 1.3 mL, 9.19 mmol) in DMF (20 mL) was added HATU (1.20 eq, 2.10 g, 5.52 mmol). The mixture was stirred at room temperature for 1 hour, then heated overnight at 80° C., and cooled to room temperature. Water (80 mL) was added, and the resulting mixture was extracted with ethyl acetate (40 mL×2). The combined organic extracts were washed with brine (80 mL), dried over anhydrous sodium sulfate, filtered and concentrated. The residue was purified by flash column chromatography on silica gel, eluting with 0-20% ethyl acetate in petroleum ether, to give 3-[1-(3-bromo-2-fluoro-phenyl)ethyl]-5-[5-[(4,6-difluoro-1H-indol-5-yl)oxy]-2-fluoro-phenyl]-1,2,4-oxadiazole (0.80 g, 1.50 mmol, 32.7%) as a solid. MS (ESI): 532, 534 m/z (M+H)⁺.

### Ethyl (E)-3-(3-(1-(5-(5-((4,6-difluoro-1H-indol-5-yl)oxy)-2-fluorophenyl)-1,2,4-oxadiazol-3-yl)ethyl)-2-fluorophenyl)acrylate

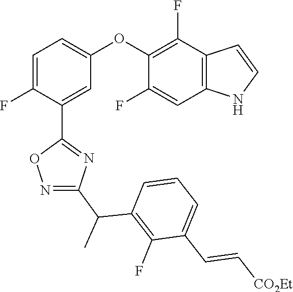 [SMILES: CCOC(=O)/C=C/c1cccc(C(C)c2noc(-c3cc(Oc4c(F)cc5[nH]ccc5c4F)ccc3F)n2)c1F]

Step C: To a solution of 3-[1-(3-bromo-2-fluoro-phenyl)ethyl]-5-[5-[(4,6-difluoro-1H-indol-5-yl)oxy]-2-fluoro-phenyl]-1,2,4-oxadiazole (1.00 eq, 100 mg, 0.19 mmol), ethyl (E)-3-(4,4,5,5-tetramethyl-1,3,2-dioxaborolan-2-yl)prop-2-enoate (1.30 eq, 55 mg, 0.24 mmol) and potassium carbonate (2.00 eq, 52 mg, 0.376 mmol) in 1,4-dioxane (5 mL) and water (1 mL) was added Pd(dppf)Cl₂ dichloromethane complex (0.10 eq, 15 mg, 0.019 mmol). The mixture was purged with argon and stirred at 100° C. for 5 hours. Water (20 mL) was added, and the resulting mixture was extracted with ethyl acetate (20 mL×2). The combined organic extracts were washed with brine (30 mL), dried over anhydrous sodium sulfate, filtered and concentrated. The residue was purified by flash column chromatography on silica gel, eluting with 0-40% ethyl acetate in petroleum ether, to give ethyl (E)-3-[3-[1-[5-[5-[(4,6-difluoro-1H-indol-5-yl)oxy]-2-fluoro-phenyl]-1,2,4-oxadiazol-3-yl]ethyl]-2-fluoro-phenyl]prop-2-enoate (42 mg, 0.076 mmol, 40.5%) as a solid. MS (ESI): 552 m/z (M+H)⁺.

### Ethyl 3-(3-(1-(5-(5-((4,6-difluoro-1H-indol-5-yl)oxy)-2-fluorophenyl)-1,2,4-oxadiazol-3-yl)ethyl)-2-fluorophenyl)propanoate

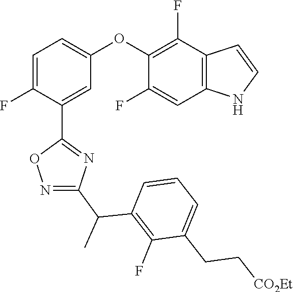 [SMILES: CCOC(=O)CCc1cccc(C(C)c2noc(-c3cc(Oc4c(F)cc5[nH]ccc5c4F)ccc3F)n2)c1F]

Step D: To a solution of ethyl (Z)-3-[3-[1-[5-[5-[(4,6-difluoro-1H-indol-5-yl)oxy]-2-fluoro-phenyl]-1,2,4-oxadiazol-3-yl]ethyl]-2-fluoro-phenyl]prop-2-enoate (1.00 eq, 42 mg, 0.076 mmol) in toluene (5 ml) was added p-toluenesulfonylhydrazide (10.0 eq, 142 mg, 0.76 mmol). The reaction mixture was stirred at 110° C. for 6 hours, then cooled to room temperature, filtered, and the solids washed with toluene (3 mL×2). The combined filtrates were concentrated. The residue was diluted with ethyl acetate (20 mL), washed with 1.0 M hydrochloric acid (20 mL×2) and brine (20 mL×2), dried over anhydrous sodium sulfate, filtered, and concentrated. The residue was purified by flash column chromatography on silica gel, eluting with 0-50% ethyl acetate in petroleum ether, to give ethyl 3-[3-[1-[5-[5-[(4,6-difluoro-1H-indol-5-yl)oxy]-2-fluoro-phenyl]-1,2,4-oxadiazol-3-yl]ethyl]-2-fluoro-phenyl]propanoate (28 mg, 0.051 mmol, 66.4%) as a solid. MS (ESI): 554 m/z (M+H)⁺.

### 3-(3-(1-(5-(5-((4,6-Difluoro-1H-indol-5-yl)oxy)-2-fluorophenyl)-1,2,4-oxadiazol-3-yl)ethyl)-2-fluorophenyl)propanoic acid

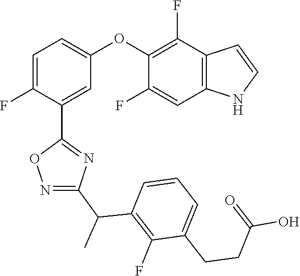 [SMILES: CC(c1noc(-c2cc(Oc3c(F)cc4[nH]ccc4c3F)ccc2F)n1)c1cccc(CCC(=O)O)c1F]

Step E: To a solution of ethyl 3-[3-[1-[5-[5-[(4,6-difluoro-1H-indol-5-yl)oxy]-2-fluoro-phenyl]-1,2,4-oxadiazol-3-yl]ethyl]-2-fluoro-phenyl]propanoate (1.00 eq, 28 mg, 0.051 mmol) in THE (1 mL) was added 1N aqueous lithium hydroxide monohydrate (9.90 eq, 0.50 mL, 0.50 mmol). The mixture was stirred overnight at room temperature. The reaction mixture was acidified with 1.0 M hydrochloric acid to pH=6 and extracted with ethyl acetate (3×10 mL). The combined organic extracts were washed with brine (20 mL×2), dried over anhydrous sodium sulfate, filtered and concentrated. The residue was purified by prep-HPLC to give 3-[3-[1-[5-[5-[(4,6-difluoro-1H-indol-5-yl)oxy]-2-fluoro-phenyl]-1,2,4-oxadiazol-3-yl]ethyl]-2-fluoro-phenyl]propanoic acid (15 mg, 0.028 mmol, 56%) as a solid. MS (ESI): 526.1 m/z (M+H)⁺. ¹H NMR (400 MHz, CD₃OD) δ 7.56 (m, 1H), 7.34-7.29 (m, 2H), 7.25-7.14 (m, 4H), 7.04 (t, 1H), 6.55 (d, 1H), 4.64 (q, 1H), 2.94 (t, 2H), 2.56 (t, 2H), 1.68 (d, 3H) ppm.

### Example 54. Synthesis of 3-(3-((4-fluoro-3-(3-((6-fluoro-4-methyl-1H-indol-5-yl)oxy)phenyl)-1H-pyrazol-1-yl)methyl)phenyl)propanoic acid

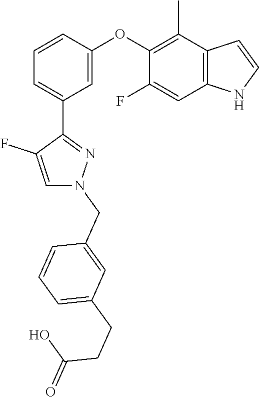 [SMILES: Cc1c(Oc2cccc(-c3nn(Cc4cccc(CCC(=O)O)c4)cc3F)c2)c(F)cc2[nH]ccc12]

### 4-Fluoro-N,N-dimethyl-1H-pyrazole-1-sulfonamide

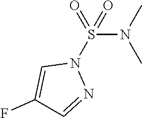

Step A: To a solution of 4-fluoro-1H-pyrazole (1.72 g, 20.0 mmol) in THE (20 mL) was added NaH (1.20 g, 30.0 mmol) at 0° C., and stirring was continued for 0.5 hours at that temperature. Dimethylsulfamoyl chloride (4.29 g, 30.0 mmol) was added dropwise, and the resulting mixture was stirred overnight at room temperature. The reaction mixture was diluted with ethyl acetate (100 mL) and washed with brine (30 mL×3), dried with anhydrous sodium sulfate, filtered, and concentrated in vacuo to give a residue, which was purified by silica gel column chromatography (eluting with 3/1 petroleum ether/ethyl acetate) to afford 4-fluoro-N,N-dimethyl-1H-pyrazole-1-sulfonamide (3.5 g, 90%). MS (ESI): 194.2 m/z (M+H)⁺.

### 3-Bromo-4-fluoro-N,N-dimethyl-1H-pyrazole-1-sulfonamide

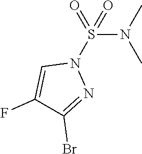

Step B: To a solution of 4-fluoro-N,N-dimethyl-1H-pyrazole-1-sulfonamide (3.5 g, 18.1 mmol) in anhydrous THE (50 mL) was added at −78° C. under nitrogen n-butyllithium (2.5N solution in THF, 8 mL, 20.0 mmol), and the resulting mixture was stirred at −78° C. for 0.5 hours. 1,2-dibromo-1,1,2,2-tetrachloroethane (6.42 g, 19.9 mmol) was added dropwise at −78° C., and stirring continued at that temperature for 4 hours. The reaction mixture was quenched with saturated aqueous NH₄Cl (50 mL) and was extracted with ethyl acetate (50 mL×3). The combined organic extracts were washed with brine (30 mL×3), dried with anhydrous sodium sulfate, filtered and concentrated in vacuo to give a residue, which was purified by silica gel column chromatography (eluting with 3/1 petroleum ether/ethyl acetate) to afford 3-bromo-4-fluoro-N,N-dimethyl-1H-pyrazole-1-sulfonamide (4.05 g, 82%). MS (ESI): 272, 274 m/z (M+H)⁺.

### Ethyl 3-(3-((3-bromo-4-fluoro-1H-pyrazol-1-yl)methyl)phenyl)propanoate and ethyl 3-(3-((5-bromo-4-fluoro-1H-pyrazol-1-yl)methyl)phenyl)propanoate

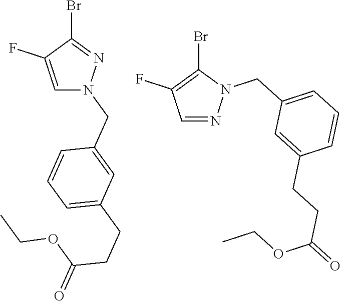

Step C: To a solution of 3-bromo-4-fluoro-N,N-dimethyl-1H-pyrazole-1-sulfonamide (4.05 g, 14.9 mmol) in DCM (50 mL) was added trifluoroacetic acid (3 mL) at 0° C., and then the resulting mixture was stirred at room temperature overnight. The resulting solution was added DCM (50 mL), and the organic phase was washed with saturated aqueous Na₂CO₃ (30 mL×2), water (30 mL), dried with anhydrous sodium sulfate, filtered, and concentrated in vacuo to give 3-bromo-4-fluoro-1H-pyrazole trifluoroacetate salt (3.9 g, crude) as a yellow oil. To a solution of 3-bromo-4-fluoro-1H-pyrazole trifluoroacetate salt (300 mg) and ethyl 3-(3-(bromomethyl)phenyl)propanoate (309 mg, 1.14 mmol) in DMF (5 mL) was added at 0° C. K₂CO₃ (315 mg, 2.28 mmol). The resulting mixture was stirred at room temperature overnight. The reaction mixture was diluted with ethyl acetate (30 mL) and washed with brine (10 mL×3), dried with anhydrous sodium sulfate, filtered, and concentrated in vacuo to give a residue. The residue was purified by silica gel column chromatography (eluting with 3/1-2/1 petroleum ether/ethyl acetate) to afford a mixture of ethyl 3-(3-((3-bromo-4-fluoro-1H-pyrazol-1-yl)methyl)phenyl)propanoate and ethyl 3-(3-((5-bromo-4-fluoro-1H-pyrazol-1-yl)methyl)phenyl)propanoate (300 mg, 54% over 2 steps). MS (ESI): 355.2, 357.2 m/z (M+H)⁺.

### Ethyl 3-(3-((4-fluoro-3-(3-((6-fluoro-4-methyl-1H-indol-5-yl)oxy)phenyl)-1H-pyrazol-1-yl)methyl)phenyl)propanoate and ethyl 3-(3-((4-fluoro-5-(3-((6-fluoro-4-methyl-1H-indol-5-yl)oxy)phenyl)-1H-pyrazol-1-yl)methyl)phenyl)propanoate

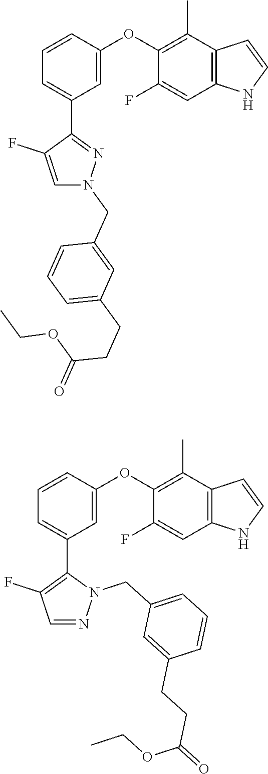 [SMILES: CCOC(=O)CCc1cccc(Cn2cc(F)c(-c3cccc(Oc4c(F)cc5[nH]ccc5c4C)c3)n2)c1 ; CCOC(=O)CCc1cccc(Cn2ncc(F)c2-c2cccc(Oc3c(F)cc4[nH]ccc4c3C)c2)c1]

Step D: To a solution of the product mixture obtained in the previous step (300 mg, 0.85 mmol) in 4/1 dioxane/water (10 mL) were added 6-fluoro-4-methyl-5-(3-(4,4,5,5-tetramethyl-1,3,2-dioxaborolan-2-yl)phenoxy)-1H-indole (Intermediate 14, 373 mg, 1.02 mmol), K₂CO₃ (234 mg, 1.69 mmol) and Pd(dppf)Cl₂ dichloromethane complex (69 mg, 0.08 mmol). The resulting mixture was heated for 2 hours at 120° C. in a microwave synthesizer, in a nitrogen filled microwave reaction tube. The reaction mixture was concentrated, and the residue dissolved in EtOAc (50 mL). The EtOAc solution was washed with water (30 mL), dried with anhydrous sodium sulfate, filtered and concentrated in vacuo to give a residue, which was purified by silica gel column chromatography (2/1/1 eluting with petroleum ether/ethyl acetate/DCM) to afford a mixture of ethyl 3-(3-((4-fluoro-3-(3-((6-fluoro-4-methyl-1H-indol-5-yl)oxy)phenyl)-1H-pyrazol-1-yl)methyl)phenyl)propanoate and ethyl 3-(3-((4-fluoro-5-(3-((6-fluoro-4-methyl-1H-indol-5-yl)oxy)phenyl)-1H-pyrazol-1-yl)methyl)phenyl)propanoate (100 mg, 23%) as a yellow solid. MS (ESI): 516.2 rn/z (M+H)⁺.

### 3-(3-((4-fluoro-3-(3-((6-fluoro-4-methyl-1H-indol-5-yl)oxy)phenyl)-1H-pyrazol-1-yl)methyl)phenyl)propanoic acid and 3-(3-((4 fluoro-5-(3-((6-fluoro-4-methyl-1H-indol-5-yl)oxy)phenyl)-1H-pyrazol-1-yl)methyl)phenyl)propanoic acid

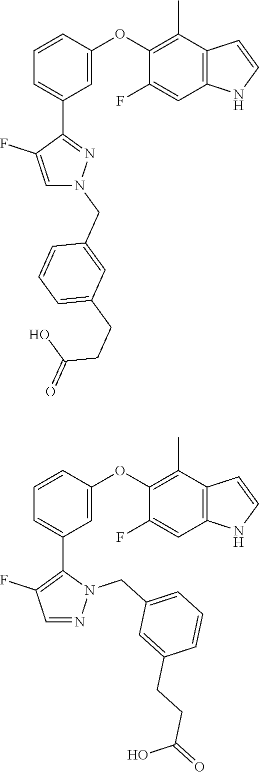 [SMILES: Cc1c(Oc2cccc(-c3nn(Cc4cccc(CCC(=O)O)c4)cc3F)c2)c(F)cc2[nH]ccc12 ; Cc1c(Oc2cccc(-c3c(F)cnn3Cc3cccc(CCC(=O)O)c3)c2)c(F)cc2[nH]ccc12]

Step E: To a solution of ethyl 3-(3-((4-fluoro-3-(3-((6-fluoro-4-methyl-1H-indol-5-yl)oxy)phenyl)-1H-pyrazol-1-yl)methyl)phenyl)propanoate and ethyl 3-(3-((4-fluoro-5-(3-((6-fluoro-4-methyl-1H-indol-5-yl)oxy)phenyl)-1H-pyrazol-1-yl)methyl)phenyl)propanoate (100 mg, 0.19 mmol) in THE (5 mL) was added a solution of LiOH (28 mg, 1.17 mmol) in H₂O (1 mL). The reaction mixture was stirred overnight at room temperature. The mixture was diluted with water (10 mL), THE was removed under reduced pressure, and the aqueous phase was acidified with 1 N hydrochloric acid until no more precipitate formed. The resulting suspension was extracted with ethyl acetate (30 mL×3). The combined organic extracts were washed with brine, dried over Na₂SO₄, concentrated in vacuo, and the residue was purified by prep-HPLC to provide 3-(3-((4-fluoro-3-(3-((6-fluoro-4-methyl-1H-indol-5-yl)oxy)phenyl)-1H-pyrazol-1-yl)methyl)phenyl)propanoic acid (46 mg, 49%) as a white solid and 3-(3-((4-fluoro-5-(3-((6-fluoro-4-methyl-1H-indol-5-yl)oxy)phenyl)-1H-pyrazol-1-yl)methyl)phenyl)propanoic acid (11 mg, 12%) as a white solid.

3-(3-((4-fluoro-3-(3-((6-fluoro-4-methyl-1H-indol-5-yl)oxy)phenyl)-1H-pyrazol-1-yl)methyl)phenyl)propanoic acid: MS (ESI): 488.2 m/z (M+H)⁺. ¹H NMR (400 MHz, MeOH-d₄) δ 7.62 (d, J=4.8 Hz, 1H), 7.44 (d, J=7.2 Hz, 1H), 7.34-7.05 (m, 8H), 6.79 (dd, J=8.0 Hz & 2.0 Hz, 1H), 6.52 (dd, J=3.2 Hz & 0.8 Hz, 1H), 5.21 (s, 2H), 2.90 (t, J=7.6 Hz, 2H), 2.57 (t, J=7.6 Hz, 2H), 2.38 (s, 3H) ppm.

3-(3-((4-fluoro-5-(3-((6-fluoro-4-methyl-1H-indol-5-yl)oxy)phenyl)-1H-pyrazol-1-yl)methyl)phenyl)propanoic acid: MS (ESI): 488.2 m/z (M+H)⁺. ¹H NMR (400 MHz, MeOH-d₄) δ 7.52 (d, J=4.4 Hz, 1H), 7.40 (dd, J=8.0 Hz & 8.0 Hz, 1H), 7.27 (d, J=3.2 Hz, 1H), 7.10-6.94 (m, 5H), 6.72-6.67 (m, 3H), 6.48 (dd, J=3.2 Hz & 0.8 Hz, 1H), 5.22 (s, 2H), 2.75 (t, J=7.6 Hz, 2H), 2.45 (t, J=7.6 Hz, 2H), 2.30 (s, 3H) ppm.

### Example 55. Synthesis of 2-(2-fluoro-3-(1-(4-fluoro-3-(4-fluoro-3-((6-fluoro-4-(methylsulfonyl)-1H-indol-5-yl)oxy)phenyl)-1H-pyrazol-1-yl)ethyl)phenyl)acetic acid

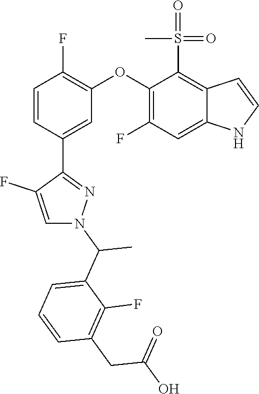 [SMILES: CC(c1cccc(CC(=O)O)c1F)n1cc(F)c(-c2ccc(F)c(Oc3c(F)cc4[nH]ccc4c3S(C)(=O)=O)c2)n1]

### 2-Fluoro-5-(4,4,5,5-tetramethyl-1,3,2-dioxaborolan-2-yl)phenol

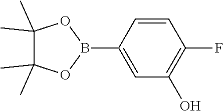 [SMILES: CC1(C)OB(c2ccc(F)c(O)c2)OC1(C)C]

Step A: A mixture of 5-bromo-2-fluoro-phenol (5.0 g, 26.2 mmol), bis(pinacolato)diboron (7.98 g, 31.4 mmol), Pd(dppf)Cl₂ dichloromethane complex (958 mg, 1.31 mmol) and potassium acetate (7.70 g, 78.5 mmol) in 1,4-dioxane (150 ml) was heated at 100° C. for 16 h under Ar. After cooling to room temperature, the reaction mixture was diluted with water (300 mL) and extracted with EtOAc (150 mL×3). The combined organic layers were dried over Na₂SO₄, filtered and concentration in vacuo. The residue was purified by silica gel column chromatography (10/1 petroleum ether/ethyl acetate) to afford 2-fluoro-5-(4,4,5,5-tetramethyl-1,3,2-dioxaborolan-2-yl)phenol (6.0 g, 96%) as white solid. MS (ESI): 239.1 m/z (M+H)⁺.

### 4-fluoro-1-(4-methoxybenzyl)-1H-pyrazole

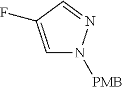

Step B: To a chilled (ice bath), stirred solution of 4-fluoropyrazole (4.0 g, 46.5 mmol) in THF (150 mL) was added NaH (60 wt. % suspension in mineral oil, 2.79 g, 69.7 mmol). After 10 min of stirring in cold bath, 4-methoxybenzyl chloride (8.68 g, 55.8 mmol) was added slowly to the reaction mixture. The resulting solution was allowed to warm to room temperature and stirred overnight. The reaction was quenched with water (200 mL) and extracted with EtOAc (150 mL×3). The organic phase was washed with brine (200 mL), dried over Na₂SO₄ and concentrated. The residue was purified by silica gel column chromatography, eluting with 4/1 petroleum ether/ethyl acetate, to afford 4-fluoro-1-[(4-methoxyphenyl)methyl]pyrazole (4.5 g, 47%) as a light-yellow oil. ¹H NMR (400 MHz, CD₃OD) δ 7.35 (d, J=4.0 Hz, 4H), 7.19-7.16 (m, 3H), 6.90-6.87 (m, 2H), 5.13 (s, 2H), 3.80 (s, 1H) ppm.

### 3-bromo-4-fluoro-1-(4-methoxybenzyl)-1H-pyrazole

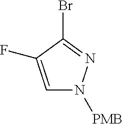

Step C: To a stirred solution of 4-fluoro-1-[(4-methoxyphenyl)methyl]pyrazole (4.42 g, 21.4 mmol) in anhydrous THE (90 mL) at −78° C. was slowly added a solution of n-butyllithium (2.5 M, 12.8 mL, 32.2 mmol), and the mixture was stirred for 20 min while maintaining the temperature below −65° C. A solution of 1,2-dibromo-tetrachloroethane (8.38 g, 25.7 mmol) in THE (25 mL) was added to the reaction mixture and the resulting solution was stirred for an additional 2 hours. The reaction was quenched with water (200 mL) and extracted with EtOAc (120 mL×3). The organic phase was washed with brine (200 mL), dried over Na₂SO₄, and concentrated in vacuo. The residue was purified by silica gel column chromatography, eluting with 20% EtOAc in petroleum ether, to afford 3-bromo-4-fluoro-1-[(4-methoxyphenyl)methyl]pyrazole (3.2 g, 52%) as a light yellow solid. ¹H NMR (400 MHz, CDCl₃) δ 7.42 (d, J=4.8 Hz, 1H), 7.20-7.17 (m, 2H), 6.87-6.85 (m, 2H), 5.22 (s, 2H), 3.78 (s, 1H) ppm.

### 2-fluoro-5-(4-fluoro-1-(4-methoxybenzyl)-1H-pyrazol-3-yl)phenol

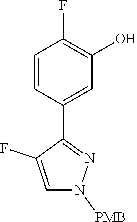

Step D: A mixture of 3-bromo-4-fluoro-1-[(4-methoxyphenyl)methyl]pyrazole (3.11 g, 8.50 mmol), 2-fluoro-5-(4,4,5,5-tetramethyl-1,3,2-dioxaborolan-2-yl)phenol (product of Step A, 2.12 g, 8.92 mmol), Pd(dppf)Cl₂ (311 mg, 0.424 mmol) and Na₂CO₃ (1.80 g, 17.0 mmol) in 1,4-dioxane (45.00 ml) and H₂O (15.00 mL) was heated at 120° C. for 16 hours under Ar. The mixture was poured into ice water, extracted with EtOAc (100 mL×3), the combined organic phase was dried over Na₂SO₄ and concentrated. The residue was purified by silica gel column chromatography, eluting with 3/1 petroleum ether/ethyl acetate, to afford 2-fluoro-5-[4-fluoro-1-[(4-methoxyphenyl) methyl]pyrazol-3-yl]phenol (2.01 g, yield 58.3%) as a white solid.

### 3-(3-(2-bromo-6-fluoro-3-methyl-4-nitrophenoxy)-4-fluorophenyl)-4-fluoro-1-(4-methoxybenzyl)-1H-pyrazole

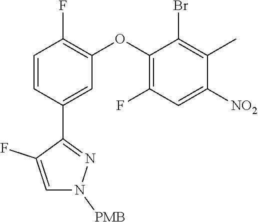

Step E: To a solution of 2-fluoro-5-[4-fluoro-1-[(4-methoxyphenyl)methyl]pyrazol-3-yl]phenol (1.5 g, 5.0 mmol) in DMF (15.0 mL) was added 3-bromo-1,2-difluoro-4-methyl-5-nitro-benzene (1.25 g, 4.98 mmol) and Cs₂CO₃ (3.09 g, 4.74 mmol). The resulting mixture was stirred at room temperature for 16 hours, diluted with H₂O (80 mL), and extracted with EtOAc (50 mL×3). The combined organic extracts were washed with brine (100 mL), dried over Na₂SO₄ and concentrated. The residue was purified by silica gel column chromatography, eluting with 20% ethyl acetate in petroleum ether, to afford methyl 2-[2-fluoro-3-[1-[3-[3-[6-fluoro-1-(p-tolylsulfonyl)-4-(2,2,2-trifluoroethyl)indol-5-yl]oxyphenyl]pyrazol-1-yl]ethyl]phenyl]acetate (2.0 g, 77%) as a white solid. MS (ESI): m/z 548.1, 550.1 (M+H)⁺.

### 4-bromo-6-fluoro-5-(2-fluoro-5-(4-fluoro-1-(4-methoxybenzyl)-1H-pyrazol-3-yl)phenoxy)-1H-indole

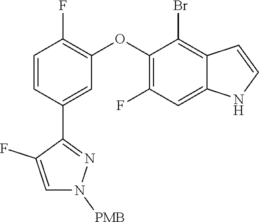

Step F: To a solution of 3-[3-(2-bromo-6-fluoro-3-methyl-4-nitro-phenoxy)-4-fluoro-phenyl]-4-fluoro-1-[(4-methoxyphenyl)methyl]pyrazole (2.3 g, 4.19 mmol) in DMF (20 mL) was added DMF-DMA (5.0 g, 41.9 mmol). The mixture was stirred at 120° C. for 2 hours. The mixture was poured into ice water (100 mL) and extracted with ethyl acetate (60 mL×3). The combined organic extracts were washed with brine (150 mL×2), dried over anhydrous Na₂SO₄, filtered and concentrated to afford (E)-2-[2-bromo-4-fluoro-3-[2-fluoro-5-[4-fluoro-1-[(4-methoxyphenyl)methyl]pyrazol-3-yl]phenoxy]-6-nitro-phenyl]-N,N-dimethyl-ethenamine (2.3 g, 90%) as a light-yellow oil. To a solution of the product thus obtained (2.30 g, 3.20 mmol) in toluene (18 mL) were added Fe powder (1.79 g, 32.8 mmol), and acetic acid (3.84 g, 64.0 mmol). The suspension was stirred overnight at 100° C. under an Ar atmosphere. The mixture was cooled to room temperature, poured into ice water, the pH adjusted to ~8 with saturated aqueous Na₂CO₃, and the solids filtered off. The filtrate was washed with H₂O (500 ml), the organic layer was dried over anhydrous Na₂SO₄, filtered, and concentrated. The residue was purified by silica gel column chromatography, eluting with 4/1 petroleum ether/ethyl acetate, to afford 4-bromo-6-fluoro-5-[2-fluoro-5-[4-fluoro-1-[(4-methoxyphenyl)methyl]pyrazol-3-yl]phenoxy]-1H-indole (1.9 g, 92%) as a white solid. MS (ESI): 528.1, 530.1 m/z (M+H)⁺.

### 4-bromo-6-fluoro-5-(2-fluoro-5-(4-fluoro-1-(4-methoxybenzyl)-1H-pyrazol-3-yl)phenoxy)-1-tosyl-1H-indole

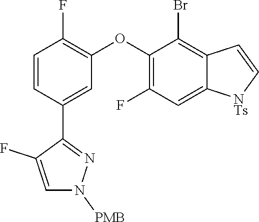

Step G: To a solution of 4-bromo-6-fluoro-5-[2-fluoro-5-[4-fluoro-1-[(4-methoxyphenyl)methyl]pyrazol-3-yl]phenoxy]-1H-indole (1.9 g, 3.42 mmol) in DMF (15 mL) at 0° C. was added NaH (60 wt. % solid dispersion in mineral oil, 205 mg, 5.12 mmol). After stirring at 0° C. for 30 minutes, 4-methylbenzenesulfonyl chloride (905 mg, 4.75 mmol) was added, and the mixture was stirred for another 2 hours at ambient temperature. The mixture was poured into ice water (80 mL) and extracted with ethyl acetate (50 mL×3). The combined organic phases were washed with brine (100 mL), dried over anhydrous Na₂SO₄, filtered and evaporated. The residue was purified by silica gel column chromatography, eluting with 5/1 petroleum ether/ethyl acetate, to afford 4-bromo-6-fluoro-5-(2-fluoro-5-(4-fluoro-1-(4-methoxybenzyl)-1H-pyrazol-3-yl)phenoxy)-1-tosyl-1H-indole (1.8 g, 73%) as a light yellow solid. MS (ESI): 682.1, 684.1 m/z (M+H)⁺.

### 6-fluoro-5-(2-fluoro-5-(4-fluoro-1-(4-methoxybenzyl)-1H-pyrazol-3-yl)phenoxy)-4-(methylthio)-1-tosyl-1H-indole

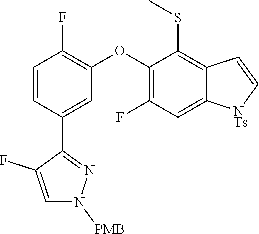

Step H: To a solution of 4-bromo-6-fluoro-5-(2-fluoro-5-(4-fluoro-1-(4-methoxybenzyl)-1H-pyrazol-3-yl)phenoxy)-1-tosyl-1H-indole (1.8 g, 2.64 mmol) in DMF (15 mL) was added tributyl(methylsulfanyl)stannane (1.78 g, 5.27 mmol) and Pd(dppf)Cl₂CH₂Cl₂ (215 mg, 0.264 mmol). The resulting mixture was stirred at 160° C. for 3 hours. The mixture was diluted with aqueous KF (200 mL), and the solids filtered off. The filtrate was extracted with EtOAc (100 mL×3). The combined organic extracts were washed with brine (200 mL), dried over anhydrous Na₂SO₄, filtered, and concentrated. The residue was purified by silica gel column chromatography, eluting with 4/1 petroleum ether/ethyl acetate, to afford 6-fluoro-5-[2-fluoro-5-[4-fluoro-1-[(4-methoxyphenyl)methyl]pyrazol-3-yl]phenoxy]-4-methylsulfanyl-1-(p-tolylsulfonyl)indole (1.0 g, 58%) as a light-yellow oil. MS (ESI): 650.2 m/z (M+H)⁺.

### 6-fluoro-5-(2-fluoro-5-(4-fluoro-1-(4-methoxybenzyl)-1H-pyrazol-3-yl)phenoxy)-4-(methylsulfonyl)-1-tosyl-1H-indole

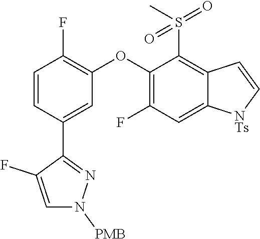

Step I: To a chilled (0° C.) solution of 6-fluoro-5-(2-fluoro-5-(4-fluoro-1-(4-methoxybenzyl)-1H-pyrazol-3-yl)phenoxy)-4-(methylthio)-1-tosyl-1H-indole (1.0 g, 1.54 mmol) in DCM (30 mL) was added 3-chloroperbenzoic acid (1.33 g, 0.77 mmol). The mixture was stirred at 0° C. for 2 hours, quenched with saturated aqueous NaHCO₃ (100 mL), and extracted with EtOAc (100 mL×3). The combined organic extracts were washed with brine (200 mL), dried over anhydrous Na₂SO₄, filtered, and concentrated. The residue was purified by silica gel column chromatography to afford 6-fluoro-5-[2-fluoro-5-[4-fluoro-1-[(4-methoxyphenyl)methyl]pyrazol-3-yl]phenoxy]-4-methylsulfonyl-1-(p-tolylsulfonyl)indole (300 mg, 28%) as a light yellow oil. MS (ESI): 682.1 m/z (M+H)⁺.

### 6-fluoro-5-(2-fluoro-5-(4-fluoro-1H-pyrazol-3-yl)phenoxy)-4-(methylsulfonyl)-1-tosyl-1H-indole

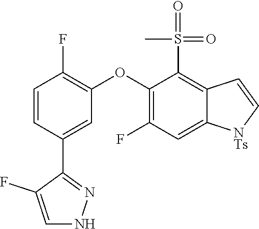 [SMILES: Cc1ccc(S(=O)(=O)n2ccc3c(S(C)(=O)=O)c(Oc4cc(-c5n[nH]cc5F)ccc4F)c(F)cc32)cc1]

Step J: To a solution of 6-fluoro-5-(2-fluoro-5-(4-fluoro-1-(4-methoxybenzyl)-1H-pyrazol-3-yl)phenoxy)-4-(methylsulfonyl)-1-tosyl-1H-indole (30 mg, 0.440 mmol) in DCM (1.5 mL) was added TFA (3.0 mL). The mixture was stirred overnight at 50° C. The pH of the mixture was adjusted to ~7.0 with saturated aqueous NaHCO₃, followed by extraction with ethyl acetate (4×10 mL). The combined organic extracts were dried over anhydrous sodium sulfate, filtered, and concentrated in vacuo. The residue was purified by silica gel column chromatography, eluting with 4/1 petroleum ether/ethyl acetate, to afford 6-fluoro-5-[2-fluoro-5-(4-fluoro-1H-pyrazol-3-yl)phenoxy]-4-methylsulfonyl-1-(p-tolylsulfonyl)indole (130 mg, 52%) as a light yellow oil. MS (ESI): 562.1 m/z (M+H)⁺.

### methyl 2-(2-fluoro-3-(1-(4-fluoro-3-(4-fluoro-3-((6-fluoro-4-(methylsulfonyl)-1-tosyl-1H-indol-5-yl)oxy)phenyl)-1H-pyrazol-1-yl)ethyl)phenyl)acetate

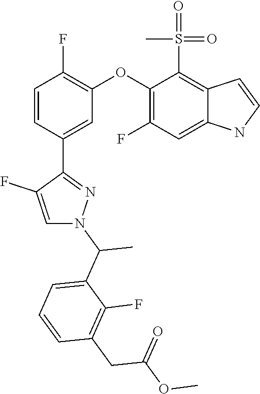

Step K: To a solution of 6-fluoro-5-[2-fluoro-5-(4-fluoro-1H-pyrazol-3-yl)phenoxy]-4-methylsulfonyl-1-(p-tolylsulfonyl)indole (130 mg, 0.232 mmol) in DMF (3.0 mL) was added at 0° C. methyl 2-[3-(1-bromoethyl)-2-fluoro-phenyl]acetate (95.5 mg, 0.347 mmol) and Cs₂CO₃ (151 mg, 0.463 mmol). The reaction mixture was stirred at 30° C. for 16 hours. 40 mL of water was added, and the resulting mixture was extracted with EtOAc (20 mL×3). The combined organic extracts were washed with brine (30 mL), dried over Na₂SO₄ and concentrated. The residue was purified by silica gel column chromatography, eluting with 3/1 petroleum ether/ethyl acetate, to afford methyl 2-(2-fluoro-3-(1-(4-fluoro-3-(4-fluoro-3-((6-fluoro-4-(methylsulfonyl)-1-tosyl-1H-indol-5-yl)oxy)phenyl)-1H-pyrazol-1-yl)ethyl)phenyl)acetate (70 mg, 40%) as a white solid. MS (ESI): 756.2 m/z (M+H)⁺.

### 2-(2-fluoro-3-(1-(4-fluoro-3-(4-fluoro-3-((6-fluoro-4-(methylsulfonyl)-1H-indol-5-yl)oxy)phenyl)-1H-pyrazol-1-yl)ethyl)phenyl)acetic acid

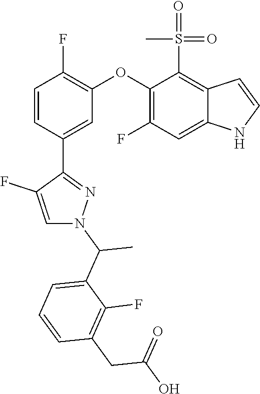 [SMILES: CC(c1cccc(CC(=O)O)c1F)n1cc(F)c(-c2ccc(F)c(Oc3c(F)cc4[nH]ccc4c3S(C)(=O)=O)c2)n1]

Step L: To a solution of methyl 2-[2-fluoro-3-[1-[4-fluoro-3-[4-fluoro-3-[6-fluoro-4-methylsulfonyl-1-(p-tolylsulfonyl)indol-5-yl]oxyphenyl]pyrazol-1-yl]ethyl]phenyl]acetate (70 mg, 0.0926 mmol) in MeOH/H₂O (v/v=5/1, 3.0 mL) was added LiOH·H₂O (77.7 mg, 1.85 mmol), and the mixture was stirred for 2 hours at room temperature. The pH of the mixture was adjusted to ~6.0 with 1N hydrochloric acid, followed by extraction with ethyl acetate (5×10 mL). The organic extracts were dried over anhydrous Na₂SO₄, filtered and concentrated in vacuo. The residue was purified by prep-HPLC to afford the title compound, 2-[2-fluoro-3-[1-[4-fluoro-3-[4-fluoro-3-[(6-fluoro-4-methylsulfonyl-1H-indol-5-yl)oxy]phenyl]pyrazol-1-yl]ethyl]phenyl]acetic acid (36 mg, 57%) as a white solid. MS (ESI): 588.2 m/z (M+H)⁺. ¹H NMR (400 MHz, CD₃OD) 7.69 (d, J=5.2 Hz, 2H), 7.55 (s, 1H), 7.49 (s, 1H), 7.28 (t, J=10.0 Hz, 1H), 7.21-7.17 (m, 3H), 7.04 (d, J=3.6 Hz, 2H), 5.72 (d, J=7.2 Hz, 1H), 3.65 (s, 2H), 3.37 (s, 3H), 1.79 (t, J=6.4 Hz, 3H) ppm.

### Example 56

### Synthesis of 3-(3-((1-(5-((4,6-difluoro-1H-indol-5-yl)oxy)-2-fluorophenyl)-5-((2-hydroxyethyl)amino)-1H-pyrazol-3-yl)methyl)phenyl) propanoic acid

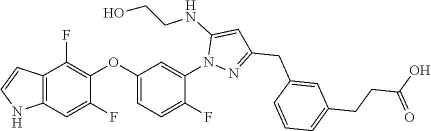 [SMILES: O=C(O)CCc1cccc(Cc2cc(NCCO)n(-c3cc(Oc4c(F)cc5[nH]ccc5c4F)ccc3F)n2)c1]

### 2-[[5-(3-Bromo-4-fluoro-phenoxy)-4,6-difluoro-indol-1-yl]methoxy]ethyl-trimethyl-silane

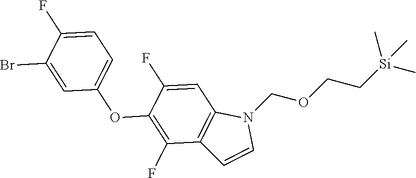 [SMILES: C[Si](C)(C)CCOCn1ccc2c(F)c(Oc3ccc(F)c(Br)c3)c(F)cc21]

Step A: To a stirred and chilled (0° C.) solution of 5-(3-bromo-4-fluoro-phenoxy)-4,6-difluoro-1H-indole (Intermediate 15) (1.00 eq, 12.00 g, 35.1 mmol) in THE (80 mL) was added NaH (1.50 eq, 1.26 g, 52.6 mmol) portion-wise over a period of 10 min. The mixture was stirred at rt for 10 min, treated with 2-(trimethylsilyl) ethoxymethyl chloride (1.20 eq, 7.5 mL, 42.1 mmol) dropwise over 5 min at 0° C., and stirred for another 1 h at room temperature. The reaction mass was quenched with ice-cold water (100 ml) and extracted with ethyl acetate (2×100 ml). The combined organic extracts were washed with brine (100 mL), dried over anhydrous Na₂SO₄, filtered, and concentrated. The crude product was purified by flash column chromatography on silica gel, eluting with 3% ethyl acetate in hexanes, to obtain 2-[[5-(3-bromo-4-fluoro-phenoxy)-4,6-difluoro-indol-1-yl]methoxy]ethyl-trimethyl-silane (13.50 g, 28.6 mmol, 81%) as a thick syrup. ¹H NMR (300 MHz, DMSO-d₆) δ 7.62 (d, 1H), 7.60 (d, 1H), 7.35 (t, 1H), 7.28-7.25 (m, 1H), 7.02-6.97 (m, 1H)

### [5-[4,6-Difluoro-1-(2-trimethylsilylethoxymethyl)indol-5-yl]oxy-2-fluoro-phenyl]hydrazine

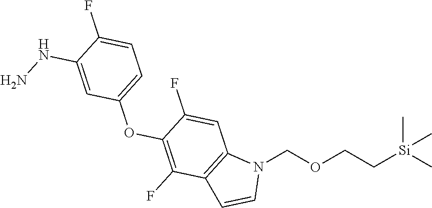 [SMILES: C[Si](C)(C)CCOCn1ccc2c(F)c(Oc3ccc(F)c(NN)c3)c(F)cc21]

Step B: To a stirred solution of 2-[[5-(3-bromo-4-fluoro-phenoxy)-4,6-difluoro-indol-1-yl]methoxy]ethyl-trimethyl-silane (1.00 eq, 3.00 g, 6.35 mmol) and hydrazine hydrate (3.00 eq, 0.95 g, 19.1 mmol) in 1,4-dioxane (30 mL) was added KOH (4.00 eq, 1.43 g, 25.4 mmol). The mixture was purged with nitrogen for 5 minutes, treated with bis[tris(2-methylphenyl)phosphine]palladium (0.0500 eq, 227 mg, 0.318 mmol) and (R)-1-[(SP)-2-(dicyclohexylphosphino)ferrocenyl]ethyldi-tert-butylphosphine (0.0500 eq, 176 mg, 0.318 mmol), and heated at 70° C. for 3 h, then cooled to 25° C. The reaction mixture was diluted with ethyl acetate (100 mL), washed with water (2×50 mL), brine (50 mL), dried over sodium sulfate, filtered, and concentrated. The residue was purified by flash column chromatography on silica, eluting with 0-30% ethyl acetate in hexanes, to afford [5-[4,6-difluoro-1-(2-trimethylsilylethoxymethyl)indol-5-yl]oxy-2-fluoro-phenyl]hydrazine (1.80 g, 3.65 mmol, 57% yield) as light brown syrup. MS (ESI): 424 m/z (M+H)⁺.

### 5-[(3-Bromophenyl)methyl]-2-[5-[4,6-difluoro-1-(2-trinethylsilylethoxynethyl)indol-5-yl]oxy-2-fluoro-phenyl]pyrazol-3-amine

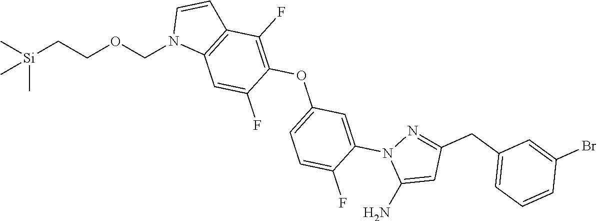 [SMILES: C[Si](C)(C)CCOCn1ccc2c(F)c(Oc3ccc(F)c(-n4nc(Cc5cccc(Br)c5)cc4N)c3)c(F)cc21]

Step C: To a stirred solution of 4-(3-bromophenyl)-3-oxo-butanenitrile (1.00 eq, 843 mg, 3.54 mmol) and [5-[4,6-difluoro-1-(2-trimethylsilylethoxymethyl)indol-5-yl]oxy-2-fluoro-phenyl]hydrazine (1.00 eq, 1.50 g, 3.54 mmol) in ethanol (25 mL) was added at rt acetic acid (2.00 eq, 0.41 mL, 7.08 mmol). The reaction mixture was heated at 100° C. for 24 h and concentrated. The crude was dissolved in ethyl acetate (20 mL), washed with saturated aqueous sodium bicarbonate solution (10 mL), brine (10 mL), dried over sodium sulfate, filtered, and concentrated. The residue was purified by flash chromatography on silica, eluting with 0-20% ethyl acetate in hexanes, to afford 5-[(3-bromophenyl)methyl]-2-[5-[4,6-difluoro-1-(2-trimethylsilylethoxymethyl)indol-5-yl]oxy-2-fluoro-phenyl]pyrazol-3-amine (1.30 g, 1.75 mmol, 49% yield) as a light-yellow semi solid. MS (ESI): 643, 645 m/z (M+H)⁺.

### Ethyl (E)-3-[3-[[5-amino-1-[5-[4,6-difluoro-]-(2-trimethylsilylethoxymethyl)indol-5-yl]oxy-2-fluoro-phenyl]pyrazol-3-yl]methyl]phenyl]prop-2-enoate

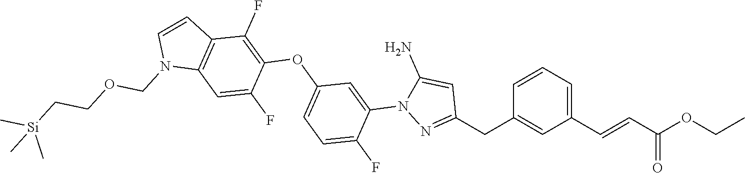 [SMILES: CCOC(=O)/C=C/c1cccc(Cc2cc(N)n(-c3cc(Oc4c(F)cc5c(ccn5COCC[Si](C)(C)C)c4F)ccc3F)n2)c1]

Step D: To a stirred, nitrogen purged solution of 5-[(3-bromophenyl)methyl]-2-[5-[4,6-difluoro-1-(2-trimethylsilylethoxymethyl)indol-5-yl]oxy-2-fluoro-phenyl]pyrazol-3-amine (1.00 eq, 1.00 g, 1.35 mmol), ethyl (E)-3-(4,4,5,5-tetramethyl-1,3,2-dioxaborolan-2-yl)prop-2-enoate (1.20 eq, 0.37 g, 1.62 mmol) in 1,4-dioxane (33 mL), and water (11 mL) was added (1,1′-Bis(diphenylphosphino)ferrocene)palladium (II) dichloride (0.0500 eq, 0.049 g, 0.0674 mmol). The reaction mixture was heated at 90° C. for 16 h, cooled to rt, and diluted with water (30 mL) and ethyl acetate (30 mL). The mixture was filtered through a pad of Celite and washed with ethyl acetate (30 mL). The separated organic phase was washed with brine (20 mL), dried over sodium sulfate, filtered, and concentrated. The crude was purified by flash column chromatography on silica gel, eluting with 15-18% EtOAc in hexanes, to give ethyl (E)-3-[3-[[5-amino-1-[5-[4,6-difluoro-1-(2-trimethylsilylethoxymethyl)indol-5-yl]oxy-2-fluoro-phenyl]pyrazol-3-yl]methyl]phenyl]prop-2-enoate (560 mg, 0.845 mmol, 62.65% yield) as a colorless syrup. MS (ESI): 663 m/z (M+H)⁺.

### Ethyl 3-[3-[[5-amino-1-[5-[4,6-difluoro-1-(2-trimethylsilylethoxymethyl)indol-5-yl]oxy-2-fluoro-phenyl]pyrazol-3-yl]methyl]phenyl]propanoate

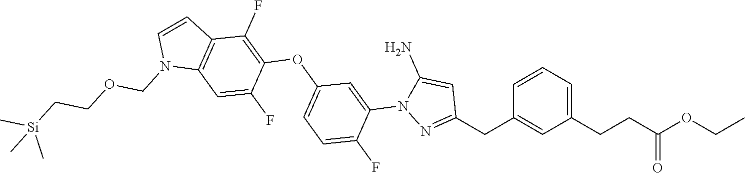 [SMILES: CCOC(=O)CCc1cccc(Cc2cc(N)n(-c3cc(Oc4c(F)cc5c(ccn5COCC[Si](C)(C)C)c4F)ccc3F)n2)c1]

Step E: To a stirred and cooled (−10° C.) solution of ethyl (E)-3-[3-[[5-amino-1-[5-[4,6-difluoro-1-(2-trimethylsilylethoxymethyl)indol-5-yl]oxy-2-fluoro-phenyl]pyrazol-3-yl]methyl]phenyl]prop-2-enoate (1.00 eq, 560 mg, 0.833 mmol) in methanol (15 mL) was added cobalt (II) chloride (0.107 eq, 12 mg, 0.0893 mmol) and sodium borohydride (1.61 eq, 51 mg, 1.34 mmol). The reaction progress was monitored by TLC and LCMS. Upon completion, the reaction was quenched with water (10 mL), then concentrated. The residue was partitioned between water (10 mL) and ethyl acetate (15 mL). The separated ethyl acetate layer was dried over sodium sulfate, filtered, and concentrated. The crude was purified by flash column chromatography on silica gel, eluting with 15-20% EtOAc in hexanes, to give ethyl 3-[3-[[5-amino-1-[5-[4,6-difluoro-1-(2-trimethylsilylethoxymethyl)indol-5-yl]oxy-2-fluoro-phenyl]pyrazol-3-yl]methyl]phenyl]propanoate (460 mg, 0.692 mmol, 83% yield) as a colorless syrup. MS (ESI): 665 m/z (M+H)⁺.

### Ethyl 3-[3-[[5-[2-[tert-butyl(dimethyl)silyl]oxyethylamino]-1-[5-[4,6-difluoro-1-(2-trimethylsilylethoxymethyl)indol-5-yl]oxy-2-fluoro-phenyl]pyrazol-3-yl]methyl]phenyl]propanoate

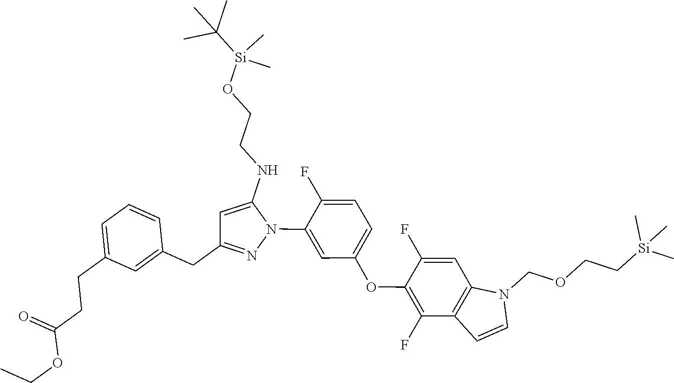 [SMILES: CCOC(=O)CCc1cccc(Cc2cc(NCCO[Si](C)(C)C(C)(C)C)n(-c3cc(Oc4c(F)cc5c(ccn5COCC[Si](C)(C)C)c4F)ccc3F)n2)c1]

Step F: To a stirred solution of ethyl 3-[3-[[5-amino-1-[5-[4,6-difluoro-1-(2-trimethylsilylethoxymethyl)indol-5-yl]oxy-2-fluoro-phenyl]pyrazol-3-yl]methyl]phenyl] propanoate (1.00 eq, 90 mg, 0.132 mmol) in methanol (5 mL) was added acetic acid (1.00 eq, 0.0076 mL, 0.132 mmol) and (tert-butyldimethylsilyloxy)acetaldehyde (1.50 eq, 0.038 mL, 0.198 mmol). The reaction mixture was stirred at ambient temperature for 16 h, then treated with sodium cyanoborohydride (2.00 eq, 17 mg, 0.264 mmol). The reaction mixture was stirred at ambient temperature for another 6 h and concentrated. The crude product was suspended in ethyl acetate (5 mL), washed with water (5 mL), brine (5 mL), dried over sodium sulfate, filtered, and concentrated. The residue was purified via column chromatography on silica gel, eluting with 3% to 15% ethyl acetate in hexanes, to obtain ethyl 3-[3-[[5-[2-[tert-butyl(dimethyl)silyl]oxyethylamino]-1-[5-[4,6-difluoro-1-(2-trimethylsilylethoxymethyl)indol-5-yl]oxy-2-fluoro-phenyl]pyrazol-3-yl]methyl]phenyl]propanoate (80 mg, 0.0962 mmol, 72.90% yield) as a colorless syrup. MS (ESI): 823 m/z (M+H)⁺.

### Ethyl 3-[3-[[1-[5-[(4,6-difluoro-1H-indol-5-yl)oxy]-2-fluoro-phenyl]-5-(2-hydroxyethylamino)pyrazol-3-yl]methyl]phenyl]propanoate

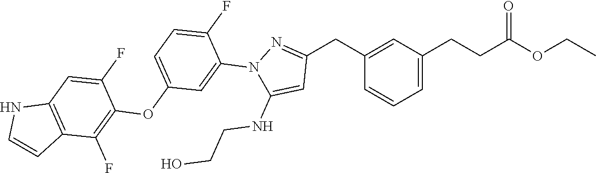 [SMILES: CCOC(=O)CCc1cccc(Cc2cc(NCCO)n(-c3cc(Oc4c(F)cc5[nH]ccc5c4F)ccc3F)n2)c1]

Step G: To a stirred solution of ethyl 3-[3-[[5-[2-[tert-butyl(dimethyl)silyl]oxyethylamino]-1-[5-[4,6-difluoro-1-(2-trimethylsilylethoxymethyl)indol-5-yl]oxy-2-fluoro-phenyl]pyrazol-3-yl]methyl]phenyl]propanoate (1.00 eq, 80 mg, 0.0962 mmol) in THE (5 mL) was added TBAF (10.0 eq, 0.30 mL, 0.298 mmol). The reaction mixture was stirred at 80° C. for 16 h, then cooled to room temperature, and concentrated. The residue was dissolved in ethyl acetate (5 mL), washed with water (5 mL), brine (5 mL), dried over sodium sulfate and concentrated to obtain ethyl 3-[3-[[1-[5-[(4,6-difluoro-1H-indol-5-yl)oxy]-2-fluoro-phenyl]-5-(2-hydroxyethylamino)pyrazol-3-yl]methyl]phenyl]propanoate (45 mg, 0.0723 mmol, 80% yield) as light-yellow syrup. MS (ESI): 579 m/z (M+H)⁺.

### 3-[3-[[1-[5-[(4,6-Difluoro-1H-indol-5-yl)oxy]-2-fluoro-phenyl]-5-(2-hydroxyethylamino)pyrazol-3-yl]methyl]phenyl]propanoic acid

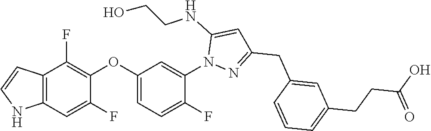 [SMILES: O=C(O)CCc1cccc(Cc2cc(NCCO)n(-c3cc(Oc4c(F)cc5[nH]ccc5c4F)ccc3F)n2)c1]

Step H: To a stirred solution of ethyl 3-[3-[[1-[5-[(4,6-difluoro-1H-indol-5-yl)oxy]-2-fluoro-phenyl]-5-(2-hydroxyethylamino)pyrazol-3-yl]methyl]phenyl]propanoate (1.00 eq, 45 mg, 0.0723 mmol) in methanol (1 mL) and THE (2 mL) was added a NaOH (2.00 eq, 5.8 mg, 0.145 mmol) solution in water (1 mL). The reaction mixture was stirred at room temperature for 2 h, concentrated, and diluted with water (5 mL). The mixture was acidified (to pH-4) with 2 N aqueous HCl, extracted with ethyl acetate (2×5 mL). The combined ethyl acetate layers were dried over sodium sulfate, filtered, and concentrated. The crude product was purified via flash column chromatography on silica gel, eluting with 30% to 80% ethyl acetate in hexanes to obtain 3-[3-[[1-[5-[(4,6-difluoro-1H-indol-5-yl)oxy]-2-fluoro-phenyl]-5-(2-hydroxyethylamino)pyrazol-3-yl]methyl]phenyl]propanoic acid (30 mg, 0.0484 mmol, 66.9% yield) as an off-white solid. MS (ESI): 551 m/z (M+H)⁺.

### Example 57

### Synthesis of 3-[3-[[1-[5-[(4,6-difluoro-1H-indol-5-yl)oxy]-2-fluoro-phenyl]-5-(2-oxo-1-piperidyl)pyrazol-3-yl]methyl]phenyl]propanoic acid

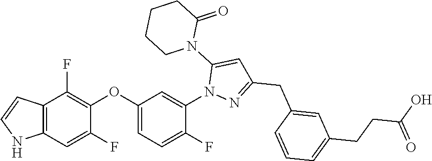 [SMILES: O=C(O)CCc1cccc(Cc2cc(N3CCCCC3=O)n(-c3cc(Oc4c(F)cc5[nH]ccc5c4F)ccc3F)n2)c1]

### Ethyl 3-(3-((5-(5-bromopentanamido)-1-(5-((4,6-difluoro-1-((2-(trimethylsilyl)ethoxy)methyl)-1H-indol-5-yl)oxy)-2-fluorophenyl)-1H-pyrazol-3-yl)methyl)phenyl)propanoate

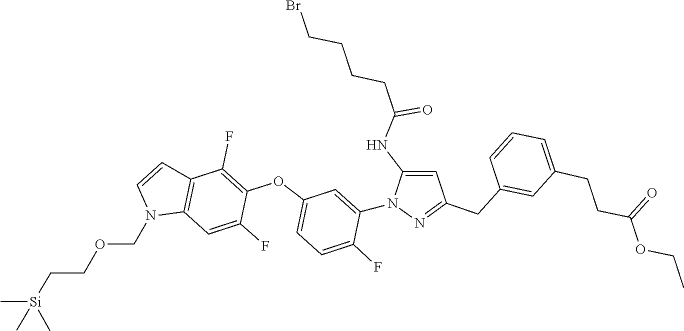 [SMILES: CCOC(=O)CCc1cccc(Cc2cc(NC(=O)CCCCBr)n(-c3cc(Oc4c(F)cc5c(ccn5COCC[Si](C)(C)C)c4F)ccc3F)n2)c1]

Step A: To a stirred solution of ethyl 3-[3-[[5-amino-1-[5-[4,6-difluoro-1-(2-trimethylsilylethoxymethyl)indol-5-yl]oxy-2-fluoro-phenyl]pyrazol-3-yl]methyl]phenyl]propanoate (Step E, Example 56) (1.00 eq, 200 mg, 0.301 mmol) in DCM (2 mL) were added potassium carbonate (1.50 eq, 62 mg, 0.451 mmol) and 5-bromovalerylchloride (1.20 eq, 51 mg, 0.361 mmol) at RT. The reaction mixture was stirred for 4 h. Another 0.5 equivalent of 5-bromovalerylchloride was added and stirring continued for an additional 24 h. The reaction mixture was diluted with water (20 mL) and extracted with DCM (2×20 mL). The combined organic extracts were washed with brine (20 mL), dried over anhydrous Na₂SO₄, filtered, and concentrated under reduced pressure to give the crude product, which was used in the next step without purification. LC-MS analysis of the crude revealed the presence of ~85% of the desired product [MS (ESI): 827, 829 m/z (M+H)⁺] and ~11% of the diacylated byproduct.

### Ethyl 3-[3-[[1-[5-[4,6-difluoro-1-(2-trimethylsilylethoxymethyl)indol-5-yl]oxy-2-fluoro-phenyl]-5-(2-oxo-1-piperidyl)pyrazol-3-yl]methyl]phenyl]propanoate

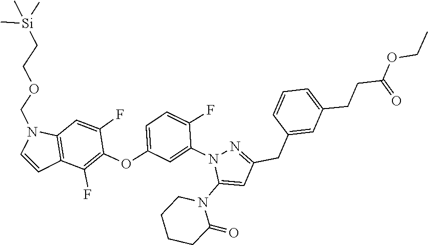 [SMILES: CCOC(=O)CCc1cccc(Cc2cc(N3CCCCC3=O)n(-c3cc(Oc4c(F)cc5c(ccn5COCC[Si](C)(C)C)c4F)ccc3F)n2)c1]

Step B: To a chilled (0° C.) and stirred solution of ethyl 3-[3-[[5-(5-bromopentanoylamino)-1-[5-[4,6-difluoro-1-(2-trimethylsilylethoxymethyl)indol-5-yl]oxy-2-fluoro-phenyl]pyrazol-3-yl]methyl]phenyl]propanoate (1.00 eq, 220 mg, 0.266 mmol) in anhydrous DMF (3 mL) was added under a nitrogen atmosphere potassium carbonate (2.00 eq, 73 mg, 0.532 mmol. The reaction mass was stirred at rt for 24 h, diluted with water (30 mL) and then extracted with EtOAc (2×20 mL). The combined organic extracts were washed with brine (20 mL), dried over anhydrous Na₂SO₄, filtered, and concentrated. The crude was purified by flash column chromatography on silica gel, eluting with 18-25% EtOAc in hexanes, to give ethyl 3-[3-[[1-[5-[4,6-difluoro-1-(2-trimethylsilylethoxymethyl)indol-5-yl]oxy-2-fluoro-phenyl]-5-(2-oxo-1-piperidyl)pyrazol-3-yl]methyl]phenyl]propanoate (90 mg, 0.120 mmol, 45% yield) as a colorless syrup. MS (ESI): 747.3 m/z (M+H)⁺.

### 3-[3-[[1-[5-[4,6-Difluoro-1-(2-trimethylsilylethoxymethyl)indol-5-yl]oxy-2-fluoro-phenyl]-5-(2-oxo-]-piperidyl)pyrazol-3-yl]methyl]phenyl]propanoic acid

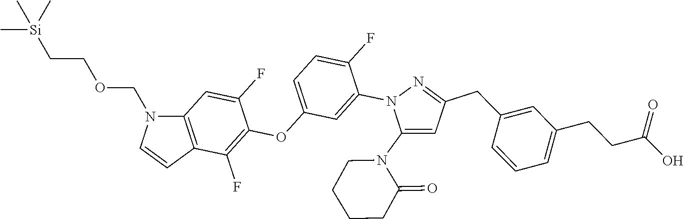 [SMILES: C[Si](C)(C)CCOCn1ccc2c(F)c(Oc3ccc(F)c(-n4nc(Cc5cccc(CCC(=O)O)c5)cc4N4CCCCC4=O)c3)c(F)cc21]

Step C: To a cooled (0° C.) and stirred solution of ethyl 3-[3-[[1-[5-[4,6-difluoro-1-(2-trimethylsilylethoxymethyl)indol-5-yl]oxy-2-fluoro-phenyl]-5-(2-oxo-1-piperidyl)pyrazol-3-yl]methyl]phenyl]propanoate (1.00 eq, 90 mg, 0.120 mmol) in THE (3 mL) and methanol (3 mL) was added 1N aqueous sodium hydroxide solution (5.00 eq, 1.0 mL, 0.602 mmol). The mixture was slowly warmed to rt and stirred for 2 h, diluted with water (10 mL), acidified (pH 2-3) with 1N aqueous HCl solution, and extracted with EtOAc (2×20 mL). The combined organic extracts were washed with brine (20 mL), dried over anhydrous Na₂SO₄, filtered, and concentrated. The crude was purified by flash column chromatography on silica gel, eluting with 50-60% EtOAc in hexanes, to afford 3-[3-[[1-[5-[4,6-difluoro-1-(2-trimethylsilylethoxymethyl)indol-5-yl]oxy-2-fluoro-phenyl]-5-(2-oxo-1-piperidyl)pyrazol-3-yl]methyl]phenyl]propanoic acid (50 mg, 0.0696 mmol, 58% yield) as a colorless syrup. MS (ESI): 719.6 m/z (M+H)⁺.

### 3-[3-[[1-[5-[(4,6-Difluoro-1H-indol-5-yl)oxy]-2-fluoro-phenyl]-5-(2-oxo-1-piperidyl)pyrazol-3-yl]methyl]phenyl]propanoic acid

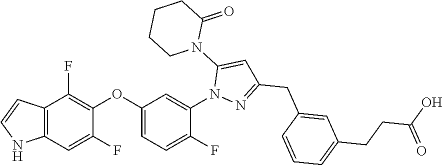 [SMILES: O=C(O)CCc1cccc(Cc2cc(N3CCCCC3=O)n(-c3cc(Oc4c(F)cc5[nH]ccc5c4F)ccc3F)n2)c1]

Step D: To a stirred solution of 3-[3-[[1-[5-[4,6-difluoro-1-(2-trimethylsilylethoxymethyl)indol-5-yl]oxy-2-fluoro-phenyl]-5-(2-oxo-1-piperidyl)pyrazol-3-yl]methyl]phenyl]propanoic acid (1.00 eq, 30 mg, 0.0417 mmol) in dry THE (1 mL) was added at rt 1.0 M tetrabutylammonium fluoride solution in THE (10.0 eq). After addition was completed, the reaction mixture was stirred at 75° C. for 6 h, then cooled to rt, quenched with water (10 mL), and extracted with EtOAc (2×10 mL). The combined organic extracts were washed with brine (10 mL), dried over anhydrous Na₂SO₄, filtered, and concentrated. The crude was purified by flash column chromatography on silica gel, eluting with 2-5% MeOH in DCM, to give 3-[3-[[1-[5-[(4,6-difluoro-1H-indol-5-yl)oxy]-2-fluoro-phenyl]-5-(2-oxo-1-piperidyl)pyrazol-3-yl]methyl]phenyl]propanoic acid (16 mg, 0.0252 mmol, 60% yield) as an off-white solid. MS (ESI): 589.4 m/z (M+H)⁺.

### Example 58

### Synthesis of 3-[3-[[1-[5-[(4,6-difluoro-1H-indol-5-yl)oxy]-2-fluoro-phenyl]-5-(2-oxopyrrolidin-1-yl)pyrazol-3-yl]methyl]phenyl]propanoic acid

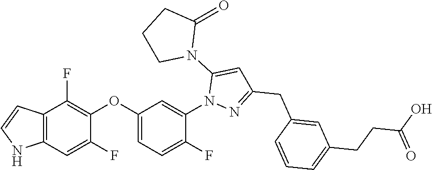 [SMILES: O=C(O)CCc1cccc(Cc2cc(N3CCCC3=O)n(-c3cc(Oc4c(F)cc5[nH]ccc5c4F)ccc3F)n2)c1]

### Ethyl 3-[3-[[5-(4-chlorobutanoylamino)-1-[5-[4,6-difluoro-1-(2-trimethylsilylethoxymethyl)indol-5-yl]oxy-2-fluoro-phenyl]pyrazol-3-yl]methyl]phenyl]propanoate

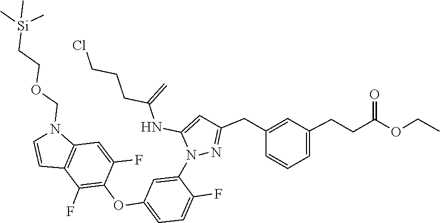 [SMILES: C=C(CCCCl)Nc1cc(Cc2cccc(CCC(=O)OCC)c2)nn1-c1cc(Oc2c(F)cc3c(ccn3COCC[Si](C)(C)C)c2F)ccc1F]

Step A: To a stirred solution of ethyl 3-[3-[[5-amino-1-[5-[4,6-difluoro-1-(2-trimethylsilylethoxymethyl)indol-5-yl]oxy-2-fluoro-phenyl]pyrazol-3-yl]methyl]phenyl]propanoate (Step E, Example 56) (1.00 eq, 200 mg, 0.301 mmol) in DCM (2 mL) was added at rt TEA (1.50 eq, 0.063 mL, 0.451 mmol) followed by 4-chlorobutyryl chloride (1.20 eq, 51 mg, 0.361 mmol). After completion of addition, the reaction mixture was stirred at rt for 4 h. A further 0.5 equivalents of 4-chlorobutyryl chloride were added and stirring continued for 24 h. The reaction mixture was diluted with water (10 mL) and extracted with EtOAc (2×10 mL). The combined organic extracts were washed with brine (20 mL), dried over anhydrous Na₂SO₄, filtered, and concentrated. The crude was purified by flash column chromatography on silica gel, eluting with 10-15% EtOAc in hexanes, to give ethyl 3-[3-[[5-(4-chlorobutanoylamino)-1-[5-[4,6-difluoro-1-(2-trimethylsilylethoxymethyl) indol-5-yl]oxy-2-fluoro-phenyl]pyrazol-3-yl]methyl]phenyl]propanoate (120 mg, 0.156 mmol, 52% yield) as a colorless syrup. MS (ESI): 769 m/z (M+H)⁺.

### Ethyl 3-(3-((1-(5-((4,6-difluoro-1-((2-(trimethylsilyl)ethoxy)methyl)-1H-indol-5-yl)oxy)-2-fluorophenyl)-5-(2-oxopyrrolidin-1-yl)-1H-pyrazol-3-yl)methyl)phenyl)propanoate

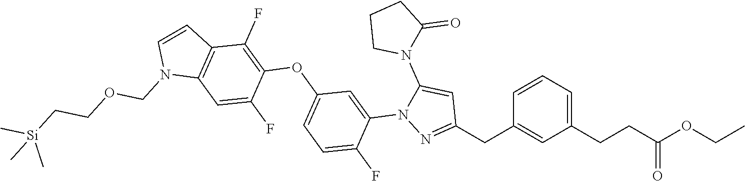 [SMILES: CCOC(=O)CCc1cccc(Cc2cc(N3CCCC3=O)n(-c3cc(Oc4c(F)cc5c(ccn5COCC[Si](C)(C)C)c4F)ccc3F)n2)c1]

Step B: To a stirred and chilled (0° C.) solution of ethyl 3-[3-[[5-(4-chlorobutanoylamino)-1-[5-[4,6-difluoro-1-(2-trimethylsilylethoxymethyl)indol-5-yl]oxy-2-fluoro-phenyl]pyrazol-3-yl]methyl]phenyl]propanoate (1.00 eq, 120 mg, 0.156 mmol) in DMF (3 mL) was added under a nitrogen atmosphere potassium carbonate (2.00 eq, 43 mg, 0.312 mmol). The reaction mixture was stirred at rt for 16 h, diluted with water (30 mL), and extracted with EtOAc (2×20 mL). The combined organic extracts were washed with brine (20 mL), dried over anhydrous Na₂SO₄, filtered, and concentrated. The crude was purified by flash column chromatography on silica gel, eluting with 18-25% EtOAc in hexanes, to afford the title compound (80 mg, 0.109 mmol, 69.9%). MS (ESI): 733 m/z (M+H)⁺.

### 3-[3-[[1-[5-[4,6-Difluoro-1-(2-trimethylsilylethoxymethyl)indol-5-yl]oxy-2-fluoro-phenyl]-5-(2-oxopyrrolidin-1-yl)pyrazol-3-yl]methyl]phenyl]propanoic acid

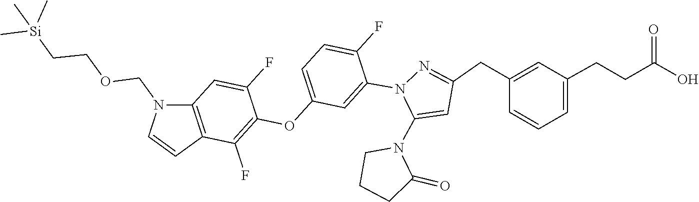 [SMILES: C[Si](C)(C)CCOCn1ccc2c(F)c(Oc3ccc(F)c(-n4nc(Cc5cccc(CCC(=O)O)c5)cc4N4CCCC4=O)c3)c(F)cc21]

Step C: To a chilled (0° C.) and stirred solution of ethyl 3-[3-[[1-[5-[4,6-difluoro-1-(2-trimethylsilylethoxymethyl)indol-5-yl]oxy-2-fluoro-phenyl]-5-(2-oxopyrrolidin-1-yl)pyrazol-3-yl]methyl]phenyl]propanoate (1.00 eq, 80 mg, 0.109 mmol) in THE (3 mL) and methanol (3 mL) was added 1N aqueous sodium hydroxide solution (5.00 eq, 1.0 mL, 0.546 mmol). The reaction solution was slowly warmed up to rt and stirred for 2 h, diluted with water (10 mL), acidified with 1N aqueous HCl to pH 2-3, and extracted with EtOAc (2×20 mL). The combined organic extracts were washed with brine (20 mL), dried over anhydrous Na₂SO₄, filtered, and concentrated. The crude was purified by flash column chromatography on silica gel, eluting with 45-55% EtOAc in hexanes, to give 3-[3-[[1-[5-[4,6-difluoro-1-(2-trimethylsilylethoxymethyl)indol-5-yl]oxy-2-fluoro-phenyl]-5-(2-oxopyrrolidin-1-yl)pyrazol-3-yl]methyl]phenyl]propanoic acid (55 mg, 0.0780 mmol, 71% yield) as a colorless syrup. MS (ESI): 705 m/z (M+H)⁺.

### 3-[3-[[1-[5-[(4,6-Difluoro-1H-indol-5-yl)oxy]-2-fluoro-phenyl]-5-(2-oxopyrrolidin-1-yl)pyrazol-3-yl]methyl]phenyl]propanoic acid

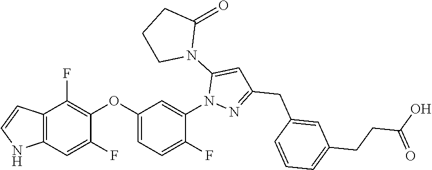 [SMILES: O=C(O)CCc1cccc(Cc2cc(N3CCCC3=O)n(-c3cc(Oc4c(F)cc5[nH]ccc5c4F)ccc3F)n2)c1]

Step D: To a stirred solution of 3-[3-[[1-[5-[4,6-difluoro-1-(2-trimethylsilylethoxymethyl)indol-5-yl]oxy-2-fluoro-phenyl]-5-(2-oxopyrrolidin-1-yl)pyrazol-3-yl]methyl]phenyl]propanoic acid (1.00 eq, 35 mg, 0.0497 mmol) in dry THF (2 mL) was added at rt a 1.0 M tetrabutylammonium fluoride solution in THE (10.0 eq). After completion of addition, the mixture was stirred at 75° C. for 6 h. The reaction was quenched with water (10 mL) and then extracted with EtOAc (2×10 mL). The combined organic extracts were washed with brine (10 mL), dried over anhydrous Na₂SO₄, filtered, and concentrated. The crude was purified by flash column chromatography on silica gel, eluting with 2-5% MeOH in DCM, to give 3-[3-[[1-[5-[(4,6-difluoro-1H-indol-5-yl)oxy]-2-fluoro-phenyl]-5-(2-oxopyrrolidin-1-yl)pyrazol-3-yl]methyl]phenyl]propanoic acid (20 mg, 0.0325 mmol, 65% yield) as a pale yellow solid. MS (ESI): 575 m/z (M+H)⁺. ¹H NMR (300 MHz, CDCl₃): δ 8.49 (s, 1H), 7.22-7.00 (m, 9H), 6.61 (t, 1H), 6.03 (s, 1H), 3.96 (s, 2H), 2.06 (t, 2H), 2.87 (t, 2H), 2.46 (t, 2H), 2.40 (t, 2H), 2.02 (m, 2H)

### Example 59

### Synthesis of 3-[3-[1-[1-[5-[(4,6-difluoro-1H-indol-5-yl)oxy]-2-fluoro-phenyl]-5,6-dihydro-4H-cyclopenta[c]pyrazol-3-yl]ethyl]-2-fluoro-phenyl]propanoic acid

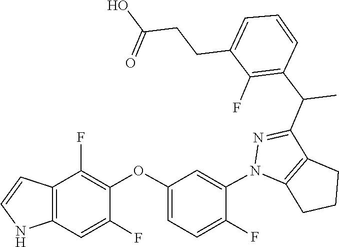 [SMILES: CC(c1cccc(CCC(=O)O)c1F)c1nn(-c2cc(Oc3c(F)cc4[nH]ccc4c3F)ccc2F)c2c1CCC2]

### 4,6-Difluoro-5-(4-fluoro-3-iodo-phenoxy)-1-(p-tolylsulfonyl)indole

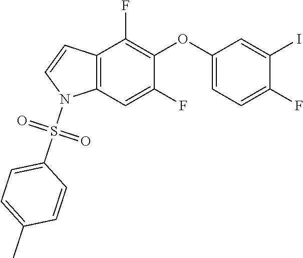 [SMILES: Cc1ccc(S(=O)(=O)n2ccc3c(F)c(Oc4ccc(F)c(I)c4)c(F)cc32)cc1]

Step A: To a stirred solution of 5-[4,6-difluoro-1-(p-tolylsulfonyl)indol-5-yl]oxy-2-fluoro-aniline (Intermediate 13 D) (1.00 eq, 1.20 g, 2.78 mmol) in THE (10 mL) was added KI (5.00 eq, 2303 mg, 13.9 mmol) and isoamyl nitrite (5.00 eq, 1.9 mL, 13.9 mmol). The mixture was stirred at 50° C. for 16 hours, poured into water (50 mL), and extracted with ethyl acetate (50 mL×2). The combined organic phases were washed with brine (50 mL×2), dried over anhydrous sodium sulfate, filtered, and concentrated. The residue was purified by flash chromatography on silica gel, eluting with 0-50% ethyl acetate in petroleum ether, to give 4,6-difluoro-5-(4-fluoro-3-iodo-phenoxy)-1-(p-tolylsulfonyl)indole (1.10 g, 2.02 mmol, 72.96% yield). MS (ESI): 544 m/z (M+H)⁺.

### 2-(3-Bromo-2-fluoro-phenyl)propanoyl chloride

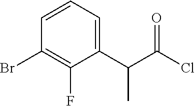

Step B: A solution of 2-(3-bromo-2-fluoro-phenyl)propanoic acid (1.00 eq, 2.00 g, 8.10 mmol) in thionyl chloride (33.8 eq, 20 mL, 274 mmol) was stirred at 80° C. for 3 h. The solvent was removed under vacuum to give 2-(3-bromo-2-fluoro-phenyl)propanoyl chloride (2.10 g, 7.91 mmol, 97.70% yield) as an oil, and used in the next step without purification.

### 2-[2-(3-Bromo-2-fluoro-phenyl)propanoyl]cyclopentanone

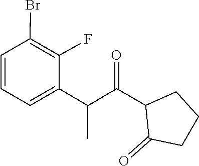 [SMILES: CC(C(=O)C1CCCC1=O)c1cccc(Br)c1F]

Step C: To a chilled (−78° C.) and stirred solution of cyclopentanone (1.00 eq, 0.67 mL, 7.53 mmol) in THE (20 mL) was added LDA (1.20 eq, 968 mg, 9.04 mmol). The mixture was stirred at −78° C. for 1 hour, treated with 2-(3-bromo-2-fluoro-phenyl)propanoyl chloride (1.00 eq, 2.00 g, 7.53 mmol) dropwise, warmed slowly to room temperature, and stirred at room temperature for another 3 hours. The reaction mixture was poured into water (50 mL) and extracted with ethyl acetate (50 mL×2). The combined organic phases were washed with brine (50 mL×2), dried over anhydrous sodium sulfate, filtered, and concentrated. The residue was purified by flash chromatography on silica gel column, eluting with 0-30% ethyl acetate in petroleum ether, to give 2-[2-(3-bromo-2-fluoro-phenyl)propanoyl]cyclopentanone (1.00 g, 3.19 mmol, 42% yield) as a white solid. MS (ESI): 313, 315 m/z (M+H)⁺.

### 3-[1-(3-Bromo-2-fluoro-phenyl)ethyl]-1,4,5,6-tetrahydrocyclopenta[c]pyrazole

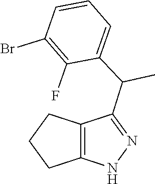 [SMILES: CC(c1cccc(Br)c1F)c1n[nH]c2c1CCC2]

Step D: To a stirred solution of 2-[2-(3-bromo-2-fluoro-phenyl)propanoyl]cyclopentanone (1.00 eq, 1.00 g, 3.19 mmol) in ethanol (10 mL) was added at rt hydrazinium hydroxide solution (2.00 eq, 0.20 g, 6.39 mmol). The mixture was stirred at 90° C. for 2 hours, poured into water (50 mL), and extracted with ethyl acetate (50 mL×2). The combined organic phases were washed with brine (50 mL×2), dried over anhydrous sodium sulfate, filtered, and concentrated. The residue was purified by flash column chromatography on silica gel, eluting with 0-40% ethyl acetate in petroleum ether, to give 3-[1-(3-bromo-2-fluoro-phenyl)ethyl]-1,4,5,6-tetrahydrocyclopenta[c]pyrazole (700 mg, 2.26 mmol, 71% yield) as an oil. MS (ESI): 309.1, 311.1 m/z (M+H)⁺.

### Ethyl (E)-3-[2-fluoro-3-[1-(1,4,5,6-tetrahydrocyclopenta[c]pyrazol-3-yl)ethyl]phenyl]prop-2-enoate

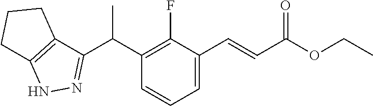 [SMILES: CCOC(=O)/C=C/c1cccc(C(C)c2n[nH]c3c2CCC3)c1F]

Step E: To a stirred solution of 3-[1-(3-bromo-2-fluoro-phenyl)ethyl]-1,4,5,6-tetrahydrocyclopenta[c]pyrazole (1.00 eq, 700 mg, 2.26 mmol) in 1,4-dioxane (10 mL) and water (2 mL) was added ethyl (E)-3-(4,4,5,5-tetramethyl-1,3,2-dioxaborolan-2-yl)prop-2-enoate (1.50 eq, 768 mg, 3.40 mmol), (1,1′-bis(diphenylphosphino)ferrocene)palladium(II) dichloride (0.100 eq, 164 mg, 0.226 mmol) and potassium carbonate (2.00 eq, 626 mg, 4.53 mmol). The mixture was stirred at 100° C. under Ar for 2 hours. The reaction mixture was concentrated. The residue was purified by flash column chromatography on silica gel, eluting with 0-40% ethyl acetate in petroleum ether), to give ethyl (E)-3-[2-fluoro-3-[1-(1,4,5,6-tetrahydrocyclopenta[c]pyrazol-3-yl)ethyl]phenyl]prop-2-enoate (550 mg, 1.67 mmol, 74% yield) as an oil. MS (ESI): 329.2 m/z (M+H)⁺.

### Ethyl 3-[2-fluoro-3-[1-(1,4,5,6-tetrahydrocyclopenta[c]pyrazol-3-yl)ethyl]phenyl]propanoate

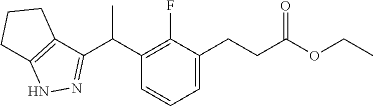 [SMILES: CCOC(=O)CCc1cccc(C(C)c2n[nH]c3c2CCC3)c1F]

Step F: To a stirred solution of ethyl (E)-3-[2-fluoro-3-[1-(1,4,5,6-tetrahydrocyclopenta[c]pyrazol-3-yl)ethyl]phenyl]prop-2-enoate (1.00 eq, 550 mg, 1.67 mmol) in ethanol (20 mL) was added Pd/C (5.00 eq, 891 mg, 8.37 mmol). The mixture was stirred overnight under a hydrogen atmosphere at ambient temperature. The reaction mixture was filtered through a pad of Celite, and the filtrate was concentrated to give ethyl 3-[2-fluoro-3-[1-(1,4,5,6-tetrahydrocyclopenta[c]pyrazol-3-yl)ethyl]phenyl]propanoate (500 mg, 1.51 mmol, 90% yield) as an oil. MS (ESI): 331.2 m/z (M+H)⁺.

### Ethyl 3-[3-[1-[1-[5-[4,6-difluoro-1-(p-tolylsulfonyl)indol-5-yl]oxy-2-fluoro-phenyl]-5,6-dihydro-4H-cyclopenta[c]pyrazol-3-yl]ethyl]-2-fluoro-phenyl]propanoate

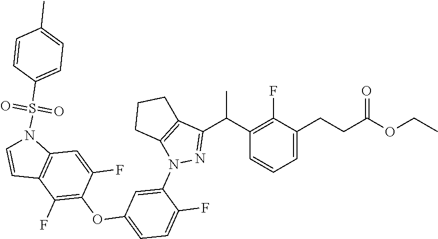 [SMILES: CCOC(=O)CCc1cccc(C(C)c2nn(-c3cc(Oc4c(F)cc5c(ccn5S(=O)(=O)c5ccc(C)cc5)c4F)ccc3F)c3c2CCC3)c1F]

Step G: To a mixture of ethyl 3-[2-fluoro-3-[1-(1,4,5,6-tetrahydrocyclopenta[c]pyrazol-3-yl)ethyl]phenyl]propanoate (1.00 eq, 300 mg, 0.908 mmol) and 4,6-difluoro-5-(4-fluoro-3-iodo-phenoxy)-1-(p-tolylsulfonyl)indole (1.00 eq, 493 mg, 0.908 mmol) in NMP (2 mL) was added copper(I) iodide (0.200 eq, 35 mg, 0.182 mmol), potassium carbonate (2.00 eq, 251 mg, 1.82 mmol) and (1R,2R)—N,N′-dimethyl-1,2-cyclohexanediamine (1.10 eq, 142 mg, 0.999 mmol). The mixture was stirred at 130° C. overnight, poured into water (50 mL) and extracted with ethyl acetate (50 mL×2). The combined organic phases were washed with brine (50 mL×2), dried over anhydrous sodium sulfate, filtered, and concentrated. The residue was purified by flash column chromatography on silica gel, eluting with 0-20% ethyl acetate in petroleum ether, to give ethyl 3-[3-[1-[1-[5-[4,6-difluoro-1-(p-tolylsulfonyl)indol-5-yl]oxy-2-fluoro-phenyl]-5,6-dihydro-4H-cyclopenta[c]pyrazol-3-yl]ethyl]-2-fluoro-phenyl]propanoate (250 mg, 0.335 mmol, 36.92% yield). MS (ESI): 746.2 m/z (M+H)⁺.

### 3-[3-[1-[1-[5-[(4,6-Difluoro-1H-indol-5-yl)oxy]-2-fluoro-phenyl]-5,6-dihydro-4H-cyclopenta[c]pyrazol-3-yl]ethyl]-2-fluoro-phenyl]propanoic acid

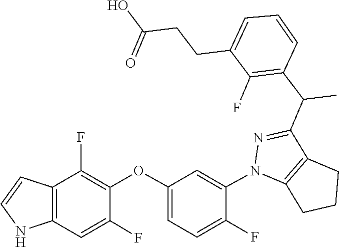 [SMILES: CC(c1cccc(CCC(=O)O)c1F)c1nn(-c2cc(Oc3c(F)cc4[nH]ccc4c3F)ccc2F)c2c1CCC2]

Step H: To a stirred solution of ethyl 3-[3-[1-[1-[5-[4,6-difluoro-1-(p-tolylsulfonyl)indol-5-yl]oxy-2-fluoro-phenyl]-5,6-dihydro-4H-cyclopenta[c]pyrazol-3-yl]ethyl]-2-fluoro-phenyl]propanoate (1.00 eq, 300 mg, 0.402 mmol) in THE (10 mL) and water (10 mL) was added lithium hydroxide (5.00 eq, 48 mg, 2.01 mmol). The reaction was stirred at 40° C. for 16 hours. The mixture was acidified with 1.0 M hydrochloric acid to a pH ~6 and extracted with ethyl acetate (20 mL×2). The combined organic phases were washed with brine (40 mL×2), dried over anhydrous sodium sulfate, filtered, and concentrated to give 3-[3-[1-[1-[5-[(4,6-difluoro-1H-indol-5-yl)oxy]-2-fluoro-phenyl]-5,6-dihydro-4H-cyclopenta[c]pyrazol-3-yl]ethyl]-2-fluoro-phenyl]propanoic acid (169 mg, 0.300 mmol, 75% yield). MS (ESI): 564.2 m/z (M+H)⁺.

### Biological Assays

### Example 60. Aggregation Analysis Using Differential Static Light Scattering (DSLS)

Purified recombinant NBD1 was produced using previously described methods (A. Schmidt, J. L. Mendoza, P. J. Thomas (2011) Biochemical and Biophysical Approaches to Probe CFTR Structure (365-376) M. D. Amaral, K. Kunzelmann (eds.), Cystic Fibrosis, Methods in Molecular Biology 741, Springer Science+Business Media). The effect of test compounds on thermal stability of NBD1 was evaluated by differential static light scattering (DSLS) using the Harbinger Stargazer-384 instrument (Epiphyte Three, Toronto, Canada). Test compounds were dissolved and diluted to desired concentrations in 100% DMSO. The compounds or DMSO controls (100 nL) were stamped into wells of a 385-well low volume optical plate (Corning Inc., Corning, NY) using the Echo 555 acoustic liquid handler (Labcyte Inc., San Jose, CA).

NBD1 protein was diluted to 0.2 mg/ml in S200 buffer (50 mM Tris-HCl, 150 mM NaCl, 5 mM MgCl₂, 2 mM ATP, 2 mM DTT, pH7.6) containing 1% glycerol. 10 uL of protein solution was aliquoted into the 384-well plate harboring the test compounds and 10 uL mineral oil was overlayed onto the protein solution, using the epMotion robotic liquid handler (Eppendorf North America, Hauppauge, NY). After placing into the Stargazer instrument, the plate was heated at 1° C. per minute to 70° C. Images were captured from 25° C. to 70° C. every 0.5° C. At the end of the experiment run, instrument software integrated image files and analyzed data automatically. A linear regression curve was generated for each well, representing the increase in light scattering over time. A temperature of aggregation (Tagg) was calculated based on the inflection point of the curve. To better compare data across experiments the average Tagg for DMSO control wells was calculated and subtracted from values for wells containing compounds to obtain a “ΔTagg” value. These ΔTagg values reflect stabilizing efficacy of the compounds.

Data for Compounds 1-55 are provided in Table 2 below.

TABLE 2

| | DSLS NBD1/3D Human |
|---|---|
| Example | Stability delta-T |
| No | (° C.) @ 100 (μM) |
| 1 | B |
| 2 | B |
| 3 | C |
| 4 | B |
| 5 | B |
| 6 | C |
| 7 | C |
| 8 | C |
| 9 | C |
| 10 | C |
| 11 | C |
| 12 | C |
| 13 | C |
| 14 | C |
| 15 | C |
| 16 | C |
| 17 | C |
| 18 | B |
| 19 | C |
| 20 | B |
| 21 | B |
| 22 | B |
| 23 | B |
| 24 | C |
| 25 | C |
| 26 | C |
| 27 | B |
| 28 | C |
| 29 | C |
| 30 | B |
| 31 | C |
| 32 | C |
| 33 | C |
| 34 | C |
| 35 | C |
| 36 | C |
| 37 | B |
| 38 | C |
| 39 | B |
| 40 | B |
| 41 | C |
| 42 | B |
| 43 | A |
| 44 | B |
| 45 | A |
| 46 | C |
| 47 | C |
| 48 | B |
| 49 | B |
| 50 | B |
| 51 | B |
| 52 | C |
| 53 | B |
| 54 | B |
| 55 | A |
| 56 | B |
| 57 | C |
| 58 | C |
| 59 | C |

*A: T ≥8° C.;
B: 4-8° C.;
C: ≤4° C.

### Example 61. TECC24 AUC Fold Over DMSO @10 μM

The effects of a test agent on CFTR-mediated transepithelial chloride transport was measured using TECC24 recording analysis. Test agents were solubilized in DMSO. Solubilized test agents were mixed with incubation medium containing DMEM/F12, Ultroser G (2%; Crescent Chemical, catalog #67042), Hyclone Fetal Clone 11 (2%; GE Healthcare, catalog #SH30066.02), bovine brain extract (0.25%; Lonza, catalog #CC-4098), insulin (2.5 μg/mL), IL-13 (10 ng/mL), hydrocortisone (20 nM), transferrin (2.5 g/mL), triiodothyronine (500 nM), ethanolamine (250 nM), epinephrine (1.5 μM), phosphoethanolamine (250 nM), and retinoic acid (10 nM). Primary human bronchial epithelial cells from a ΔF508 homozygous CF donor (CF-HBE cells; from University of North Carolina Cystic Fibrosis Tissue Procurement Center), grown on Transwell HTS 24-well cell culture inserts (Costar, catalog #3378), were exposed to test agents or controls dissolved in incubation medium. The CF-HBE cells were cultured at 36.5° C. for 48 hours before TECC24 recordings were performed in the presence or absence of test agent, a positive control or vehicle (DMSO).

Following incubation, the transwell cell culture inserts containing the test agent or control-treated CF-HBE cells were loaded onto a TECC24 apparatus (TECC v7 or MTECC v2; EP Design) to record the transepithelial voltage (VT) and resistance (TEER) using 4 AgCl electrodes per well configured in current-clamp mode. The apical and basolateral bath solutions both contained (in mM) 140 NaCl, 5 KCl, 2 CaCl₂), 1 MgCl₂, 10 Hepes, and 10 glucose (adjusted to pH 7.4 with NaOH). To inhibit basal Na+ absorption, the ENaC inhibitor benzamil (10 μM) was added to the bath. Then, the adenylate cyclase activator, forskolin (10 M), was added to the bath to activate CFTR. The forskolin-stimulated Cl— transport was halted by addition of CFTR inhibitor-172 (20 M) to the bath at the end of the experiment to confirm specificity. VT and TEER recordings were digitally acquired at routine intervals using TECC or MTECC software (EP Design). VT and TEER were transformed into equivalent transepithelial Cl— current (IEQ), and the Area Under the Curve (AUC) of the IEQ time course between forskolin and CFTR inhibitor-172 addition is generated using Excel (Microsoft). Efficacy is expressed as the ratio of the test agent AUC divided by vehicle AUC. EC50s based on AUC are generated using the non-linear regression log(agonist) vs. response function in Prism software (Graphpad) with HillSlope fixed=1.

If a test agent increased the AUC of the forskolin-stimulated IEQ relative to vehicle in CF-HBE cells, and this increase was inhibited by CFTR inhibitor-172, then the test agent was considered a CFTR corrector. The data is shown in Table 3 below.

TABLE 3

| Example | TECC24 @ 10 uM |
|---|---|
| No. | over DMSO |
| 4 | C |
| 10 | C |
| 44 | C |

ND = Not determined; “A” refers to AUC @ 10 uM >=12; “B” refers to AUC @ 10 uM between 4-12; “C” refers to AUC @ 10 uM <=4.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context.

The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 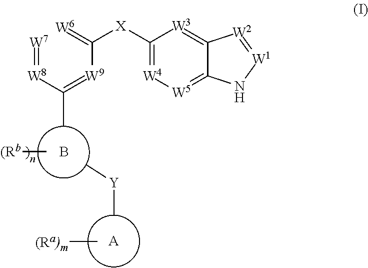
or a pharmaceutically acceptable salt thereof
wherein
W¹ is selected from the group consisting of —C(H)═, and —N═;
W² is selected from the group consisting of —C(H)═, —C(R^{d})═, and —N═;
W³ is selected from the group consisting of —C(H)═, —C(R^{d})═, and —N═;
W⁴ is selected from the group consisting of —C(H)═, —C(R^{d4})═, and —N═;
W⁵ is selected from the group consisting of —C(H)═, —C(R^{d5})═, and —N═;
W⁶ is selected from the group consisting of —C(H)═, —C(R′)═, and —N═;
W⁷ is selected from the group consisting of —C(H)═, —C(R^{c})═, and —N═;
W⁸ is selected from the group consisting of —C(H)═, —C(R^{c})═, and —N═;
W⁹ is selected from the group consisting of —C(H)═, —C(R^{c9})═, and —N═;
Ring A is optionally substituted 5-membered heteroaryl, containing 1-2 heteroatoms selected from N, S, and O;
Ring B is optionally substituted 5-membered heteroaryl containing up to three heteroatoms selected from N, S, and O;
each R^{a} is independently selected from the group consisting of halogen, —CN, —NO₂—OR¹, —SR¹, —N(R¹)₂, —C(O)OR¹, C(O)N(R′)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa},
each R^{aa} is independently selected from the group consisting of halogen, —COOH, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂,
wherein two instances of R^{aa} are optionally taken together with any intervening atoms to form an optionally substituted 4-6 membered carbocyclyl or optionally substituted 4-6 membered heterocyclyl ring;
each R^{b} is independently selected from the group consisting of halogen, oxo, —CN, —NO₂—OR¹, —SR¹, —N(R′)₂, —C(O)OR¹, C(O)N(R′)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl,
wherein two instances of R^{b} are optionally taken together with any intervening atoms to form an optionally substituted 5-6 membered carbocyclyl or optionally substituted 5-6 membered heterocyclyl ring;
each R^{c} is independently selected from the group consisting of halogen, —CN, —NO₂—OR¹, —SR¹, —N(R′)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{d} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R′)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl; R^{c9} is halogen;
R^{d4} is halogen;
R^{d5} is halogen;
each R¹ is independently selected from the group consisting of hydrogen, —(CH₂)₁₋₃R², —C(O)R², —(CH₂)₁₋₃OR², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
wherein two instances of R¹ are optionally taken together with any intervening atoms to form an optionally substituted 3-7 membered heterocyclyl ring;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R³ is independently selected from the group consisting of hydrogen, unsubstituted alkyl or haloalkyl
X is selected from the group consisting of —O—, —S—, —S(O)—, —S(O)₂—, —C(R¹)(R²)—, —C(O)—, and —CH(OH)—;
Y is selected from the group consisting of optionally substituted C₁-C₃ alkylene, —O—, —S—, —S(O)—, —SO₂N(R²)—, and —S(O)₂—;
n is 0, 1, 2, or 3;
m is 1, 2, or 3,
wherein the compound is not

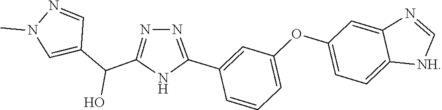 [SMILES: Cn1cc(C(O)c2nnc(-c3cccc(Oc4ccc5[nH]cnc5c4)c3)[nH]2)cn1]

2. The compound of claim 1, wherein W¹ is —N═.

3. The compound of claim 1, wherein W² is —N═.

4. The compound of claim 1, wherein W³ is —N═.

5. The compound of claim 1, wherein W⁴ is —N═.

6. The compound of claim 1, wherein when W⁸ is —N═, W¹, W², W³, and W⁴ are each not —N═.

7. The compound of claim 1, wherein the compound is of Formula (I-a), (I-b), (I-c), (I-d), (I-e), or (I-f): 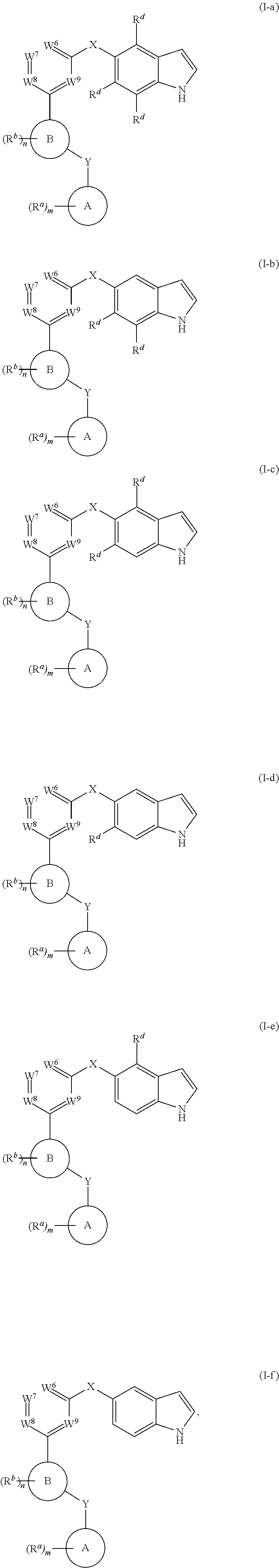
or a pharmaceutically acceptable salt thereof.

8. The compound of any of claims 1-7, wherein
W⁶ is —C(H)═;
W⁷ is —C(H)═;
W⁸ is —C(H)═; and
W⁹ is —N═.

9. The compound of any of claims 1-7, wherein
W⁶ is —C(H)═;
W⁷ is —C(H)═;
W⁸ is —N═; and
W⁹ is —C(H)═.

10. The compound of any of claims 1-7, wherein
W⁶ is —C(H)═;
W⁷ is —C(H)═;
W⁸ is —C(H)═; and
W⁹ is —C(H)═.

11. The compound of any of claims 1-7, wherein
W⁶ is —C(R^{c})═;
W⁷ is —C(H)═;
W⁸ is —C(H)═; and
W⁹ is —C(H)═.

12. The compound of any of claims 1-7, wherein
W⁶ is —C(H)═;
W⁷ is —C(H)═;
W⁸ is —C(R^{c})═; and
W⁹ is —C(H)═.

13. The compound of any of claims 1-12, wherein X is —O—.

14. The compound of any of claims 1-12, wherein X is —S—.

15. The compound of any of claims 1-12, wherein X is —S(O)—.

16. The compound of any of claims 1-12, wherein X is —S(O)₂—.

17. The compound of any of claims 1-16, wherein Y is —O—.

18. The compound of any of claims 1-16, wherein Y is optionally substituted C₁-C₃ alkylene.

19. The compound of any of claims 1-16, wherein Y is —S—.

20. The compound of any of claims 1-16, wherein Y is —S(O)—.

21. The compound of any of claims 1-16, wherein Y is —S(O)₂—.

22. The compound of any of claims 1-16, wherein Y is —S(O)₂N(R²)—.

23. The compound of any of claims 1-21, wherein Ring A is an optionally substituted 5-membered heteroaryl selected from the group consisting of thiophenyl, pyrazolyl, pyrrolyl, and thiazolyl.

24. The compound of claim 23, wherein Ring A is selected from the group consisting of

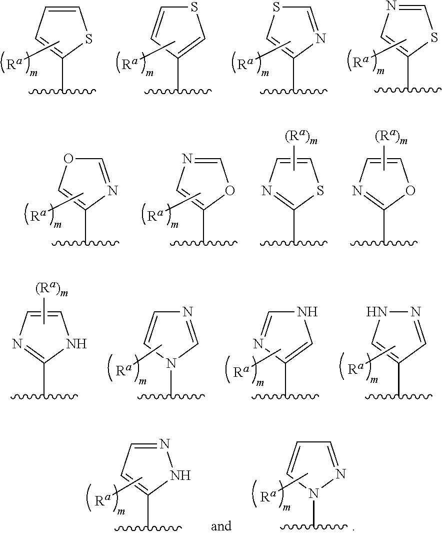

25. The compound of claim 23, wherein Ring A is selected from the group consisting of

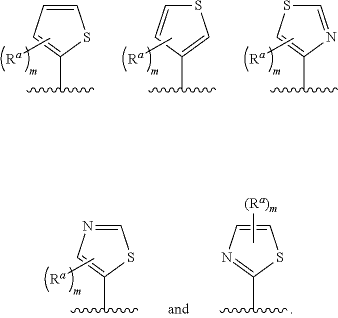

26. The compound of any of claims 1-25, wherein Ring B is selected from the group consisting of pyrazolyl, triazolyl, imidazolyl, oxazolyl, thiazolyl, oxadiazolyl, thiadiazolyl, isooxadiazolyl and isothiadiazolyl.

27. The compound of claim 26, wherein Ring B is pyrazolyl.

28. The compound of claim 26, wherein Ring B is

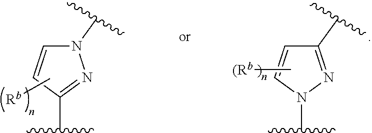

29. The compound of claim 26, wherein Ring B is

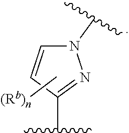

30. The compound of claim 26, wherein Ring B is triazolyl.

31. The compound of claim 26, wherein Ring B is 1,2,4 triazolyl.

32. The compound of claim 26, wherein Ring B is 1, 2, 3 triazolyl.

33. The compound of claim 26, wherein Ring B is selected from the group consisting of

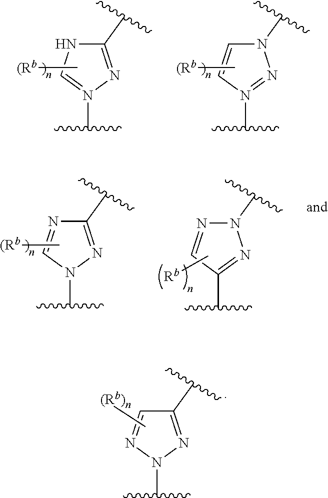

34. The compound of claim 26, wherein Ring B is

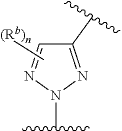

35. The compound of claim 26, wherein Ring B is pyrrolyl.

36. The compound of claim 26, wherein Ring B is selected from the group consisting of

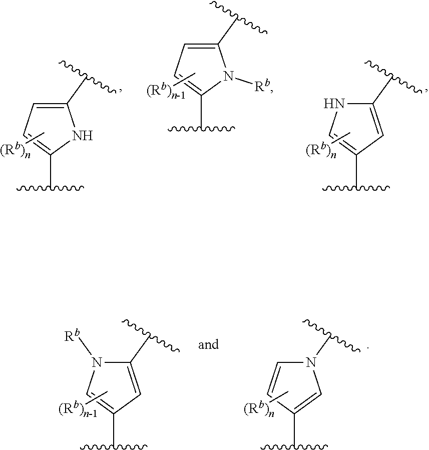

37. The compound of claim 26, wherein Ring B is imidazolyl.

38. The compound of claim 26, wherein Ring B is selected from the group consisting of

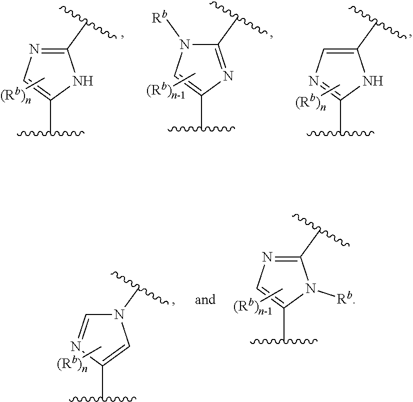

39. The compound of claim 26, wherein Ring B is selected from the group consisting of

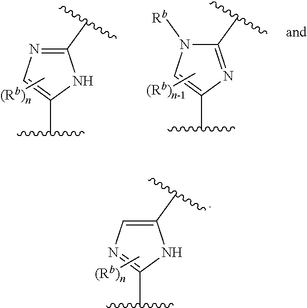

40. The compound of claim 26, wherein Ring B is oxazolyl.

41. The compound of claim 26, wherein Ring B is selected from the group consisting of

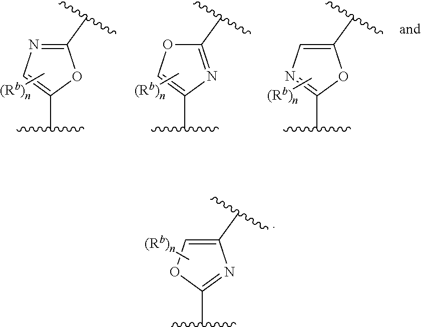

42. The compound of claim 26, wherein Ring B is thiazolyl.

43. The compound of claim 26, wherein Ring B is selected from the group consisting of

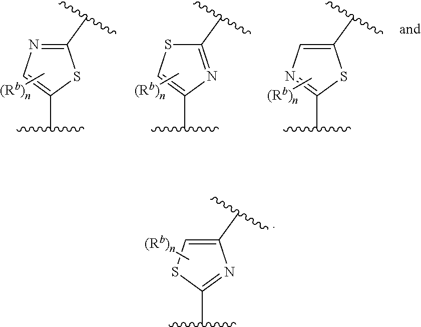

44. A compound of Formula (II): 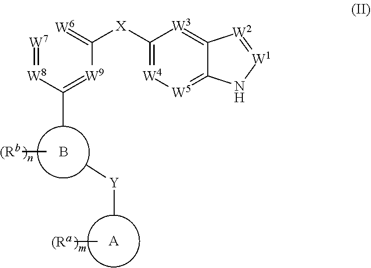
or a pharmaceutically acceptable salt thereof wherein
W¹ is selected from the group consisting of —C(R^{d})═, and —N═;
W² is selected from the group consisting of —C(H)═, —C(R^{d})═, and —N═;
W³ is selected from the group consisting of —C(R^{d3})═, and —N═;
W⁴ is selected from the group consisting of —C(R^{d4})═, and —N═;
W⁵ is selected from the group consisting of —C(H)═, —C(R^{d5})═, and —N═;
W⁶ is selected from the group consisting of —C(H)═, —C(R^{e})═, and —N═;
W⁷ is selected from the group consisting of —C(H)═, —C(R^{e})═, and —N═;
W⁸ is selected from the group consisting of —C(H)═, —C(R^{e})═, and —N═;
W⁹ is selected from the group consisting of —C(H)═, —C(R^{e9})═, and —N═;
Ring A is optionally substituted phenyl or optionally substituted 6-membered heteroaryl, containing 1-2 nitrogen atoms;
Ring B is optionally substituted 5-membered heteroaryl containing up to three heteroatoms selected from N, S, and O;
each R^{a} is selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa};
each R^{aa} is independently selected from the group consisting of halogen, —COOH, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂,
wherein two instances of R^{aa} are optionally taken together with any intervening atoms to form an optionally substituted 4-6 membered carbocyclyl or optionally substituted 5-6 membered heterocyclyl ring;
each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{c} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{d} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —SOR¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
R^{c9} is halogen;
R^{d3} is hydrogen or R^{d};
R^{d4} is hydrogen or halogen;
wherein when R^{d4} is fluoro, R^{d3} is not hydrogen, fluoro, chloro, methyl, cyclopropyl, —CH₂CF₃, —CH₂CHF₂, —(CH₂)₀₋₃SO₂R¹, —(CH₂)₀₋₃SOR¹, —(CH₂)₀₋₃P(O)Me₂, —SR¹, thiazolyl, pyrazolyl, or (CH₂)₀₋₃ (3-7 membered heterocyclyl);
wherein when R^{d4} is hydrogen, R^{d3} is not hydrogen, methyl, —CH₂OH, —CH₂NH(tBu), or —C(O)NHMe;
R^{d5} is halogen;
each R¹ is independently selected from the group consisting of hydrogen, —(CH₂)₁₋₃R², —C(O)R², —(CH₂)₁₋₃OR², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
wherein two instances of R¹ are optionally taken together with any intervening atoms to form an optionally substituted 3-7 membered heterocyclyl ring;
each R² is independently selected from the group consisting of hydrogen, optionally substituted C₁-C₆ aliphatic, optionally substituted phenyl, optionally substituted 5-6-membered heteroaryl,
optionally substituted 3-7 membered carbocyclyl, and optionally substituted 3-7 membered heterocyclyl;
each R⁴ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa},
Y is selected from the group consisting of optionally substituted C₁-C₃ alkylene, optionally substituted C₃-C₇ cycloalkylene, optionally substituted 3-7 membered heterocyclylene, —O—, —S—, —S(O)—, —S(O)N(R⁴)—, —SO₂N(R²)—, and —S(O)₂—;
n is 0 or 1.
m is 1, 2, or 3.

45. The compound of claim 44, wherein W¹ is —N═.

46. The compound of claim 44, wherein W² is —N═.

47. The compound of claim 44, wherein W³ is —N═.

48. The compound of claim 44, wherein W⁴ is —N═.

49. The compound of claim 44, wherein the compound is of Formula (II-a), (II-b), (II-c), (II-d), (II-e), or (IT-f): 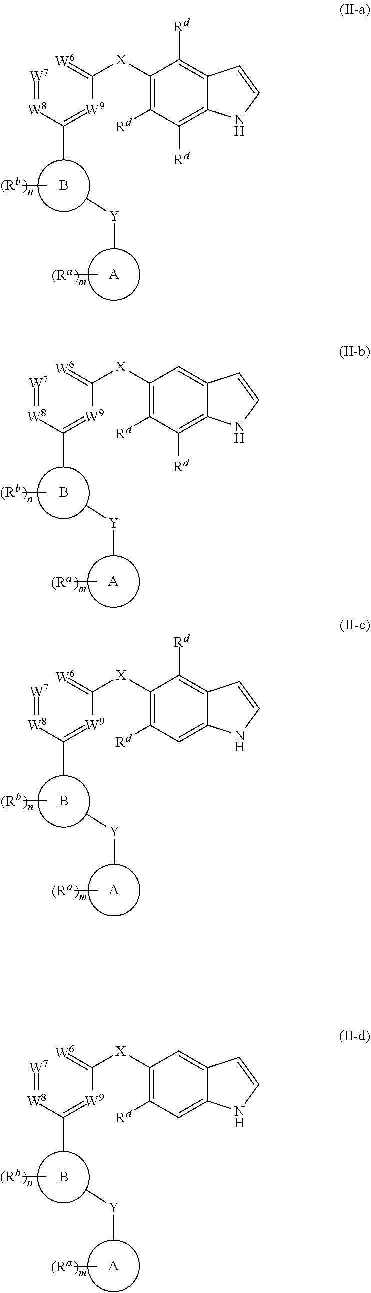 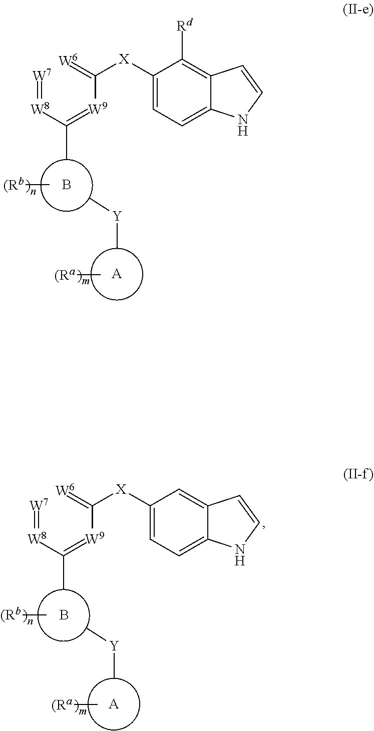
or a pharmaceutically acceptable salt thereof.

50. The compound of any of claims 44-49, wherein
W⁶ is —C(H)═;
W⁷ is —C(H)═;
W⁸ is —C(H)═; and
W⁹ is —N═.

51. The compound of any of claims 44-49, wherein
W⁶ is —C(H)═;
W⁷ is —C(H)═;
W⁸ is —N═; and
W⁹ is —C(H)═.

52. The compound of any of claims 44-49, wherein
W⁶ is —C(H)═;
W⁷ is —C(H)═;
W⁸ is —C(H)═; and
W⁹ is —C(H)═.

53. The compound of any of claims 44-49, wherein
W⁶ is —C(R^{e})═;
W⁷ is —C(H)═;
W⁸ is —C(H)═; and
W⁹ is —C(H)═.

54. The compound of any of claims 44-49, wherein
W⁶ is —C(H)═;
W⁷ is —C(H)═;
W⁸ is —C(R^{e})═; and
W⁹ is —C(H)═.

55. The compound of any of claims 44-54, wherein Y is —O—.

56. The compound of any of claims 44-54, wherein Y is —S—.

57. The compound of any of claims 44-54, wherein Y is —S(O)—.

58. The compound of any of claims 44-54, wherein Y is —S(O)₂—.

59. The compound of any of claims 44-54, wherein Y is an optionally substituted C₁-C₃ alkylene.

60. The compound of any of claims 44-59, wherein Ring A is optionally substituted phenyl.

61. The compound of any of claims 44-59, wherein Ring A is optionally substituted pyridinyl.

62. The compound of any of claims 44-61, wherein Ring B is selected from the group consisting of pyrazolyl, triazolyl, imidazolyl, oxazolyl, thiazolyl, oxadiazolyl, thiadiazolyl, isooxadiazolyl and isothiadiazolyl.

63. The compound of claim 62, wherein Ring B is pyrazolyl.

64. The compound of claim 62, wherein Ring B is

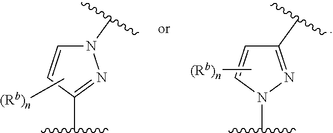

65. The compound of claim 62, wherein Ring B is

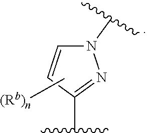

66. The compound of claim 62, wherein Ring B is triazolyl.

67. The compound of claim 62, wherein Ring B is 1,2,4 triazolyl.

68. The compound of claim 62, wherein Ring B is 1, 2, 3 triazolyl.

69. The compound of claim 62, wherein Ring B is selected from the group consisting of

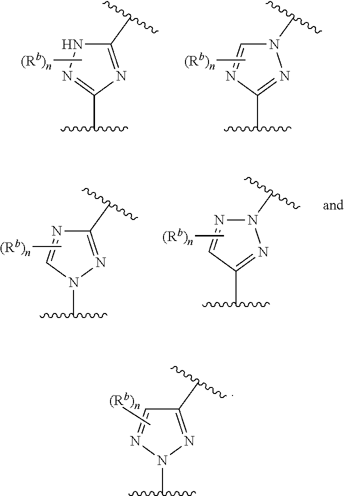

70. The compound of claim 62, wherein Ring B is

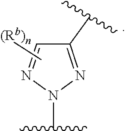

71. The compound of claim 62, wherein Ring B is pyrroiyi.

72. The compound of claim 62, wherein Ring B is selected from the group consisting of

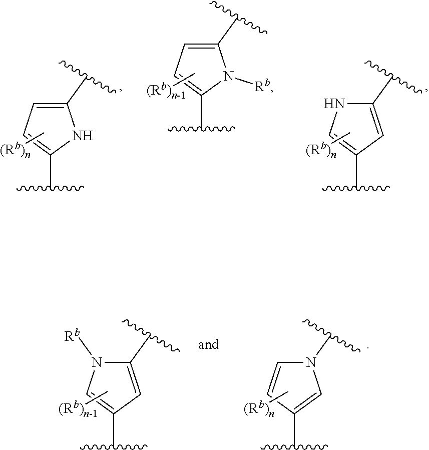

73. The compound of claim 62, wherein Ring B is imidazolyl.

74. The compound of claim 62, wherein Ring B is selected from the group consisting of

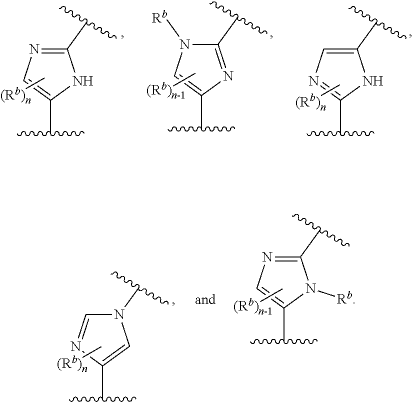

75. The compound of claim 62, wherein Ring B is selected from the group consisting of

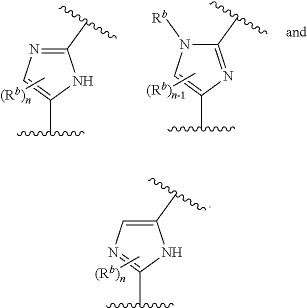

76. The compound of claim 62, wherein Ring B is oxazolyl.

77. The compound of claim 62, wherein Ring B is selected from the group consisting of

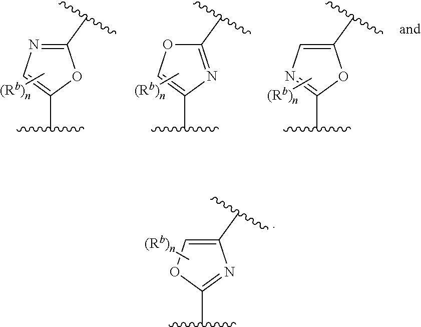

78. The compound of claim 62, wherein Ring B is thiazolyl.

79. The compound of claim 62, wherein Ring B is selected from the group consisting of

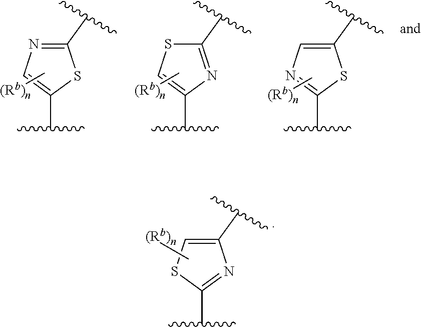

80. A compound of Formula (III): 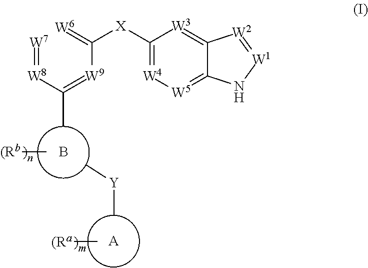
or a pharmaceutically acceptable salt thereof wherein
W¹ is selected from the group consisting of —C(H)═, and —N═;
W² is selected from the group consisting of —C(H)═, —C(R^{d})═, and —N═;
W³ is selected from the group consisting of —C(H)═, —C(R^{d})═, and —N═;
W⁴ is selected from the group consisting of —C(H)═, —C(R^{d4})═, and —N═;
W⁵ is selected from the group consisting of —C(H)═, —C(R^{d5})═, and —N═;
W⁶ is selected from the group consisting of —C(H)═, —C(R^{c})═, and —N═;
W⁷ is selected from the group consisting of —C(H)═, —C(R^{c})═, and —N═;
W⁸ is selected from the group consisting of —C(H)═, —C(R^{c})═, and —N═;
W⁹ is selected from the group consisting of —C(H)═, —C(R^{c9})═, and —N═;
Ring A is optionally substituted phenyl or optionally substituted 6-membered heteroaryl, containing 1-2 nitrogen atoms;
each R^{a} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa};
each R^{aa} is independently selected from the group consisting of halogen, —COOH, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂,
wherein two instances of R^{aa} are optionally taken together with any intervening atoms to form an optionally substituted 4-6 membered carbocyclyl or heterocyclyl ring;
each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{c} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{d} is independently selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
R^{c9} is halogen;
R^{d5} is halogen;
R^{d5} is halogen;
each R¹ is independently selected from the group consisting of hydrogen, —(CH₂)₁₋₃R², —C(O)R², —(CH₂)₁₋₃OR², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
wherein two instances of R¹ are optionally taken together with any intervening atoms to form an optionally substituted 3-7 membered heterocyclyl ring;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
X is selected from the group consisting of optionally substituted C₁-C₃ alkylene, C═O, —S—, —S(O)—, —SO(NR²)—, and —S(O)₂—;
Y is optionally substituted C₁-C₃ alkylene optionally substituted C₃-C₇ cycloalkylene, or optionally substituted 3-7 membered heterocyclylene;
p is 0, 1, or 2;
n is 0, or 1;
mis 1, 2, or 3.

81. The compound of claim 80, wherein W¹ is —N═.

82. The compound of claim 80, wherein W² is —N═.

83. The compound of claim 80, wherein W³ is —N═.

84. The compound of claim 80, wherein W⁴ is —N═.

85. The compound of claim 80, wherein the compound is of Formula (III-a), (III-b), (III-c), (III-d), (III-e), or (III-f): 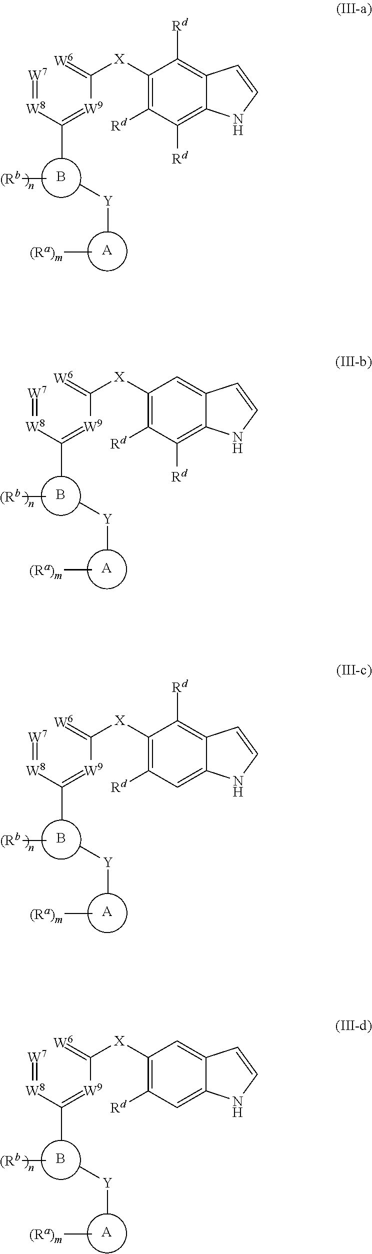 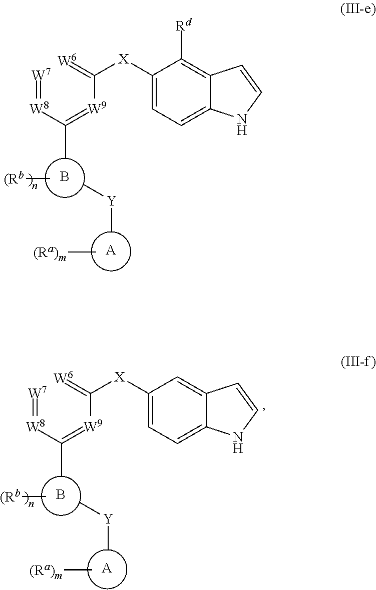
or a pharmaceutically acceptable salt thereof.

86. The compound of any of claims 80-85, wherein
W⁶ is —C(H)═;
W⁷ is —C(H)═;
W⁸ is —C(H)═; and
W⁹ is —N═.

87. The compound of any of claims 80-85, wherein
W⁶ is —C(H)═;
W⁷ is —C(H)═;
W⁸ is —N═; and
W⁹ is —C(H)═.

88. The compound of any of claims 80-85, wherein
W⁶ is —C(H)═;
W⁷ is —C(H)═;
W⁸ is —C(H)═; and
W⁹ is —C(H)═.

89. The compound of any of claims 80-85, wherein
W⁶ is —C(R^{c})═;
W⁷ is —C(H)═;
W⁸ is —C(H)═; and
W⁹ is —C(H)═.

90. The compound of any of claims 80-85, wherein
W⁶ is —C(H)═;
W⁷ is —C(H)═;
W⁸ is —C(R^{c})═; and
W⁹ is —C(H)═.

91. The compound of any of claims 80-85, wherein X is —O—.

92. The compound of any of claims 80-85, wherein X is —S—.

93. The compound of any of claims 80-85, wherein X is —S(O)—.

94. The compound of any of claims 80-85, wherein X is —S(O)₂—.

95. The compound of any of claims 80-94, wherein Ring B is 1,2,4-triazolyl or imidazolyl.

96. The compound of any of claims 80-95, wherein Ring A is optionally substituted phenyl.

97. The compound of any of claims 80-95, wherein Ring A is optionally substituted pyridinyl.

98. The compound of any of claims 80-97, wherein Ring B is selected from the group consisting of pyrazolyl, triazolyl, imidazolyl, oxazolyl, thiazolyl, oxadiazolyl, thiadiazolyl, isooxadiazolyl and isothiadiazolyl.

99. The compound of claim 98, wherein Ring B is pyrazolyl.

100. The compound of claim 98, wherein Ring B is

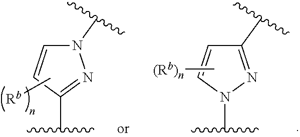

101. The compound of claim 98, wherein Ring B is

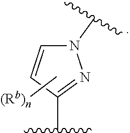

102. The compound of claim 98, wherein Ring B is triazolyl.

103. The compound of claim 98, wherein Ring B is 1,2,4 triazolyl.

104. The compound of claim 98, wherein Ring B is 1, 2, 3 triazolyl.

105. The compound of claim 98, wherein Ring B is selected from the group consisting of

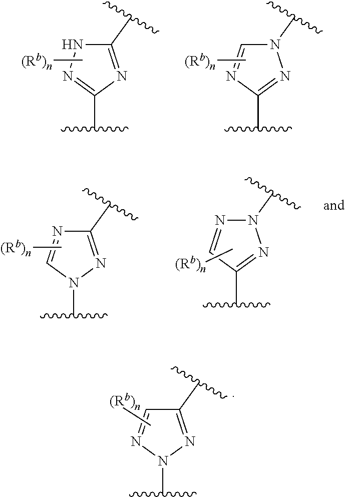

106. The compound of claim 98, wherein Ring B is

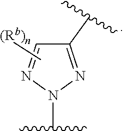

107. The compound of claim 98, wherein Ring B is pyrrolyl.

108. The compound of claim 98, wherein Ring B is selected from the group consisting of

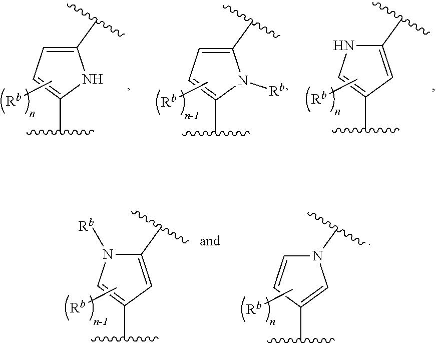

109. The compound of claim 98, wherein Ring B is imidazolyl.

110. The compound of claim 98, wherein Ring B is selected from the group consisting of

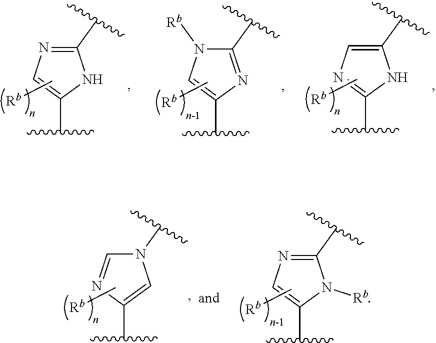

111. The compound of claim 98, wherein Ring B is selected from the group consisting of

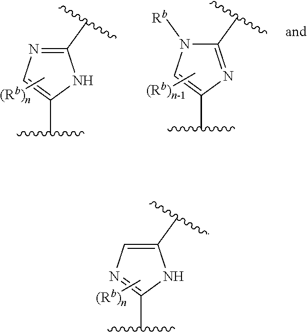

112. The compound of claim 98, wherein Ring B is oxazolyl.

113. The compound of claim 98, wherein Ring B is selected from the group consisting of

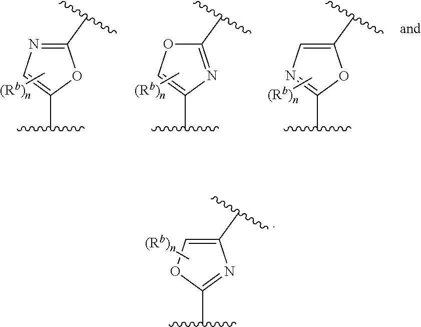

114. The compound of claim 98, wherein Ring B is thiazolyl.

115. The compound of claim 98, wherein Ring B is selected from the group consisting of

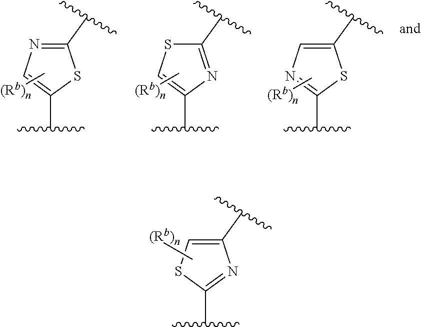

116. The compound of any of claims 1-115, wherein each R^{a} is independently selected from halogen or optionally substituted C₁-C₆ alkyl, wherein each R^{a} is independently substituted with 0-4 instances of R^{aa}.

117. The compound of claim 116, wherein each R^{a} is independently selected from fluoro, chloro, bromo, methyl, CH₂CH(Me)COOH, CH₂CH(OH)CH₂(OH) and —CH₂CH₂COOH.

118. The compound of claim 116, wherein R^{a} is —CH₂CH₂COOH.

119. The compound of claim 116, wherein R^{a} is —F.

120. The compound of any of claims 1-119, wherein R^{d} is halogen.

121. The compound of claim 120, wherein R^{d} is fluoro.

122. The compound of any of claims 1-121, wherein R^{d4} is halogen.

123. The compound of claim 122, wherein R^{d4} is fluoro.

124. The compound of any of claims 1-123, wherein R^{d5} is halogen.

125. The compound of claim 125, wherein Ras is fluoro.

126. A compound selected from the group consisting of

| Com- | |
|---|---|
| pound | |
| No. | Structure |
| 1 | 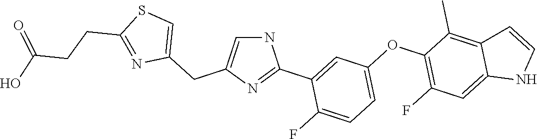 [SMILES: Cc1c(Oc2ccc(F)c(-c3nc(Cc4csc(CCC(=O)O)n4)c[nH]3)c2)c(F)cc2[nH]ccc12] |
| 2 | 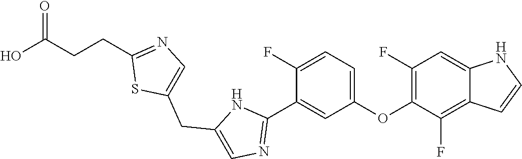 [SMILES: O=C(O)CCc1ncc(Cc2cnc(-c3cc(Oc4c(F)cc5[nH]ccc5c4F)ccc3F)[nH]2)s1] |
| 3 | 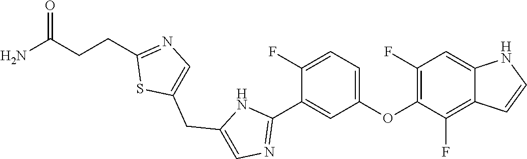 [SMILES: NC(=O)CCc1ncc(Cc2cnc(-c3cc(Oc4c(F)cc5[nH]ccc5c4F)ccc3F)[nH]2)s1] |
| 4 | 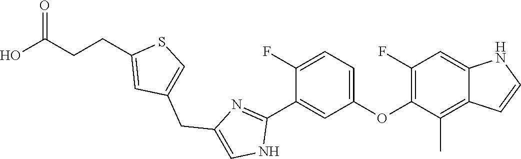 [SMILES: Cc1c(Oc2ccc(F)c(-c3nc(Cc4csc(CCC(=O)O)c4)c[nH]3)c2)c(F)cc2[nH]ccc12] |
| 5 | 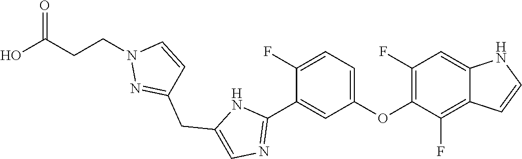 [SMILES: O=C(O)CCn1ccc(Cc2cnc(-c3cc(Oc4c(F)cc5[nH]ccc5c4F)ccc3F)[nH]2)n1] |
| 6 | 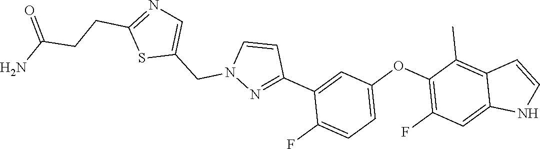 [SMILES: Cc1c(Oc2ccc(F)c(-c3ccn(Cc4cnc(CCC(N)=O)s4)n3)c2)c(F)cc2[nH]ccc12] |
| 7 | 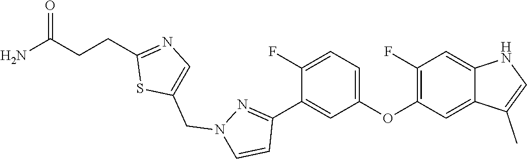 [SMILES: Cc1c[nH]c2cc(F)c(Oc3ccc(F)c(-c4ccn(Cc5cnc(CCC(N)=O)s5)n4)c3)cc12] |
| 8 | 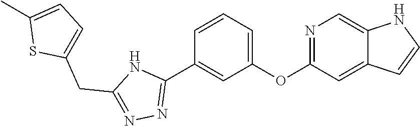 [SMILES: Cc1ccc(Cc2nnc(-c3cccc(Oc4cc5cc[nH]c5cn4)c3)[nH]2)s1] |
| 9 | 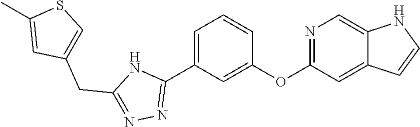 [SMILES: Cc1cc(Cc2nnc(-c3cccc(Oc4cc5cc[nH]c5cn4)c3)[nH]2)cs1] |
| 10 | 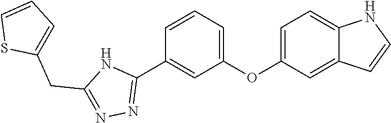 [SMILES: c1cc(Oc2ccc3[nH]ccc3c2)cc(-c2nnc(Cc3cccs3)[nH]2)c1] |
| 11 | 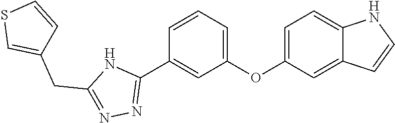 [SMILES: c1cc(Oc2ccc3[nH]ccc3c2)cc(-c2nnc(Cc3ccsc3)[nH]2)c1] |
| 12 | 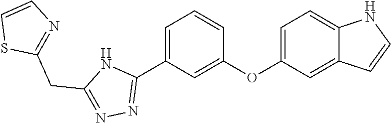 [SMILES: c1cc(Oc2ccc3[nH]ccc3c2)cc(-c2nnc(Cc3nccs3)[nH]2)c1] |
| 13 | 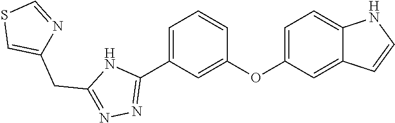 [SMILES: c1cc(Oc2ccc3[nH]ccc3c2)cc(-c2nnc(Cc3cscn3)[nH]2)c1] |
| 14 | 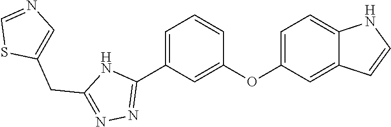 [SMILES: c1cc(Oc2ccc3[nH]ccc3c2)cc(-c2nnc(Cc3cncs3)[nH]2)c1] |
| 15 | 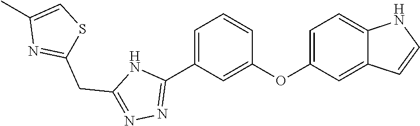 [SMILES: Cc1csc(Cc2nnc(-c3cccc(Oc4ccc5[nH]ccc5c4)c3)[nH]2)n1] |
| 16 | 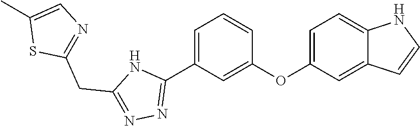 [SMILES: Cc1cnc(Cc2nnc(-c3cccc(Oc4ccc5[nH]ccc5c4)c3)[nH]2)s1] |
| 17 | 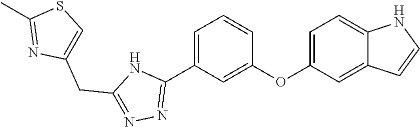 [SMILES: Cc1nc(Cc2nnc(-c3cccc(Oc4ccc5[nH]ccc5c4)c3)[nH]2)cs1] |
| 18 | 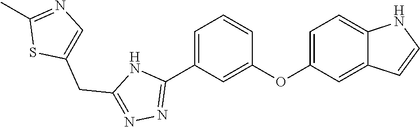 [SMILES: Cc1ncc(Cc2nnc(-c3cccc(Oc4ccc5[nH]ccc5c4)c3)[nH]2)s1] |
| 19 | 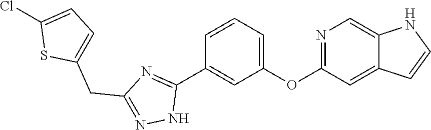 [SMILES: Clc1ccc(Cc2n[nH]c(-c3cccc(Oc4cc5cc[nH]c5cn4)c3)n2)s1] |
| 20 | 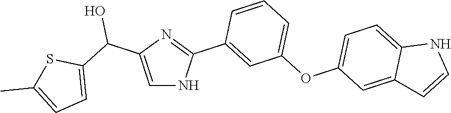 [SMILES: Cc1ccc(C(O)c2c[nH]c(-c3cccc(Oc4ccc5[nH]ccc5c4)c3)n2)s1] |
| 21 | 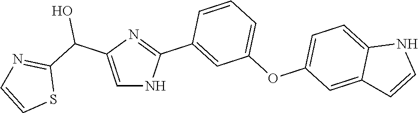 [SMILES: OC(c1c[nH]c(-c2cccc(Oc3ccc4[nH]ccc4c3)c2)n1)c1nccs1] |
| 22 | 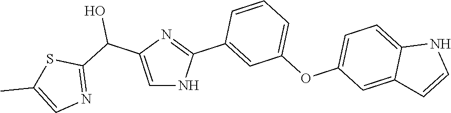 [SMILES: Cc1cnc(C(O)c2c[nH]c(-c3cccc(Oc4ccc5[nH]ccc5c4)c3)n2)s1] |
| 23 | 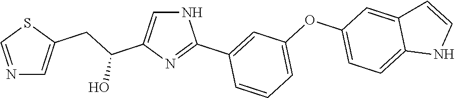 [SMILES: O[C@H](Cc1cncs1)c1c[nH]c(-c2cccc(Oc3ccc4[nH]ccc4c3)c2)n1] |
| 24 | 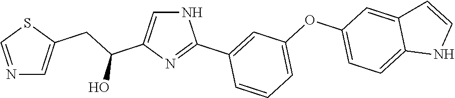 [SMILES: O[C@@H](Cc1cncs1)c1c[nH]c(-c2cccc(Oc3ccc4[nH]ccc4c3)c2)n1] |
| 25 | 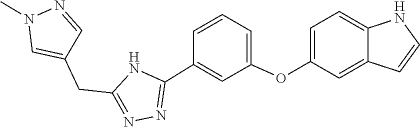 [SMILES: Cn1cc(Cc2nnc(-c3cccc(Oc4ccc5[nH]ccc5c4)c3)[nH]2)cn1] |
| 26 | 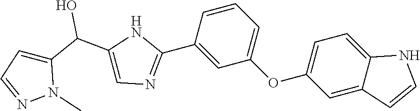 [SMILES: Cn1nccc1C(O)c1cnc(-c2cccc(Oc3ccc4[nH]ccc4c3)c2)[nH]1] |
| 27 | 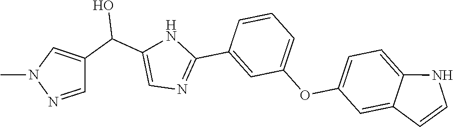 [SMILES: Cn1cc(C(O)c2cnc(-c3cccc(Oc4ccc5[nH]ccc5c4)c3)[nH]2)cn1] |
| 28 | 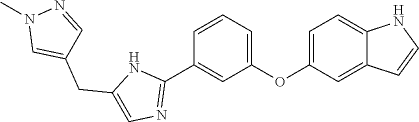 [SMILES: Cn1cc(Cc2cnc(-c3cccc(Oc4ccc5[nH]ccc5c4)c3)[nH]2)cn1] |
| 29 | 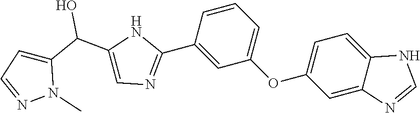 [SMILES: Cn1nccc1C(O)c1cnc(-c2cccc(Oc3ccc4[nH]cnc4c3)c2)[nH]1] |
| 30 | 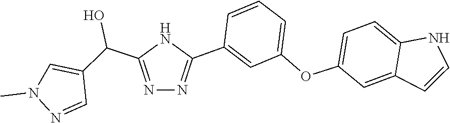 [SMILES: Cn1cc(C(O)c2nnc(-c3cccc(Oc4ccc5[nH]ccc5c4)c3)[nH]2)cn1] |
| 31 | 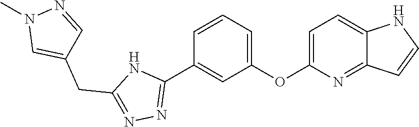 [SMILES: Cn1cc(Cc2nnc(-c3cccc(Oc4ccc5[nH]ccc5n4)c3)[nH]2)cn1] |
| 32 | 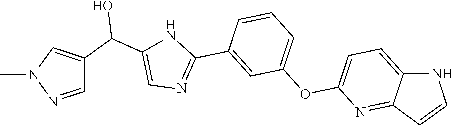 [SMILES: Cn1cc(C(O)c2cnc(-c3cccc(Oc4ccc5[nH]ccc5n4)c3)[nH]2)cn1] |
| 33 | 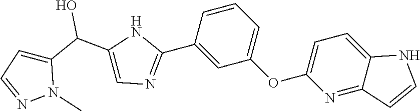 [SMILES: Cn1nccc1C(O)c1cnc(-c2cccc(Oc3ccc4[nH]ccc4n3)c2)[nH]1] |
| 34 | 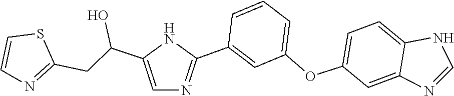 [SMILES: OC(Cc1nccs1)c1cnc(-c2cccc(Oc3ccc4[nH]cnc4c3)c2)[nH]1] |
| 35 | 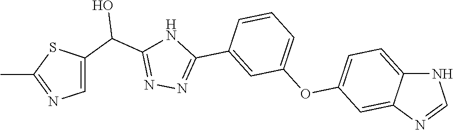 [SMILES: Cc1ncc(C(O)c2nnc(-c3cccc(Oc4ccc5[nH]cnc5c4)c3)[nH]2)s1] |
| 36 | 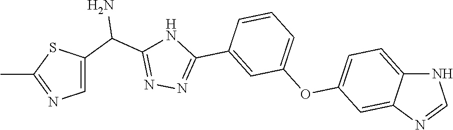 [SMILES: Cc1ncc(C(N)c2nnc(-c3cccc(Oc4ccc5[nH]cnc5c4)c3)[nH]2)s1] |
| 37 | 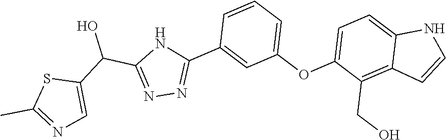 [SMILES: Cc1ncc(C(O)c2nnc(-c3cccc(Oc4ccc5[nH]ccc5c4CO)c3)[nH]2)s1] |
| 38 | 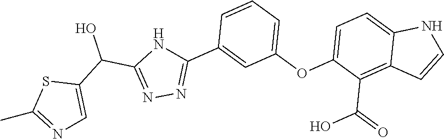 [SMILES: Cc1ncc(C(O)c2nnc(-c3cccc(Oc4ccc5[nH]ccc5c4C(=O)O)c3)[nH]2)s1] |
| 39 | 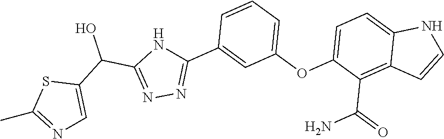 [SMILES: Cc1ncc(C(O)c2nnc(-c3cccc(Oc4ccc5[nH]ccc5c4C(N)=O)c3)[nH]2)s1] |
| 40 | 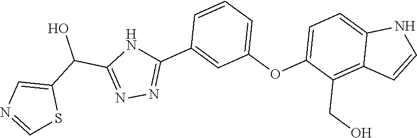 [SMILES: OCc1c(Oc2cccc(-c3nnc(C(O)c4cncs4)[nH]3)c2)ccc2[nH]ccc12] |
| 41 | 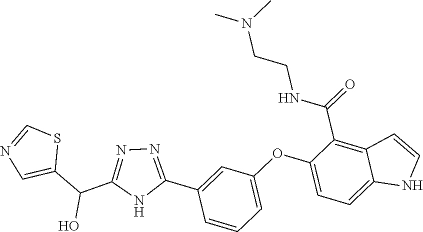 [SMILES: CN(C)CCNC(=O)c1c(Oc2cccc(-c3nnc(C(O)c4cncs4)[nH]3)c2)ccc2[nH]ccc12] |
| 42 | 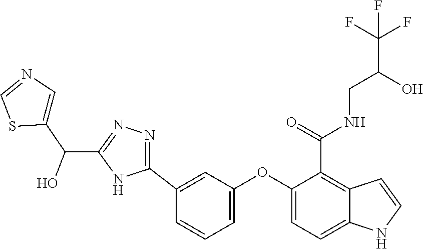 [SMILES: O=C(NCC(O)C(F)(F)F)c1c(Oc2cccc(-c3nnc(C(O)c4cncs4)[nH]3)c2)ccc2[nH]ccc12] |
| 43 | 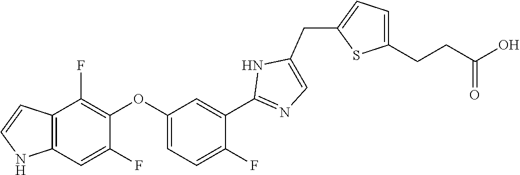 [SMILES: O=C(O)CCc1ccc(Cc2cnc(-c3cc(Oc4c(F)cc5[nH]ccc5c4F)ccc3F)[nH]2)s1] |
| 44 | 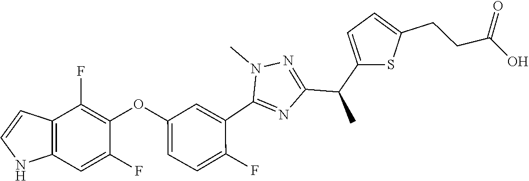 [SMILES: C[C@@H](c1nc(-c2cc(Oc3c(F)cc4[nH]ccc4c3F)ccc2F)n(C)n1)c1ccc(CCC(=O)O)s1] |
| 45 | 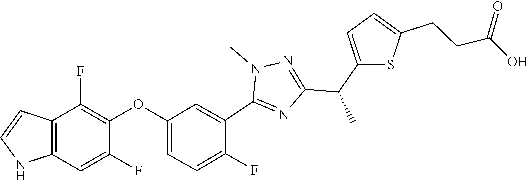 [SMILES: C[C@H](c1nc(-c2cc(Oc3c(F)cc4[nH]ccc4c3F)ccc2F)n(C)n1)c1ccc(CCC(=O)O)s1] |
| 46 | 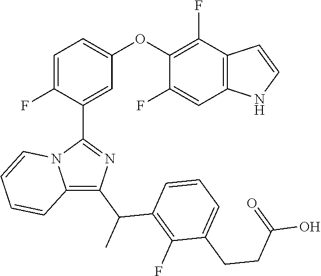 [SMILES: CC(c1cccc(CCC(=O)O)c1F)c1nc(-c2cc(Oc3c(F)cc4[nH]ccc4c3F)ccc2F)n2ccccc12] |
| 47 | 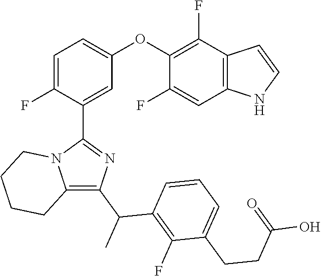 [SMILES: CC(c1cccc(CCC(=O)O)c1F)c1nc(-c2cc(Oc3c(F)cc4[nH]ccc4c3F)ccc2F)n2c1CCCC2] |
| 48 | 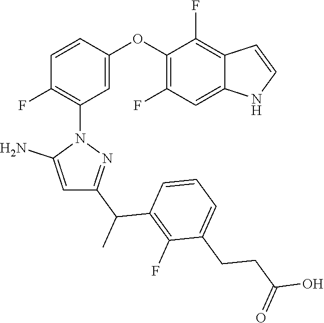 [SMILES: CC(c1cc(N)n(-c2cc(Oc3c(F)cc4[nH]ccc4c3F)ccc2F)n1)c1cccc(CCC(=O)O)c1F] |
| 49 | 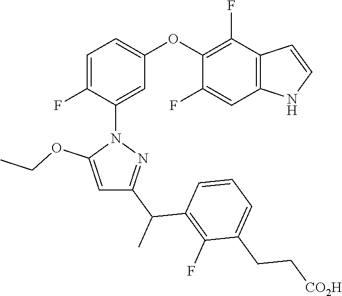 [SMILES: CCOc1cc(C(C)c2cccc(CCC(=O)O)c2F)nn1-c1cc(Oc2c(F)cc3[nH]ccc3c2F)ccc1F] |
| 50 | 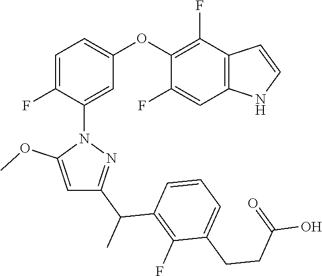 [SMILES: COc1cc(C(C)c2cccc(CCC(=O)O)c2F)nn1-c1cc(Oc2c(F)cc3[nH]ccc3c2F)ccc1F] |
| 51 | 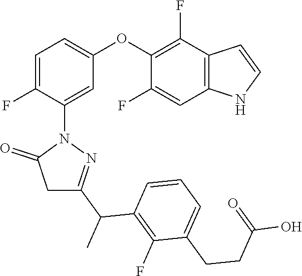 [SMILES: CC(C1=NN(c2cc(Oc3c(F)cc4[nH]ccc4c3F)ccc2F)C(=O)C1)c1cccc(CCC(=O)O)c1F] |
| 52 | 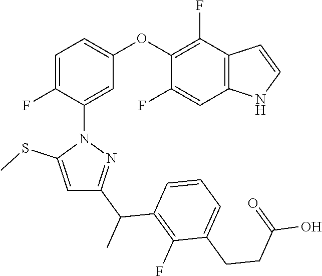 [SMILES: CSc1cc(C(C)c2cccc(CCC(=O)O)c2F)nn1-c1cc(Oc2c(F)cc3[nH]ccc3c2F)ccc1F] |
| 53 | 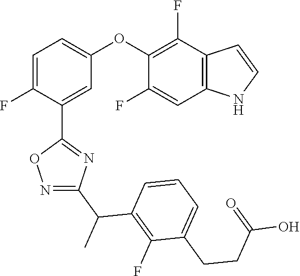 [SMILES: CC(c1noc(-c2cc(Oc3c(F)cc4[nH]ccc4c3F)ccc2F)n1)c1cccc(CCC(=O)O)c1F] |
| 54 | 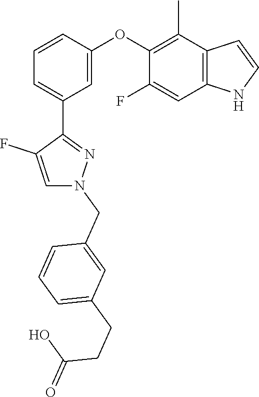 [SMILES: Cc1c(Oc2cccc(-c3nn(Cc4cccc(CCC(=O)O)c4)cc3F)c2)c(F)cc2[nH]ccc12] |
| 55 | 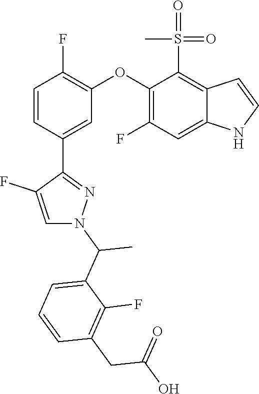 [SMILES: CC(c1cccc(CC(=O)O)c1F)n1cc(F)c(-c2ccc(F)c(Oc3c(F)cc4[nH]ccc4c3S(C)(=O)=O)c2)n1] |
| 56 | 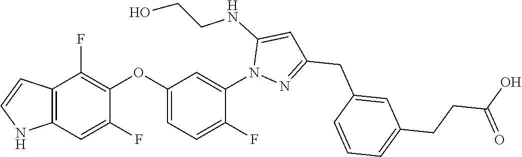 [SMILES: O=C(O)CCc1cccc(Cc2cc(NCCO)n(-c3cc(Oc4c(F)cc5[nH]ccc5c4F)ccc3F)n2)c1] |
| 57 | 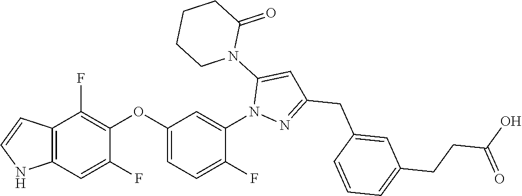 [SMILES: O=C(O)CCc1cccc(Cc2cc(N3CCCCC3=O)n(-c3cc(Oc4c(F)cc5[nH]ccc5c4F)ccc3F)n2)c1] |
| 58 | 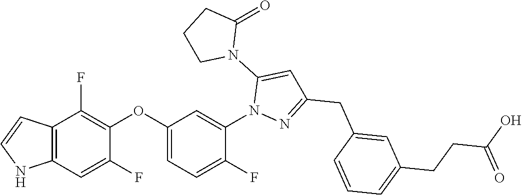 [SMILES: O=C(O)CCc1cccc(Cc2cc(N3CCCC3=O)n(-c3cc(Oc4c(F)cc5[nH]ccc5c4F)ccc3F)n2)c1] |
| 59 | 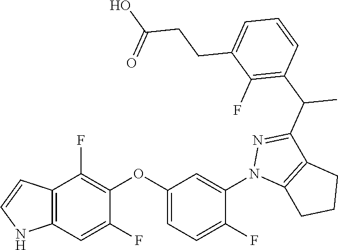 [SMILES: CC(c1cccc(CCC(=O)O)c1F)c1nn(-c2cc(Oc3c(F)cc4[nH]ccc4c3F)ccc2F)c2c1CCC2] |

or a pharmaceutically acceptable salt thereof.

127. A pharmaceutical composition comprising a compound of any of the previous claims and a pharmaceutically acceptable excipient.

128. A method of treating a CFTR-mediated disease or disorder comprising administering a patient in need there of a compound any of claims 1-126 or a pharmaceutical composition of claim 127.

129. The method of claim 128, wherein the disease or condition is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear palsy, Pick's disease, several polyglutamine neurological disorders, Huntington's, spinocerebellar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

130. The method of claim 128 or 129, wherein the disease or condition is selected from cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type I hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

131. The method of any one of claims 128-130, wherein the disease or condition is cystic fibrosis.

132. A method of treating kidney disease in a subject, comprising administering to the subject a therapeutically effective amount of a compound of any of claims 1-126 or a pharmaceutical composition of claim 127.

133. The method of claim 132, wherein the kidney disease is autosomal dominant polycystic kidney disease or autosomal recessive polycystic kidney disease.

134. The method of claim 132, wherein the kidney disease is autosomal dominant polycystic kidney disease.

135. The method of claim 132, wherein the kidney disease is autosomal recessive polycystic kidney disease.

136. A method of treating cystic fibrosis in a subject, comprising administering to the subject a therapeutically effective amount of a compound of any of claims 1-126 or a pharmaceutical composition of claim 127.

137. The method of claim 136, wherein the subject is human.
